SEQUENCE LISTING (408 sequences)

SEQ ID NO: 1 - protein, 5 aa, synthetic construct
DYAMT

SEQ ID NO: 2 - protein, 5 aa, synthetic construct
DFAMT

SEQ ID NO: 3 - protein, 5 aa, synthetic construct
TYAMT

SEQ ID NO: 4 - protein, 5 aa, synthetic construct
DSAMT

SEQ ID NO: 5 - protein, 17 aa, synthetic construct
SISGSGGNTYYADSVKG

SEQ ID NO: 6 - protein, 17 aa, synthetic construct
SISGSGGNIYYADSVKG

SEQ ID NO: 7 - protein, 17 aa, synthetic construct
SISGSGSSTYYADSVKG

SEQ ID NO: 8 - protein, 17 aa, synthetic construct
SISGSGDNIYYADSVKG

SEQ ID NO: 9 - protein, 17 aa, synthetic construct
SISGDGSNTYYADSVKG

SEQ ID NO: 10 - protein, 17 aa, synthetic construct
SVSGSGGNTYYADSVKG

SEQ ID NO: 11 - protein, 17 aa, synthetic construct
SISGSGDNTYYADSVKG

SEQ ID NO: 12 - protein, 17 aa, synthetic construct
SISGGGGSTYYADSVKG

SEQ ID NO: 13 - protein, 17 aa, synthetic construct
SISGSGGNKYYADSVKG

SEQ ID NO: 14 - protein, 17 aa, synthetic construct
SVSGDGGNTYYADSVKG

SEQ ID NO: 15 - protein, 17 aa, synthetic construct
SISGGGGNTYYADSVKG

SEQ ID NO: 16 - protein, 17 aa, synthetic construct
SISGSGDGTYYADSVKG

SEQ ID NO: 17 - protein, 16 aa, synthetic construct
DRLSITIRPRYYGLDV

SEQ ID NO: 18 - protein, 16 aa, synthetic construct
DRLSKTIRPRYYGLDV

SEQ ID NO: 19 - protein, 16 aa, synthetic construct
DRVSITIRPRYYGLDV

SEQ ID NO: 20 - protein, 16 aa, synthetic construct
DRLSVTVRPRYYGLDV

SEQ ID NO: 21 - protein, 16 aa, synthetic construct
DRLSVTIRPRYYGLDV

SEQ ID NO: 22 - protein, 16 aa, synthetic construct
DRLSITVRPRYYGLDV

SEQ ID NO: 23 - protein, 16 aa, synthetic construct
DRVSIYVRPRYYGLDV

SEQ ID NO: 24 - protein, 16 aa, synthetic construct
DRLSIAVRPRYYGLDV

SEQ ID NO: 25 - protein, 16 aa, synthetic construct
DRLSIYIRPRYYGLDV

SEQ ID NO: 26 - protein, 16 aa, synthetic construct
RSSQSLLYSIGYNYLD

SEQ ID NO: 27 - protein, 16 aa, synthetic construct
RSSQSVLYSIGYNYVD

SEQ ID NO: 28 - protein, 16 aa, synthetic construct
TASQSLLYSIGYNYVD

SEQ ID NO: 29 - protein, 16 aa, synthetic construct
RSSQSLLYSIGYNYVD

SEQ ID NO: 30 - protein, 16 aa, synthetic construct
RSSQSLLYSIGKNYVD

SEQ ID NO: 31 - protein, 16 aa, synthetic construct
RSSQGLLYSIGYNYVD

SEQ ID NO: 32 - protein, 16 aa, synthetic construct
RSTQSLLYSIGKNYVD

SEQ ID NO: 33 - protein, 16 aa, synthetic construct
TSSQSLLYSIGKNYVD

SEQ ID NO: 34 - protein, 16 aa, synthetic construct
TSSQSLLYSIGYNYVD

SEQ ID NO: 35 - protein, 16 aa, synthetic construct
RSSQSVLYSIGKNYVD

SEQ ID NO: 36 - protein, 16 aa, synthetic construct
TASQSLLYSIGKNYVD

SEQ ID NO: 37 - protein, 16 aa, synthetic construct
TASQSLLYSIGYNYLD

SEQ ID NO: 38 - protein, 16 aa, synthetic construct
RSSQSLLYSNRNNYLD

SEQ ID NO: 39 - protein, 16 aa, synthetic construct
RSSQSLLYSNGYNYLD

SEQ ID NO: 40 - protein, 16 aa, synthetic construct
RASQSLLYSVGYNYVD

SEQ ID NO: 41 - protein, 7 aa, synthetic construct
LGSNRAS

SEQ ID NO: 42 - protein, 7 aa, synthetic construct
LGSKRAS

SEQ ID NO: 43 - protein, 7 aa, synthetic construct
LGNKRAS

SEQ ID NO: 44 - protein, 7 aa, synthetic construct
LGSQRAT

SEQ ID NO: 45 - protein, 7 aa, synthetic construct
LGNNRAT

SEQ ID NO: 46 - protein, 7 aa, synthetic construct
KGNNRAT

SEQ ID NO: 47 - protein, 7 aa, synthetic construct
EGNKRAS

SEQ ID NO: 48 - protein, 7 aa, synthetic construct
SASIRAS

SEQ ID NO: 49 - protein, 7 aa, synthetic construct
LSSNRAS

SEQ ID NO: 50 - protein, 7 aa, synthetic construct
EGNNRAT

SEQ ID NO: 51 - protein, 7 aa, synthetic construct
EGNNRAS

SEQ ID NO: 52 - protein, 7 aa, synthetic construct
LGNNRAS

SEQ ID NO: 53 - protein, 9 aa, synthetic construct
MQALQTPYT

SEQ ID NO: 54 - protein, 9 aa, synthetic construct
MQSLDTPYT

SEQ ID NO: 55 - protein, 9 aa, synthetic construct
MQALTTPYT

SEQ ID NO: 56 - protein, 9 aa, synthetic construct
MQSLQTPYV

SEQ ID NO: 57 - protein, 9 aa, synthetic construct
MQSLQTPYV

SEQ ID NO: 58 - protein, 9 aa, synthetic construct
MQALDTPYT

SEQ ID NO: 59 - protein, 9 aa, synthetic construct
MQALTTPYA

SEQ ID NO: 60 - protein, 9 aa, synthetic construct
MQALGSPYT

SEQ ID NO: 61 - protein, 9 aa, synthetic construct
MQSLTSPYT

SEQ ID NO: 62 - protein, 9 aa, synthetic construct
MQAYNSPYT

SEQ ID NO: 63 - protein, 9 aa, synthetic construct
MQAFQAPYT

SEQ ID NO: 64 - protein, 9 aa, synthetic construct
MQALQAPYT

SEQ ID NO: 65 - protein, 9 aa, synthetic construct
MQAYQSPYV

SEQ ID NO: 66 - protein, 7 aa, synthetic construct
GFTFRDY

SEQ ID NO: 67 - protein, 7 aa, synthetic construct
GFTFRDF

SEQ ID NO: 68 - protein, 7 aa, synthetic construct
GFTFTDF

SEQ ID NO: 69 - protein, 7 aa, synthetic construct
GFTFRTY

SEQ ID NO: 70 - protein, 7 aa, synthetic construct
GFTFRDS

SEQ ID NO: 71 - protein, 6 aa, synthetic construct
SGSGGN

SEQ ID NO: 72 - protein, 6 aa, synthetic construct
SGSGSS

SEQ ID NO: 73 - protein, 6 aa, synthetic construct
SGSGDN

SEQ ID NO: 74 - protein, 6 aa, synthetic construct
SGGGGS

SEQ ID NO: 75 - protein, 6 aa, synthetic construct
SGDGGN

SEQ ID NO: 76 - protein, 6 aa, synthetic construct
SGGGGN

SEQ ID NO: 77 - protein, 6 aa, synthetic construct
SGDGSN

SEQ ID NO: 78 - protein, 6 aa, synthetic construct
SGSGDG

SEQ ID NO: 79 - protein, 8 aa, synthetic construct
ISGSGGNT

SEQ ID NO: 80 - protein, 8 aa, synthetic construct
ISGSGGNI

SEQ ID NO: 81 - protein, 8 aa, synthetic construct
ISGSGSST

SEQ ID NO: 82 - protein, 8 aa, synthetic construct
ISGSGDNI

SEQ ID NO: 83 - protein, 8 aa, synthetic construct
ISGDGSNT

SEQ ID NO: 84 - protein, 8 aa, synthetic construct
VSGSGGNT

SEQ ID NO: 85 - protein, 8 aa, synthetic construct
ISGSGDNT

SEQ ID NO: 86 - protein, 8 aa, synthetic construct
ISGGGGST

SEQ ID NO: 87 - protein, 8 aa, synthetic construct
ISGSGGNK

SEQ ID NO: 88 - protein, 8 aa, synthetic construct
VSGDGGNT

SEQ ID NO: 89 - protein, 8 aa, synthetic construct
ISGGGGNT

SEQ ID NO: 90 - protein, 8 aa, synthetic construct
ISGSGDGT

SEQ ID NO: 91 - protein, 18 aa, synthetic construct
AKDRLSITIRPRYYGLDV

SEQ ID NO: 92 - protein, 18 aa, synthetic construct
AKDRLSKTIRPRYYGLDV

SEQ ID NO: 93 - protein, 18 aa, synthetic construct
AKDRLSITVRPRYYGLDV

SEQ ID NO: 94 - protein, 18 aa, synthetic construct
AKDRVSITIRPRYYGLDV

SEQ ID NO: 95 - protein, 18 aa, synthetic construct
AKDRLSVTVRPRYYGLDV

SEQ ID NO: 96 - protein, 18 aa, synthetic construct
AKDRLSVTIRPRYYGLDV

SEQ ID NO: 97 - protein, 18 aa, synthetic construct
AKDRVSIYVRPRYYGLDV

SEQ ID NO: 98 - protein, 18 aa, synthetic construct
AKDRLSIAVRPRYYGLDV

SEQ ID NO: 99 - protein, 18 aa, synthetic construct
AKDRLSIYIRPRYYGLDV

SEQ ID NO: 100 - protein, 11 aa, synthetic construct
QSLLYSIGYNY

SEQ ID NO: 101 - protein, 11 aa, synthetic construct
QSVLYSIGYNY

SEQ ID NO: 102 - protein, 11 aa, synthetic construct
QSLLYSIGKNY

SEQ ID NO: 103 - protein, 11 aa, synthetic construct
QGLLYSIGYNY

SEQ ID NO: 104 - protein, 11 aa, synthetic construct
QSVLYSIGKNY

SEQ ID NO: 105 - protein, 11 aa, synthetic construct
QSLLYSNRNNY

SEQ ID NO: 106 - protein, 11 aa, synthetic construct
QSLLYSNGYNY

SEQ ID NO: 107 - protein, 11 aa, synthetic construct
QSLLYSVGYNY

SEQ ID NO: 108-112 - skipped (5 entries)

SEQ ID NO: 113 - protein, 125 aa, synthetic construct [sequence omitted]

SEQ ID NO: 114 - protein, 125 aa, synthetic construct
EVQLVESGGGLEQPGGSLRLSCAGSGFTFRDFAMTWVRQAPGKGLEWVSSISGSGGNIYYADSVKGRFTISRDNSKNTLYLQMNSLRAEDTAVYYCAKDRLSKTIRPRYYGLDVWGQGTTVTVSS

SEQ ID NO: 115 - protein, 125 aa, synthetic construct
EVQLVESGGGLEQPGGSLRLSCAGSGFTFRDFAMTWVRQAPGKGLEWVSSISGSGSSTYYADSVKGRFTISRDNSKNTLYLQMNSLRAEDTAVYYCAKDRLSITVRPRYYGLDVWGQGTTVTVSS

SEQ ID NO: 116 - protein, 125 aa, synthetic construct
EVQLVESGGGLEQPGGSLRLSCAGSGFTFRDFAMTWVRQAPGKGLEWVSSISGSGDNIYYADSVKGRFTISRDNSKNTLYLQMNSLRAEDTAVYYCAKDRVSITIRPRYYGLDVWGQGTTVTVSS

SEQ ID NO: 117 - protein, 125 aa, synthetic construct
EVQLVESGGGLEQPGGSLRLSCAGSGFTFRDFAMTWVRQAPGKGLEWVSSISGSGDNIYYADSVKGRFTISRDNSKNTLYLQMNSLRAEDTAVYYCAKDRLSVTVRPRYYGLDVWGQGTTVTVSS

SEQ ID NO: 118 - protein, 125 aa, synthetic construct
EVQLVESGGGLEQPGGSLRLSCAGSGFTFRDFAMTWVRQAPGKGLEWVSSISGSGGNIYYADSVKGRFTISRDNSKNTLYLQMNSLRAEDTAVYYCAKDRLSITIRPRYYGLDVWGQGTTVTVSS

SEQ ID NO: 119 - protein, 125 aa, synthetic construct
EVQLVESGGGLEQPGGSLRLSCAGSGFTFTDFAMTWVRQAPGKGLEWVSSISGSGGNIYYADSVKGRFTISRDNSKNTLYLQMNSLRAEDTAVYYCAKDRLSVTVRPRYYGLDVWGQGTTVTVSS

SEQ ID NO: 120 - protein, 125 aa, synthetic construct
EVQLVESGGGLEQPGGSLRLSCAGSGFTFRDFAMTWVRQAPGKGLEWVSSISGDGSNTYYADSVKGRFTISRDNSKNTLYLQMNSLRAEDTAVYYCAKDRLSKTIRPRYYGLDVWGQGTTVTVSS

SEQ ID NO: 121 - protein, 125 aa, synthetic construct
EVQLVESGGGLEQPGGSLRLSCAGSGFTFRDFAMTWVRQAPGKGLEWVSSVSGSGGNTYYADSVKGRFTISRDNSKNTLYLQMNSLRAEDTAVYYCAKDRLSVTVRPRYYGLDVWGQGTTVTVSS

SEQ ID NO: 122 - protein, 125 aa, synthetic construct
EVQLVESGGGLEQPGGSLRLSCAGSGFTFRTYAMTWVRQAPGKGLEWVSSVSGSGGNTYYADSVKGRFTISRDNSKNTLYLQMNSLRAEDTAVYYCAKDRLSVTVRPRYYGLDVWGQGTTVTVSS

SEQ ID NO: 123 - protein, 125 aa, synthetic construct
EVQLVESGGGLEQPGGSLRLSCAGSGFTFRTYAMTWVRQAPGKGLEWVSSISGSGDNIYYADSVKGRFTISRDNSKNTLYLQMNSLRAEDTAVYYCAKDRLSVTIRPRYYGLDVWGQGTTVTVSS

SEQ ID NO: 124 - protein, 125 aa, synthetic construct
EVQLVESGGGLEQPGGSLRLSCAGSGFTFRDFAMTWVRQAPGKGLEWVSSISGSGSSTYYADSVKGRFTISRDNSKNTLYLQMNSLRAEDTAVYYCAKDRLSVTVRPRYYGLDVWGQGTTVTVSS

SEQ ID NO: 125 - protein, 125 aa, synthetic construct
EVQLVESGGGLEQPGGSLRLSCAGSGFTFRDFAMTWVRQAPGKGLEWVSSISGSGDNTYYADSVKGRFTISRDNSKNTLYLQMNSLRAEDTAVYYCAKDRLSKTIRPRYYGLDVWGQGTTVTVSS

SEQ ID NO: 126 - protein, 125 aa, synthetic construct
EVQLVESGGGLEQPGGSLRLSCAGSGFTFRTYAMTWVRQAPGKGLEWVSSISGSGDNTYYADSVKGRFTISRDNSKNTLYLQMNSLRAEDTAVYYCAKDRVSITIRPRYYGLDVWGQGTTVTVSS

SEQ ID NO: 127 - protein, 125 aa, synthetic construct
EVQLVESGGGLEQPGGSLRLSCAGSGFTFRDFAMTWVRQAPGKGLEWVSSISGSGDNTYYADSVKGRFTISRDNSKNTLYLQMNSLRAEDTAVYYCAKDRLSITVRPRYYGLDVWGQGTTVTVSS

SEQ ID NO: 128 - protein, 125 aa, synthetic construct
EVQLVESGGGLEQPGGSLRLSCAGSGFTFRTYAMTWVRQAPGKGLEWVSSISGSGGNIYYADSVKGRFTISRDNSKNTLYLQMNSLRAEDTAVYYCAKDRVSIYVRPRYYGLDVWGQGTTVTVSS

SEQ ID NO: 129 - protein, 125 aa, synthetic construct
EVQLVESGGGLEQPGGSLRLSCAGSGFTFRDFAMTWVRQAPGKGLEWVSSISGGGGSTYYADSVKGRFTISRDNSKNTLYLQMNSLRAEDTAVYYCAKDRLSVTVRPRYYGLDVWGQGTTVTVSS

SEQ ID NO: 130 - protein, 125 aa, synthetic construct
EVQLVESGGGLEQPGGSLRLSCAGSGFTFRDFAMTWVRQAPGKGLEWVSSISGSGGNIYYADSVKGRFTISRDNSKNTLYLQMNSLRAEDTAVYYCAKDRLSIAVRPRYYGLDVWGQGTTVTVSS

SEQ ID NO: 131 - protein, 125 aa, synthetic construct
EVQLVESGGGLEQPGGSLRLSCAGSGFTFRDFAMTWVRQAPGKGLEWVSSISGSGDNIYYADSVKGRFTISRDNSKNTLYLQMNSLRAEDTAVYYCAKDRLSKTIRPRYYGLDVWGQGTTVTVSS

SEQ ID NO: 132 - protein, 125 aa, synthetic construct
EVQLVESGGGLEQPGGSLRLSCAGSGFTFRDFAMTWVRQAPGKGLEWVSSISGGGGSTYYADSVKGRFTISRDNSKNTLYLQMNSLRAEDTAVYYCAKDRLSIAVRPRYYGLDVWGQGTTVTVSS

SEQ ID NO: 133 - protein, 125 aa, synthetic construct
EVQLVESGGGLEQPGGSLRLSCAGSGFTFTDFAMTWVRQAPGKGLEWVSSISGSGGNKYYADSVKGRFTISRDNSKNTLYLQMNSLRAEDTAVYYCAKDRLSVTVRPRYYGLDVWGQGTTVTVSS

SEQ ID NO: 134 - protein, 125 aa, synthetic construct
EVQLVESGGGLEQPGGSLRLSCAGSGFTFRDFAMTWVRQAPGKGLEWVSSISGSGGNIYYADSVKGRFTISRDNSKNTLYLQMNSLRAEDTAVYYCAKDRLSVTVRPRYYGLDVWGQGTTVTVSS

SEQ ID NO: 135 - protein, 125 aa, synthetic construct
EVQLVESGGGLEQPGGSLRLSCAGSGFTFRDFAMTWVRQAPGKGLEWVSSVSGDGGNTYYADSVKGRFTISRDNSKNTLYLQMNSLRAEDTAVYYCAKDRLSVTVRPRYYGLDVWGQGTTVTVSS

SEQ ID NO: 136 - protein, 125 aa, synthetic construct
EVQLVESGGGLEQPGGSLRLSCAGSGFTFRDFAMTWVRQAPGKGLEWVSSISGSGGNIYYADSVKGRFTISRDNSKNTLYLQMNSLRAEDTAVYYCAKDRVSITIRPRYYGLDVWGQGTTVTVSS

SEQ ID NO: 137 - protein, 125 aa, synthetic construct
EVQLVESGGGLEQPGGSLRLSCAGSGFTFRTYAMTWVRQAPGKGLEWVSSISGSGGNTYYADSVKGRFTISRDNSKNTLYLQMNSLRAEDTAVYYCAKDRVSITIRPRYYGLDVWGQGTTVTVSS

SEQ ID NO: 138 - protein, 125 aa, synthetic construct
EVQLVESGGGLEQPGGSLRLSCAGSGFTFRTYAMTWVRQAPGKGLEWVSSISGGGGNTYYADSVKGRFTISRDNSKNTLYLQMNSLRAEDTAVYYCAKDRLSVTVRPRYYGLDVWGQGTTVTVSS

SEQ ID NO: 139 - protein, 125 aa, synthetic construct
EVQLVESGGGLEQPGGSLRLSCAGSGFTFRDSAMTWVRQAPGKGLEWVSSISGSGDNTYYADSVKGRFTISRDNSKNTLYLQMNSLRAEDTAVYYCAKDRLSITVRPRYYGLDVWGQGTTVTVSS

SEQ ID NO: 140 - protein, 125 aa, synthetic construct
EVQLVESGGGLEQPGGSLRLSCAGSGFTFRDFAMTWVRQAPGKGLEWVSSVSGDGGNTYYADSVKGRFTISRDNSKNTLYLQMNSLRAEDTAVYYCAKDRLSKTIRPRYYGLDVWGQGTTVTVSS

SEQ ID NO: 141 - protein, 125 aa, synthetic construct
EVQLVESGGGLEQPGGSLRLSCAGSGFTFRDFAMTWVRQAPGKGLEWVSSISGGGGNTYYADSVKGRFTISRDNSKNTLYLQMNSLRAEDTAVYYCAKDRVSITIRPRYYGLDVWGQGTTVTVSS

SEQ ID NO: 142 - protein, 125 aa, synthetic construct
EVQLVESGGGLEQPGGSLRLSCAGSGFTFRTYAMTWVRQAPGKGLEWVSSISGDGSNTYYADSVKGRFTISRDNSKNTLYLQMNSLRAEDTAVYYCAKDRVSITIRPRYYGLDVWGQGTTVTVSS

SEQ ID NO: 143 - protein, 125 aa, synthetic construct
EVQLVESGGGLEQPGGSLRLSCAGSGFTFRDFAMTWVRQAPGKGLEWVSSISGGGGNTYYADSVKGRFTISRDNSKNTLYLQMNSLRAEDTAVYYCAKDRLSIAVRPRYYGLDVWGQGTTVTVSS

SEQ ID NO: 144 - protein, 125 aa, synthetic construct
EVQLVESGGGLEQPGGSLRLSCAGSGFTFRDFAMTWVRQAPGKGLEWVSSISGSGDGTYYADSVKGRFTISRDNSKNTLYLQMNSLRAEDTAVYYCAKDRVSITIRPRYYGLDVWGQGTTVTVSS

SEQ ID NO: 145 - protein, 125 aa, synthetic construct
EVQLVESGGGLEQPGGSLRLSCAGSGFTFRDSAMTWVRQAPGKGLEWVSSISGSGDGTYYADSVKGRFTISRDNSKNTLYLQMNSLRAEDTAVYYCAKDRLSIYIRPRYYGLDVWGQGTTVTVSS

SEQ ID NO: 146 - protein, 112 aa, synthetic construct [sequence omitted]

SEQ ID NO: 147 - protein, 112 aa, synthetic construct
DIVMTQSPLSLPVTPGEPASISCRSSQSVLYSIGYNYVDWYLQKSGQSPQLLIYLGSKRASGVPDRFSGSGSGTDFTLKISRVEAEDVGFYYCMQSLDTPYTFGQGTKLEIK

SEQ ID NO: 148 - protein, 112 aa, synthetic construct
DIVMTQSPLSLPVTPGEPASISCTASQSLLYSIGYNYVDWYLQKSGQSPQLLIYLGSKRASGVPDRFSGSGSGTDFTLKISRVEAEDVGFYYCMQALTTPYTFGQGTKLEIK

SEQ ID NO: 149 - protein, 112 aa, synthetic construct
DIVMTQSPLSLPVTPGEPASISCRSSQSLLYSIGYNYVDWYLQKSGQSPQLLIYLGNKRASGVPDRFSGSGSGTDFTLKISRVEAEDVGFYYCMQALTTPYTFGQGTKLEIK

SEQ ID NO: 150 - protein, 112 aa, synthetic construct
DIVMTQSPLSLPVTPGEPASISCTASQSLLYSIGYNYVDWYLQKSGQSPQLLIYLGNNRATGVPDRFSGSGSGTDFTLKISRVEAEDVGFYYCMQALTTPYTFGQGTKLEIK

SEQ ID NO: 151 - protein, 112 aa, synthetic construct
DIVMTQSPLSLPVTPGEPASISCRSSQSLLYSIGKNYVDWYLQKSGQSPQLLIYLGNKRASGVPDRFSGSGSGTDFTLKISRVEAEDVGFYYCMQSLQTPYVFGQGTKLEIK

SEQ ID NO: 152 - protein, 112 aa, synthetic construct
DIVMTQSPLSLPVTPGEPASISCRSSQGLLYSIGYNYVDWYLQKSGQSPQLLIYLGNKRASGVPDRFSGSGSGTDFTLKISRVEAEDVGFYYCMQSLQTPYTFGQGTKLEIK

SEQ ID NO: 153 - protein, 112 aa, synthetic construct
DIVMTQSPLSLPVTPGEPASISCRSTQSLLYSIGKNYVDWYLQKSGQSPQLLIYKGNNRATGVPDRFSGSGSGTDFTLKISRVEAEDVGFYYCMQALDTPYTFGQGTKLEIK

SEQ ID NO: 154 - protein, 112 aa, synthetic construct
DIVMTQSPLSLPVTPGEPASISCRSTQSLLYSIGKNYVDWYLQKSGQSPQLLIYLGNNRATGVPDRFSGSGSGTDFTLKISRVEAEDVGFYYCMQALTTPYAFGQGTKLEIK

SEQ ID NO: 155 - protein, 112 aa, synthetic construct
DIVMTQSPLSLPVTPGEPASISCRSSQSLLYSIGKNYVDWYLQKSGQSPQLLIYLGNNRATGVPDRFSGSGSGTDFTLKISRVEAEDVGFYYCMQALGSPYTFGQGTKLEIK

SEQ ID NO: 156 - protein, 112 aa, synthetic construct
DIVMTQSPLSLPVTPGEPASISCRSSQSLLYSIGYNYVDWYLQKSGQSPQLLIYLGNKRASGVPDRFSGSGSGTDFTLKISRVEAEDVGFYYCMQALGSPYTFGQGTKLEIK

SEQ ID NO: 157 - protein, 112 aa, synthetic construct
DIVMTQSPLSLPVTPGEPASISCRSSQSVLYSIGYNYVDWYLQKSGQSPQLLIYLGNKRASGVPDRFSGSGSGTDFTLKISRVEAEDVGFYYCMQSLQTPYTFGQGTKLEIK

SEQ ID NO: 158 - protein, 112 aa, synthetic construct
DIVMTQSPLSLPVTPGEPASISCRSSQSVLYSIGYNYVDWYLQKSGQSPQLLIYLGNKRASGVPDRFSGSGSGTDFTLKISRVEAEDVGFYYCMQSLTSPYTFGQGTKLEIK

SEQ ID NO: 159 - protein, 112 aa, synthetic construct
DIVMTQSPLSLPVTPGEPASISCTASQSLLYSIGYNYVDWYLQKSGQSPQLLIYLGNKRASGVPDRFSGSGSGTDFTLKISRVEAEDVGFYYCMQSLTSPYTFGQGTKLEIK

SEQ ID NO: 160 - protein, 112 aa, synthetic construct
DIVMTQSPLSLPVTPGEPASISCTASQSLLYSIGYNYVDWYLQKSGQSPQLLIYLGSKRASGVPDRFSGSGSGTDFTLKISRVEAEDVGFYYCMQAYNSPYTFGQGTKLEIK

SEQ ID NO: 161 - protein, 112 aa, synthetic construct
DIVMTQSPLSLPVTPGEPASISCTSSQSLLYSIGKNYVDWYLQKSGQSPQLLIYLGNKRASGVPDRFSGSGSGTDFTLKISRVEAEDVGFYYCMQSLTSPYTFGQGTKLEIK

SEQ ID NO: 162 - protein, 112 aa, synthetic construct
DIVMTQSPLSLPVTPGEPASISCTSSQSLLYSIGYNYVDWYLQKSGQSPQLLIYLGNKRASGVPDRFSGSGSGTDFTLKISRVEAEDVGFYYCMQALGSPYTFGQGTKLEIK

SEQ ID NO: 163 - protein, 112 aa, synthetic construct
DIVMTQSPLSLPVTPGEPASISCTSSQSLLYSIGYNYVDWYLQKSGQSPQLLIYLGNKRASGVPDRFSGSGSGTDFTLKISRVEAEDVGFYYCMQSLQTPYVFGQGTKLEIK

SEQ ID NO: 164 - protein, 112 aa, synthetic construct
DIVMTQSPLSLPVTPGEPASISCTASQSLLYSIGYNYVDWYLQKSGQSPQLLIYLGSKRASGVPDRFSGSGSGTDFTLKISRVEAEDVGFYYCMQSLTSPYTFGQGTKLEIK

SEQ ID NO: 165 - protein, 112 aa, synthetic construct
DIVMTQSPLSLPVTPGEPASISCRSSQSVLYSIGKNYVDWYLQKSGQSPQLLIYLGSQRATGVPDRFSGSGSGTDFTLKISRVEAEDVGFYYCMQSLTSPYTFGQGTKLEIK

SEQ ID NO: 166 - protein, 112 aa, synthetic construct
DIVMTQSPLSLPVTPGEPASISCTASQSLLYSIGKNYVDWYLQKSGQSPQLLIYLGNNRATGVPDRFSGSGSGTDFTLKISRVEAEDVGFYYCMQSLTSPYTFGQGTKLEIK

SEQ ID NO: 167 - protein, 112 aa, synthetic construct
DIVMTQSPLSLPVTPGEPASISCTASQSLLYSIGYNYLDWYLQKSGQSPQLLIYEGNKRASGVPDRFSGSGSGTDFTLKISRVEAEDVGFYYCMQSLTSPYTFGQGTKLEIK

SEQ ID NO: 168 - protein, 112 aa, synthetic construct
DIVMTQSPLSLPVTPGEPASISCTSSQSLLYSIGYNYVDWYLQKSGQSPQLLIYLGNKRASGVPDRFSGSGSGTDFTLKISRVEAEDVGFYYCMQSLQTPYTFGQGTKLEIK

SEQ ID NO: 169 - protein, 112 aa, synthetic construct
DIVMTQSPLSLPVTPGEPASISCTSSQSLLYSIGYNYVDWYLQKSGQSPQLLIYLGNKRASGVPDRFSGSGSGTDFTLKISRVEAEDVGFYYCMQSLTSPYTFGQGTKLEIK

SEQ ID NO: 170 - protein, 112 aa, synthetic construct
DIVMTQSPLSLPVTPGEPASISCRSSQSLLYSNRNNYLDWYLQKSGQSPQLLIYSASIRASGVPDRFSGSGSGTDFTLKISRVEAEDVGFYYCMQAFQAPYTFGQGTKLEIK

SEQ ID NO: 171 - protein, 112 aa, synthetic construct
DIVMTQSPLSLPVTPGEPASISCRSSQSLLYSNGYNYLDWYLQKSGQSPQLLIYLGSNRASGVPDRFSGSGSGTDFTLKISRVEAEDVGFYYCMQALQAPYTFGQGTKLEIK

SEQ ID NO: 172 - protein, 112 aa, synthetic construct
DIVMTQSPLSLPVTPGEPASISCRSSQSLLYSNGYNYLDWYLQKSGQSPQLLIYLSSNRASGVPDRFSGSGSGTDFTLKISRVEAEDVGFYYCMQAFQAPYTFGQGTKLEIK

SEQ ID NO: 173 - protein, 112 aa, synthetic construct
DIVMTQSPLSLPVTPGEPASISCTASQSLLYSIGYNYVDWYLQKSGQSPQLLIYLGSKRASGVPDRFSGSGSGTDFTLKISRVEAEDVGFYYCMQALGSPYTFGQGTKLEIK

SEQ ID NO: 174 - protein, 112 aa, synthetic construct
DIVMTQSPLSLPVTPGEPASISCRSSQGLLYSIGYNYVDWYLQKSGQSPQLLIYLGNKRASGVPDRFSGSGSGTDFTLKISRVEAEDVGFYYCMQALDTPYTFGQGTKLEIK

SEQ ID NO: 175 - protein, 112 aa, synthetic construct
DIVMTQSPLSLPVTPGEPASISCTSSQSLLYSIGKNYVDWYLQKSGQSPQLLIYEGNNRATGVPDRFSGSGSGTDFTLKISRVEAEDVGFYYCMQSLTSPYTFGQGTKLEIK

SEQ ID NO: 176 - protein, 112 aa, synthetic construct
DIVMTQSPLSLPVTPGEPASISCRSSQSLLYSIGKNYVDWYLQKSGQSPQLLIYEGNNRASGVPDRFSGSGSGTDFTLKISRVEAEDVGFYYCMQSLTSPYTFGQGTKLEIK

SEQ ID NO: 177 - protein, 112 aa, synthetic construct
DIVMTQSPLSLPVTPGEPASISCRASQSLLYSVGYNYVDWYLQKSGQSPQLLIYLGNNRASGVPDRFSGSGSGTDFTLKISRVEAEDVGFYYCMQSLTSPYTFGQGTKLEIK

SEQ ID NO: 178 - protein, 112 aa, synthetic construct
DIVMTQSPLSLPVTPGEPASISCRSSQSLLYSIGKNYVDWYLQKSGQSPQLLIYEGNKRASGVPDRFSGSGSGTDFTLKISRVEAEDVGFYYCMQSLTSPYTFGQGTKLEIK

SEQ ID NO: 179 - protein, 112 aa, synthetic construct
DIVMTQSPLSLPVTPGEPASISCRSSQSLLYSIGKNYVDWYLQKSGQSPQLLIYLGNKRASGVPDRFSGSGSGTDFTLKISRVEAEDVGFYYCMQALGSPYTFGQGTKLEIK

SEQ ID NO: 180 - protein, 112 aa, synthetic construct
DIVMTQSPLSLPVTPGEPASISCRSSQSVLYSIGYNYVDWYLQKSGQSPQLLIYLGNKRASGVPDRFSGSGSGTDFTLKISRVEAEDVGFYYCMQAYQSPYVFGQGTKLEIK

SEQ ID NO: 181 - protein, 112 aa, synthetic construct
DIVMTQSPLSLPVTPGEPASISCTASQSLLYSIGYNYVDWYLQKSGQSPQLLIYLGNKRASGVPDRFSGSGSGTDFTLKISRVEAEDVGFYYCMQAYNSPYTFGQGTKLEIK

SEQ ID NO: 182 - protein, 112 aa, synthetic construct
DIVMTQSPLSLPVTPGEPASISCTASQSLLYSIGYNYVDWYLQKSGQSPQLLIYLGNKRASGVPDRFSGSGSGTDFTLKISRVEAEDVGFYYCMQALTTPYTFGQGTKLEIK

SEQ ID NO: 183 - protein, 112 aa, synthetic construct
DIVMTQSPLSLPVTPGEPASISCTSSQSLLYSIGYNYVDWYLQKSGQSPQLLIYLGSKRASGVPDRFSGSGSGTDFTLKISRVEAEDVGFYYCMQSLTSPYTFGQGTKLEIK

SEQ ID NO: 184 - protein, 112 aa, synthetic construct
DIVMTQSPLSLPVTPGEPASISCRSTQSLLYSIGKNYVDWYLQKSGQSPQLLIYEGNNRASGVPDRFSGSGSGTDFTLKISRVEAEDVGFYYCMQSLTSPYTFGQGTKLEIK

SEQ ID NO: 185 - protein, 112 aa, synthetic construct
DIVMTQSPLSLPVTPGEPASISCTASQSLLYSIGYNYVDWYLQKSGQSPQLLIYLGNNRATGVPDRFSGSGSGTDFTLKISRVEAEDVGFYYCMQSLQTPYTFGQGTKLEIK

SEQ ID NO: 186 - protein, 112 aa, synthetic construct
DIVMTQSPLSLPVTPGEPASISCTSSQSLLYSIGYNYVDWYLQKSGQSPQLLIYLGNKRASGVPDRFSGSGSGTDFTLKISRVEAEDVGFYYCMQALQTPYTFGQGTKLEIK

SEQ ID NO: 187 - protein, 8 aa, synthetic construct
GFTFRDYA

SEQ ID NO: 188 - protein, 8 aa, synthetic construct
GFTFRDFA

SEQ ID NO: 189 - protein, 8 aa, synthetic construct
GFTFTDFA

SEQ ID NO: 190 - protein, 8 aa, synthetic construct
GFTFRTYA

SEQ ID NO: 191 - protein, 8 aa, synthetic construct
GFTFRDSA

SEQ ID NO: 192 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 193 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 194 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 195 - protein, 324 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSP

SEQ ID NO: 196 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 197 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 198 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 199 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 200 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 201 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 202 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 203 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 204 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 205 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 206 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 207 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 208 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 209 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 210 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 211 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 212 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 213 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 214 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 215 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 216 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 217 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 218 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 219 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 220 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 221 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 222 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 223 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 224 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 225 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 226 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSYTQKSLSLSLGK

SEQ ID NO: 227 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 228 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 229 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 230 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 231 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 232 - protein, 324 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLYITREPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSP

SEQ ID NO: 233 - protein, 324 aa, synthetic construct; identical to SEQ ID NO: 195

SEQ ID NO: 234 - protein, 324 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVDHHDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHSHYTQKSLSLSP

SEQ ID NO: 235 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 236 - protein, 107 aa, Homo sapiens [sequence omitted]

SEQ ID NO: 237 - protein, 825 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 238 - protein, 825 aa, Macaca fascicularis
MGWLCSGLLFPVSCLVLLQVASSGSMKVLQEPACVSDYMSISTCEWKMGGPTNCSAELRLLYQLVFQSSETHTCVPENNGGVGCVCHLLMDDVVSMDNYTLDLWAGQQLLWKGSFKPSEHVKPRAPGNLTVHTNVSDTVLLTWSNPYPPDNYLYNDLTYAVNIWSENDPAYSRIHNVTYLKPTLRIPASTLKSGISYRARVRAWAQHYNTTWSEWSPSTKWYNSYREPFEQRLLWGVSAACVFILFFCLSCYFSVTKIKKEWWDQIPNPARSHLVAIIIQDAQESQWEKRSRGQEAAKCPYWKNCLTKLLPCFLEHNMKRDEDPHKAVKDLPFRGSGKSAWCPVEISKTVLWPESISVVRCVELFEAPVECKEEEEVEEEKGSFCTSSESNRDDFQEGREGIVARLTESLFLDLLGGENGGFFQQDMGESCLLPPLGSTSAHVPWDEFPSAGPKEVPPWGKEQPLHQEPSPPASPTQSPDNPTCTEMPLVISSNPAYRSFSNSLSQSPCPRELGPDPLLARHLEEVDPEMPCAPQLSEPTTVAPAEPETWEQILRRNVLQHGAAAAPASAPTSGYREFVHAVQQGGIQASAVAGLGPPGEAGYKAFSSLLASSAVSPGECGFGASSGEEGYKPFQDLTPGCPGDPAPVPVPLFTFGLDREPPHSPQSSHLPSNSPEHLALEPGEKVEDMQKPPLPPEQATDPLGDNLGSGIVYSALTCHLCGHLKQCHGQEDGGQAPVVASPCCGCCCGDRSSPPTTPLRAPDPSLGGVPLEASLCLASLAPSGISEKSKSSLSFHPAPGSAQSSSQTPQIVNFVSVGPTCMRVS

SEQ ID NO: 239 - protein, 810 aa, Mus musculus
MGRLCTKFLTSVGCLILLLVTGSGSIKVLGEPTCFSDYIRTSTCEWFLDSAVDCSSQLCLHYRLMFFEFSENLTCIPRNSASTVCVCHMEMNRPVQSDRYQMELWAEHRQLWQGSFSPSGNVKPLAPDNLTLHTNVSDEWLLTWNNLYPSNNLLYKDLISMVNISREDNPAEFIVYNVTYKEPRLSFPINILMSGVYYTARVRVRSQILTGTWSEWSPSITWYNHFQLPLIQRLPLGVTISCLCIPLFCLFCYFSITKIKKIWWDQIPTPARSPLVAIIIQDAQVPLWDKQTRSQESTKYPHWKTCLDKLLPCLLKHRVKKKTDFPKAAPTKSLQSPGKAGWCPMEVSRTVLWPENVSVSVVRCMELFEAPVQNVEEEEDEIVKEDLSMSPENSGGCGFQESQADIMARLTENLFSDLLEAENGGLGQSALAESCSPLPSGSGQASVSWACLPMGPSEEATCQVTEQPSHPGPLSGSPAQSAPTLACTQVPLVLADNPAYRSFSDCCSPAPNPGELAPEQQQADHLEEEEPPSPADPHSSGPPMQPVESWEQILHMSVLQHGAAAGSTPAPAGGYQEFVQAVKQGAAQDPGVPGVRPSGDPGYKAFSSLLSSNGIRGDTAAAGTDDGHGGYKPFQNPVPNQSPSSVPLFTFGLDTELSPSPLNSDPPKSPPECLGLELGLKGGDWVKAPPPADQVPKPFGDDLGFGIVYSSLTCHLCGHLKQHHSQEEGGQSPIVASPGCGCCYDDRSPSLGSLSGALESCPEGIPPEANLMSAPKTPSNLSGEGKGPGHSPVPSQTTEVPVGALGIAVS

SEQ ID NO: 240 - protein, 801 aa, Rattus sp.
MGWLCTKFLSSVSCLILLWVTGSGGIKVLGDPTCFSDYIRTSTCEWQLDSTVDCSSQLLLDYRLLFEFSENLTCTPKNSADTVCVCQMAIEEPIQADTYWLELWSERGQLWQGSFKPSDNVKPPAPDNLTLHTNVSNALLLMWSNPYPSNNFLHKGLICMVNISREDNPAEFKVYNVTYTEPKLSFPVNTLTSGVRYRARVRVLSQSFPGIWSEWSPSITWYNHFQLPLLQRLPLGVSISCICILLFCLTCYFSIIKIKKIWWDQIPTPARSPLAAIIIQDTKVSLWEKQTRSQESTKSRHWKTCLTKLLPCLLEHRVKKERESPKAAKTKPLQSPEKAGWYPAEVSRTVLWPENVHVSVVRCMELFEAPVQNVEEEEDEMVKGDLSMSPENSGGGFQESQADIMARLTENLFSDLLGAENGGVGQSSMAESSSLLPSESGQASTSWACFPTGPSETTCQVTGQQPPHPDPERATGTACTQVPLVISDNPAYRSFSDFSSPAPNPGELASEQKQAGHLEEGDLLSPVDPHSSGPPMQQAESWEQILHMSVLQHGTAGSTPAPTSGYQEFVQAVKQGASQDAGVPGVGPSGDTGYKAFSSLLSSSGVCTDTAAAGTDSGCGGYKPFQNPVPNQSPNSMPLFTFGLDMELPPSPLNSAPPNSTPECLGLELGLKGGTWLKAPPPSEQVPKPFGDDLGLGIVYSSLTCHLCGHLKQHHSQEEDGQIHVVASPGCGCCYDEKSPSLGNLSGTLESCPGEMSQEANLTLAPRTPSNLSGVGKGPGHSPVPSQTTEVPVGTLGVTVS

SEQ ID NO: 241 - protein, 25 aa, Homo sapiens
MGWLCSGLLFPVSCLVLLQVASSGN

SEQ ID NO: 242 - protein, 25 aa, Macaca fascicularis
MGWLCSGLLFPVSCLVLLQVASSGS

SEQ ID NO: 243 - protein, 25 aa, Mus musculus
MGRLCTKFLTSVGCLILLLVTGSGS

SEQ ID NO: 244 - protein, 25 aa, Rattus sp.
MGWLCTKFLSSVSCLILLWVTGSGG

SEQ ID NO: 245 - protein, 800 aa, Homo sapiens
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 246 - protein, 800 aa, Macaca fascicularis
MKVLQEPACVSDYMSISTCEWKMGGPTNCSAELRLLYQLVFQSSETHTCVPENNGGVGCVCHLLMDDVVSMDNYTLDLWAGQQLLWKGSFKPSEHVKPRAPGNLTVHTNVSDTVLLTWSNPYPPDNYLYNDLTYAVNIWSENDPAYSRIHNVTYLKPTLRIPASTLKSGISYRARVRAWAQHYNTTWSEWSPSTKWYNSYREPFEQRLLWGVSAACVFILFFCLSCYFSVTKIKKEWWDQIPNPARSHLVAIIIQDAQESQWEKRSRGQEAAKCPYWKNCLTKLLPCFLEHNMKRDEDPHKAVKDLPFRGSGKSAWCPVEISKTVLWPESISVVRCVELFEAPVECKEEEEVEEEKGSFCTSSESNRDDFQEGREGIVARLTESLFLDLLGGENGGFFQQDMGESCLLPPLGSTSAHVPWDEFPSAGPKEVPPWGKEQPLHQEPSPPASPTQSPDNPTCTEMPLVISSNPAYRSFSNSLSQSPCPRELGPDPLLARHLEEVDPEMPCAPQLSEPTTVAPAEPETWEQILRRNVLQHGAAAAPASAPTSGYREFVHAVQQGGIQASAVAGLGPPGEAGYKAFSSLLASSAVSPGECGFGASSGEEGYKPFQDLTPGCPGDPAPVPVPLFTFGLDREPPHSPQSSHLPSNSPEHLALEPGEKVEDMQKPPLPPEQATDPLGDNLGSGIVYSALTCHLCGHLKQCHGQEDGGQAPVVASPCCGCCCGDRSSPPTTPLRAPDPSLGGVPLEASLCLASLAPSGISEKSKSSLSFHPAPGSAQSSSQTPQIVNFVSVGPTCMRVS

SEQ ID NO: 247 - protein, 785 aa, Mus musculus
IKVLGEPTCFSDYIRTSTCEWFLDSAVDCSSQLCLHYRLMFFEFSENLTCIPRNSASTVCVCHMEMNRPVQSDRYQMELWAEHRQLWQGSFSPSGNVKPLAPDNLTLHTNVSDEWLLTWNNLYPSNNLLYKDLISMVNISREDNPAEFIVYNVTYKEPRLSFPINILMSGVYYTARVRVRSQILTGTWSEWSPSITWYNHFQLPLIQRLPLGVTISCLCIPLFCLFCYFSITKIKKIWWDQIPTPARSPLVAIIIQDAQVPLWDKQTRSQESTKYPHWKTCLDKLLPCLLKHRVKKKTDFPKAAPTKSLQSPGKAGWCPMEVSRTVLWPENVSVSVVRCMELFEAPVQNVEEEEDEIVKEDLSMSPENSGGCGFQESQADIMARLTENLFSDLLEAENGGLGQSALAESCSPLPSGSGQASVSWACLPMGPSEEATCQVTEQPSHPGPLSGSPAQSAPTLACTQVPLVLADNPAYRSFSDCCSPAPNPGELAPEQQQADHLEEEEPPSPADPHSSGPPMQPVESWEQILHMSVLQHGAAAGSTPAPAGGYQEFVQAVKQGAAQDPGVPGVRPSGDPGYKAFSSLLSSNGIRGDTAAAGTDDGHGGYKPFQNPVPNQSPSSVPLFTFGLDTELSPSPLNSDPPKSPPECLGLELGLKGGDWVKAPPPADQVPKPFGDDLGFGIVYSSLTCHLCGHLKQHHSQEEGGQSPIVASPGCGCCYDDRSPSLGSLSGALESCPEGIPPEANLMSAPKTPSNLSGEGKGPGHSPVPSQTTEVPVGALGIAVS

SEQ ID NO: 248 - protein, 776 aa, Rattus sp.
IKVLGDPTCFSDYIRTSTCEWQLDSTVDCSSQLLLDYRLLFEFSENLTCTPKNSADTVCVCQMAIEEPIQADTYWLELWSERGQLWQGSFKPSDNVKPPAPDNLTLHTNVSNALLLMWSNPYPSNNFLHKGLICMVNISREDNPAEFKVYNVTYTEPKLSFPVNTLTSGVRYRARVRVLSQSFPGIWSEWSPSITWYNHFQLPLLQRLPLGVSISCICILLFCLTCYFSIIKIKKIWWDQIPTPARSPLAAIIIQDTKVSLWEKQTRSQESTKSRHWKTCLTKLLPCLLEHRVKKERESPKAAKTKPLQSPEKAGWYPAEVSRTVLWPENVHVSVVRCMELFEAPVQNVEEEEDEMVKGDLSMSPENSGGGFQESQADIMARLTENLFSDLLGAENGGVGQSSMAESSSLLPSESGQASTSWACFPTGPSETTCQVTGQQPPHPDPERATGTACTQVPLVISDNPAYRSFSDFSSPAPNPGELASEQKQAGHLEEGDLLSPVDPHSSGPPMQQAESWEQILHMSVLQHGTAGSTPAPTSGYQEFVQAVKQGASQDAGVPGVGPSGDTGYKAFSSLLSSSGVCTDTAAAGTDSGCGGYKPFQNPVPNQSPNSMPLFTFGLDMELPPSPLNSAPPNSTPECLGLELGLKGGTWLKAPPPSEQVPKPFGDDLGLGIVYSSLTCHLCGHLKQHHSQEEDGQIHVVASPGCGCCYDEKSPSLGNLSGTLESCPGEMSQEANLTLAPRTPSNLSGVGKGPGHSPVPSQTTEVPVGTLGVTVS

SEQ ID NO: 249 - protein, 451 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 250 - protein, 219 aa, synthetic construct
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 251 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 252 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 253 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 254 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 255 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 256 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 257 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 258 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 259 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 260 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 261 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 262 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 263 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 264 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 265 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 266 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 267 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 268 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 269 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 270 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 271 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 272 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 273 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 274 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 275 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 276 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 277 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 278 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 279 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 280 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 281 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 282 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 283 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 284 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 285 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 286 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 287 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 288 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 289 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 290 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 291 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 292 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 293 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 294 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 295 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 296 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 297 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 298 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 299 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 300 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 301 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYASTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 302 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 303 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 304 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 305 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 306 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 307 - protein, 330 aa, synthetic construct [sequence omitted]

SEQ ID NO: 308 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 309 - protein, 327 aa, synthetic construct; identical to SEQ ID NO: 235

SEQ ID NO: 310 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 311 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPK

SEQ ID NO: 312 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 313 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 314 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 315 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 316 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 317 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 318 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 319 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 320 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 321 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 323 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 324 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 325 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 326 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 327 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 328 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 329 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 330 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 331 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 332 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 333 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 334 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 335 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 336 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 337 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 338 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 339 - protein, 327 aa, synthetic construct; identical to SEQ ID NO: 223

SEQ ID NO: 340 - protein, 327 aa, synthetic construct; identical to SEQ ID NO: 224

SEQ ID NO: 341 - protein, 327 aa, synthetic construct; identical to SEQ ID NO: 225

SEQ ID NO: 342 - protein, 326 aa, synthetic construct; identical to SEQ ID NO: 226

SEQ ID NO: 343 - protein, 327 aa, synthetic construct; identical to SEQ ID NO: 227

SEQ ID NO: 344 - protein, 327 aa, synthetic construct; identical to SEQ ID NO: 228

SEQ ID NO: 345 - protein, 327 aa, synthetic construct; identical to SEQ ID NO: 229

SEQ ID NO: 346 - protein, 327 aa, synthetic construct; identical to SEQ ID NO: 230

SEQ ID NO: 347 - protein, 327 aa, synthetic construct; identical to SEQ ID NO: 231

SEQ ID NO: 348 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLYITREPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPK

SEQ ID NO: 349 - protein, 325 aa, synthetic construct; identical to SEQ ID NO: 311

SEQ ID NO: 350 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVDHHDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHSHYTQKSLSLSPK

SEQ ID NO: 351 - protein, 327 aa, synthetic construct; identical to SEQ ID NO: 235

SEQ ID NO: 352 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 353 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 354 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 355 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 356 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 357 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 358 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 359 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 360 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 361 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 362 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 363 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 364 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 365 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 366 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 367 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 368 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 369 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 370 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 371 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 372 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 373 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 374 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 375 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 376 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 377 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 378 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 379 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 380 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 381 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 382 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 383 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 384 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 385 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 386 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 387 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 388 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 389 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 390 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 391 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 392 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 393 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 394 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 395 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 396 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 397 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 398 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 399 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 400 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 401 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 402 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYASTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 403 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 404 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 405 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 406 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 407 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 408 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

(19) United States
(12) Patent Application Publication
(10) Pub. No.: US 2026/0193366 A1
(43) Pub. Date: Jul. 9, 2026

(54) ANTIBODIES THAT BIND INTERLEUKIN 4 RECEPTOR ALPHA AND METHODS OF USE

(71) Applicant: Apogee Therapeutics, Inc., Waltham, MA (US)

(72) Inventors: Eric Franklin ZHU, Arlington, MA (US); Shawn Michael RUSSELL, Waltham, MA (US); Jason Zee Seug OH, Shrewsbury, MA (US); Kenneth Evan THOMPSON, Arlington, MA (US); Hussam SHAHEEN, Vega Alta, PR (US); Peter Evan HARWIN, Dorado, PR (US); Tomas KISELAK, Dorado, PR (US)

(21) Appl. No.: 19/425,771

(22) Filed: Dec. 18, 2025

Related U.S. Application Data
(63) Continuation of application No. 19/157,017, filed on Feb. 16, 2024.
(60) Provisional application No. 63/446,763, filed on Feb. 17, 2023.
(60) Provisional application No. 63/462,864, filed on Apr. 28, 2023.
(60) Provisional application No. 63/596,504, filed on Nov. 6, 2023.
(60) Provisional application No. 63/596,501, filed on Nov. 6, 2023.

(51) Int. Cl. C07K 16/28; A61K 39/00; A61P 29/00

(52) U.S. Cl. CPC C07K 16/2866; A61P 29/00; A61K 2039/505; A61K 2039/54; C07K 2317/14; C07K 2317/24; C07K 2317/52; C07K 2317/565; C07K 2317/76; C07K 2317/92; C07K 2317/94

(57) ABSTRACT

Described herein are novel and improved antibodies that bind Interleukin 4α Receptor (IL-4Rα) and methods of use thereof. In certain aspects, described herein are methods of inhibiting IL-4Rα biological activity. In certain aspects, described herein are pharmaceutical compositions comprising the anti-IL-4Rα antibodies. In certain aspects, the antibodies and methods described herein are used for treatment of an inflammatory disease or disorder associated with elevated levels of IL-4 and/or IgE.

### CROSS REFERENCE TO RELATED APPLICATIONS

This application is a Continuation of U.S. patent application Ser. No. 19/157,017, filed Aug. 15, 2025, pending, which application is a 35 U.S.C. § 371 national stage filing of International Application No. PCT/US2024/016235, filed on Feb. 16, 2024, which claims the benefit of and priority to U.S. Provisional Application No. 63/596,504, filed Nov. 6, 2023, U.S. Provisional Application No. 63/596,501, filed Nov. 6, 2023, U.S. Provisional Application No. 63/462,864, filed Apr. 28, 2023 and U.S. Provisional Application No. 63/446,763, filed Feb. 17, 2023. The entire contents of the aforementioned applications are incorporated herein by reference.

### SEQUENCE LISTING

The instant application contains a Sequence Listing which has been submitted electronically in XML format via EFS-Web and is hereby incorporated by reference in its entirety. Said XML copy, created on Mar. 13, 2024, is named AOJ-026USCN_SL.xml and is 431,847 bytes in size.

### FIELD OF THE INVENTION

Described herein are novel and improved antibodies that bind Interleukin 4a Receptor (IL-4Rα) and methods of use thereof, for example, in the treatment of an inflammatory disease or disorder associated with elevated levels of IL-4 and/or IgE.

### BACKGROUND

Interleukin 4 (IL-4) and interleukin 13 (IL-13) share a common receptor component, the Interleukin-4 Receptor Alpha (IL-4Rα) chain, that pairs with distinct subunits (Nelms, K., et al. (1999) Annu. Rev. Immunol. 17:701-738) and (Jensen, P. L. (2000) Stem Cells 18:61-62). IL-4Rα pairs with the common γc chain to form a type I IL-4R complex that is found predominantly in hematopoietic cells and is exclusive for IL-4. IL-4Rα also pairs with the Interleukin-13 Receptor alpha 1 (IL-13Rα1) subunit to form a type II IL-4R that binds both IL-4 and IL-13. The type II receptor is expressed on both hematopoietic and nonhematopoietic cells. IL-4, (also known as B cell stimulating factor or BSF-1) was originally characterized by its ability to stimulate the proliferation of B cells in response to low concentrations of antibodies directed to surface immunoglobulin. IL-4 has been shown to possess a broad spectrum of biological activities, including growth stimulation of T cells, mast cells, granulocytes, megakaryocytes and erythrocytes. IL-4 induces the expression of class II major histocompatibility complex molecules in resting B cells, and enhances the secretion of IgE and IgG1 isotypes by stimulated B cells.

The biological and immunological functions of β-lymphocytes, monocytes, dendritic cells, and fibroblasts are all affected by IL-4 and IL-13. These cytokines interact with IL-4R to initiate the type 2 inflammatory pathway, which results in Th2 cell differentiation, inflammation, and mucus production. The type-2 inflammatory pathway is first activated in allergic illnesses by aberrant cytokine release resulting from an imbalance of Th1 and Th2 differentiation. Th2 cells that have been activated release cytokines, including IL-4, IL-13, and IL-31, which prompt downstream B cells to undergo a change and produce IgE antibodies. Mast cells and basophils are then called upon to degranulate and release inflammatory substances. Simultaneously, the secreted IL-4 and IL-13 continue to bind to their respective receptors, such as IL-4Rα, that repeatedly promotes TH2 differentiation and subsequent inflammation.

The important role of IL-4 and IL-13 in the type-2 inflammation pathway identifies IL-4Rα as a potential target for individuals who exhibit aberrant type-2 inflammatory responses. Unlike medications that target IL-4 and IL-13 exclusively, targeting IL-4Rα would target both IL-4 and IL-13. Therefore, novel antibodies that bind IL-4Rα that modify type-2 inflammation to treat immune disorders are needed.

### SUMMARY

In a first aspect, disclosed herein are antibodies, such as recombinant human antibodies, that specifically bind human interleukin-4 receptor alpha (hIL-4Rα). In certain embodiments, the antibodies are characterized by binding to hIL-4Rα with high affinity and by the ability to neutralize hIL-4Rα activity. In specific embodiments, the human antibodies are capable of blocking hIL-13/hIL-13R1 complex binding to hIL-4Rα, and thus inhibit signaling by hIL-13. The antibodies can be full-length (for example, an IgG1 or IgG4 antibody) or may comprise only an antigen-binding portion (for example, a Fab, F(ab′)2 or scFv fragment), and may be modified to effect functionality, e.g., to abrogate residual effector functions (Reddy et al. (2000) J. Immunol. 164:1925-1933) and/or extend half-life.

In certain aspects, described herein is an isolated antibody that binds Interleukin (IL)-4 Receptor alpha (IL-4Rα) comprising a) comprising a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3; wherein: a. CDR-H1 comprises a sequence selected from the sequences set forth in SEQ ID NO: 1-4, 66-70 and 187-191; b. CDR-H2 comprises a sequence selected from the sequences set forth in SEQ ID NO: 5-16, and 71-90; c. CDR-H3 comprises a sequence selected from the sequences set forth in SEQ ID NO: 17-25 and 92-99, d. CDR-L1 comprises a sequence selected from the sequences set forth in SEQ ID NO: 26-40 and 100-107, e. CDR-L2 comprises a sequence selected from the sequences set forth in SEQ ID NO: 41-52 and 108-112; and f. CDR-L3 comprises a sequence selected from the sequences set forth in SEQ ID NO: 53-65.

In some embodiments, the antibody comprises: a. CDR-H1 comprising a sequence selected from the sequences set forth in SEQ ID NO: 1-4; b. CDR-H2 comprises a sequence selected from the sequences set forth in SEQ ID NO: 5-16; c. CDR-H3 comprises a sequence selected from the sequences set forth in SEQ ID NO: 17-25, d. CDR-L1 comprises a sequence selected from the sequences set forth in SEQ ID NO: 26-40, e. CDR-L2 comprises a sequence selected from the sequences set forth in SEQ ID NO: 41-52; and f. CDR-L3 comprises a sequence selected from the sequences set forth in SEQ ID NO: 53-65.

In some embodiments, the antibody comprises: a. CDR-H1 comprising a sequence selected from the sequences set forth in SEQ ID NO: 66-70; b. CDR-H2 comprises a sequence selected from the sequences set forth in SEQ ID NO: 71-78; c. CDR-H3 comprises a sequence selected from the sequences set forth in SEQ ID NO: 17-25, d. CDR-L1 comprises a sequence selected from the sequences set forth in SEQ ID NO: 26-40, e. CDR-L2 comprises a sequence selected from the sequences set forth in SEQ ID NO: 41-52; and f. CDR-L3 comprises a sequence selected from the sequences set forth in SEQ ID NO: 53-65.

In some embodiments, the antibody comprises: a. CDR-H1 comprising a sequence selected from the sequences set forth in SEQ ID NO: 187-191; b. CDR-H2 comprises a sequence selected from the sequences set forth in SEQ ID NO: 79-90; c. CDR-H3 comprises a sequence selected from the sequences set forth in SEQ ID NO: 92-99, d. CDR-L1 comprises a sequence selected from the sequences set forth in SEQ ID NO: 100-107, e. CDR-L2 comprises a sequence selected from the sequences set forth in SEQ ID NO: 108-112; and f. CDR-L3 comprises a sequence selected from the sequences set forth in SEQ ID NO: 53-65.

In some embodiments, the antibody does not comprise:
a. CDR-H1 set forth in SEQ ID NO: 1; CDR-H2 set forth in SEQ ID NO: 5; CDR-H3 set forth in SEQ ID NO: 17; CDR-L1 set forth in SEQ ID NO: 26; CDR-L2 set forth in SEQ ID NO: 41; and CDR-L3 set forth in SEQ ID NO: 53; or
b. CDR-H1 set forth in SEQ ID NO: 67; CDR-H2 set forth in SEQ ID NO: 71; CDR-H3 set forth in SEQ ID NO: 17; CDR-L1 set forth in SEQ ID NO: 26; CDR-L2 set forth in SEQ ID NO: 41; and CDR-L3 set forth in SEQ ID NO: 53; or
c. CDR-H1 set forth in SEQ ID NO: 187; CDR-H2 set forth in SEQ ID NO: 79; CDR-H3 set forth in SEQ ID NO: 91; CDR-L1 set forth in SEQ ID NO: 100; CDR-L2 set forth in SEQ ID NO: 108; and CDR-L3 set forth in SEQ ID NO: 53.

In some embodiments, the antibody does not comprise any combination of: a. a CDR-H1 set forth in any of SEQ ID NOs: 1, 67, or 187; b. a CDR-H2 set forth in any of SEQ ID NOs: 5, 71, or 79; c. a CDR-H3 set forth in any of SEQ ID NOs: 17 or 91; d. a CDR-L1 set forth in any of SEQ ID NOs: 26 or 100; e. a CDR-L2 set forth in any of SEQ ID NOs: 41 or 108; and f. a CDR-L3 set forth in SEQ ID NO: 53.

In some embodiments, the antibody comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in SEQ ID NO: 18 or 92; a CDR-L1 comprising the sequence set forth in any of SEQ ID NOs: 27 or 101; a CDR-L2 comprising the sequence set forth in any of SEQ ID NOs: 42 or 108; and a CDR-L3 comprising the sequence set forth in SEQ ID NO: 54.

In some embodiments, the antibody comprises a CDR-H1 comprising the sequence set forth in any of SEQ ID NOs: 2, 62, or 188; a CDR-H2 comprising the sequence set forth in any of SEQ ID NOs: 6, 71, or 80; a CDR-H3 comprising the sequence set forth in any of SEQ ID NOs: 18 or 92; a CDR-L1 comprising the sequence set forth in any of SEQ ID NOs: 28 or 100; a CDR-L2 comprising the sequence set forth in any of SEQ ID NOs: 42 or 108; and a CDR-L3 comprising the sequence set forth in SEQ ID NO: 55.

In some embodiments, the antibody comprises a CDR-H1 comprising the sequence set forth in any of SEQ ID NOs: 2, 67, or 188; a CDR-H2 comprising the sequence set forth in any of SEQ ID NOs: 7, 72, or 81; a CDR-H3 comprising the sequence set forth in any of SEQ ID NOs: 22 or 93; a CDR-L1 comprising the sequence set forth in any of SEQ ID NOs: 29 or 100; a CDR-L2 comprising the sequence set forth in any of SEQ ID NOs: 43 or 108; and a CDR-L3 comprising the sequence set forth in SEQ ID NO: 55.

In some embodiments, the antibody comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 18 or 92; a CDR-L1 comprising the sequence set forth in any of SEQ ID NOs: 28 or 100; a CDR-L2 comprising the sequence set forth in any of SEQ ID NOs: 45 or 108; and a CDR-L3 comprising the sequence set forth in SEQ ID NO: 55.

In some embodiments, the antibody comprises a CDR-H1 comprising the sequence set forth in any of SEQ ID NOs: 2, 67, or 188; a CDR-H2 comprising the sequence set forth in any of SEQ ID NOs: 8, 73, or 82; a CDR-H3 comprising the sequence set forth in any of SEQ ID NOs: 19 or 94; a CDR-L1 comprising the sequence set forth in any of SEQ ID NOs: 30 or 102; a CDR-L2 comprising the sequence set forth in any of SEQ ID NOs: 43 or 108; and a CDR-L3 comprising the sequence set forth in SEQ ID NO: 56.

In some embodiments, the antibody comprises a CDR-H1 comprising the sequence set forth in any of SEQ ID NOs: 2, 67, or 188; a CDR-H2 comprising the sequence set forth in any of SEQ ID NOs: 8, 73, or 82; a CDR-H3 comprising the sequence set forth in any of SEQ ID NOs: 20 or 95; a CDR-L1 comprising the sequence set forth in any of SEQ ID NOs: 31 or 103; a CDR-L2 comprising the sequence set forth in any of SEQ ID NOs: 43 or 108; and a CDR-L3 comprising the sequence set forth in SEQ ID NO: 57.

In some embodiments, the antibody comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 17 or 91; a CDR-L1 comprising the sequence set forth in any of SEQ ID NOs: 32 or 102; a CDR-L2 comprising the sequence set forth in any of SEQ ID NOs: 46 or 109; and a CDR-L3 comprising the sequence set forth in SEQ ID NO: 58.

In some embodiments, the antibody comprises a CDR-H1 comprising the sequence set forth in any of SEQ ID NOs: 2, 67, or 188; a CDR-H2 comprising the sequence set forth in any of SEQ ID NOs: 8, 73, or 82; a CDR-H3 comprising the sequence set forth in any of SEQ ID NOs: 20 or 95; a CDR-L1 comprising the sequence set forth in any of SEQ ID NOs: 32 or 102; a CDR-L2 comprising the sequence set forth in any of SEQ ID NOs: 45 or 108; and a CDR-L3 comprising the sequence set forth in SEQ ID NO: 59.

In some embodiments, the antibody comprises a CDR-H1 comprising the sequence set forth in any of SEQ ID NOs: 2, 68, or 189; a CDR-H2 comprising the sequence set forth in any of SEQ ID NOs: 6, 71, or 80; a CDR-H3 comprising the sequence set forth in any of SEQ ID NOs: 20 or 95; a CDR-L1 comprising the sequence set forth in any of SEQ ID NOs: 32 or 102; a CDR-L2 comprising the sequence set forth in any of SEQ ID NOs: 45 or 108; and a CDR-L3 comprising the sequence set forth in SEQ ID NO: 60.

In some embodiments, the antibody comprises a CDR-H1 comprising the sequence set forth in any of SEQ ID NOs: 2, 67, or 188; a CDR-H2 comprising the sequence set forth in any of SEQ ID NOs: 9, 77, or 83; a CDR-H3 comprising the sequence set forth in any of SEQ ID NOs: 18 or 92; a CDR-L1 comprising the sequence set forth in any of SEQ ID NOs: 29 or 100; a CDR-L2 comprising the sequence set forth in any of SEQ ID NOs: 43 or 108; and a CDR-L3 comprising the sequence set forth in SEQ ID NO: 60.

In some embodiments, the antibody comprises a CDR-H1 comprising the sequence set forth in any of SEQ ID NOs: 2, 67, or 188; a CDR-H2 comprising the sequence set forth in any of SEQ ID NOs: 9, 77, or 83; a CDR-H3 comprising the sequence set forth in any of SEQ ID NOs: 18 or 92; a CDR-L1 comprising the sequence set forth in any of SEQ ID NOs: 27 or 101; a CDR-L2 comprising the sequence set forth in any of SEQ ID NOs: 43 or 108; and a CDR-L3 comprising the sequence set forth in SEQ ID NO: 57.

In some embodiments, the antibody comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 18 or 92; a CDR-L1 comprising the sequence set forth in any of SEQ ID NOs: 27 or 101; a CDR-L2 comprising the sequence set forth in any of SEQ ID NOs: 43 or 108; and a CDR-L3 comprising the sequence set forth in SEQ ID NO: 61.

In some embodiments, the antibody comprises a CDR-H1 comprising the sequence set forth in any of SEQ ID NOs: 2, 67, or 188; a CDR-H2 comprising the sequence set forth in any of SEQ ID NOs: 10, 71, or 84; a CDR-H3 comprising the sequence set forth in any of SEQ ID NOs: 20 or 95; a CDR-L1 comprising the sequence set forth in any of SEQ ID NOs: 27 or 101; a CDR-L2 comprising the sequence set forth in any of SEQ ID NOs: 43 or 108; and a CDR-L3 comprising the sequence set forth in SEQ ID NO: 61.

In some embodiments, the antibody comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 18 or 92; a CDR-L1 comprising the sequence set forth in any of SEQ ID NOs: 28 or 100; a CDR-L2 comprising the sequence set forth in any of SEQ ID NOs: 43 or 108; and a CDR-L3 comprising the sequence set forth in SEQ ID NO: 61.

In some embodiments, the antibody comprises a CDR-H1 comprising the sequence set forth in any of SEQ ID NOs: 3, 69, or 190; a CDR-H2 comprising the sequence set forth in any of SEQ ID NOs: 8, 73, or 82; a CDR-H3 comprising the sequence set forth in any of SEQ ID NOs: 21 or 96; a CDR-L1 comprising the sequence set forth in any of SEQ ID NOs: 28 or 100; a CDR-L2 comprising the sequence set forth in any of SEQ ID NOs: 43 or 108; and a CDR-L3 comprising the sequence set forth in SEQ ID NO: 61.

In some embodiments, the antibody comprises a CDR-H1 comprising the sequence set forth in any of SEQ ID NOs: 2, 67, or 188; a CDR-H2 comprising the sequence set forth in any of SEQ ID NOs: 7, 72, or 81; a CDR-H3 comprising the sequence set forth in any of SEQ ID NOs: 20 or 95; a CDR-L1 comprising the sequence set forth in any of SEQ ID NOs: 28 or 100; a CDR-L2 comprising the sequence set forth in any of SEQ ID NOs: 42 or 108; and a CDR-L3 comprising the sequence set forth in SEQ ID NO: 62.

In some embodiments, the antibody comprises a CDR-H1 comprising the sequence set forth in any of SEQ ID NOs: 2, 67, or 188; a CDR-H2 comprising the sequence set forth in any of SEQ ID NOs: 11, 73, or 85; a CDR-H3 comprising the sequence set forth in any of SEQ ID NOs: 18 or 92; a CDR-L1 comprising the sequence set forth in any of SEQ ID NOs: 33 or 102; a CDR-L2 comprising the sequence set forth in any of SEQ ID NOs: 43 or 108; and a CDR-L3 comprising the sequence set forth in SEQ ID NO: 61.

In some embodiments, the antibody comprises a CDR-H1 comprising the sequence set forth in any of SEQ ID NOs: 23, 69, or 190; a CDR-H2 comprising the sequence set forth in any of SEQ ID NOs: 11, 73, or 85; a CDR-H3 comprising the sequence set forth in any of SEQ ID NOs: 19 or 94; a CDR-L1 comprising the sequence set forth in any of SEQ ID NOs: 34 or 100; a CDR-L2 comprising the sequence set forth in any of SEQ ID NOs: 43 or 108; and a CDR-L3 comprising the sequence set forth in SEQ ID NO: 60.

In some embodiments, the antibody comprises a CDR-H1 comprising the sequence set forth in any of SEQ ID NOs: 2, 67, or 188; a CDR-H2 comprising the sequence set forth in any of SEQ ID NOs: 11, 73, or 85; a CDR-H3 comprising the sequence set forth in any of SEQ ID NOs: 22 or 93; a CDR-L1 comprising the sequence set forth in any of SEQ ID NOs: 34 or 100; a CDR-L2 comprising the sequence set forth in any of SEQ ID NOs: 43 or 108; and a CDR-L3 comprising the sequence set forth in SEQ ID NO: 56.

In some embodiments, the antibody comprises a CDR-H1 comprising the sequence set forth in any of SEQ ID NOs: 3, 69, or 190; a CDR-H2 comprising the sequence set forth in any of SEQ ID NOs: 6, 71, or 80; a CDR-H3 comprising the sequence set forth in any of SEQ ID NOs: 23 or 97; a CDR-L1 comprising the sequence set forth in any of SEQ ID NOs: 28 or 100; a CDR-L2 comprising the sequence set forth in any of SEQ ID NOs: 42 or 108; and a CDR-L3 comprising the sequence set forth in SEQ ID NO: 61.

In some embodiments, the antibody comprises a CDR-H1 comprising the sequence set forth in any of SEQ ID NOs: 2, 67, or 188; a CDR-H2 comprising the sequence set forth in any of SEQ ID NOs: 7, 72, or 81; a CDR-H3 comprising the sequence set forth in any of SEQ ID NOs: 20, 18, or 95; a CDR-L1 comprising the sequence set forth in any of SEQ ID NOs: 27 or 101; a CDR-L2 comprising the sequence set forth in any of SEQ ID NOs: 43 or 108; and a CDR-L3 comprising the sequence set forth in SEQ ID NO: 61.

In some embodiments, the antibody comprises a CDR-H1 comprising the sequence set forth in any of SEQ ID NOs: 2, 67, or 188; a CDR-H2 comprising the sequence set forth in any of SEQ ID NOs: 7, 71, or 80; a CDR-H3 comprising the sequence set forth in any of SEQ ID NOs: 18 or 92; a CDR-L1 comprising the sequence set forth in any of SEQ ID NOs: 27 or 101; a CDR-L2 comprising the sequence set forth in any of SEQ ID NOs: 43 or 108; and a CDR-L3 comprising the sequence set forth in SEQ ID NO: 61.

In some embodiments, the antibody comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 24 or 98; a CDR-L1 comprising the sequence set forth in any of SEQ ID NOs: 36 or 102; a CDR-L2 comprising the sequence set forth in any of SEQ ID NOs: 45 or 108; and a CDR-L3 comprising the sequence set forth in SEQ ID NO: 61.

In some embodiments, the antibody comprises a CDR-H1 comprising the sequence set forth in any of SEQ ID NOs: 2, 67, or 188; a CDR-H2 comprising the sequence set forth in any of SEQ ID NOs: 8, 73, or 82; a CDR-H3 comprising the sequence set forth in any of SEQ ID NOs: 18 or 92; a CDR-L1 comprising the sequence set forth in any of SEQ ID NOs: 37 or 100; a CDR-L2 comprising the sequence set forth in any of SEQ ID NOs: 47 or 110; and a CDR-L3 comprising the sequence set forth in SEQ ID NO: 61.

In some embodiments, the antibody comprises a CDR-H1 comprising the sequence set forth in any of SEQ ID NOs: 2, 67, or 188; a CDR-H2 comprising the sequence set forth in any of SEQ ID NOs: 12, 74, or 86; a CDR-H3 comprising the sequence set forth in any of SEQ ID NOs: 24 or 98; a CDR-L1 comprising the sequence set forth in any of SEQ ID NOs: 28 or 100; a CDR-L2 comprising the sequence set forth in any of SEQ ID NOs: 43 or 108; and a CDR-L3 comprising the sequence set forth in SEQ ID NO: 61.

In some embodiments, the antibody comprises a CDR-H1 comprising the sequence set forth in any of SEQ ID NOs: 2, 67, or 189; a CDR-H2 comprising the sequence set forth in any of SEQ ID NOs: 13, 71, or 87; a CDR-H3 comprising the sequence set forth in any of SEQ ID NOs: 20 or 95; a CDR-L1 comprising the sequence set forth in any of SEQ ID NOs: 28 or 100; a CDR-L2 comprising the sequence set forth in any of SEQ ID NOs: 43 or 108; and a CDR-L3 comprising the sequence set forth in SEQ ID NO: 61.

In some embodiments, the antibody comprises a CDR-H1 comprising the sequence set forth in any of SEQ ID NOs: 2, 67, or 188; a CDR-H2 comprising the sequence set forth in any of SEQ ID NOs: 10, 71, or 84; a CDR-H3 comprising the sequence set forth in any of SEQ ID NOs: 20 or 95; a CDR-L1 comprising the sequence set forth in any of SEQ ID NOs: 34 or 100; a CDR-L2 comprising the sequence set forth in any of SEQ ID NOs: 43 or 108; and a CDR-L3 comprising the sequence set forth in SEQ ID NO: 57.

In some embodiments, the antibody comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 20 or 95; a CDR-L1 comprising the sequence set forth in any of SEQ ID NOs: 34 or 100; a CDR-L2 comprising the sequence set forth in any of SEQ ID NOs: 43 or 108; and a CDR-L3 comprising the sequence set forth in SEQ ID NO: 61.

In some embodiments, the antibody comprises a CDR-H1 comprising the sequence set forth in any of SEQ ID NOs: 1, 66, or 187; a CDR-H2 comprising the sequence set forth in any of SEQ ID NOs: 5, 71, or 79; a CDR-H3 comprising the sequence set forth in any of SEQ ID NOs: 17 or 91; a CDR-L1 comprising the sequence set forth in any of SEQ ID NOs: 38 or 105; a CDR-L2 comprising the sequence set forth in any of SEQ ID NOs: 48 or 111; and a CDR-L3 comprising the sequence set forth in SEQ ID NO: 63.

In some embodiments, the antibody comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 20 or 95; a CDR-L1 comprising the sequence set forth in any of SEQ ID NOs: 39 or 106; a CDR-L2 comprising the sequence set forth in any of SEQ ID NOs: 41 or 108; and a CDR-L3 comprising the sequence set forth in SEQ ID NO: 64.

In some embodiments, the antibody comprises a CDR-H1 comprising the sequence set forth in any of SEQ ID NOs: 2, 67, or 188; a CDR-H2 comprising the sequence set forth in any of SEQ ID NOs: 14, 75, or 88; a CDR-H3 comprising the sequence set forth in any of SEQ ID NOs: 20 or 95; a CDR-L1 comprising the sequence set forth in any of SEQ ID NOs: 39 or 106; a CDR-L2 comprising the sequence set forth in any of SEQ ID NOs: 49 or 112; and a CDR-L3 comprising the sequence set forth in SEQ ID NO: 63.

In some embodiments, the antibody comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 19 or 94; a CDR-L1 comprising the sequence set forth in any of SEQ ID NOs: 28 or 100; a CDR-L2 comprising the sequence set forth in any of SEQ ID NOs: 42 or 108; and a CDR-L3 comprising the sequence set forth in SEQ ID NO: 60.

In some embodiments, the antibody comprises a CDR-H1 comprising the sequence set forth in any of SEQ ID NOs: 2, 67, or 188; a CDR-H2 comprising the sequence set forth in any of SEQ ID NOs: 8, 73, or 82; a CDR-H3 comprising the sequence set forth in any of SEQ ID NOs: 18 or 92; a CDR-L1 comprising the sequence set forth in any of SEQ ID NOs: 31 or 103; a CDR-L2 comprising the sequence set forth in any of SEQ ID NOs: 43 or 108; and a CDR-L3 comprising the sequence set forth in SEQ ID NO: 58.

In some embodiments, the antibody comprises a CDR-H1 comprising the sequence set forth in any of SEQ ID NOs: 3, 69, or 190; a CDR-H2 comprising the sequence set forth in any of SEQ ID NOs: 5, 71, or 79; a CDR-H3 comprising the sequence set forth in any of SEQ ID NOs: 19 or 94; a CDR-L1 comprising the sequence set forth in any of SEQ ID NOs: 33 or 102; a CDR-L2 comprising the sequence set forth in any of SEQ ID NOs: 50 or 110; and a CDR-L3 comprising the sequence set forth in SEQ ID NO: 61.

In some embodiments, the antibody comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 18 or 92; a CDR-L1 comprising the sequence set forth in any of SEQ ID NOs: 30 or 101; a CDR-L2 comprising the sequence set forth in any of SEQ ID NOs: 51 or 108; and a CDR-L3 comprising the sequence set forth in SEQ ID NO: 61.

In some embodiments, the antibody comprises a CDR-H1 comprising the sequence set forth in any of SEQ ID NOs: 3, 67, or 190; a CDR-H2 comprising the sequence set forth in any of SEQ ID NOs: 15, 67, or 89; a CDR-H3 comprising the sequence set forth in any of SEQ ID NOs: 20 or 95; a CDR-L1 comprising the sequence set forth in any of SEQ ID NOs: 40 or 107; a CDR-L2 comprising the sequence set forth in any of SEQ ID NOs: 52 or 108; and a CDR-L3 comprising the sequence set forth in SEQ ID NO: 61.

In some embodiments, the antibody comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 20 or 95; a CDR-L1 comprising the sequence set forth in any of SEQ ID NOs: 30 or 102; a CDR-L2 comprising the sequence set forth in any of SEQ ID NOs: 47 or 110; and a CDR-L3 comprising the sequence set forth in SEQ ID NO: 61.

In some embodiments, the antibody comprises a CDR-H1 comprising the sequence set forth in any of SEQ ID NOs: 4, 70, or 191; a CDR-H2 comprising the sequence set forth in any of SEQ ID NOs: 11, 73, or 85; a CDR-H3 comprising the sequence set forth in any of SEQ ID NOs: 22, 18, or 93; a CDR-L1 comprising the sequence set forth in any of SEQ ID NOs: 30 or 102; a CDR-L2 comprising the sequence set forth in any of SEQ ID NOs: 43 or 108; and a CDR-L3 comprising the sequence set forth in SEQ ID NO: 60.

In some embodiments, the antibody comprises a CDR-H1 comprising the sequence set forth in any of SEQ ID NOs: 2, 67, or 188; a CDR-H2 comprising the sequence set forth in any of SEQ ID NOs: 14, 75, or 88; a CDR-H3 comprising the sequence set forth in any of SEQ ID NOs: 18 or 92; a CDR-L1 comprising the sequence set forth in any of SEQ ID NOs: 27 or 101; a CDR-L2 comprising the sequence set forth in any of SEQ ID NOs: 43 or 108; and a CDR-L3 comprising the sequence set forth in SEQ ID NO: 65.

In some embodiments, the antibody comprises a CDR-H1 comprising the sequence set forth in any of SEQ ID NOs: 2, 67, or 188; a CDR-H2 comprising the sequence set forth in any of SEQ ID NOs: 15, 76, or 89; a CDR-H3 comprising the sequence set forth in any of SEQ ID NOs: 19 or 94; a CDR-L1 comprising the sequence set forth in any of SEQ ID NOs: 28 or 100; a CDR-L2 comprising the sequence set forth in any of SEQ ID NOs: 43 or 108; and a CDR-L3 comprising the sequence set forth in SEQ ID NO: 62.

In some embodiments, the antibody comprises a CDR-H1 comprising the sequence set forth in any of SEQ ID NOs: 3, 69, or 190; a CDR-H2 comprising the sequence set forth in any of SEQ ID NOs: 9, 77, or 83; a CDR-H3 comprising the sequence set forth in any of SEQ ID NOs: 19 or 94; a CDR-L1 comprising the sequence set forth in any of SEQ ID NOs: 28 or 100; a CDR-L2 comprising the sequence set forth in any of SEQ ID NOs: 43 or 108; and a CDR-L3 comprising the sequence set forth in SEQ ID NO: 55.

In some embodiments, the antibody comprises a CDR-H1 comprising the sequence set forth in any of SEQ ID NOs: 2, 67, or 188; a CDR-H2 comprising the sequence set forth in any of SEQ ID NOs: 15, 76, or 89; a CDR-H3 comprising the sequence set forth in any of SEQ ID NOs: 24 or 98; a CDR-L1 comprising the sequence set forth in any of SEQ ID NOs: 34 or 100; a CDR-L2 comprising the sequence set forth in any of SEQ ID NOs: 42 or 108; and a CDR-L3 comprising the sequence set forth in SEQ ID NO: 61.

In some embodiments, the antibody comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 18 or 92; a CDR-L1 comprising the sequence set forth in any of SEQ ID NOs: 32 or 102; a CDR-L2 comprising the sequence set forth in any of SEQ ID NOs: 51 or 110; and a CDR-L3 comprising the sequence set forth in SEQ ID NO: 61.

In some embodiments, the antibody comprises a CDR-H1 comprising the sequence set forth in any of SEQ ID NOs: 2, 67, or 188; a CDR-H2 comprising the sequence set forth in any of SEQ ID NOs: 16, 78, or 90; a CDR-H3 comprising the sequence set forth in any of SEQ ID NOs: 19 or 94; a CDR-L1 comprising the sequence set forth in any of SEQ ID NOs: 28 or 100; a CDR-L2 comprising the sequence set forth in any of SEQ ID NOs: 45 or 108; and a CDR-L3 comprising the sequence set forth in SEQ ID NO: 57.

In some embodiments, the antibody comprises a CDR-H1 comprising the sequence set forth in any of SEQ ID NOs: 4, 70, or 191; a CDR-H2 comprising the sequence set forth in any of SEQ ID NOs: 16, 78, or 90; a CDR-H3 comprising the sequence set forth in any of SEQ ID NOs: 25 or 99; a CDR-L1 comprising the sequence set forth in any of SEQ ID NOs: 34 or 100; a CDR-L2 comprising the sequence set forth in any of SEQ ID NOs: 43 or 108; and a CDR-L3 comprising the sequence set forth in SEQ ID NO: 53.

In some embodiments, wherein the antibody comprises a heavy chain variable domain (VH) sequence selected from the sequences set forth in SEQ ID NO: 113-145.

In some embodiments, the antibody comprises a light chain variable domain (VL) sequence selected from the sequences set forth in SEQ ID NO: 146-186.

In some embodiments, the antibody comprises a VH sequence selected from the sequences set forth in SEQ ID NO: 113-145, and a VL sequence selected from the sequences set forth in SEQ ID NO: 146-186.

In certain aspects, described herein is an isolated antibody that binds IL-4Rα, wherein the antibody comprises a VH sequence selected from the sequences set forth in SEQ ID NO: 113-145, and a VL sequence selected from the sequences set forth in SEQ ID NO: 146-186.

In some embodiments, the antibody comprises a VH sequence set forth in SEQ ID NO: 114 and a VL sequence set forth in SEQ ID NO: 147.

In some embodiments, the antibody comprises a VH sequence set forth in SEQ ID NO: 114 and a VL sequence set forth in SEQ ID NO: 148.

In some embodiments, the antibody comprises a VH sequence set forth in SEQ ID NO: 115 and a VL sequence set forth in SEQ ID NO: 149.

In some embodiments, the antibody comprises a VH sequence set forth in SEQ ID NO: 114 and a VL sequence set forth in SEQ ID NO: 150.

In some embodiments, the antibody comprises a VH sequence set forth in SEQ ID NO: 116 and a VL sequence set forth in SEQ ID NO: 151.

In some embodiments, the antibody comprises a VH sequence set forth in SEQ ID NO: 117 and a VL sequence set forth in SEQ ID NO: 152.

In some embodiments, the antibody comprises a VH sequence set forth in SEQ ID NO: 118 and a VL sequence set forth in SEQ ID NO: 153.

In some embodiments, the antibody comprises a VH sequence set forth in SEQ ID NO: 117 and a VL sequence set forth in SEQ ID NO: 154.

In some embodiments, the antibody comprises a VH sequence set forth in SEQ ID NO: 119 and a VL sequence set forth in SEQ ID NO: 155.

In some embodiments, the antibody comprises a VH sequence set forth in SEQ ID NO: 120 and a VL sequence set forth in SEQ ID NO: 156.

In some embodiments, the antibody comprises a VH sequence set forth in SEQ ID NO: 120 and a VL sequence set forth in SEQ ID NO: 157.

In some embodiments, the antibody comprises a VH sequence set forth in SEQ ID NO: 114 and a VL sequence set forth in SEQ ID NO: 158.

In some embodiments, the antibody comprises a VH sequence set forth in SEQ ID NO: 121 and a VL sequence set forth in SEQ ID NO: 158.

In some embodiments, the antibody comprises a VH sequence set forth in SEQ ID NO: 114 and a VL sequence set forth in SEQ ID NO: 159.

In some embodiments, the antibody comprises a VH sequence set forth in SEQ ID NO: 123 and a VL sequence set forth in SEQ ID NO: 159.

In some embodiments, the antibody comprises a VH sequence set forth in SEQ ID NO: 124 and a VL sequence set forth in SEQ ID NO: 160.

In some embodiments, the antibody comprises a VH sequence set forth in SEQ ID NO: 125 and a VL sequence set forth in SEQ ID NO: 161.

In some embodiments, the antibody comprises a VH sequence set forth in SEQ ID NO: 126 and a VL sequence set forth in SEQ ID NO: 162.

In some embodiments, the antibody comprises a VH sequence set forth in SEQ ID NO: 127 and a VL sequence set forth in SEQ ID NO: 163.

In some embodiments, the antibody comprises a VH sequence set forth in SEQ ID NO: 128 and a VL sequence set forth in SEQ ID NO: 164.

In some embodiments, the antibody comprises a VH sequence set forth in SEQ ID NO: 124 and a VL sequence set forth in SEQ ID NO: 158.

In some embodiments, the antibody comprises a VH sequence set forth in SEQ ID NO: 129 and a VL sequence set forth in SEQ ID NO: 165.

In some embodiments, the antibody comprises a VH sequence set forth in SEQ ID NO: 130 and a VL sequence set forth in SEQ ID NO: 166.

In some embodiments, the antibody comprises a VH sequence set forth in SEQ ID NO: 131 and a VL sequence set forth in SEQ ID NO: 167.

In some embodiments, the antibody comprises a VH sequence set forth in SEQ ID NO: 132 and a VL sequence set forth in SEQ ID NO: 159.

In some embodiments, the antibody comprises a VH sequence set forth in SEQ ID NO: 133 and a VL sequence set forth in SEQ ID NO: 159.

In some embodiments, the antibody comprises a VH sequence set forth in SEQ ID NO: 121 and a VL sequence set forth in SEQ ID NO: 168.

In some embodiments, the antibody comprises a VH sequence set forth in SEQ ID NO: 134 and a VL sequence set forth in SEQ ID NO: 169.

In some embodiments, antibody comprises a VH sequence set forth in SEQ ID NO: 113 and a VL sequence set forth in SEQ ID NO: 170.

In some embodiments, the antibody comprises a VH sequence set forth in SEQ ID NO: 134 and a VL sequence set forth in SEQ ID NO: 171.

In some embodiments, the antibody comprises a VH sequence set forth in SEQ ID NO: 135 and a VL sequence set forth in SEQ ID NO: 172.

In some embodiments, the antibody comprises a VH sequence set forth in SEQ ID NO: 136 and a VL sequence set forth in SEQ ID NO: 173.

In some embodiments, the antibody comprises a VH sequence set forth in SEQ ID NO: 131 and a VL sequence set forth in SEQ ID NO: 174.

In some embodiments, the antibody comprises a VH sequence set forth in SEQ ID NO: 137 and a VL sequence set forth in SEQ ID NO: 175.

In some embodiments, the antibody comprises a VH sequence set forth in SEQ ID NO: 114 and a VL sequence set forth in SEQ ID NO: 176.

In some embodiments, the antibody comprises a VH sequence set forth in SEQ ID NO: 138 and a VL sequence set forth in SEQ ID NO: 177.

In some embodiments, the antibody comprises a VH sequence set forth in SEQ ID NO: 134 and a VL sequence set forth in SEQ ID NO: 178.

In some embodiments, the antibody comprises a VH sequence set forth in SEQ ID NO: 139 and a VL sequence set forth in SEQ ID NO: 179.

In some embodiments, the antibody comprises a VH sequence set forth in SEQ ID NO: 140 and a VL sequence set forth in SEQ ID NO: 180.

In some embodiments, the antibody comprises a VH sequence set forth in SEQ ID NO: 141 and a VL sequence set forth in SEQ ID NO: 181.

In some embodiments, the antibody comprises a VH sequence set forth in SEQ ID NO: 142 and a VL sequence set forth in SEQ ID NO: 182.

In some embodiments, the antibody comprises a VH sequence set forth in SEQ ID NO: 143 and a VL sequence set forth in SEQ ID NO: 183.

In some embodiments, the antibody comprises a VH sequence set forth in SEQ ID NO: 114 and a VL sequence set forth in SEQ ID NO: 184.

In some embodiments, the antibody comprises a VH sequence set forth in SEQ ID NO: 144 and a VL sequence set forth in SEQ ID NO: 185.

In some embodiments, the antibody comprises a VH sequence set forth in SEQ ID NO: 145 and a VL sequence set forth in SEQ ID NO: 186.

In some embodiments, the antibody is a humanized, fully human or chimeric antibody.

In some embodiments, the antibody is a fully human antibody.

In some embodiments, the antibody comprises a heavy chain human constant region of a class selected from IgG, IgA, IgD, IgE, and IgM.

In some embodiments, the human Fc region comprises a human heavy chain constant region of the class IgG and a subclass selected from IgG1, IgG2, IgG3, and IgG4.

In some embodiments, the human Fc region comprises a human IgG1 Fc.

In some embodiments, the human Fc region comprises a human IgG4 Fc.

In some embodiments, the human Fc region comprises a human IgG2 Fc.

In some embodiments, the heavy chain comprises a constant heavy chain sequence selected from the sequences set forth in SEQ ID NO: 192-235 and 251-407.

In certain aspects, described herein is an isolated antibody that binds Interleukin (IL)-4 Receptor alpha (IL-4Rα), comprising: a) a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3; wherein the antibody comprises a CDR-H1 comprising the sequence set forth in any of SEQ ID NOs: 1, 66, or 187; a CDR-H2 comprising the sequence set forth in any of SEQ ID NOs: 5, 71, or 79; a CDR-H3 comprising the sequence set forth in any of SEQ ID NOs: 17 or 91; a CDR-L1 comprising the sequence set forth in any of SEQ ID NOs: 26 or 100; a CDR-L2 comprising the sequence set forth in any of SEQ ID NOs: 41 or 108; and a CDR-L3 comprising the sequence set forth in SEQ ID NO: 53; and wherein the heavy chain comprises a constant heavy chain sequence selected from the sequences set forth in SEQ ID NO: 192-235 and 251-407. In some embodiments, the antibody comprises a VH sequence set forth in SEQ ID NO: 113. In some embodiments, the antibody comprises a VL sequence set forth in SEQ ID NO: 146.

In some embodiments, the light chain comprises a constant light chain sequence comprising a sequence set forth in SEQ ID NO: 236.

In some embodiments, the Fc region comprises one or more amino acid substitutions, wherein the one or more substitutions result in an increase in one or more of antibody half-life, ADCC activity, ADCP activity, and/or CDC activity compared with the Fc without the one or more substitutions.

In some embodiments, the Fc region comprises one or more amino acid substitutions, wherein the one or more substitutions result in a decrease in one or more of ADCC activity, ADCP activity, and/or CDC activity compared with the Fc without the one or more substitutions.

In some embodiments, the one or more amino acid substitutions results in increased antibody half-life compared to an antibody comprising a wild-type Fc region.

In some embodiments, the one or more amino acid substitutions is selected from the group consisting of S228P (SP), M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W. In some embodiments, the one or more amino acid substitutions is a combination of amino acid substitutions selected from the group consisting of M428L/N434S (LS); M252Y/S254T/T256E (YTE); T250Q/M428L; T307A/E380A/N434A; T256D/T307Q (DQ); T256D/T307W (DW); M252Y/T256D (YD); T307Q/Q311V/A378V (QVV); T256D/H285D/T307R/Q311V/A378V (DDRVV); L309D/Q311H/N434S (DHS); S228P/L235E (SPLE); L234A/L235A (LALA), M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS; D265A/LS; LALA/LS; LALAGA/LS; LALAPG/LS; N297A/DHS; D265A/DHS; LALA/DHS; LAGA/DHS; LALAGA/DHS; LALAPG/DHS; SP/YTE; SPLE/YTE; SP/LS; SPLE/LS, SP/DHS; SPLE/DHS; N297A/LA; D265A/LA, LALA/LA, LAGA/LA, LALAGA/LA, LALAPG/LA, N297A/N434A; D265A/N434A; LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.

In some embodiments, the Fe region binds to Neonatal Fe receptor (FcRn).

In some embodiments, the Fe region binds an FcRn with higher affinity at pH 6.0 compared to an antibody comprising a wild-type Fe region.

In some embodiments, the Fe region binds to FcRn with a KD of <1×10⁻⁷ M at pH 6.0.

In some embodiments, the antibody is a monoclonal antibody.

In some embodiments, the antibody binds an IL-4Rα sequence set forth in SEQ ID NO: 237-240.

In some embodiments, the antibody binds to an IL-4Rα sequence set forth in SEQ ID NO: 237-240 with a KD of less than or equal to about 1, 2, 3, 4, 5, 6, 7, 8, 9×10⁻⁹ M, as measured by surface plasmon resonance (SPR).

In some embodiments, the antibody binds to an IL-4Rα sequence set forth in SEQ ID NO: 237-240 with a KD of less than or equal to about 1×10⁻¹⁰ M, as measured by surface plasmon resonance (SPR).

In some embodiments, the antibody binds to human IL-4Rα with a KD of less than or equal to about 1×10⁻⁹ M, as measured by surface plasmon resonance (SPR).

In some embodiments, the antibody exhibits a melting temperature greater than 68° C. as measured by Differential Scanning Fluorometry (DSF).

In some embodiments, the antibody exhibits a melting temperature greater than 75° C. as measured by Differential Scanning Fluorometry (DSF).

In some embodiments, the antibody exhibits a aggregation temperature equal to or greater than 71.2° C. as measured by Differential Scanning Fluorometry (DSF).

In some embodiments, the antibody has a retention time of 15.2 minutes or less as measured by hydrophobic interaction chromatography.

In some embodiments, the antibody does not have a heavy chain variable region sequence set forth in SEQ ID NO. 113.

In some embodiments, the antibody does not have a heavy chain sequence set forth in SEQ ID NO. 146.

In certain embodiments, the isolated antibody is used in the treatment of an inflammatory disorder or disease. In certain embodiments, the isolated antibody is used in the treatment of atopic dermatitis. In certain embodiments, the isolated antibody is used in the treatment of idiopathic pulmonary fibrosis. In certain embodiments, the isolated antibody is used in the treatment of alopecia areata. In certain embodiments, the isolated antibody is used in the treatment of chronic sinusitis with nasal polyps. In certain embodiments, the isolated antibody is used in the treatment of Chronic Rhinosinusitis without Nasal Polyps (CRSsNP). In certain embodiments, the isolated antibody is used in the treatment of eosinophilic esophagitis (EoE). In certain embodiments, the isolated antibody is used in the treatment of Eosinophilic gastrointestinal disorder or disease (ENID) selected from the group consisting of Eosinophilic Gastritis (EoG), Eosinophilic Enteritis (EoN), Eosinophilic Colitis (EoC), and Eosinophilic Gastroenteritis (EGE). In certain embodiments, the isolated antibody is used in the treatment of Churg-Strauss syndrome/Eosinophilic granulomatosis with polyangiitis (EGPA). In certain embodiments, the isolated antibody is used in the treatment of Prurigo Nodularis (PN). In certain embodiments, the isolated antibody is used in the treatment of Chronic Spontaneous Urticaria (CSU). In certain embodiments, the isolated antibody is used in the treatment of Chronic Pruritis of Unknown Origin (CPUO). In certain embodiments, the isolated antibody is used in the treatment of Bullous Pemphigoid (BP). In certain embodiments, the isolated antibody is used in the treatment of Cold Inducible Urticaria (ColdU). In certain embodiments, the isolated antibody is used in the treatment of Allergic Fungal Rhinosinusitis (AFRS). In certain embodiments, the isolated antibody is used in the treatment of Allergic Bronchopulmonary Aspergillosis (ABPA). In certain embodiments, the isolated antibody is used in the treatment of Chronic Obstructive Pulmonary Disease (COPD). In certain embodiments, the isolated antibody is used in the treatment of inflammatory bowel disease, such as Crohn disease or ulcerative colitis. In certain embodiments, the isolated antibody is used in the treatment of lupus. In certain embodiments, the isolated antibody is used in the treatment of rheumatoid arthritis.

In certain aspects, disclosed herein is an isolated polynucleotide or set of polynucleotides encoding the antibody of any of the above claims, a VH thereof, a VL thereof, a light chain thereof, a heavy chain thereof, or an antigen-binding portion thereof, and optionally, wherein the polynucleotide or set of polynucleotides comprises cDNA.

In certain aspects, disclosed herein is a vector or set of vectors comprising the polynucleotide or set of polynucleotides discloses herein.

In certain aspects, disclosed herein is a host cell comprising the polynucleotide or set of polynucleotides or the vector or set of vectors disclosed herein.

In certain aspects, disclosed herein is a method of producing an antibody, the method comprising expressing the antibody with the host cell disclosed herein and isolating the expressed antibody.

In certain aspects, disclosed herein is a pharmaceutical composition comprising the antibody of any one of the embodiments disclosed herein and a pharmaceutically acceptable excipient.

In certain aspects, disclosed herein is a kit comprising the antibody of any one of the embodiments discloses herein or a pharmaceutical composition disclosed herein and instructions for use.

In certain aspects, described herein is a method for treating an inflammatory disorder or disease in a mammalian subject in need thereof, the method comprising administering to the mammalian subject a therapeutically effective amount an antibody described herein or a pharmaceutical composition described herein. In certain embodiments of the methods described herein, the inflammatory disorder or disease is atopic dermatitis. In certain embodiments, the inflammatory disorder or disease is asthma. In certain embodiments, the inflammatory disorder or disease is idiopathic pulmonary fibrosis. In certain embodiments of the methods described herein, the inflammatory disorder or disease is alopecia areata. In certain embodiments, the inflammatory disorder or disease is chronic sinusitis with nasal polyps. In certain embodiments, the inflammatory disorder or disease is Chronic Rhinosinusitis without Nasal Polyps (CRSsNP). In certain embodiments, the inflammatory disorder or disease is eosinophilic esophagitis (EoE). In certain embodiments, the inflammatory disorder or disease is an Eosinophilic gastrointestinal disorder or disease (ENID) selected from the group consisting of Eosinophilic Gastritis (EoG), Eosinophilic Enteritis (EoN), Eosinophilic Colitis (EoC), and Eosinophilic Gastroenteritis (EGE). In certain embodiments, the inflammatory disorder or disease is Churg-Strauss syndrome/Eosinophilic granulomatosis with polyangiitis (EGPA). In certain embodiments, the inflammatory disorder or disease is Prurigo Nodularis (PN). In certain embodiments, the inflammatory disorder or disease is Chronic Spontaneous Urticaria (CSU). In certain embodiments, the inflammatory disorder or disease is Chronic Pruritis of Unknown Origin (CPUO). In certain embodiments, the inflammatory disorder or disease is Bullous Pemphigoid (BP). In certain embodiments, the inflammatory disorder or disease is Cold Inducible Urticaria (ColdU). In certain embodiments, the inflammatory disorder or disease is Allergic Fungal Rhinosinusitis (AFRS). In certain embodiments, the inflammatory disorder or disease is Allergic Bronchopulmonary Aspergillosis (ABPA). In certain embodiments, the inflammatory disorder or disease is Chronic Obstructive Pulmonary Disease (COPD). In certain embodiments, the inflammatory disorder or disease is inflammatory bowel disease, such as Crohn disease or ulcerative colitis. In certain embodiments, the inflammatory disorder or disease is lupus. In certain embodiments, the inflammatory disorder or disease is rheumatoid arthritis.

In certain aspects, disclosed herein is a method for treating a pathology associated with elevated levels of IL-4Rα in a mammalian subject in need thereof, the method comprising administering to the mammalian subject a therapeutically effective amount the antibody of any one of the embodiments disclosed herein or a pharmaceutical composition disclosed herein.

In certain aspects, disclosed herein is a method of reducing biological activity of IL-4Rα in a mammalian subject in need thereof, the method comprising administering to the mammalian subject a therapeutically effective amount the antibody of any one of the embodiments disclosed herein or a pharmaceutical composition disclosed herein.

In certain aspects, disclosed herein is a method of inhibiting the TH2 type allergic response in a mammalian subject in need thereof, the method comprising administering to the mammalian subject a therapeutically effective amount the antibody of any one of the embodiments disclosed herein or a pharmaceutical composition disclosed herein.

In certain aspects, disclosed herein is a method of preventing an inflammatory disorder or disease in a mammalian subject in need thereof, the method comprising administering to the mammalian subject a therapeutically effective amount the antibody of any one of the embodiments disclosed herein or a pharmaceutical composition disclosed herein.

### BRIEF DESCRIPTION OF FIGURES

FIG. 1A and FIG. 1B show the ability of exemplary antibodies and dupilumab to inhibit IL-4 and IL-13 binding.

FIG. 2A and FIG. 2B show the ability of exemplary antibodies and dupilumab to inhibit IL-4-induced and IL-13-induced STAT6 phosphorylation (pSTAT6).

FIG. 3A and FIG. 3B show the ability of exemplary antibodies and dupilumab to inhibit IL-4- and/or IL-13-induced TARC secretion.

FIG. 4A and FIG. 4B show the ability of exemplary antibodies and dupilumab to inhibit IL-4-induced and IL-134-induced proliferation of TF-1 cells.

FIG. 5A and FIG. 5B show the serum concentration of exemplary antibodies (Construct 13 (mAb422), Construct 38 (mAb471), and dupilumab) over time after a single intravenous (FIG. 5A) or subcutaneous (FIG. 5B) administration of 25 mg/kg in non-human primates.

### DETAILED DESCRIPTION

### Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular form “a”, “an”, and “the” includes plural references unless indicated otherwise.

It is understood that aspects and embodiments of the invention described herein include “comprising,” “consisting,” and “consisting essentially of” aspects and embodiments.

For all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as ‘consisting essentially of’ a component, the composition may additionally contain any amount of pharmaceutically acceptable carriers, vehicles, or diluents and other such components which do not substantially affect the condition being treated.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations. A “recombinant host cell” or “host cell” refers to a cell that includes an exogenous polynucleotide, regardless of the method used for insertion, for example, direct uptake, transduction, f-mating, or other methods known in the art to create recombinant host cells.

As used herein, the term “eukaryote” refers to organisms belonging to the phylogenetic domain Eucarya such as animals (including but not limited to, mammals, insects, reptiles, birds, etc.), ciliates, plants (including but not limited to, monocots, dicots, algae, etc.), fungi, yeasts, flagellates, microsporidia, protists, etc.

As used herein, the term “prokaryote” refers to prokaryotic organisms. For example, a non-eukaryotic organism can belong to the Eubacteria (including but not limited to, Escherichia coli, Thermus thermophilus, Bacillus stearothermophilus, Pseudomonas fluorescens, Pseudomonas aeruginosa, Pseudomonas putida, etc.) phylogenetic domain, or the Archaea (including but not limited to, Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, etc.) phylogenetic domain.

An “effective amount” or “therapeutically effective amount” as used herein refers to an amount of therapeutic compound, such as an anti-IL-4R antibody, administered to an individual, either as a single dose or as part of a series of doses, which is effective to produce or contribute to a desired therapeutic effect, either alone or in combination with another therapeutic modality. Examples of a desired therapeutic effect is reducing an aberrant immune response, slowing or delaying disease development, stabilization of disease, and amelioration of one or more symptoms. An effective amount may be given in one or more dosages.

The term “treating” (and variations thereof such as “treat” or “treatment”) refers to clinical intervention in an attempt to alter the natural course of a disease or condition in a subject in need thereof. Treatment can be performed during the course of clinical pathology. Desirable effects of treatment include preventing recurrence of disease, alleviation of symptoms, diminishment of any direct or indirect pathological consequences of the disease, decreasing the rate of disease progression, amelioration or palliation of the disease state, and remission or improved prognosis.

The term “sufficient amount” means an amount sufficient to produce a desired effect, e.g., an amount sufficient to modulate an immune response in a subject.

As used herein, the term “subject” or “individual” means a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with an antibody provided herein. In some aspects, the disease or condition is a cancer. In some aspects, the disease or condition is a viral infection.

The term “in vitro” refers to processes that occur in a living cell growing separate from a living organism, e.g., growing in tissue culture.

The term “in vivo” refers to processes that occur in a living organism.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

The term “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The terms “co-administration”, “co-administer”, and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to the administration of a second therapeutic agent.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “about” indicates and encompasses an indicated value and a range above and below that value. In certain embodiments, the term “about” indicates the designated value 10%, 5%, or 1%. In certain embodiments, where applicable, the term “about” indicates the designated value(s) one standard deviation of that value(s).

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

For any of the structural and functional characteristics described herein, methods of determining these characteristics are known in the art.

The term “optionally” is meant, when used sequentially, to include from one to all of the enumerated combinations and contemplates all sub-combinations.

The term “amino acid” refers to, for example, the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “affinity” refers to the strength of the sum total of non-covalent interactions between a single binding site of a molecule (e.g., an antibody) and its binding partner (e.g., an antigen or epitope). Unless indicated otherwise, as used herein, “affinity” refers to intrinsic binding affinity, which reflects a 1:1 interaction between members of a binding pair (e.g., antibody and antigen or epitope).

The term “kd” (sec-1), as used herein, refers to the dissociation rate constant of a particular antibody-antigen interaction. This value is also referred to as the koff value.

The term “ka” (M-1×sec-1), as used herein, refers to the association rate constant of a particular antibody-antigen interaction. This value is also referred to as the kon value.

The term “KD” (M), as used herein, refers to the dissociation equilibrium constant of a particular antibody-antigen interaction. KD=kd/ka. In some embodiments, the affinity of an antibody is described in terms of the KD for an interaction between such antibody and its antigen. For clarity, as known in the art, a smaller KD value indicates a higher affinity interaction, while a larger KD value indicates a lower affinity interaction.

The term “KA” (M-1), as used herein, refers to the association equilibrium constant of a particular antibody-antigen interaction. KA=ka/kd.

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies.

A “anti-IL-4Rα antibody,” “IL-4Rα antibody,” or “IL-4Rα specific antibody” is an antibody, as provided herein, which specifically binds to the antigen IL-4Rα.

The term “epitope” means a portion of an antigen that specifically binds to an antibody.

The term “hypervariable region” or “HVR”, as used herein, refers to each of the regions of an antibody variable domain which are hypervariable in sequence and/or form structurally defined loops (“hypervariable loops”).

The term “antigen-binding domain” means the portion of an antibody that is capable of specifically binding to an antigen or epitope.

The term “chimeric antibody” refers to an antibody in which a portion of the heavy and/or light chain is derived from a particular source or species, while the remainder of the heavy and/or light chain is derived from a different source or species.

The term “human antibody” or “fully human antibody” refers to an antibody which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

The term “humanized antibody” refers to a protein having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject.

The term “multispecific antibody” refers to an antibody that comprises two or more different antigen-binding domains that collectively specifically bind two or more different epitopes.

A “monospecific antibody” is an antibody that comprises one or more binding sites that specifically bind to a single epitope. An example of a monospecific antibody is a naturally occurring IgG molecule which, while divalent (i.e., having two antigen-binding domains), recognizes the same epitope at each of the two antigen-binding domains. The binding specificity may be present in any suitable valency.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “single-chain” refers to a molecule comprising amino acid monomers linearly linked by peptide bonds. In a particular such embodiment, the C-terminus of the Fab light chain is connected to the N-terminus of the Fab heavy chain in the single-chain Fab molecule. As described in more detail herein, an scFv has a variable domain of light chain (VL) connected from its C-terminus to the N-terminal end of a variable domain of heavy chain (VH) by a polypeptide chain. Alternately, the scFv comprises a polypeptide chain wherein the C-terminal end of the VH is connected to the N-terminal end of VL by a polypeptide chain.

The “Fab fragment” (also referred to as fragment antigen-binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region (HVR)) that are involved in antigen-binding. Fab′ fragments differ from Fab fragments by the addition of a few residues at the carboxy terminus of the heavy chain CH1 domain including one or more cysteines from the antibody hinge region.

“F(ab′)2” fragments contain two Fab′ fragments joined, near the hinge region, by disulfide bonds. F(ab′)2 fragments may be generated, for example, by recombinant methods or by pepsin digestion of an intact antibody. The F(ab′) fragments can be dissociated, for example, by treatment with β-mercaptoethanol.

“Fv” fragments comprise a non-covalently-linked dimer of one heavy chain variable domain and one light chain variable domain.

“Single-chain Fv” or “sFv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding. For a review of scFv, see Pluckthun in The Pharmacology of Monoclonal Antibodies, vol. 113, Rosenburg and Moore eds., Springer-Verlag, New York, pp. 269-315 (1994). HER2 antibody scFv fragments are described in WO93/16185; U.S. Pat. Nos. 5,571,894; and 5,587,458.

“scFv-Fc” fragments comprise an scFv attached to an Fc domain. For example, an Fc domain may be attached to the C-terminal of the scFv. The Fc domain may follow the VH or VL, depending on the orientation of the variable domains in the scFv (i.e., VH-VL or VL-VH). Any suitable Fc domain known in the art or described herein may be used. In some cases, the Fc domain comprises an IgG4 Fc domain.

The term “single domain antibody” or “sdAb” refers to a molecule in which one variable domain of an antibody specifically binds to an antigen without the presence of the other variable domain. Single domain antibodies, and fragments thereof, are described in Arabi Ghahroudi et al. (1998) FEBS Letters 414:521-526 and Muyldermans et al. (2001) Trends in Biochem. Sci. 26:230-245, each of which is incorporated by reference in its entirety. Single domain antibodies are also known as sdAbs or nanobodies. SdAbs are fairly stable and easy to express as fusion partner with the Fc chain of an antibody (Harmsen M M, De Haard H J (2007) “Properties, production, and applications of camelid single-domain antibody fragments”. Appl. Microbiol Biotechnol. 77(1): 13-22).

The terms “full length antibody,” “intact antibody,” and “whole antibody” are used herein interchangeably to refer to an antibody having a structure substantially similar to a naturally occurring antibody structure and having heavy chains that comprise an Fe region. For example, when used to refer to an IgG molecule, a “full length antibody” is an antibody that comprises two heavy chains and two light chains.

The term “antibody fragment” refers to an antibody that comprises a portion of an intact antibody, such as the antigen-binding or variable region of an intact antibody. Antibody fragments include, for example, Fv fragments, Fab fragments, F(ab′)2 fragments, Fab′ fragments, scFv (sFv) fragments, and scFv-Fc fragments.

The term “Fc domain” or “Fe region” herein is used to define a C-terminal region of an immunoglobulin heavy chain that contains at least a portion of the constant region. The term includes native sequence Fe regions and variant Fe regions.

The term “substantially purified” refers to a construct described herein, or variant thereof that may be substantially or essentially free of components that normally accompany or interact with the protein as found in its naturally occurring environment, i.e. a native cell, or host cell in the case of recombinantly produced antibody that in certain embodiments, is substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., using publicly available computer software such as BLAST, BLASTP, BLASTN, BLAST-2, ALIGN, MEGALIGN (DNASTAR), CLUSTALW, CLUSTAL OMEGA, or MUSCLE software or other algorithms available to persons of skill) or by visual inspection. Software for performing BLAST analyses is publicly available through the National Center for Biotechnology Information (ncbi.nlm.nih.gov). Those skilled in the art can determine appropriate parameters for aligning sequences, including any algorithms needed to achieve maximal alignment over the full length of the sequences being compared. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman (1981) Adv. Appl. Math. 2:482, by the homology alignment algorithm of Needleman & Wunsch (1970) J. Mol. Biol. 48:443, by the search for similarity method of Pearson & Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of an algorithm that is suitable for determining percent sequence identity and sequence similarity is the BLAST algorithm, which is described in Altschul et al. (1990) J. Mol. Biol. 215:403-410. Software for performing BLAST analyses is publicly available through the National Center for Biotechnology Information (www.ncbi.nlm.nih.gov/).

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

It must be noted that, as used in the specification and the appended claims, the singular forms “a,” “an” and “the” include plural referents unless the context clearly dictates otherwise.

### Anti-IL-4Rα Antibodies

### Antibody Structure

The present application provides antibodies and compositions comprising an antibody which binds IL-4 Receptor alpha (IL-4Rα).

The recognized immunoglobulin genes include the kappa, lambda, alpha, gamma, delta, epsilon and mu constant region genes, as well as the myriad immunoglobulin variable region genes. Light chains are classified as either kappa or lambda. The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

An exemplary immunoglobulin (antibody) structural unit is composed of two pairs of polypeptide chains, each pair having one “light” (about 25 kD) and one “heavy” chain (about 50-70 kD). The N-terminal domain of each chain defines a variable region of about 100 to 110 or more amino acids primarily responsible for antigen recognition. The terms variable light chain (VL) and variable heavy chain (VH) refer to these light and heavy chain domains respectively. The IgG1 heavy chain comprises the VH, CH1, CH2, and CH3 domains, respectively, from the N to C-terminus. The light chain comprises the VL and CL domains from N to C terminus. The IgG1 heavy chain comprises a hinge between the CH1 and CH2 domains. In certain embodiments, the immunoglobulin constructs comprise at least one immunoglobulin domain from IgG, IgM, IgA, IgD, or IgE connected to a therapeutic polypeptide. In some embodiments, the immunoglobulin domain found in an antibody provided herein, is from or is derived from an immunoglobulin based construct such as a diabody or a nanobody. In certain embodiments, the immunoglobulin constructs described herein comprise at least one immunoglobulin domain from a heavy chain antibody such as a camelid antibody. In certain embodiments, the immunoglobulin constructs provided herein comprise at least one immunoglobulin domain from a mammalian antibody such as a bovine antibody, a human antibody, a camelid antibody, a mouse antibody or any chimeric antibody.

In some embodiments, the antibodies provided herein comprise a heavy chain. In one embodiment, the heavy chain is an IgA. In one embodiment, the heavy chain is an IgD. In one embodiment, the heavy chain is an IgE. In one embodiment, the heavy chain is an IgG. In one embodiment, the heavy chain is an IgM. In one embodiment, the heavy chain is an IgG1. In one embodiment, the heavy chain is an IgG2. In one embodiment, the heavy chain is an IgG3. In one embodiment, the heavy chain is an IgG4. In one embodiment, the heavy chain is an IgA1. In one embodiment, the heavy chain is an IgA2.

In some embodiments, an antibody is an IgG1 antibody. In some embodiments, an antibody is an IgG3 antibody. In some embodiments, an antibody is an IgG2 antibody. In some embodiments, an antibody is an IgG4 antibody.

Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen-binding regions. This particular region has been described by Kabat et al., U.S. Dept. of Health and Human Services, Sequences of Proteins of Immunological Interest (1983) and by Chothia et al. (1987) J Mol Biol 196:901-917, where the definitions include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

The amino acid sequence boundaries of a CDR can be determined by one of skill in the art using any of a number of known numbering schemes, including those described by Kabat et al., supra (“Kabat” numbering scheme); Al-Lazikani et al. (1997) J. Mol. Biol. 273:927-948 (“Chothia” numbering scheme); MacCallum et al. (1996) J. Mol. Biol. 262:732-745 (“Contact” numbering scheme); Lefranc et al. (2003) Dev. Comp. Immunol. 27:55-77 (“IMGT” numbering scheme); and Honegge and Plückthun, (2001) J. Mol. Biol. 309:657-70 (“AHo” numbering scheme); each of which is incorporated by reference in its entirety.

TABLE 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

CDRs may be assigned, for example, using antibody numbering software, such as Abnum, available at www.bioinf.org.uk/abs/abnum/, and described in Abhinandan and Martin, (2008) Immunology 45:3832-3839, incorporated by reference in its entirety.

TABLE 1
TABLE 1: Residues in CDRs according to
Kabat and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

One example of an antigen-binding domain is an antigen-binding domain formed by a VH-VL dimer of an antibody. Another example of an antigen-binding domain is an antigen-binding domain formed by diversification of certain loops from the tenth fibronectin type III domain of an Adnectin. An antigen-binding domain can include CDRs 1, 2, and 3 from a heavy chain in that order; and CDRs 1, 2, and 3 from a light chain in that order.

Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to IL-4Rα variants with different point-mutations, or to chimeric IL-4Rα variants.

To screen for antibodies which bind to an epitope on a target antigen bound by an antibody of interest (e.g., IL-4Rα), a routine cross-blocking assay such as that described in Antibodies, A Laboratory Manual, Cold Spring Harbor Laboratory, Ed Harlow and David Lane (1988), can be performed. Alternatively, or additionally, epitope mapping can be performed by methods known in the art.

Chimeric antibodies are antibodies in which a portion of the heavy and/or light chain is derived from a particular source or species, while the remainder of the heavy and/or light chain is derived from a different source or species.

Human, or fully human, antibodies are antibodies which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies can be found in U.S. Pat. Nos. 6,054,297; 5,886,152; and 5,877,293. For further details, see Jones et al. (1986) Nature 321:522-525; Riechmann et al. (1988) Nature 332:323-329; and Presta (1992) Curr. Op. Struct. Biol. 2:593-596, each of which is incorporated by reference in its entirety.

The two or more different epitopes may be epitopes on the same antigen (e.g., a single IL-4Rα) or on different antigens (e.g., different IL-4Rα molecules, or a IL-4Rα molecule and a non-IL-4Rα molecule). In some aspects, a multi-specific antibody binds two different epitopes (i.e., a “bispecific antibody”). In some aspects, a multi-specific antibody binds three different epitopes (i.e., a “trispecific antibody”).

Anti-IL-4Rα antibodies can include those described herein such as the clones set forth in the drawings and/or tables. In some embodiments, the antibody comprises an alternative scaffold. In some embodiments, the antibody consists of an alternative scaffold. In some embodiments, the antibody consists essentially of an alternative scaffold. In some embodiments, the antibody comprises an antibody fragment. In some embodiments, the antibody consists of an antibody fragment. In some embodiments, the antibody consists essentially of an antibody fragment.

In some embodiments, the antibodies are monoclonal antibodies.

In some embodiments, the antibodies are polyclonal antibodies.

In some embodiments, the antibodies are produced by hybridomas. In other embodiments, the antibodies are produced by recombinant cells engineered to express the desired variable and constant domains.

In some embodiments, the antibodies may be single chain antibodies or other antibody derivatives retaining the antigen specificity and the lower hinge region or a variant thereof.

In some embodiments, the antibodies may be polyfunctional antibodies, recombinant antibodies, fully human antibodies, humanized antibodies, fragments or variants thereof. In particular embodiments, the antibody fragment or a derivative thereof is selected from a Fab fragment, a Fab′2 fragment, a CDR and scFv.

TABLE 2
Sequences of IL-4Rα Antibody Constructs - vH, vL, and associated CDRs

| Identifier | VH and Heavy chain CDRs | VL and Light chain CDRs |
|---|---|---|
| Dupilumab | VH (SEQ ID NO: 113) | VL (SEQ ID NO: 146) |
| | CDR1: Kabat (SEQ ID NO: 1) | CDR1: Kabat (SEQ ID NO: 26) |
| | Chothia (SEQ ID NO: 66) | Chothia (SEQ ID NO: 26) |
| | IMGT (SEQ ID NO: 187) | IMGT (SEQ ID NO: 100) |
| | CDR2: Kabat (SEQ ID NO: 5) | CDR2: Kabat (SEQ ID NO: 41) |
| | Chothia (SEQ ID NO: 71) | Chothia (SEQ ID NO: 41) |
| | IMGT (SEQ ID NO: 79) | IMGT (SEQ ID NO: 108) |
| | CDR3: Kabat (SEQ ID NO: 17) | CDR3: Kabat (SEQ ID NO: 53) |
| | Chothia (SEQ ID NO: 17) | Chothia (SEQ ID NO: 53) |
| | IMGT (SEQ ID NO: 91) | IMGT (SEQ ID NO: 53) |
| mAb410 | VH (SEQ ID NO: 114) | VL (SEQ ID NO: 147) |
| | CDR1: Kabat (SEQ ID NO: 2) | CDR1: Kabat (SEQ ID NO: 27) |
| | Chothia (SEQ ID NO: 67) | Chothia (SEQ ID NO: 27) |
| | IMGT (SEQ ID NO: 188) | IMGT (SEQ ID NO: 101) |
| | CDR2: Kabat (SEQ ID NO: 6) | CDR2: Kabat (SEQ ID NO: 42) |
| | Chothia (SEQ ID NO: 71) | Chothia (SEQ ID NO: 42) |
| | IMGT (SEQ ID NO: 80) | IMGT (SEQ ID NO: 108) |
| | CDR3: Kabat (SEQ ID NO: 18) | CDR3: Kabat (SEQ ID NO: 54) |
| | Chothia (SEQ ID NO: 18) | Chothia (SEQ ID NO: 54) |
| | IMGT (SEQ ID NO: 92) | IMGT (SEQ ID NO: 54) |
| mAb411 | VH (SEQ ID NO: 114) | VL (SEQ ID NO: 148) |
| | CDR1: Kabat (SEQ ID NO: 2) | CDR1: Kabat (SEQ ID NO: 28) |
| | Chothia (SEQ ID NO: 62) | Chothia (SEQ ID NO: 28) |
| | IMGT (SEQ ID NO: 188) | IMGT (SEQ ID NO: 100) |
| | CDR2: Kabat (SEQ ID NO: 6) | CDR2: Kabat (SEQ ID NO: 42) |
| | Chothia (SEQ ID NO: 71) | Chothia (SEQ ID NO: 42) |
| | IMGT (SEQ ID NO: 80) | IMGT (SEQ ID NO: 108) |
| | CDR3: Kabat (SEQ ID NO: 18) | CDR3: Kabat (SEQ ID NO: 55) |
| | Chothia (SEQ ID NO: 18) | Chothia (SEQ ID NO: 55) |
| | IMGT (SEQ ID NO: 92) | IMGT (SEQ ID NO: 55) |
| mAb412 | VH (SEQ ID NO: 115) | VL (SEQ ID NO: 149) |
| | CDR1: Kabat (SEQ ID NO: 2) | CDR1: Kabat (SEQ ID NO: 29) |
| | Chothia (SEQ ID NO: 67) | Chothia (SEQ ID NO: 29) |
| | IMGT (SEQ ID NO: 188) | IMGT (SEQ ID NO: 100) |
| | CDR2: Kabat (SEQ ID NO: 7) | CDR2: Kabat (SEQ ID NO: 43) |
| | Chothia (SEQ ID NO: 72) | Chothia (SEQ ID NO: 43) |
| | IMGT (SEQ ID NO: 81) | IMGT (SEQ ID NO: 108) |
| | CDR3: Kabat (SEQ ID NO: 22) | CDR3: Kabat (SEQ ID NO: 55) |
| | Chothia (SEQ ID NO: 22) | Chothia (SEQ ID NO: 55) |
| | IMGT (SEQ ID NO: 93) | IMGT (SEQ ID NO: 55) |
| mAb413 | VH (SEQ ID NO: 114) | VL (SEQ ID NO: 150) |
| | CDR1: Kabat (SEQ ID NO: 2) | CDR1: Kabat (SEQ ID NO: 28) |
| | Chothia (SEQ ID NO: 67) | Chothia (SEQ ID NO: 28) |
| | IMGT (SEQ ID NO: 188) | IMGT (SEQ ID NO: 100) |
| | CDR2: Kabat (SEQ ID NO: 6) | CDR2: Kabat (SEQ ID NO: 45) |
| | Chothia (SEQ ID NO: 71) | Chothia (SEQ ID NO: 45) |
| | IMGT (SEQ ID NO: 80) | IMGT (SEQ ID NO: 108) |
| | CDR3: Kabat (SEQ ID NO: 18) | CDR3: Kabat (SEQ ID NO: 55) |
| | Chothia (SEQ ID NO: 18) | Chothia (SEQ ID NO: 55) |
| | IMGT (SEQ ID NO: 92) | IMGT (SEQ ID NO: 55) |
| mAb414 | VH (SEQ ID NO: 116) | VL (SEQ ID NO: 151) |
| | CDR1: Kabat (SEQ ID NO: 2) | CDR1: Kabat (SEQ ID NO: 30) |
| | Chothia (SEQ ID NO: 67) | Chothia (SEQ ID NO: 30) |
| | IMGT (SEQ ID NO: 188) | IMGT (SEQ ID NO: 102) |
| | CDR2: Kabat (SEQ ID NO: 8) | CDR2: Kabat (SEQ ID NO: 43) |
| | Chothia (SEQ ID NO: 73) | Chothia (SEQ ID NO: 43) |
| | IMGT (SEQ ID NO: 82) | IMGT (SEQ ID NO: 108) |
| | CDR3: Kabat (SEQ ID NO: 19) | CDR3: Kabat (SEQ ID NO: 56) |
| | Chothia (SEQ ID NO: 19) | Chothia (SEQ ID NO: 56) |
| | IMGT (SEQ ID NO: 94) | IMGT (SEQ ID NO: 56) |
| mAb415 | VH (SEQ ID NO: 117) | VL (SEQ ID NO: 152) |
| | CDR1: Kabat (SEQ ID NO: 2) | CDR1: Kabat (SEQ ID NO: 31) |
| | Chothia (SEQ ID NO: 67) | Chothia (SEQ ID NO: 31) |
| | IMGT (SEQ ID NO: 188) | IMGT (SEQ ID NO: 103) |
| | CDR2: Kabat (SEQ ID NO: 8) | CDR2: Kabat (SEQ ID NO: 43) |
| | Chothia (SEQ ID NO: 73) | Chothia (SEQ ID NO: 43) |
| | IMGT (SEQ ID NO: 82) | IMGT (SEQ ID NO: 108) |
| | CDR3: Kabat (SEQ ID NO: 20) | CDR3: Kabat (SEQ ID NO: 57) |
| | Chothia (SEQ ID NO: 20) | Chothia (SEQ ID NO: 57) |
| | IMGT (SEQ ID NO: 95) | IMGT (SEQ ID NO: 57) |
| mAb416 | VH (SEQ ID NO: 118) | VL (SEQ ID NO: 153) |
| | CDR1: Kabat (SEQ ID NO: 2) | CDR1: Kabat (SEQ ID NO: 32) |
| | Chothia (SEQ ID NO: 67) | Chothia (SEQ ID NO: 32) |
| | IMGT (SEQ ID NO: 188) | IMGT (SEQ ID NO: 102) |
| | CDR2: Kabat (SEQ ID NO: 6) | CDR2: Kabat (SEQ ID NO: 46) |
| | Chothia (SEQ ID NO: 71) | Chothia (SEQ ID NO: 46) |
| | IMGT (SEQ ID NO: 80) | IMGT (SEQ ID NO: 109) |
| | CDR3: Kabat (SEQ ID NO: 17) | CDR3: Kabat (SEQ ID NO: 58) |
| | Chothia (SEQ ID NO: 17) | Chothia (SEQ ID NO: 58) |
| | IMGT (SEQ ID NO: 91) | IMGT (SEQ ID NO: 58) |
| mAb417 | VH (SEQ ID NO: 117) | VL (SEQ ID NO: 154) |
| | CDR1: Kabat (SEQ ID NO: 2) | CDR1: Kabat (SEQ ID NO: 32) |
| | Chothia (SEQ ID NO: 67) | Chothia (SEQ ID NO: 32) |
| | IMGT (SEQ ID NO: 188) | IMGT (SEQ ID NO: 102) |
| | CDR2: Kabat (SEQ ID NO: 8) | CDR2: Kabat (SEQ ID NO: 45) |
| | Chothia (SEQ ID NO: 73) | Chothia (SEQ ID NO: 45) |
| | IMGT (SEQ ID NO: 82) | IMGT (SEQ ID NO: 108) |
| | CDR3: Kabat (SEQ ID NO: 20) | CDR3: Kabat (SEQ ID NO: 59) |
| | Chothia (SEQ ID NO: 20) | Chothia (SEQ ID NO: 59) |
| | IMGT (SEQ ID NO: 95) | IMGT (SEQ ID NO: 59) |
| mAb418 | VH (SEQ ID NO: 119) | VL (SEQ ID NO: 155) |
| | CDR1: Kabat (SEQ ID NO: 2) | CDR1: Kabat (SEQ ID NO: 30) |
| | Chothia (SEQ ID NO: 68) | Chothia (SEQ ID NO: 30) |
| | IMGT (SEQ ID NO: 189) | IMGT (SEQ ID NO: 102) |
| | CDR2: Kabat (SEQ ID NO: 6) | CDR2: Kabat (SEQ ID NO: 45) |
| | Chothia (SEQ ID NO: 71) | Chothia (SEQ ID NO: 45) |
| | IMGT (SEQ ID NO: 80) | IMGT (SEQ ID NO: 108) |
| | CDR3: Kabat (SEQ ID NO: 20) | CDR3: Kabat (SEQ ID NO: 60) |
| | Chothia (SEQ ID NO: 20) | Chothia (SEQ ID NO: 60) |
| | IMGT (SEQ ID NO: 95) | IMGT (SEQ ID NO: 60) |
| mAb419 | VH (SEQ ID NO: 120) | VL (SEQ ID NO: 156) |
| | CDR1: Kabat (SEQ ID NO: 2) | CDR1: Kabat (SEQ ID NO: 29) |
| | Chothia (SEQ ID NO: 67) | Chothia (SEQ ID NO: 29) |
| | IMGT (SEQ ID NO: 188) | IMGT (SEQ ID NO: 100) |
| | CDR2: Kabat (SEQ ID NO: 9) | CDR2: Kabat (SEQ ID NO: 43) |
| | Chothia (SEQ ID NO: 77) | Chothia (SEQ ID NO: 43) |
| | IMGT (SEQ ID NO: 83) | IMGT (SEQ ID NO: 108) |
| | CDR3: Kabat (SEQ ID NO: 18) | CDR3: Kabat (SEQ ID NO: 60) |
| | Chothia (SEQ ID NO: 18) | Chothia (SEQ ID NO: 60) |
| | IMGT (SEQ ID NO: 92) | IMGT (SEQ ID NO: 60) |
| mAb420 | VH (SEQ ID NO: 120) | VL (SEQ ID NO: 157) |
| | CDR1: Kabat (SEQ ID NO: 2) | CDR1: Kabat (SEQ ID NO: 27) |
| | Chothia (SEQ ID NO: 67) | Chothia (SEQ ID NO: 27) |
| | IMGT (SEQ ID NO: 188) | IMGT (SEQ ID NO: 101) |
| | CDR2: Kabat (SEQ ID NO: 9) | CDR2: Kabat (SEQ ID NO: 43) |
| | Chothia (SEQ ID NO: 77) | Chothia (SEQ ID NO: 43) |
| | IMGT (SEQ ID NO: 83) | IMGT (SEQ ID NO: 108) |
| | CDR3: Kabat (SEQ ID NO: 18) | CDR3: Kabat (SEQ ID NO: 57) |
| | Chothia (SEQ ID NO: 18) | Chothia (SEQ ID NO: 57) |
| | IMGT (SEQ ID NO: 92) | IMGT (SEQ ID NO: 57) |
| mAb421 | VH (SEQ ID NO: 114) | VL (SEQ ID NO: 158) |
| | CDR1: Kabat (SEQ ID NO: 2) | CDR1: Kabat (SEQ ID NO: 27) |
| | Chothia (SEQ ID NO: 67) | Chothia (SEQ ID NO: 27) |
| | IMGT (SEQ ID NO: 188) | IMGT (SEQ ID NO: 101) |
| | CDR2: Kabat (SEQ ID NO: 6) | CDR2: Kabat (SEQ ID NO: 43) |
| | Chothia (SEQ ID NO: 71) | Chothia (SEQ ID NO: 43) |
| | IMGT (SEQ ID NO: 80) | IMGT (SEQ ID NO: 108) |
| | CDR3: Kabat (SEQ ID NO: 18) | CDR3: Kabat (SEQ ID NO: 61) |
| | Chothia (SEQ ID NO: 18) | Chothia (SEQ ID NO: 61) |
| | IMGT (SEQ ID NO: 92) | IMGT (SEQ ID NO: 61) |
| mAb422 | VH (SEQ ID NO: 121) | VL (SEQ ID NO: 158) |
| | CDR1: Kabat (SEQ ID NO: 2) | CDR1: Kabat (SEQ ID NO: 27) |
| | Chothia (SEQ ID NO: 67) | Chothia (SEQ ID NO: 27) |
| | IMGT (SEQ ID NO: 188) | IMGT (SEQ ID NO: 101) |
| | CDR2: Kabat (SEQ ID NO: 10) | CDR2: Kabat (SEQ ID NO: 43) |
| | Chothia (SEQ ID NO: 71) | Chothia (SEQ ID NO: 43) |
| | IMGT (SEQ ID NO: 84) | IMGT (SEQ ID NO: 108) |
| | CDR3: Kabat (SEQ ID NO: 20) | CDR3: Kabat (SEQ ID NO: 61) |
| | Chothia (SEQ ID NO: 20) | Chothia (SEQ ID NO: 61) |
| | IMGT (SEQ ID NO: 95) | IMGT (SEQ ID NO: 61) |
| mAb422B | VH (SEQ ID NO: 122) | VL (SEQ ID NO: 158) |
| | CDR1: Kabat (SEQ ID NO: 3) | CDR1: Kabat (SEQ ID NO: 27) |
| | Chothia (SEQ ID NO: 69) | Chothia (SEQ ID NO: 27) |
| | IMGT (SEQ ID NO: 190) | IMGT (SEQ ID NO: 101) |
| | CDR2: Kabat (SEQ ID NO: 10) | CDR2: Kabat (SEQ ID NO: 43) |
| | Chothia (SEQ ID NO: 71) | Chothia (SEQ ID NO: 43) |
| | IMGT (SEQ ID NO: 84) | IMGT (SEQ ID NO: 108) |
| | 4 | |
| | CDR3: Kabat (SEQ ID NO: 20) | CDR3: Kabat (SEQ ID NO: 61) |
| | Chothia (SEQ ID NO: 20) | Chothia (SEQ ID NO: 61) |
| | IMGT (SEQ ID NO: 95) | IMGT (SEQ ID NO: 61) |
| mAb423 | VH (SEQ ID NO: 114) | VL (SEQ ID NO: 159) |
| | CDR1: Kabat (SEQ ID NO: 2) | CDR1: Kabat (SEQ ID NO: 28) |
| | Chothia (SEQ ID NO: 67) | Chothia (SEQ ID NO: 28) |
| | IMGT (SEQ ID NO: 188) | IMGT (SEQ ID NO: 100) |
| | CDR2: Kabat (SEQ ID NO: 6) | CDR2: Kabat (SEQ ID NO: 43) |
| | Chothia (SEQ ID NO: 71) | Chothia (SEQ ID NO: 43) |
| | IMGT (SEQ ID NO: 80) | IMGT (SEQ ID NO: 108) |
| | CDR3: Kabat (SEQ ID NO: 18) | CDR3: Kabat (SEQ ID NO: 61) |
| | Chothia (SEQ ID NO: 18) | Chothia (SEQ ID NO: 61) |
| | IMGT (SEQ ID NO: 92) | IMGT (SEQ ID NO: 61) |
| mAb424 | VH (SEQ ID NO: 123) | VL (SEQ ID NO: 159) |
| | CDR1: Kabat (SEQ ID NO: 3) | CDR1: Kabat (SEQ ID NO: 28) |
| | Chothia (SEQ ID NO: 69) | Chothia (SEQ ID NO: 28) |
| | IMGT (SEQ ID NO: 190) | IMGT (SEQ ID NO: 100) |
| | CDR2: Kabat (SEQ ID NO: 8) | CDR2: Kabat (SEQ ID NO: 43) |
| | Chothia (SEQ ID NO: 73) | Chothia (SEQ ID NO: 43) |
| | IMGT (SEQ ID NO: 82) | IMGT (SEQ ID NO: 108) |
| | CDR3: Kabat (SEQ ID NO: 21) | CDR3: Kabat (SEQ ID NO: 61) |
| | Chothia (SEQ ID NO: 21) | Chothia (SEQ ID NO: 61) |
| | IMGT (SEQ ID NO: 96) | IMGT (SEQ ID NO: 61) |
| mAb425 | VH (SEQ ID NO: 124) | VL (SEQ ID NO: 160) |
| | CDR1: Kabat (SEQ ID NO: 2) | CDR1: Kabat (SEQ ID NO: 28) |
| | Chothia (SEQ ID NO: 67) | Chothia (SEQ ID NO: 28) |
| | IMGT (SEQ ID NO: 188) | IMGT (SEQ ID NO: 100) |
| | CDR2: Kabat (SEQ ID NO: 7) | CDR2: Kabat (SEQ ID NO: 42) |
| | Chothia (SEQ ID NO: 72) | Chothia (SEQ ID NO: 42) |
| | IMGT (SEQ ID NO: 81) | IMGT (SEQ ID NO: 108) |
| | CDR3: Kabat (SEQ ID NO: 20) | CDR3: Kabat (SEQ ID NO: 62) |
| | Chothia (SEQ ID NO: 20) | Chothia (SEQ ID NO: 62) |
| | IMGT (SEQ ID NO: 95) | IMGT (SEQ ID NO: 62) |
| mAb426 | VH (SEQ ID NO: 125) | VL (SEQ ID NO: 161) |
| | CDR1: Kabat (SEQ ID NO: 2) | CDR1: Kabat (SEQ ID NO: 33) |
| | Chothia (SEQ ID NO: 67) | Chothia (SEQ ID NO: 33) |
| | IMGT (SEQ ID NO: 188) | IMGT (SEQ ID NO: 102) |
| | CDR2: Kabat (SEQ ID NO: 11) | CDR2: Kabat (SEQ ID NO: 43) |
| | Chothia (SEQ ID NO: 73) | Chothia (SEQ ID NO: 43) |
| | IMGT (SEQ ID NO: 85) | IMGT (SEQ ID NO: 108) |
| | CDR3: Kabat (SEQ ID NO: 18) | CDR3: Kabat (SEQ ID NO: 61) |
| | Chothia (SEQ ID NO: 18) | Chothia (SEQ ID NO: 61) |
| | IMGT (SEQ ID NO: 92) | IMGT (SEQ ID NO: 61) |
| mAb427 | VH (SEQ ID NO: 126) | VL (SEQ ID NO: 162) |
| | CDR1: Kabat (SEQ ID NO: 23 | CDR1: Kabat (SEQ ID NO: 34) |
| | Chothia (SEQ ID NO: 69) | Chothia (SEQ ID NO: 34) |
| | IMGT (SEQ ID NO: 190) | IMGT (SEQ ID NO: 100) |
| | CDR2: Kabat (SEQ ID NO: 11) | CDR2: Kabat (SEQ ID NO: 43) |
| | Chothia (SEQ ID NO: 73) | Chothia (SEQ ID NO: 43) |
| | IMGT (SEQ ID NO: 85) | IMGT (SEQ ID NO: 108) |
| | CDR3: Kabat (SEQ ID NO: 19) | CDR3: Kabat (SEQ ID NO: 60) |
| | Chothia (SEQ ID NO: 19) | Chothia (SEQ ID NO: 60) |
| | IMGT (SEQ ID NO: 94) | IMGT (SEQ ID NO: 60) |
| mAb428 | VH (SEQ ID NO: 127) | VL (SEQ ID NO: 163) |
| | CDR1: Kabat (SEQ ID NO: 2) | CDR1: Kabat (SEQ ID NO: 34) |
| | Chothia (SEQ ID NO: 67) | Chothia (SEQ ID NO: 34) |
| | IMGT (SEQ ID NO: 188) | IMGT (SEQ ID NO: 100) |
| | CDR2: Kabat (SEQ ID NO: 11) | CDR2: Kabat (SEQ ID NO: 43) |
| | Chothia (SEQ ID NO: 73) | Chothia (SEQ ID NO: 43) |
| | IMGT (SEQ ID NO: 85) | IMGT (SEQ ID NO: 108) |
| | CDR3: Kabat (SEQ ID NO: 22) | CDR3: Kabat (SEQ ID NO: 56) |
| | Chothia (SEQ ID NO: 22) | Chothia (SEQ ID NO: 56) |
| | IMGT (SEQ ID NO: 93) | IMGT (SEQ ID NO: 56) |
| mAb429 | VH (SEQ ID NO: 128) | VL (SEQ ID NO: 164) |
| | CDR1: Kabat (SEQ ID NO: 3) | CDR1: Kabat (SEQ ID NO: 28) |
| | Chothia (SEQ ID NO: 69) | Chothia (SEQ ID NO: 28) |
| | IMGT (SEQ ID NO: 190) | IMGT (SEQ ID NO: 100) |
| | CDR2: Kabat (SEQ ID NO: 6) | CDR2: Kabat (SEQ ID NO: 42) |
| | Chothia (SEQ ID NO: 71) | Chothia (SEQ ID NO: 42) |
| | IMGT (SEQ ID NO: 80) | IMGT (SEQ ID NO: 108) |
| | CDR3: Kabat (SEQ ID NO: 23) | CDR3: Kabat (SEQ ID NO: 61) |
| | Chothia (SEQ ID NO: 23) | Chothia (SEQ ID NO: 61) |
| | IMGT (SEQ ID NO: 97) | IMGT (SEQ ID NO: 61) |
| mAb430 | VH (SEQ ID NO: 124) | VL (SEQ ID NO: 158) |
| | CDR1: Kabat (SEQ ID NO: 2) | CDR1: Kabat (SEQ ID NO: 27) |
| | Chothia (SEQ ID NO: 67) | Chothia (SEQ ID NO: 27) |
| | IMGT (SEQ ID NO: 188) | IMGT (SEQ ID NO: 101) |
| | CDR2: Kabat (SEQ ID NO: 7) | CDR2: Kabat (SEQ ID NO: 43) |
| | Chothia (SEQ ID NO: 72) | Chothia (SEQ ID NO: 43) |
| | IMGT (SEQ ID NO: 81) | IMGT (SEQ ID NO: 108) |
| | CDR3: Kabat (SEQ ID NO: 20) | CDR3: Kabat (SEQ ID NO: 61) |
| | Chothia (SEQ ID NO: 18) | Chothia (SEQ ID NO: 61) |
| | IMGT (SEQ ID NO: 95) | IMGT (SEQ ID NO: 61) |
| mAb431 | VH (SEQ ID NO: 129) | VL (SEQ ID NO: 165) |
| | CDR1: Kabat (SEQ ID NO: 2) | CDR1: Kabat (SEQ ID NO: 27) |
| | Chothia (SEQ ID NO: 67) | Chothia (SEQ ID NO: 27) |
| | IMGT (SEQ ID NO: 188) | IMGT (SEQ ID NO: 101) |
| | CDR2: Kabat (SEQ ID NO: 6) | CDR2: Kabat (SEQ ID NO: 43) |
| | Chothia (SEQ ID NO: 71) | Chothia (SEQ ID NO: 43) |
| | IMGT (SEQ ID NO: 80) | IMGT (SEQ ID NO: 108) |
| | CDR3: Kabat (SEQ ID NO: 18) | CDR3: Kabat (SEQ ID NO: 61) |
| | Chothia (SEQ ID NO: 18) | Chothia (SEQ ID NO: 61) |
| | IMGT (SEQ ID NO: 92) | IMGT (SEQ ID NO: 61) |
| mAb432 | VH (SEQ ID NO: 130) | VL (SEQ ID NO: 166) |
| | CDR1: Kabat (SEQ ID NO: 2) | CDR1: Kabat (SEQ ID NO: 36) |
| | Chothia (SEQ ID NO: 67) | Chothia (SEQ ID NO: 36) |
| | IMGT (SEQ ID NO: 188) | IMGT (SEQ ID NO: 102) |
| | CDR2: Kabat (SEQ ID NO: 6) | CDR2: Kabat (SEQ ID NO: 45) |
| | Chothia (SEQ ID NO: 71) | Chothia (SEQ ID NO: 45) |
| | IMGT (SEQ ID NO: 80) | IMGT (SEQ ID NO: 108) |
| | CDR3: Kabat (SEQ ID NO: 24) | CDR3: Kabat (SEQ ID NO: 61) |
| | Chothia (SEQ ID NO: 24) | Chothia (SEQ ID NO: 61) |
| | IMGT (SEQ ID NO: 98) | IMGT (SEQ ID NO: 61) |
| mAb433 | VH (SEQ ID NO: 131) | VL (SEQ ID NO: 167) |
| | CDR1: Kabat (SEQ ID NO: 2) | CDR1: Kabat (SEQ ID NO: 37) |
| | Chothia (SEQ ID NO: 67) | Chothia (SEQ ID NO: 37) |
| | IMGT (SEQ ID NO: 188) | IMGT (SEQ ID NO: 100) |
| | CDR2: Kabat (SEQ ID NO: 8) | CDR2: Kabat (SEQ ID NO: 47) |
| | Chothia (SEQ ID NO: 73) | Chothia (SEQ ID NO: 47) |
| | IMGT (SEQ ID NO: 82) | IMGT (SEQ ID NO: 110) |
| | CDR3: Kabat (SEQ ID NO: 18) | CDR3: Kabat (SEQ ID NO: 61) |
| | Chothia (SEQ ID NO: 18) | Chothia (SEQ ID NO: 61) |
| | IMGT (SEQ ID NO: 92) | IMGT (SEQ ID NO: 61) |
| mAb434 | VH (SEQ ID NO: 132) | VL (SEQ ID NO: 159) |
| | CDR1: Kabat (SEQ ID NO: 2) | CDR1: Kabat (SEQ ID NO: 28) |
| | Chothia (SEQ ID NO: 67) | Chothia (SEQ ID NO: 28) |
| | IMGT (SEQ ID NO: 188) | IMGT (SEQ ID NO: 100) |
| | CDR2: Kabat (SEQ ID NO: 12) | CDR2: Kabat (SEQ ID NO: 43) |
| | Chothia (SEQ ID NO: 74) | Chothia (SEQ ID NO: 43) |
| | IMGT (SEQ ID NO: 86) | IMGT (SEQ ID NO: 108) |
| | CDR3: Kabat (SEQ ID NO: 24) | CDR3: Kabat (SEQ ID NO: 61) |
| | Chothia (SEQ ID NO: 24) | Chothia (SEQ ID NO: 61) |
| | IMGT (SEQ ID NO: 98) | IMGT (SEQ ID NO: 61) |
| mAb435 | VH (SEQ ID NO: 133) | VL (SEQ ID NO: 159) |
| | CDR1: Kabat (SEQ ID NO: 2) | CDR1: Kabat (SEQ ID NO: 28) |
| | Chothia (SEQ ID NO: 67) | Chothia (SEQ ID NO: 28) |
| | IMGT (SEQ ID NO: 189) | IMGT (SEQ ID NO: 100) |
| | CDR2: Kabat (SEQ ID NO: 13) | CDR2: Kabat (SEQ ID NO: 43) |
| | Chothia (SEQ ID NO: 71) | Chothia (SEQ ID NO: 43) |
| | IMGT (SEQ ID NO: 87) | IMGT (SEQ ID NO: 108) |
| | CDR3: Kabat (SEQ ID NO: 20) | CDR3: Kabat (SEQ ID NO: 61) |
| | Chothia (SEQ ID NO: 20) | Chothia (SEQ ID NO: 61) |
| | IMGT (SEQ ID NO: 95) | IMGT (SEQ ID NO: 61) |
| mAb436 | VH (SEQ ID NO: 121) | VL (SEQ ID NO: 168) |
| | CDR1: Kabat (SEQ ID NO: 2) | CDR1: Kabat (SEQ ID NO: 34) |
| | Chothia (SEQ ID NO: 67) | Chothia (SEQ ID NO: 34) |
| | IMGT (SEQ ID NO: 188) | IMGT (SEQ ID NO: 100) |
| | CDR2: Kabat (SEQ ID NO: 10) | CDR2: Kabat (SEQ ID NO: 43) |
| | Chothia (SEQ ID NO: 71) | Chothia (SEQ ID NO: 43) |
| | IMGT (SEQ ID NO: 84) | IMGT (SEQ ID NO: 108) |
| | CDR3: Kabat (SEQ ID NO: 20) | CDR3: Kabat (SEQ ID NO: 57) |
| | Chothia (SEQ ID NO: 20) | Chothia (SEQ ID NO: 57) |
| | IMGT (SEQ ID NO: 95) | IMGT (SEQ ID NO: 57) |
| mAb437 | VH (SEQ ID NO: 134) | VL (SEQ ID NO: 169) |
| | CDR1: Kabat (SEQ ID NO: 2) | CDR1: Kabat (SEQ ID NO: 34) |
| | Chothia (SEQ ID NO: 67) | Chothia (SEQ ID NO: 34) |
| | IMGT (SEQ ID NO: 188) | IMGT (SEQ ID NO: 100) |
| | CDR2: Kabat (SEQ ID NO: 6) | CDR2: Kabat (SEQ ID NO: 43) |
| | Chothia (SEQ ID NO: 71) | Chothia (SEQ ID NO: 43) |
| | IMGT (SEQ ID NO: 80) | IMGT (SEQ ID NO: 108) |
| | CDR3: Kabat (SEQ ID NO: 20) | CDR3: Kabat (SEQ ID NO: 61) |
| | Chothia (SEQ ID NO: 20) | Chothia (SEQ ID NO: 61) |
| | IMGT (SEQ ID NO: 95) | IMGT (SEQ ID NO: 61) |
| mAb439 | VH (SEQ ID NO: 113) | VL (SEQ ID NO: 170) |
| | CDR1: Kabat (SEQ ID NO: 1) | CDR1: Kabat (SEQ ID NO: 38) |
| | Chothia (SEQ ID NO: 66) | Chothia (SEQ ID NO: 38) |
| | IMGT (SEQ ID NO: 187) | IMGT (SEQ ID NO: 105) |
| | CDR2: Kabat (SEQ ID NO: 5) | CDR2: Kabat (SEQ ID NO: 48) |
| | Chothia (SEQ ID NO: 71) | Chothia (SEQ ID NO: 48) |
| | IMGT (SEQ ID NO: 79) | IMGT (SEQ ID NO: 111) |
| | CDR3: Kabat (SEQ ID NO: 17) | CDR3: Kabat (SEQ ID NO: 63) |
| | Chothia (SEQ ID NO: 17) | Chothia (SEQ ID NO: 63) |
| | IMGT (SEQ ID NO: 91) | IMGT (SEQ ID NO: 63) |
| mAb443 | VH (SEQ ID NO: 134) | VL (SEQ ID NO: 171) |
| | CDR1: Kabat (SEQ ID NO: 2) | CDR1: Kabat (SEQ ID NO: 39) |
| | Chothia (SEQ ID NO: 67) | Chothia (SEQ ID NO: 39) |
| | IMGT (SEQ ID NO: 188) | IMGT (SEQ ID NO: 106) |
| | CDR2: Kabat (SEQ ID NO: 6) | CDR2: Kabat (SEQ ID NO: 41) |
| | Chothia (SEQ ID NO: 71) | Chothia (SEQ ID NO: 41) |
| | IMGT (SEQ ID NO: 80) | IMGT (SEQ ID NO: 108) |
| | CDR3: Kabat (SEQ ID NO: 20) | CDR3: Kabat (SEQ ID NO: 64) |
| | Chothia (SEQ ID NO: 20) | Chothia (SEQ ID NO: 64) |
| | IMGT (SEQ ID NO: 95) | IMGT (SEQ ID NO: 64) |
| mAb446 | VH (SEQ ID NO: 135) | VL (SEQ ID NO: 172) |
| | CDR1: Kabat (SEQ ID NO: 2) | CDR1: Kabat (SEQ ID NO: 39) |
| | Chothia (SEQ ID NO: 67) | Chothia (SEQ ID NO: 39) |
| | IMGT (SEQ ID NO: 188) | IMGT (SEQ ID NO: 106) |
| | CDR2: Kabat (SEQ ID NO: 14) | CDR2: Kabat (SEQ ID NO: 49) |
| | Chothia (SEQ ID NO: 75) | Chothia (SEQ ID NO: 49) |
| | IMGT (SEQ ID NO: 88) | IMGT (SEQ ID NO: 112) |
| | CDR3: Kabat (SEQ ID NO: 20) | CDR3: Kabat (SEQ ID NO: 63) |
| | Chothia (SEQ ID NO: 20) | Chothia (SEQ ID NO: 63) |
| | IMGT (SEQ ID NO: 95) | IMGT (SEQ ID NO: 63) |
| mAb450 | VH (SEQ ID NO: 136) | VL (SEQ ID NO: 173) |
| | CDR1: Kabat (SEQ ID NO: 2) | CDR1: Kabat (SEQ ID NO: 28) |
| | Chothia (SEQ ID NO: 67) | Chothia (SEQ ID NO: 28) |
| | IMGT (SEQ ID NO: 188) | IMGT (SEQ ID NO: 100) |
| | CDR2: Kabat (SEQ ID NO: 6) | CDR2: Kabat (SEQ ID NO: 42) |
| | Chothia (SEQ ID NO: 71) | Chothia (SEQ ID NO: 42) |
| | IMGT (SEQ ID NO: 80) | IMGT (SEQ ID NO: 108) |
| | CDR3: Kabat (SEQ ID NO: 19) | CDR3: Kabat (SEQ ID NO: 60) |
| | Chothia (SEQ ID NO: 19) | Chothia (SEQ ID NO: 60) |
| | IMGT (SEQ ID NO: 94) | IMGT (SEQ ID NO: 60) |
| mAb459 | VH (SEQ ID NO: 131) | VL (SEQ ID NO: 174) |
| | CDR1: Kabat (SEQ ID NO: 2) | CDR1: Kabat (SEQ ID NO: 31) |
| | Chothia (SEQ ID NO: 67) | Chothia (SEQ ID NO: 31) |
| | IMGT (SEQ ID NO: 188) | IMGT (SEQ ID NO: 103) |
| | CDR2: Kabat (SEQ ID NO: 8) | CDR2: Kabat (SEQ ID NO: 43) |
| | Chothia (SEQ ID NO: 73) | Chothia (SEQ ID NO: 43) |
| | IMGT (SEQ ID NO: 82) | IMGT (SEQ ID NO: 108) |
| | CDR3: Kabat (SEQ ID NO: 18) | CDR3: Kabat (SEQ ID NO: 58) |
| | Chothia (SEQ ID NO: 18) | Chothia (SEQ ID NO: 58) |
| | IMGT (SEQ ID NO: 92) | IMGT (SEQ ID NO: 58) |
| mAb467 | VH (SEQ ID NO: 137) | VL (SEQ ID NO: 175) |
| | CDR1: Kabat (SEQ ID NO: 3) | CDR1: Kabat (SEQ ID NO: 33) |
| | Chothia (SEQ ID NO: 69) | Chothia (SEQ ID NO: 33) |
| | IMGT (SEQ ID NO: 190) | IMGT (SEQ ID NO: 102) |
| | CDR2: Kabat (SEQ ID NO: 5) | CDR2: Kabat (SEQ ID NO: 50) |
| | Chothia (SEQ ID NO: 71) | Chothia (SEQ ID NO: 50) |
| | IMGT (SEQ ID NO: 79) | IMGT (SEQ ID NO: 110) |
| | CDR3: Kabat (SEQ ID NO: 19) | CDR3: Kabat (SEQ ID NO: 61) |
| | Chothia (SEQ ID NO: 19) | Chothia (SEQ ID NO: 61) |
| | IMGT (SEQ ID NO: 94) | IMGT (SEQ ID NO: 61) |
| mAb469 | VH (SEQ ID NO: 114) | VL (SEQ ID NO: 176) |
| | CDR1: Kabat (SEQ ID NO: 2) | CDR1: Kabat (SEQ ID NO: 30) |
| | Chothia (SEQ ID NO: 67) | Chothia (SEQ ID NO: 30) |
| | IMGT (SEQ ID NO: 188) | IMGT (SEQ ID NO: 101) |
| | CDR2: Kabat (SEQ ID NO: 6) | CDR2: Kabat (SEQ ID NO: 51) |
| | Chothia (SEQ ID NO: 71) | Chothia (SEQ ID NO: 51) |
| | IMGT (SEQ ID NO: 80) | IMGT (SEQ ID NO: 108) |
| | CDR3: Kabat (SEQ ID NO: 18) | CDR3: Kabat (SEQ ID NO: 61) |
| | Chothia (SEQ ID NO: 18) | Chothia (SEQ ID NO: 61) |
| | IMGT (SEQ ID NO: 92) | IMGT (SEQ ID NO: 61) |
| mAb470 | VH (SEQ ID NO: 138) | VL (SEQ ID NO: 177) |
| | CDR1: Kabat (SEQ ID NO: 3) | CDR1: Kabat (SEQ ID NO: 40) |
| | Chothia (SEQ ID NO: 67) | Chothia (SEQ ID NO: 40) |
| | IMGT (SEQ ID NO: 190) | IMGT (SEQ ID NO: 107) |
| | CDR2: Kabat (SEQ ID NO: 15) | CDR2: Kabat (SEQ ID NO: 52) |
| | Chothia (SEQ ID NO: 76) | Chothia (SEQ ID NO: 52) |
| | IMGT (SEQ ID NO: 89) | IMGT (SEQ ID NO: 108) |
| | CDR3: Kabat (SEQ ID NO: 20) | CDR3: Kabat (SEQ ID NO: 61) |
| | Chothia (SEQ ID NO: 20) | Chothia (SEQ ID NO: 61) |
| | IMGT (SEQ ID NO: 95) | IMGT (SEQ ID NO: 61) |
| mAb471 | VH (SEQ ID NO: 134) | VL (SEQ ID NO: 178) |
| | CDR1: Kabat (SEQ ID NO: 2) | CDR1: Kabat (SEQ ID NO: 30) |
| | Chothia (SEQ ID NO: 67) | Chothia (SEQ ID NO: 30) |
| | IMGT (SEQ ID NO: 188) | IMGT (SEQ ID NO: 102) |
| | CDR2: Kabat (SEQ ID NO: 6) | CDR2: Kabat (SEQ ID NO: 47) |
| | Chothia (SEQ ID NO: 71) | Chothia (SEQ ID NO: 47) |
| | IMGT (SEQ ID NO: 80) | IMGT (SEQ ID NO: 110) |
| | CDR3: Kabat (SEQ ID NO: 20) | CDR3: Kabat (SEQ ID NO: 61) |
| | Chothia (SEQ ID NO: 20) | Chothia (SEQ ID NO: 61) |
| | IMGT (SEQ ID NO: 95) | IMGT (SEQ ID NO: 61) |
| mAb473 | VH (SEQ ID NO: 139) | VL (SEQ ID NO: 179) |
| | CDR1: Kabat (SEQ ID NO: 4) | CDR1: Kabat (SEQ ID NO: 30) |
| | Chothia (SEQ ID NO: 70) | Chothia (SEQ ID NO: 30) |
| | IMGT (SEQ ID NO: 191) | IMGT (SEQ ID NO: 102) |
| | CDR2: Kabat (SEQ ID NO: 11) | CDR2: Kabat (SEQ ID NO: 43) |
| | Chothia (SEQ ID NO: 73) | Chothia (SEQ ID NO: 43) |
| | IMGT (SEQ ID NO: 85) | IMGT (SEQ ID NO: 108) |
| | CDR3: Kabat (SEQ ID NO: 22) | CDR3: Kabat (SEQ ID NO: 60) |
| | Chothia (SEQ ID NO: 18) | Chothia (SEQ ID NO: 60) |
| | IMGT (SEQ ID NO: 93) | IMGT (SEQ ID NO: 60) |
| mAb476 | VH (SEQ ID NO: 140) | VL (SEQ ID NO: 180) |
| | CDR1: Kabat (SEQ ID NO: 2) | CDR1: Kabat (SEQ ID NO: 27) |
| | Chothia (SEQ ID NO: 67) | Chothia (SEQ ID NO: 27) |
| | IMGT (SEQ ID NO: 188) | IMGT (SEQ ID NO: 101) |
| | CDR2: Kabat (SEQ ID NO: 14) | CDR2: Kabat (SEQ ID NO: 43) |
| | Chothia (SEQ ID NO: 75) | Chothia (SEQ ID NO: 43) |
| | IMGT (SEQ ID NO: 88) | IMGT (SEQ ID NO: 108) |
| | CDR3: Kabat (SEQ ID NO: 18) | CDR3: Kabat (SEQ ID NO: 65) |
| | Chothia (SEQ ID NO: 18) | Chothia (SEQ ID NO: 65) |
| | IMGT (SEQ ID NO: 92) | IMGT (SEQ ID NO: 65) |
| mAb478 | VH (SEQ ID NO: 141) | VL (SEQ ID NO: 181) |
| | CDR1: Kabat (SEQ ID NO: 2) | CDR1: Kabat (SEQ ID NO: 28) |
| | Chothia (SEQ ID NO: 67) | Chothia (SEQ ID NO: 28) |
| | IMGT (SEQ ID NO: 188) | IMGT (SEQ ID NO: 100) |
| | CDR2: Kabat (SEQ ID NO: 15) | CDR2: Kabat (SEQ ID NO: 43) |
| | Chothia (SEQ ID NO: 76) | Chothia (SEQ ID NO: 43) |
| | IMGT (SEQ ID NO: 89) | IMGT (SEQ ID NO: 108) |
| | CDR3: Kabat (SEQ ID NO: 19) | CDR3: Kabat (SEQ ID NO: 62) |
| | Chothia (SEQ ID NO: 19) | Chothia (SEQ ID NO: 62) |
| | IMGT (SEQ ID NO: 94) | IMGT (SEQ ID NO: 62) |
| mAb481 | VH (SEQ ID NO: 142) | VL (SEQ ID NO: 182) |
| | CDR1: Kabat (SEQ ID NO: 3) | CDR1: Kabat (SEQ ID NO: 28) |
| | Chothia (SEQ ID NO: 69) | Chothia (SEQ ID NO: 28) |
| | IMGT (SEQ ID NO: 190) | IMGT (SEQ ID NO: 100) |
| | CDR2: Kabat (SEQ ID NO: 9) | CDR2: Kabat (SEQ ID NO: 43) |
| | Chothia (SEQ ID NO: 77) | Chothia (SEQ ID NO: 43) |
| | IMGT (SEQ ID NO: 83) | IMGT (SEQ ID NO: 108) |
| | CDR3: Kabat (SEQ ID NO: 19) | CDR3: Kabat (SEQ ID NO: 55) |
| | Chothia (SEQ ID NO: 19) | Chothia (SEQ ID NO: 55) |
| | IMGT (SEQ ID NO: 94) | IMGT (SEQ ID NO: 55) |
| mAb482 | VH (SEQ ID NO: 143) | VL (SEQ ID NO: 183) |
| | CDR1: Kabat (SEQ ID NO: 2) | CDR1: Kabat (SEQ ID NO: 34) |
| | Chothia (SEQ ID NO: 67) | Chothia (SEQ ID NO: 34) |
| | IMGT (SEQ ID NO: 188) | IMGT (SEQ ID NO: 100) |
| | CDR2: Kabat (SEQ ID NO: 15) | CDR2: Kabat (SEQ ID NO: 42) |
| | Chothia (SEQ ID NO: 76) | Chothia (SEQ ID NO: 42) |
| | IMGT (SEQ ID NO: 89) | IMGT (SEQ ID NO: 108) |
| | CDR3: Kabat (SEQ ID NO: 24) | CDR3: Kabat (SEQ ID NO: 61) |
| | Chothia (SEQ ID NO: 24) | Chothia (SEQ ID NO: 61) |
| | IMGT (SEQ ID NO: 98) | IMGT (SEQ ID NO: 61) |
| mAb485 | VH (SEQ ID NO: 114) | VL (SEQ ID NO: 184) |
| | CDR1: Kabat (SEQ ID NO: 2) | CDR1: Kabat (SEQ ID NO: 32) |
| | Chothia (SEQ ID NO: 67) | Chothia (SEQ ID NO: 32) |
| | IMGT (SEQ ID NO: 188) | IMGT (SEQ ID NO: 102) |
| | CDR2: Kabat (SEQ ID NO: 6) | CDR2: Kabat (SEQ ID NO: 51) |
| | Chothia (SEQ ID NO: 71) | Chothia (SEQ ID NO: 51) |
| | IMGT (SEQ ID NO: 80) | IMGT (SEQ ID NO: 110) |
| | CDR3: Kabat (SEQ ID NO: 18) | CDR3: Kabat (SEQ ID NO: 61) |
| | Chothia (SEQ ID NO: 18) | Chothia (SEQ ID NO: 61) |
| | IMGT (SEQ ID NO: 92) | IMGT (SEQ ID NO: 61) |
| mAb494 | VH (SEQ ID NO: 144) | VL (SEQ ID NO: 185) |
| | CDR1: Kabat (SEQ ID NO: 2) | CDR1: Kabat (SEQ ID NO: 28) |
| | Chothia (SEQ ID NO: 67) | Chothia (SEQ ID NO: 28) |
| | IMGT (SEQ ID NO: 188) | IMGT (SEQ ID NO: 100) |
| | CDR2: Kabat (SEQ ID NO: 16) | CDR2: Kabat (SEQ ID NO: 45) |
| | Chothia (SEQ ID NO: 78) | Chothia (SEQ ID NO: 45) |
| | IMGT (SEQ ID NO: 90) | IMGT (SEQ ID NO: 108) |
| | CDR3: Kabat (SEQ ID NO: 19) | CDR3: Kabat (SEQ ID NO: 57) |
| | Chothia (SEQ ID NO: 19) | Chothia (SEQ ID NO: 57) |
| | IMGT (SEQ ID NO: 94) | IMGT (SEQ ID NO: 57) |
| mAb501 | VH (SEQ ID NO: 145) | VL (SEQ ID NO: 186) |
| | CDR1: Kabat (SEQ ID NO: 4) | CDR1: Kabat (SEQ ID NO: 34) |
| | Chothia (SEQ ID NO: 70) | Chothia (SEQ ID NO: 34) |
| | IMGT (SEQ ID NO: 191) | IMGT (SEQ ID NO: 100) |
| | CDR2: Kabat (SEQ ID NO: 16) | CDR2: Kabat (SEQ ID NO: 43) |
| | Chothia (SEQ ID NO: 78) | Chothia (SEQ ID NO: 43) |
| | IMGT (SEQ ID NO: 90) | IMGT (SEQ ID NO: 108) |
| | CDR3: Kabat (SEQ ID NO: 25) | CDR3: Kabat (SEQ ID NO: 53) |
| | Chothia (SEQ ID NO: 25) | Chothia (SEQ ID NO: 53) |
| | IMGT (SEQ ID NO: 99) | IMGT (SEQ ID NO: 53) |

### VH Domains

In some embodiments, an antibody provided herein comprises a VH sequence selected from SEQ ID NOs: 113-145.

In some embodiments, an antibody provided herein comprises a VH sequence having at least about 80%, 90%, 91%, 92%, 93%, 94%, 95%, 96%, 97%, 98% or 99% identity to an illustrative VH sequence provided in SEQ ID NOs: 113-145. In some embodiments, an antibody provided herein comprises a VH sequence provided in SEQ ID NOs: 113-145, with up to 1, 2, 3, 4, 5, 6, 7, 8, 9, 10, 11, 12, 13, 14, 15, 16, 17, 18, 19, or 20 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### VL Domains

In some embodiments, an antibody provided herein comprises a VL sequence selected from SEQ ID NOs: 146-186.

In some embodiments, an antibody provided herein comprises a VL sequence having at least about 80%, 90%, 91%, 92%, 93%, 94%, 95%, 96%, 97%, 98% or 99% identity to an illustrative VL sequence provided in SEQ ID NO: 146-186. In some embodiments, an antibody provided herein comprises a VL sequence provided in SEQ ID NO: 146-186, with up to 1, 2, 3, 4, 5, 6, 7, 8, 9, 10, 11, 12, 13, 14, 15, 16, 17, 18, 19, or 20 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### VH-VL Combinations

In some embodiments, an antibody provided herein comprises a VH sequence selected from SEQ ID NOs: 113-145; and a VL sequence selected from SEQ ID NO: 146-186.

In certain aspects, any of SEQ ID NOs: 113-145 can be combined with any of SEQ ID NO: 146-186.

In certain aspects, an antibody provided herein comprises a VH sequence and a VL sequence of a construct provided in TABLE 2.

In some embodiments, an antibody provided herein comprises a VH sequence having at least about 80%, 90%, 91%, 92%, 93%, 94%, 95%, 96%, 97%, 98% or 99% identity to a VH sequence provided in SEQ ID NOs: 113-145; and a VL sequence having at least about 80%, 90%, 91%, 92%, 93%, 94%, 95%, 96%, 97%, 98% or 99% identity to a VL sequence provided in SEQ ID NO: 146-186. In some embodiments, an antibody provided herein comprises a VH sequence provided in SEQ ID NOs: 113-145, with up to 1, 2, 3, 4, 5, 6, 7, 8, 9, 10, 11, 12, 13, 14, 15, 16, 17, 18, 19, or 20 amino acid substitutions, and a VL sequence provided in SEQ ID NO: 146-186, with up to 1, 2, 3, 4, 5, 6, 7, 8, 9, 10, 11, 12, 13, 14, 15, 16, 17, 18, 19, or 20 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In certain embodiments, the antibody comprises a VH sequence set forth in SEQ ID NO: 114 and a VL sequence set forth in SEQ ID NO: 147. In certain embodiments, the antibody comprises a VH sequence set forth in SEQ ID NO: 114 and a VL sequence set forth in SEQ ID NO: 148. In certain embodiments, the antibody comprises a VH sequence set forth in SEQ ID NO: 115 and a VL sequence set forth in SEQ ID NO: 149. In certain embodiments, the antibody comprises a VH sequence set forth in SEQ ID NO: 114 and a VL sequence set forth in SEQ ID NO: 150. In certain embodiments, the antibody comprises a VH sequence set forth in SEQ ID NO: 116 and a VL sequence set forth in SEQ ID NO: 151. In certain embodiments, the antibody comprises a VH sequence set forth in SEQ ID NO: 117 and a VL sequence set forth in SEQ ID NO: 152. In certain embodiments, the antibody comprises a VH sequence set forth in SEQ ID NO: 118 and a VL sequence set forth in SEQ ID NO: 153. In certain embodiments, the antibody comprises a VH sequence set forth in SEQ ID NO: 117 and a VL sequence set forth in SEQ ID NO: 154. In certain embodiments, the antibody comprises a VH sequence set forth in SEQ ID NO: 119 and a VL sequence set forth in SEQ ID NO: 155. In certain embodiments, the antibody comprises a VH sequence set forth in SEQ ID NO: 120 and a VL sequence set forth in SEQ ID NO: 156. In certain embodiments, the antibody comprises a VH sequence set forth in SEQ ID NO: 120 and a VL sequence set forth in SEQ ID NO: 157. In certain embodiments, the antibody comprises a VH sequence set forth in SEQ ID NO: 114 and a VL sequence set forth in SEQ ID NO: 158. In certain embodiments, the antibody comprises a VH sequence set forth in SEQ ID NO: 121 and a VL sequence set forth in SEQ ID NO: 158. In certain embodiments, the antibody comprises a VH sequence set forth in SEQ ID NO: 122 and a VL sequence set forth in SEQ ID NO: 158. In certain embodiments, the antibody comprises a VH sequence set forth in SEQ ID NO: 114 and a VL sequence set forth in SEQ ID NO: 159. In certain embodiments, the antibody comprises a VH sequence set forth in SEQ ID NO: 123 and a VL sequence set forth in SEQ ID NO: 159. In certain embodiments, the antibody comprises a VH sequence set forth in SEQ ID NO: 124 and a VL sequence set forth in SEQ ID NO: 160. In certain embodiments, the antibody comprises a VH sequence set forth in SEQ ID NO: 125 and a VL sequence set forth in SEQ ID NO: 161. In certain embodiments, the antibody comprises a VH sequence set forth in SEQ ID NO: 126, and a VL sequence set forth in SEQ ID NO: 162. In certain embodiments, the antibody comprises a VH sequence set forth in SEQ ID NO: 127 and a VL sequence set forth in SEQ ID NO: 163. In certain embodiments, the antibody comprises a VH sequence set forth in SEQ ID NO: 128 and a VL sequence set forth in SEQ ID NO: 164. In certain embodiments, the antibody comprises a VH sequence set forth in SEQ ID NO: 124 and a VL sequence set forth in SEQ ID NO: 158. In certain embodiments, the antibody comprises a VH sequence set forth in SEQ ID NO: 129 and a VL sequence set forth in SEQ ID NO: 165. In certain embodiments, the antibody comprises a VH sequence set forth in SEQ ID NO: 130, and a VL sequence set forth in SEQ ID NO: 166. In certain embodiments, the antibody comprises a VH sequence set forth in SEQ ID NO: 131 and a VL sequence set forth in SEQ ID NO: 167. In certain embodiments, the antibody comprises a VH sequence set forth in SEQ ID NO: 132 and a VL sequence set forth in SEQ ID NO: 159. In certain embodiments, the antibody comprises a VH sequence set forth in SEQ ID NO: 133 and a VL sequence set forth in SEQ ID NO: 159. In certain embodiments, the antibody comprises a VH sequence set forth in SEQ ID NO: 121 and a VL sequence set forth in SEQ ID NO: 168. In certain embodiments, the antibody comprises a VH sequence set forth in SEQ ID NO: 134 and a VL sequence set forth in SEQ ID NO: 169. In certain embodiments, the antibody comprises a VH sequence set forth in SEQ ID NO: 113 and a VL sequence set forth in SEQ ID NO: 170. In certain embodiments, the antibody comprises a VH sequence set forth in SEQ ID NO: 134 and a VL sequence set forth in SEQ ID NO: 171. In certain embodiments, the antibody comprises a VH sequence set forth in SEQ ID NO: 135 and a VL sequence set forth in SEQ ID NO: 172. In certain embodiments, the antibody comprises a VH sequence set forth in SEQ ID NO: 136 and a VL sequence set forth in SEQ ID NO: 173. In certain embodiments, the antibody comprises a VH sequence set forth in SEQ ID NO: 131 and a VL sequence set forth in SEQ ID NO: 174. In certain embodiments, the antibody comprises a VH sequence set forth in SEQ ID NO: 137 and a VL sequence set forth in SEQ ID NO: 175. In certain embodiments, the antibody comprises a VH sequence set forth in SEQ ID NO: 114 and a VL sequence set forth in SEQ ID NO: 176. In certain embodiments, the antibody comprises a VH sequence set forth in SEQ ID NO: 138 and a VL sequence set forth in SEQ ID NO: 177. In certain embodiments, the antibody comprises a VH sequence set forth in SEQ ID NO: 134 and a VL sequence set forth in SEQ ID NO: 178. In certain embodiments, the antibody comprises a VH sequence set forth in SEQ ID NO: 139 and a VL sequence set forth in SEQ ID NO: 179. In certain embodiments, the antibody comprises a VH sequence set forth in SEQ ID NO: 140 and a VL sequence set forth in SEQ ID NO: 180. In certain embodiments, the antibody comprises a VH sequence set forth in SEQ ID NO: 141 and a VL sequence set forth in SEQ ID NO: 181. In certain embodiments, the antibody comprises a VH sequence set forth in SEQ ID NO: 142 and a VL sequence set forth in SEQ ID NO: 182. In certain embodiments, the antibody comprises a VH sequence set forth in SEQ ID NO: 143 and a VL sequence set forth in SEQ ID NO: 183. In certain embodiments, the antibody comprises a VH sequence set forth in SEQ ID NO: 114 and a VL sequence set forth in SEQ ID NO: 184. In certain embodiments, the antibody comprises a VH sequence set forth in SEQ ID NO: 144 and a VL sequence set forth in SEQ ID NO: 185. In certain embodiments, the antibody comprises a VH sequence set forth in SEQ ID NO: 145 and a VL sequence set forth in SEQ ID NO: 186.

In certain embodiments of any of the antibodies described above, the antibody further comprises a heavy chain comprising a human IgG sequence selected from a sequence set forth in SEQ ID NO: 192-235 and 251-407.

In certain embodiments, the antibody comprises a VH sequence set forth in SEQ ID NO: 113; a VL sequence set forth in SEQ ID NO: 146; and the antibody further comprises a heavy chain comprising a human IgG sequence selected from a sequence set forth in SEQ ID NO: 192-235 and 251-407. In certain embodiments of any of the antibodies described above, the antibody further comprises a constant light chain sequence comprising a sequence set forth in SEQ ID NO: 236.

In certain embodiments, the antibody comprises a VH sequence set forth in SEQ ID NO: 134, a VL sequence set forth in SEQ ID NO: 178, a heavy chain constant region comprising LALA/YTE substitutions, and a human kappa light chain constant region. In certain embodiments, the antibody comprises a VH sequence set forth in SEQ ID NO: 134, a VL sequence set forth in SEQ ID NO: 178, a heavy chain constant region comprising a sequence set forth in SEQ ID NO: 205 or SEQ ID NO: 321, and a light chain constant region comprising a sequence set forth in SEQ ID NO: 236. In certain embodiments, the antibody comprises a VH sequence having at least about 80%, 90%, 91%, 92%, 93%, 94%, 95%, 96%, 97%, 98% or 99% identity to a VH sequence provided in SEQ ID NO: 134; and a VL sequence having at least about 80%, 90%, 91%, 92%, 93%, 94%, 95%, 96%, 97%, 98% or 99% identity to a VL sequence provided in SEQ ID NO: 178, a heavy chain constant region comprising LALA/YTE substitutions, and a human kappa light chain constant region. In certain embodiments, the antibody comprises a VH sequence having at least about 80%, 90%, 91%, 92%, 93%, 94%, 95%, 96%, 97%, 98% or 99% identity to a VH sequence provided in SEQ ID NO: 134; and a VL sequence having at least about 80%, 90%, 91%, 92%, 93%, 94%, 95%, 96%, 97%, 98% or 99% identity to a VL sequence provided in SEQ ID NO: 178, a heavy chain constant region comprising a sequence set forth in SEQ ID NO: 205 or SEQ ID NO: 321 or a sequence having at least about 80%, 90%, 91%, 92%, 93%, 94%, 95%, 96%, 97%, 98% or 99% identity to SEQ ID NO: 205 or SEQ ID NO: 321, and a light chain constant region comprising a sequence set forth in SEQ ID NO: 236 or a sequence having at least about 80%, 90%, 91%, 92%, 93%, 94%, 95%, 96%, 97%, 98% or 99% identity to SEQ ID NO: 236.

In certain embodiments, the antibody comprises a VH sequence set forth in SEQ ID NO: 130, a VL sequence set forth in SEQ ID NO: 166, a heavy chain constant region comprising LALA/YTE substitutions, and a human kappa light chain constant region. In certain embodiments, the antibody comprises a VH sequence set forth in SEQ ID NO: 130, a VL sequence set forth in SEQ ID NO: 166, a heavy chain constant region comprising a sequence set forth in SEQ ID NO: 205 or SEQ ID NO: 321, and a light chain constant region comprising a sequence set forth in SEQ ID NO: 236. In certain embodiments, the antibody comprises a VH sequence having at least about 80%, 90%, 91%, 92%, 93%, 94%, 95%, 96%, 97%, 98% or 99% identity to a VH sequence provided in SEQ ID NO: 130; and a VL sequence having at least about 80%, 90%, 91%, 92%, 93%, 94%, 95%, 96%, 97%, 98% or 99% identity to a VL sequence provided in SEQ ID NO: 166, a heavy chain constant region comprising LALA/YTE substitutions, and a human kappa light chain constant region. In certain embodiments, the antibody comprises a VH sequence having at least about 80%, 90%, 91%, 92%, 93%, 94%, 95%, 96%, 97%, 98% or 99% identity to a VH sequence provided in SEQ ID NO: 130; and a VL sequence having at least about 80%, 90%, 91%, 92%, 93%, 94%, 95%, 96%, 97%, 98% or 99% identity to a VL sequence provided in SEQ ID NO: 166, a heavy chain constant region comprising a sequence set forth in SEQ ID NO: 205 or SEQ ID NO: 321 or a sequence having at least about 80%, 90%, 91%, 92%, 93%, 94%, 95%, 96%, 97%, 98% or 99% identity to SEQ ID NO: 205 or SEQ ID NO: 321, and a light chain constant region comprising a sequence set forth in SEQ ID NO: 236 or a sequence having at least about 80%, 90%, 91%, 92%, 93%, 94%, 95%, 96%, 97%, 98% or 99% identity to SEQ ID NO: 236.

In certain embodiments, the antibody comprises a VH sequence set forth in SEQ ID NO: 121, a VL sequence set forth in SEQ ID NO: 158, a heavy chain constant region comprising LALA/YTE substitutions, and a human kappa light chain constant region. In certain embodiments, the antibody comprises a VH sequence set forth in SEQ ID NO: 121, a VL sequence set forth in SEQ ID NO: 158, a heavy chain constant region comprising a sequence set forth in SEQ ID NO: 205 or SEQ ID NO: 321, and a light chain constant region comprising a sequence set forth in SEQ ID NO: 236. In certain embodiments, the antibody comprises a VH sequence having at least about 80%, 90%, 91%, 92%, 93%, 94%, 95%, 96%, 97%, 98% or 99% identity to a VH sequence provided in SEQ ID NO: 121; and a VL sequence having at least about 80%, 90%, 91%, 92%, 93%, 94%, 95%, 96%, 97%, 98% or 99% identity to a VL sequence provided in SEQ ID NO: 158, a heavy chain constant region comprising LALA/YTE substitutions, and a human kappa light chain constant region. In certain embodiments, the antibody comprises a VH sequence having at least about 80%, 90%, 91%, 92%, 93%, 94%, 95%, 96%, 97%, 98% or 99% identity to a VH sequence provided in SEQ ID NO: 121; and a VL sequence having at least about 80%, 90%, 91%, 92%, 93%, 94%, 95%, 96%, 97%, 98% or 99% identity to a VL sequence provided in SEQ ID NO: 158, a heavy chain constant region comprising a sequence set forth in SEQ ID NO: 205 or SEQ ID NO: 321 or a sequence having at least about 80%, 90%, 91%, 92%, 93%, 94%, 95%, 96%, 97%, 98% or 99% identity to SEQ ID NO: 205 or SEQ ID NO: 321, and a light chain constant region comprising a sequence set forth in SEQ ID NO: 236 or a sequence having at least about 80%, 90%, 91%, 92%, 93%, 94%, 95%, 96%, 97%, 98% or 99% identity to SEQ ID NO: 236.

### CDRs

In some embodiments, disclosed herein is an antibodies comprising 1, 2, 3, 4, 5, or 6 of the CDRs of TABLE 2. In some embodiments, disclosed herein is an antibodies comprising 6 of the Kabat CDRs of TABLE 2, 6 of the Chothia CDRs of TABLE 2, or 6 of the IMGT CDRs of TABLE 2.

In some embodiments, an antibody provided herein comprises one to three CDRs of a VH domain selected from SEQ ID NOs: 113-145. In some embodiments, an antibody provided herein comprises two to three CDRs of a V_{H} domain selected from SEQ ID NOs: 113-145. In some embodiments, an antibody provided herein comprises three CDRs of a VH domain selected from SEQ ID NOs: 113-145. In some aspects, the CDRs are Exemplary CDRs. In some aspects, the CDRs are Kabat CDRs. In some aspects, the CDRs are Chothia CDRs. In some aspects, the CDRs are IMGT CDRs. In some aspects, the CDRs are AbM CDRs. In some aspects, the CDRs are Contact CDRs.

In some embodiments, the CDRs are CDRs having at least about 80%, 90%, 91%, 92%, 93%, 94%, 95%, 96%, 97%, 98%, 99% or 100% identity with a CDR-H1, CDR-H2, or CDR-H3 of SEQ ID NOs: 1-112 and 187-191. In some embodiments, the CDR-H1 is a CDR-H1 of a VH domain selected from SEQ ID NOs: 113-145, with up to 1, 2, 3, 4, or 5 amino acid substitutions. In some embodiments, the CDR-H2 is a CDR-H2 of a VH domain of SEQ ID NO: 113-145, with up to 1, 2, 3, 4, 5, 6, 7, or 8 amino acid substitutions. In some embodiments, the CDR-H3 is a CDR-H3 of a VH domain selected from SEQ ID NOs: 113-145, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises one to three CDRs of a VL domain of SEQ ID NO: 146-186. In some embodiments, an antibody provided herein comprises two to three CDRs of a VL domain of SEQ ID NO: 146-186. In some embodiments, an antibody provided herein comprises three CDRs of a VL domain of SEQ ID NO: 146-186. In some aspects, the CDRs are Exemplary CDRs. In some aspects, the CDRs are Kabat CDRs. In some aspects, the CDRs are Chothia CDRs. In some aspects, the CDRs are IMGT CDRs. In some aspects, the CDRs are AbM CDRs. In some aspects, the CDRs are Contact CDRs.

In some embodiments, the CDRs are CDRs having at least about 80%, 90%, 91%, 92%, 93%, 94%, 95%, 96%, 97%, 98%, 99% or 100% identity with a CDR-L1, CDR-L2, or CDR-L3 of SEQ ID NO: 26-65 and 100-112. In some embodiments, the CDR-L1 is a CDR-L1 of a VL domain of SEQ ID NO: 146-186, with up to 1, 2, 3, 4, or 5 amino acid substitutions. In some embodiments, the CDR-L2 is a CDR-L2 of a VL domain of SEQ ID NO: 41-52 and 108-112, with up to 1, 2, 3, 4, 5, 6, 7, or 8 amino acid substitutions. In some embodiments, the CDR-L3 is a CDR-L3 of a VL domain of SEQ ID NO: 146-186, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises one to three CDRs of a VH domain selected from SEQ ID NOs: 113-145 and one to three CDRs of a VL domain selected from SEQ ID NO: 146-186. In some embodiments, an antibody provided herein comprises two to three CDRs of a VH domain selected from SEQ ID NOs: 113-145 and two to three CDRs of a VL domain selected from SEQ ID NO: 146-186. In some embodiments, an antibody provided herein comprises three CDRs of a VH domain selected from SEQ ID NOs: 113-145 and three CDRs of a VL domain selected from SEQ ID NO: 146-186. In some aspects, the CDRs are Exemplary CDRs. In some aspects, the CDRs are Kabat CDRs. In some aspects, the CDRs are Chothia CDRs. In some aspects, the CDRs are IMGT CDRs. In some aspects, the CDRs are AbM CDRs. In some aspects, the CDRs are Contact CDRs.

In some embodiments, an antibody provided herein comprises a CDR-H3 selected from SEQ ID NOs: 17-25 and 92-99. In some aspects, the CDR-H3 has at least about 80%, 90%, 91%, 92%, 93%, 94%, 95%, 96%, 97%, 98% or 99% identity with a CDR-H3 selected from SEQ ID NOs: 17-25 and 92-99. In some embodiments, the CDR-H3 is a CDR-H3 selected from SEQ ID NO: 17-25 and 92-99, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H1 selected from SEQ ID NOs: 1-4, 66-70, and 187-191. In some aspects, the CDR-H1 has at least about 80%, 90%, 91%, 92%, 93%, 94%, 95%, 96%, 97%, 98% or 99% identity with a CDR-H1 selected from SEQ ID NOs: 1-4, 66-70, and 187-191. In some embodiments, the CDR-H1 is a CDR-H1 selected from SEQ ID NO: 1-4, 66-70, and 187-191, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H2 selected from SEQ ID NOs: 5-16 and 71-90. In some aspects, the CDR-H2 has at least about 80%, 90%, 91%, 92%, 93%, 94%, 95%, 96%, 97%, 98% or 99% identity with a CDR-H2 selected from SEQ ID NOs: 5-16 and 71-90. In some embodiments, the CDR-H2 is a CDR-H2 selected from SEQ ID NOs: 5-16 and 71-90, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L3 selected from SEQ ID NOs: 53-65. In some aspects, the CDR-L3 has at least about 80%, 90%, 91%, 92%, 93%, 94%, 95%, 96%, 97%, 98% or 99% identity with a CDR-L3 of SEQ ID NOs: 53-65. In some embodiments, the CDR-L3 is a CDR-L3 selected from SEQ ID NOs: 53-65, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L2 selected from SEQ ID NOs: 41-52 and 108-112. In some aspects, the CDR-L2 has at least about 80%, 90%, 91%, 92%, 93%, 94%, 95%, 96%, 97%, 98% or 99% identity with a CDR-L2 selected from SEQ ID NOs: 41-52 and 108-112. In some embodiments, the CDR-L2 is a CDR-L2 selected from SEQ ID NOs: 41-52 and 108-112, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L1 selected from SEQ ID NOs: 26-40 and 100-107. In some aspects, the CDR-L1 has at least about 80%, 90%, 91%, 92%, 93%, 94%, 95%, 96%, 97%, 98% or 99% identity with a CDR-L1 selected from SEQ ID NOs: 26-40 and 100-107. In some embodiments, the CDR-L1 is a CDR-L1 selected from SEQ ID NOs: 26-40 and 100-107, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 selected from SEQ ID NOs: 17-25 and 92-99, a CDR-H2 of SEQ ID NOs: 5-16 and 71-90, a CDR-H1 selected from SEQ ID NOs: 1-4, 66-70, and 187-191, a CDR-L3 selected from SEQ ID NOs: 53-65, a CDR-L2 selected from SEQ ID NOs: 41-52 and 108-112, and a CDR-L1 selected from SEQ ID NOs: 26-40 and 100-107. In some embodiments, the CDR-H3 has at least about 80%, 90%, 91%, 92%, 93%, 94%, 95%, 96%, 97%, 98% or 99% identity with a CDR-H3 selected from SEQ ID NOs: 17-25 and 92-99, the CDR-H2 has at least about 80%, 90%, 91%, 92%, 93%, 94%, 95%, 96%, 97%, 98% or 99% identity with a CDR-H2 of SEQ ID NOs:: 5-16 and 71-90, the CDR-H1 has at least about 80%, 90%, 91%, 92%, 93%, 94%, 95%, 96%, 97%, 98% or 99% identity with a CDR-H1 selected from SEQ ID NOs: 1-4, 66-70, and 187-191, the CDR-L3 has at least about 80%, 90%, 91%, 92%, 93%, 94%, 95%, 96%, 97%, 98% or 99% identity with a CDR-L3 selected from SEQ ID NOs: 53-65, the CDR-L2 has at least about 80%, 90%, 91%, 92%, 93%, 94%, 95%, 96%, 97%, 98% or 99% identity with a CDR-L2 selected from SEQ ID NOs: 41-52 and 108-112, and the CDR-L1 has at least about 80%, 90%, 91%, 92%, 93%, 94%, 95%, 96%, 97%, 98% or 99% identity with a CDR-L1 selected from SEQ ID NOs: 26-40 and 100-107. In some embodiments, the CDR-H3 is a CDR-H3 selected from SEQ ID NOs: 17-25 and 92-99, with up to 1, 2, 3, 4, 5, 6, 7, or 8 amino acid substitutions; the CDR-H2 is a CDR-H2 of SEQ ID NOs: 5-16 and 71-90, with up to 1, 2, 3, 4, 5, 6, 7, or 8 amino acid substitutions; the CDR-H1 is a CDR-H1 selected from SEQ ID NOs: 1-4, 66-70, and 187-191, with up to 1, 2, 3, 4, or 5 amino acid substitutions; the CDR-L3 is a CDR-L3 selected from SEQ ID NOs: 53-65, with up to 1, 2, 3, 4, or 5 amino acid substitutions; the CDR-L2 is a CDR-L2 selected from SEQ ID NOs: 41-52 and 108-112, with up to 1, 2, 3, or 4 amino acid substitutions; and the CDR-L1 is a CDR-L1 selected from SEQ ID NOs: 26-40 and 100-107, with up to 1, 2, 3, 4, 5, or 6 amino acid substitutions.

In some embodiments, an antibody provided herein comprises a CDR-H1 comprising the sequence set forth in any of SEQ ID NOs: 1, 67, or 187; a CDR-H2 comprising the sequence set forth in any of SEQ ID NOs: 5, 71, or 79; a CDR-H3 comprising the sequence set forth in SEQ ID NO: 17 or 91; a CDR-L1 comprising the sequence set forth in any of SEQ ID NOs: 26 or 100; a CDR-L2 comprising the sequence set forth in any of SEQ ID NOs: 41 or 108; and a CDR-L3 comprising the sequence set forth in SEQ ID NO: 53. In some embodiments, the CDR-H3 has at least about 80%, 90%, 91%, 92%, 93%, 94%, 95%, 96%, 97%, 98% or 99% identity with a CDR-H3 of SEQ ID NOs: 17 or 91, the CDR-H2 has at least about 80%, 90%, 91%, 92%, 93%, 94%, 95%, 96%, 97%, 98% or 99% identity with a CDR-H2 of SEQ ID NO: 5, 71, or 79, the CDR-H1 has at least about 80%, 90%, 91%, 92%, 93%, 94%, 95%, 96%, 97%, 98% or 99% identity with a CDR-H1 of SEQ ID NOs: 1, 67, or 187, the CDR-L3 has at least about 80%, 90%, 91%, 92%, 93%, 94%, 95%, 96%, 97%, 98% or 99% identity with a CDR-L3 of SEQ ID NO: 53, the CDR-L2 has at least about 80%, 90%, 91%, 92%, 93%, 94%, 95%, 96%, 97%, 98% or 99% identity with a CDR-L2 of SEQ ID NOs: 41 or 108, and the CDR-L1 has at least about 80%, 90%, 91%, 92%, 93%, 94%, 95%, 96%, 97%, 98% or 99% identity with a CDR-L1 of SEQ ID NOs: 26 or 100. In some embodiments, the CDR-H3 is a CDR-H3 of SEQ ID NOs: 17 or 91, with up to 1, 2, 3, 4, 5, 6, 7, or 8 amino acid substitutions; the CDR-H2 is a CDR-H2 of SEQ ID NOs: 5, 71, or 79, with up to 1, 2, 3, 4, 5, 6, 7, or 8 amino acid substitutions; the CDR-H1 is a CDR-H1 of SEQ ID NOs: 1, 67, or 187, with up to 1, 2, 3, 4, or 5 amino acid substitutions; the CDR-L3 is a CDR-L3 of SEQ ID NO: 53 with up to 1, 2, 3, 4, or 5 amino acid substitutions; the CDR-L2 is a CDR-L2 of SEQ ID NOs: 41 or 108, with up to 1, 2, 3, or 4 amino acid substitutions; and the CDR-L1 is a CDR-L1 of SEQ ID NOs: 26 or 100, with up to 1, 2, 3, 4, 5, or 6 amino acid substitutions.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in SEQ ID NO: 18 or 92; a CDR-L1 comprising the sequence set forth in any of SEQ ID NOs: 27 or 101; a CDR-L2 comprising the sequence set forth in any of SEQ ID NOs: 42 or 108; and a CDR-L3 comprising the sequence set forth in SEQ ID NO: 54.

In some embodiments, an antibody provided herein comprises a CDR-H1 comprising the sequence set forth in any of SEQ ID NOs: 2, 62, or 188; a CDR-H2 comprising the sequence set forth in any of SEQ ID NOs: 6, 71, or 80; a CDR-H3 comprising the sequence set forth in any of SEQ ID NOs: 18 or 92; a CDR-L1 comprising the sequence set forth in any of SEQ ID NOs: 28 or 100; a CDR-L2 comprising the sequence set forth in any of SEQ ID NOs: 42 or 108; and a CDR-L3 comprising the sequence set forth in SEQ ID NO: 55.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 7, 72, or 81; a CDR-H3 comprising the sequence set forth in any of SEQ ID NOs: 22 or 93; a CDR-L1 comprising the sequence set forth in any of SEQ ID NOs: 29 or 100; a CDR-L2 comprising the sequence set forth in any of SEQ ID NOs: 43 or 108; and a CDR-L3 comprising the sequence set forth in SEQ ID NO: 55.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 18 or 92; a CDR-L1 comprising the sequence set forth in any of SEQ ID NOs: 28 or 100; a CDR-L2 comprising the sequence set forth in any of SEQ ID NOs: 45 or 108; and a CDR-L3 comprising the sequence set forth in SEQ ID NO: 55.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 8, 73, or 82; a CDR-H3 comprising the sequence set forth in any of SEQ ID NOs: 19 or 94; a CDR-L1 comprising the sequence set forth in any of SEQ ID NOs: 30 or 102; a CDR-L2 comprising the sequence set forth in any of SEQ ID NOs: 43 or 108; and a CDR-L3 comprising the sequence set forth in SEQ ID NO: 56.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 8, 73, or 82; a CDR-H3 comprising the sequence set forth in any of SEQ ID NOs: 20 or 95; a CDR-L1 comprising the sequence set forth in any of SEQ ID NOs: 31 or 103; a CDR-L2 comprising the sequence set forth in any of SEQ ID NOs: 43 or 108; and a CDR-L3 comprising the sequence set forth in SEQ ID NO: 57.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 17 or 91; a CDR-L1 comprising the sequence set forth in any of SEQ ID NOs: 32 or 102; a CDR-L2 comprising the sequence set forth in any of SEQ ID NOs: 46 or 109; and a CDR-L3 comprising the sequence set forth in SEQ ID NO: 58.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 8, 73, or 82; a CDR-H3 comprising the sequence set forth in any of SEQ ID NOs: 20 or 95; a CDR-L1 comprising the sequence set forth in any of SEQ ID NOs: 32 or 102; a CDR-L2 comprising the sequence set forth in any of SEQ ID NOs: 45 or 108; and a CDR-L3 comprising the sequence set forth in SEQ ID NO: 59.

In some embodiments, an antibody provided herein comprises a CDR-H1 comprising the sequence set forth in any of SEQ ID NOs: 2, 68, or 189; a CDR-H2 comprising the sequence set forth in any of SEQ ID NOs: 6, 71, or 80; a CDR-H3 comprising the sequence set forth in any of SEQ ID NOs: 20 or 95; a CDR-L1 comprising the sequence set forth in any of SEQ ID NOs: 32 or 102; a CDR-L2 comprising the sequence set forth in any of SEQ ID NOs: 45 or 108; and a CDR-L3 comprising the sequence set forth in SEQ ID NO: 60.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 9, 77, or 83; a CDR-H3 comprising the sequence set forth in any of SEQ ID NOs: 18 or 92; a CDR-L1 comprising the sequence set forth in any of SEQ ID NOs: 29 or 100; a CDR-L2 comprising the sequence set forth in any of SEQ ID NOs: 43 or 108; and a CDR-L3 comprising the sequence set forth in SEQ ID NO: 60.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 9, 77, or 83; a CDR-H3 comprising the sequence set forth in any of SEQ ID NOs: 18 or 92; a CDR-L1 comprising the sequence set forth in any of SEQ ID NOs: 27 or 101; a CDR-L2 comprising the sequence set forth in any of SEQ ID NOs: 43 or 108; and a CDR-L3 comprising the sequence set forth in SEQ ID NO: 57.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 18 or 92; a CDR-L1 comprising the sequence set forth in any of SEQ ID NOs: 27 or 101; a CDR-L2 comprising the sequence set forth in any of SEQ ID NOs: 43 or 108; and a CDR-L3 comprising the sequence set forth in SEQ ID NO: 61.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 10, 71, or 84; a CDR-H3 comprising the sequence set forth in any of SEQ ID NOs: 20 or 95; a CDR-L1 comprising the sequence set forth in any of SEQ ID NOs: 27 or 101; a CDR-L2 comprising the sequence set forth in any of SEQ ID NOs: 43 or 108; and a CDR-L3 comprising the sequence set forth in SEQ ID NO: 61.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 18 or 92; a CDR-L1 comprising the sequence set forth in any of SEQ ID NOs: 28 or 100; a CDR-L2 comprising the sequence set forth in any of SEQ ID NOs: 43 or 108; and a CDR-L3 comprising the sequence set forth in SEQ ID NO: 61.

In some embodiments, an antibody provided herein comprises a CDR-H1 comprising the sequence set forth in any of SEQ ID NOs: 3, 69, or 190; a CDR-H2 comprising the sequence set forth in any of SEQ ID NOs: 8, 73, or 82; a CDR-H3 comprising the sequence set forth in any of SEQ ID NOs: 21 or 96; a CDR-L1 comprising the sequence set forth in any of SEQ ID NOs: 28 or 100; a CDR-L2 comprising the sequence set forth in any of SEQ ID NOs: 43 or 108; and a CDR-L3 comprising the sequence set forth in SEQ ID NO: 61.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 7, 72, or 81; a CDR-H3 comprising the sequence set forth in any of SEQ ID NOs: 20 or 95; a CDR-L1 comprising the sequence set forth in any of SEQ ID NOs: 28 or 100; a CDR-L2 comprising the sequence set forth in any of SEQ ID NOs: 42 or 108; and a CDR-L3 comprising the sequence set forth in SEQ ID NO: 62.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 11, 73, or 85; a CDR-H3 comprising the sequence set forth in any of SEQ ID NOs: 18 or 92; a CDR-L1 comprising the sequence set forth in any of SEQ ID NOs: 33 or 102; a CDR-L2 comprising the sequence set forth in any of SEQ ID NOs: 43 or 108; and a CDR-L3 comprising the sequence set forth in SEQ ID NO: 61.

In some embodiments, an antibody provided herein comprises a CDR-H1 comprising the sequence set forth in any of SEQ ID NOs: 23, 69, or 190; a CDR-H2 comprising the sequence set forth in any of SEQ ID NOs: 11, 73, or 85; a CDR-H3 comprising the sequence set forth in any of SEQ ID NOs: 19 or 94; a CDR-L1 comprising the sequence set forth in any of SEQ ID NOs: 34 or 100; a CDR-L2 comprising the sequence set forth in any of SEQ ID NOs: 43 or 108; and a CDR-L3 comprising the sequence set forth in SEQ ID NO: 60.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 11, 73, or 85; a CDR-H3 comprising the sequence set forth in any of SEQ ID NOs: 22 or 93; a CDR-L1 comprising the sequence set forth in any of SEQ ID NOs: 34 or 100; a CDR-L2 comprising the sequence set forth in any of SEQ ID NOs: 43 or 108; and a CDR-L3 comprising the sequence set forth in SEQ ID NO: 56.

In some embodiments, an antibody provided herein comprises a CDR-H1 comprising the sequence set forth in any of SEQ ID NOs: 3, 69, or 190; a CDR-H2 comprising the sequence set forth in any of SEQ ID NOs: 6, 71, or 80; a CDR-H3 comprising the sequence set forth in any of SEQ ID NOs: 23 or 97; a CDR-L1 comprising the sequence set forth in any of SEQ ID NOs: 28 or 100; a CDR-L2 comprising the sequence set forth in any of SEQ ID NOs: 42 or 108; and a CDR-L3 comprising the sequence set forth in SEQ ID NO: 61.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 7, 72, or 81; a CDR-H3 comprising the sequence set forth in any of SEQ ID NOs: 20, 18, or 95; a CDR-L1 comprising the sequence set forth in any of SEQ ID NOs: 27 or 101; a CDR-L2 comprising the sequence set forth in any of SEQ ID NOs: 43 or 108; and a CDR-L3 comprising the sequence set forth in SEQ ID NO: 61.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 7, 71, or 80; a CDR-H3 comprising the sequence set forth in any of SEQ ID NOs: 18 or 92; a CDR-L1 comprising the sequence set forth in any of SEQ ID NOs: 27 or 101; a CDR-L2 comprising the sequence set forth in any of SEQ ID NOs: 43 or 108; and a CDR-L3 comprising the sequence set forth in SEQ ID NO: 61.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 24 or 98; a CDR-L1 comprising the sequence set forth in any of SEQ ID NOs: 36 or 102; a CDR-L2 comprising the sequence set forth in any of SEQ ID NOs: 45 or 108; and a CDR-L3 comprising the sequence set forth in SEQ ID NO: 61.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 8, 73, or 82; a CDR-H3 comprising the sequence set forth in any of SEQ ID NOs: 18 or 92; a CDR-L1 comprising the sequence set forth in any of SEQ ID NOs: 37 or 100; a CDR-L2 comprising the sequence set forth in any of SEQ ID NOs: 47 or 110; and a CDR-L3 comprising the sequence set forth in SEQ ID NO: 61.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 12, 74, or 86; a CDR-H3 comprising the sequence set forth in any of SEQ ID NOs: 24 or 98; a CDR-L1 comprising the sequence set forth in any of SEQ ID NOs: 28 or 100; a CDR-L2 comprising the sequence set forth in any of SEQ ID NOs: 43 or 108; and a CDR-L3 comprising the sequence set forth in SEQ ID NO: 61.

In some embodiments, an antibody provided herein comprises a CDR-H1 comprising the sequence set forth in any of SEQ ID NOs: 2, 67, or 189; a CDR-H2 comprising the sequence set forth in any of SEQ ID NOs: 13, 71, or 87; a CDR-H3 comprising the sequence set forth in any of SEQ ID NOs: 20 or 95; a CDR-L1 comprising the sequence set forth in any of SEQ ID NOs: 28 or 100; a CDR-L2 comprising the sequence set forth in any of SEQ ID NOs: 43 or 108; and a CDR-L3 comprising the sequence set forth in SEQ ID NO: 61.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 10, 71, or 84; a CDR-H3 comprising the sequence set forth in any of SEQ ID NOs: 20 or 95; a CDR-L1 comprising the sequence set forth in any of SEQ ID NOs: 34 or 100; a CDR-L2 comprising the sequence set forth in any of SEQ ID NOs: 43 or 108; and a CDR-L3 comprising the sequence set forth in SEQ ID NO: 57.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 20 or 95; a CDR-L1 comprising the sequence set forth in any of SEQ ID NOs: 34 or 100; a CDR-L2 comprising the sequence set forth in any of SEQ ID NOs: 43 or 108; and a CDR-L3 comprising the sequence set forth in SEQ ID NO: 61.

In some embodiments, an antibody provided herein comprises a CDR-H1 comprising the sequence set forth in any of SEQ ID NOs: 1, 66, or 187; a CDR-H2 comprising the sequence set forth in any of SEQ ID NOs: 5, 71, or 79; a CDR-H3 comprising the sequence set forth in any of SEQ ID NOs: 17 or 91; a CDR-L1 comprising the sequence set forth in any of SEQ ID NOs: 38 or 105; a CDR-L2 comprising the sequence set forth in any of SEQ ID NOs: 48 or 111; and a CDR-L3 comprising the sequence set forth in SEQ ID NO: 63.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 20 or 95; a CDR-L1 comprising the sequence set forth in any of SEQ ID NOs: 39 or 106; a CDR-L2 comprising the sequence set forth in any of SEQ ID NOs: 41 or 108; and a CDR-L3 comprising the sequence set forth in SEQ ID NO: 64.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 14, 75, or 88; a CDR-H3 comprising the sequence set forth in any of SEQ ID NOs: 20 or 95; a CDR-L1 comprising the sequence set forth in any of SEQ ID NOs: 39 or 106; a CDR-L2 comprising the sequence set forth in any of SEQ ID NOs: 49 or 112; and a CDR-L3 comprising the sequence set forth in SEQ ID NO: 63.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 19 or 94; a CDR-L1 comprising the sequence set forth in any of SEQ ID NOs: 28 or 100; a CDR-L2 comprising the sequence set forth in any of SEQ ID NOs: 42 or 108; and a CDR-L3 comprising the sequence set forth in SEQ ID NO: 60.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 8, 73, or 82; a CDR-H3 comprising the sequence set forth in any of SEQ ID NOs: 18 or 92; a CDR-L1 comprising the sequence set forth in any of SEQ ID NOs: 31 or 103; a CDR-L2 comprising the sequence set forth in any of SEQ ID NOs: 43 or 108; and a CDR-L3 comprising the sequence set forth in SEQ ID NO: 58.

In some embodiments, an antibody provided herein comprises a CDR-H1 comprising the sequence set forth in any of SEQ ID NOs: 3, 69, or 190; a CDR-H2 comprising the sequence set forth in any of SEQ ID NOs: 5, 71, or 79; a CDR-H3 comprising the sequence set forth in any of SEQ ID NOs: 19 or 94; a CDR-L1 comprising the sequence set forth in any of SEQ ID NOs: 33 or 102; a CDR-L2 comprising the sequence set forth in any of SEQ ID NOs: 50 or 110; and a CDR-L3 comprising the sequence set forth in SEQ ID NO: 61.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 18 or 92; a CDR-L1 comprising the sequence set forth in any of SEQ ID NOs: 30 or 101; a CDR-L2 comprising the sequence set forth in any of SEQ ID NOs: 51 or 108; and a CDR-L3 comprising the sequence set forth in SEQ ID NO: 61.

In some embodiments, an antibody provided herein comprises a CDR-H1 comprising the sequence set forth in any of SEQ ID NOs: 3, 67, or 190; a CDR-H2 comprising the sequence set forth in any of SEQ ID NOs: 15, 67, or 89; a CDR-H3 comprising the sequence set forth in any of SEQ ID NOs: 20 or 95; a CDR-L1 comprising the sequence set forth in any of SEQ ID NOs: 40 or 107; a CDR-L2 comprising the sequence set forth in any of SEQ ID NOs: 52 or 108; and a CDR-L3 comprising the sequence set forth in SEQ ID NO: 61.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 20 or 95; a CDR-L1 comprising the sequence set forth in any of SEQ ID NOs: 30 or 102; a CDR-L2 comprising the sequence set forth in any of SEQ ID NOs: 47 or 110; and a CDR-L3 comprising the sequence set forth in SEQ ID NO: 61.

In some embodiments, an antibody provided herein comprises a CDR-H1 comprising the sequence set forth in any of SEQ ID NOs: 4, 70, or 191; a CDR-H2 comprising the sequence set forth in any of SEQ ID NOs: 11, 73, or 85; a CDR-H3 comprising the sequence set forth in any of SEQ ID NOs: 22, 18, or 93; a CDR-L1 comprising the sequence set forth in any of SEQ ID NOs: 30 or 102; a CDR-L2 comprising the sequence set forth in any of SEQ ID NOs: 43 or 108; and a CDR-L3 comprising the sequence set forth in SEQ ID NO: 60.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 14, 75, or 88; a CDR-H3 comprising the sequence set forth in any of SEQ ID NOs: 18 or 92; a CDR-L1 comprising the sequence set forth in any of SEQ ID NOs: 27 or 101; a CDR-L2 comprising the sequence set forth in any of SEQ ID NOs: 43 or 108; and a CDR-L3 comprising the sequence set forth in SEQ ID NO: 65.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 15, 76, or 89; a CDR-H3 comprising the sequence set forth in any of SEQ ID NOs: 19 or 94; a CDR-L1 comprising the sequence set forth in any of SEQ ID NOs: 28 or 100; a CDR-L2 comprising the sequence set forth in any of SEQ ID NOs: 43 or 108; and a CDR-L3 comprising the sequence set forth in SEQ ID NO: 62.

In some embodiments, an antibody provided herein comprises a CDR-H1 comprising the sequence set forth in any of SEQ ID NOs: 3, 69, or 190; a CDR-H2 comprising the sequence set forth in any of SEQ ID NOs: 9, 77, or 83; a CDR-H3 comprising the sequence set forth in any of SEQ ID NOs: 19 or 94; a CDR-L1 comprising the sequence set forth in any of SEQ ID NOs: 28 or 100; a CDR-L2 comprising the sequence set forth in any of SEQ ID NOs: 43 or 108; and a CDR-L3 comprising the sequence set forth in SEQ ID NO: 55.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 15, 76, or 89; a CDR-H3 comprising the sequence set forth in any of SEQ ID NOs: 24 or 98; a CDR-L1 comprising the sequence set forth in any of SEQ ID NOs: 34 or 100; a CDR-L2 comprising the sequence set forth in any of SEQ ID NOs: 42 or 108; and a CDR-L3 comprising the sequence set forth in SEQ ID NO: 61.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 18 or 92; a CDR-L1 comprising the sequence set forth in any of SEQ ID NOs: 32 or 102; a CDR-L2 comprising the sequence set forth in any of SEQ ID NOs: 51 or 110; and a CDR-L3 comprising the sequence set forth in SEQ ID NO: 61.

In some embodiments, an antibody provided herein comprises a CDR-H1 comprising the sequence set forth in any of SEQ ID NOs: 2, 67, or 188; a CDR-H2 comprising the sequence set forth in any of SEQ ID NOs: 16, 78, or 90; a CDR-H3 comprising the sequence set forth in any of SEQ ID NOs: 19 or 94; a CDR-L1 comprising the sequence set forth in any of SEQ ID NOs: 28 or 100; a CDR-L2 comprising the sequence set forth in any of SEQ ID NOs: 45 or 108; and a CDR-L3 comprising the sequence set forth in SEQ ID NO: 57.

In some embodiments, an antibody provided herein comprises a CDR-H1 comprising the sequence set forth in any of SEQ ID NOs: 4, 70, or 191; a CDR-H2 comprising the sequence set forth in any of SEQ ID NOs: 16, 78, or 90; a CDR-H3 comprising the sequence set forth in any of SEQ ID NOs: 25 or 99; a CDR-L1 comprising the sequence set forth in any of SEQ ID NOs: 34 or 100; a CDR-L2 comprising the sequence set forth in any of SEQ ID NOs: 43 or 108; and a CDR-L3 comprising the sequence set forth in SEQ ID NO: 53.

In some embodiments, the CDR-H3 has at least about 80%, 90%, 91%, 92%, 93%, 94%, 95%, 96%, 97%, 98% or 99% identity with a CDR-H3 of TABLE 2; the CDR-H2 has at least about 80%, 90%, 91%, 92%, 93%, 94%, 95%, 96%, 97%, 98% or 99% identity with a CDR-H2 of TABLE 2, the CDR-H1 has at least about 80%, 90%, 91%, 92%, 93%, 94%, 95%, 96%, 97%, 98% or 99% identity with a CDR-H1 of TABLE 2, the CDR-L3 has at least about 80%, 90%, 91%, 92%, 93%, 94%, 95%, 96%, 97%, 98% or 99% identity with a CDR-L3 of TABLE 2, the CDR-L2 has at least about 80%, 90%, 91%, 92%, 93%, 94%, 95%, 96%, 97%, 98% or 99% identity with a CDR-L2 of TABLE 2, and the CDR-L1 has at least about 80%, 90%, 91%, 92%, 93%, 94%, 95%, 96%, 97%, 98% or 99% identity with a CDR-L1 of TABLE 2. In some embodiments, the CDR-H3 is a CDR-H3 of TABLE 2, with up to 1, 2, 3, 4, 5, 6, 7, or 8 amino acid substitutions; the CDR-H2 is a CDR-H2 of TABLE 2, with up to 1, 2, 3, 4, 5, 6, 7, or 8 amino acid substitutions; the CDR-H1 is a CDR-H1 of TABLE 2, with up to 1, 2, 3, 4, or 5 amino acid substitutions; the CDR-L3 is a CDR-L3 of TABLE 2 with up to 1, 2, 3, 4, or 5 amino acid substitutions; the CDR-L2 is a CDR-L2 of TABLE 2, with up to 1, 2, 3, or 4 amino acid substitutions; and the CDR-L1 is a CDR-L1 of TABLE 2, with up to 1, 2, 3, 4, 5, or 6 amino acid substitutions.

In certain embodiments of any of the antibodies described above, the antibody further comprises a heavy chain comprising a human IgG sequence selected from a sequence set forth in SEQ ID NO: 192-235 and 251-407. In certain embodiments of any of the antibodies described above, the antibody further comprises a constant light chain sequence comprising a sequence set forth in SEQ ID NO: 236. In certain embodiments of any of the antibodies described above, the antibody further comprises a heavy chain comprising a human IgG sequence selected from SEQ ID NO: 205 and SEQ ID NO: 321 and a constant light chain sequence comprising a sequence set forth in SEQ ID NO: 236.

In some embodiments, the antibody comprises a CDR-H1 comprising the sequence set forth in any of SEQ ID NOs: 2, 67, or 188; a CDR-H2 comprising the sequence set forth in any of SEQ ID NOs: 10, 71, or 84; a CDR-H3 comprising the sequence set forth in any of SEQ ID NOs: 20 or 95; a CDR-L1 comprising the sequence set forth in any of SEQ ID NOs: 27 or 101; a CDR-L2 comprising the sequence set forth in any of SEQ ID NOs: 43 or 108; and a CDR-L3 comprising the sequence set forth in SEQ ID NO: 61. In certain embodiments, the antibody further comprises a heavy chain constant region (e.g., an IgG1 constant region) comprising LALA/YTE substitutions. In certain embodiments, the antibody comprises a heavy chain comprising a heavy chain constant region selected from SEQ ID NO: 205 and SEQ ID NO: 321. In certain embodiments, the antibody comprises a human kappa light chain constant region. In some embodiments, the antibody comprises a constant light chain sequence comprising a sequence set forth in SEQ ID NO: 236.

In some embodiments, the antibody comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 24 or 98; a CDR-L1 comprising the sequence set forth in any of SEQ ID NOs: 36 or 102; a CDR-L2 comprising the sequence set forth in any of SEQ ID NOs: 45 or 108; and a CDR-L3 comprising the sequence set forth in SEQ ID NO: 61. In certain embodiments, the antibody further comprises a heavy chain constant region (e.g., an IgG1 constant region) comprising LALA/YTE substitutions. In certain embodiments, the antibody comprises a heavy chain comprising a heavy chain constant region selected from SEQ ID NO: 205 and SEQ ID NO: 321. In certain embodiments, the antibody comprises a human kappa light chain constant region. In some embodiments, the antibody comprises a constant light chain sequence comprising a sequence set forth in SEQ ID NO: 236.

In some embodiments, the antibody comprises a CDR-H1 comprising the sequence set forth in any of SEQ ID NOs: 2, 67, or 188; a CDR-H2 comprising the sequence set forth in any of SEQ ID NOs: 6, 71, or 80; a CDR-H3 comprising the sequence set forth in any of SEQ ID NOs: 20 or 95; a CDR-L1 comprising the sequence set forth in any of SEQ ID NOs: 30 or 102; a CDR-L2 comprising the sequence set forth in any of SEQ ID NOs: 47 or 110; and a CDR-L3 comprising the sequence set forth in SEQ ID NO: 61. In certain embodiments, the antibody further comprises a heavy chain constant region (e.g., an IgG1 constant region) comprising LALA/YTE substitutions. In certain embodiments, the antibody comprises a heavy chain comprising a heavy chain constant region selected from SEQ ID NO: 205 and SEQ ID NO: 321. In certain embodiments, the antibody comprises a human kappa light chain constant region. In some embodiments, the antibody comprises a constant light chain sequence comprising a sequence set forth in SEQ ID NO: 236.

In some aspects, the amino acid substitutions are conservative amino acid substitutions. In some embodiments, the antibodies described in this disclosure are referred to herein as “variants” or “clones”. In some embodiments, such variants or clones are derived from a sequence provided herein, for example, by affinity maturation, site directed mutagenesis, random mutagenesis, or any other method known in the art or described herein. In some embodiments, such variants or cones are not derived from a sequence provided herein and may, for example, be isolated de novo according to the methods provided herein for obtaining antibodies.

### Fc Region

The structures of the Fe regions of various immunoglobulins, and the glycosylation sites contained therein, are known in the art. See Schroeder and Cavacini, J. (2010) Allergy Clin. Immunol. 125:S41-52, incorporated by reference in its entirety. The Fc region may be a naturally occurring Fc region, or an Fc region modified as described in the art or elsewhere in this disclosure.

Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, MD, 1991. An “Fc polypeptide” of a dimeric Fc as used herein refers to one of the two polypeptides forming the dimeric Fc domain, i.e. a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence. An Fc can be of the class IgA, IgD, IgE, IgG, and IgM, and several of these may be further divided into subclasses (isotypes), e.g., IgG₁, IgG₂, IgG₃, IgG₄, IgA₁, and IgA₂.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daëron, (1997) Annu. Rev. Immunol. 15:203-234). FcRs are reviewed in Ravetch and Kinet, (1991) Annu. Rev. Immunol 9:457-92; Capel et al. (1994) Immunomethods 4:25-34; and de Haas et al. (1995) J. Lab. Clin. Med. 126:330-41. Other FcRs, including those to be identified in the future, are encompassed by the term “FcR” herein. The term also includes the neonatal receptor, FcRn, which is responsible for the transfer of maternal IgGs to the fetus (Guyer et al. (1976) J. Immunol. 117:587; and Kim et al. (1994) J. Immunol. 24:249).

Modifications in the CH2 domain can affect the binding of FcRs to the Fc. A number of amino acid modifications in the Fc region are known in the art for selectively altering the affinity of the Fc for different Fcgamma receptors. In some aspects, the Fc comprises one or more modifications to promote selective binding of Fc-gamma receptors.

Exemplary mutations that alter the binding of FcRs to the Fc are listed below:
S298A/E333A/K334A, S298A/E333A/K334A/K326A (Lu et al. (2011) J Immunol Methods 365(1-2):132-41);
F243L/R292P/Y300L/V305I/P396L, F243L/R292P/Y300L/L235V/P396L (Stavenhagen et al. (2007) Cancer Res. 67(18):8882-90; Nordstrom et al. (2011) Breast Cancer Res. 13(6):R123);
F243L (Stewart et al. (2011) Protein Eng Des Sel. 24(9):671-8.), S298A/E333A/K334A (Shields et al. (2001) J Biol Chem. 276(9):6591-604);
S239D/I332E/A330L, S239D/I332E (Lazar et al. (2006) Proc Natl Acad Sci USA. 103(11):4005-10);
S239D/S267E, S267E/L328F (Chu et al. (2008) Mol Immunol. 45(15):3926-33);
S239D/D265S/S298A/I332E, S239E/S298A/K326A/A327H, G237F/S298A/A330L/I332E, S239D/1332E/S298A, S239D/K326E/A330L/1332E/S298A, G236A/S239D/D270 L/1332E, S239E/S267E/H268D, L234F/S267E/N325L, G237F/V266L/S267D and other mutations listed in WO2011/120134 and WO2011/120135, herein incorporated by reference. Therapeutic Antibody Engineering (by William R. Strohl and Lila M. Strohl, Woodhead Publishing series in Biomedicine No 11, ISBN 1 907568 37 9, October 2012) lists mutations on page 283.

In some embodiments, an antibody described herein includes modifications to improve its ability to mediate effector function. Such modifications are known in the art and include afucosylation, or engineering of the affinity of the Fc towards an activating receptor, mainly FCGR3a for ADCC, and towards C1q for CDC. The following TABLE 3 summarizes various designs reported in the literature for effector function engineering.

Methods of producing antibodies with little or no fucose on the Fc glycosylation site (Asn 297 EU numbering) without altering the amino acid sequence are well known in the art. The GlymaX® technology (ProBioGen AG) is based on the introduction of a gene for an enzyme which deflects the cellular pathway of fucose biosynthesis into cells used for antibody production. This prevents the addition of the sugar “fucose” to the N-linked antibody carbohydrate part by antibody-producing cells. (von Horsten et al. (2010) Glycobiology. 2010 December; 20 (12):1607-18. Another approach to obtaining antibodies with lowered levels of fucosylation can be found in U.S. Pat. No. 8,409,572, which teaches selecting cell lines for antibody production for their ability to yield lower levels of fucosylation on antibodies can be fully afucosylated (meaning they contain no detectable fucose) or they can be partially afucosylated, meaning that the isolated antibody contains less than 95%, less than 85%, less than 75%, less than 65%, less than 55%, less than 45%, less than 35%, less than 25%, less than 15% or less than 5% of the amount of fucose normally detected for a similar antibody produced by a mammalian expression system.

Thus, in one embodiment, an antibody described herein can include a dimeric Fc that comprises one or more amino acid modifications as noted in TABLE 3 that confer improved effector function. In another embodiment, the antibody can be afucosylated to improve effector function.

TABLE 3
TABLE 3: CH2 domains and effector function engineering

| Reference | Mutations | Effect |
|---|---|---|
| Lu (2011), supra; Ferrara | Afucosylated | Increased ADCC |
| (2011), supra; Mizushima | | |
| et al. (2011) Genes Cells. | | |
| 16(11): 1071-1080. | | |
| Lu (2011), supra | S298A/E333A/K334A | Increased ADCC |
| Lu (2011), supra | S298A/E333A/K334A/K326A | Increased ADCC |
| Stavenhagen (2007), supra | F243L/R292P/Y300L/V305I/P396L | Increased ADCC |
| Nordstrom (2011), supra | F243L/R292P/Y300L/L235V/P396L | Increased ADCC |
| Stewart (2011), supra | F243L | Increased ADCC |
| Shields (2001), supra | S298A/E333A/K334A | Increased ADCC |
| Lazar (2006), supra | S239D/I332E/A330L | Increased ADCC |
| Lazar (2006), supra | S239D/I332E | Increased ADCC |
| Bowles et al. (2006) Blood | AME-D, not specified | Increased ADCC |
| 108(8): 2648-54 | mutations | |
| Heider et al. (2011) Blood | 37.1, mutations not disclosed | Increased ADCC |
| 118(15): 4159-68 | | |
| Moore et al. (2010) MAbs | S267E/H268F/S324T | Increased CDC |
| 2(2): 181-9 | | |

Fe modifications reducing FcgR and/or complement binding and/or effector function are known in the art. Recent publications describe strategies that have been used to engineer antibodies with reduced or silenced effector activity (see Strohl, W R (2009), Curr Opin Biotech 20:685-691, and Strohl, W R and Strohl L M, “Antibody Fc engineering for optimal antibody performance” In Therapeutic Antibody Engineering, Cambridge: Woodhead Publishing (2012), pp 225-249). These strategies include reduction of effector function through modification of glycosylation, use of IgG2/IgG4 scaffolds, or the introduction of mutations in the hinge or CH2 regions of the Fc. For example, U.S. Patent Publication No. 2011/0212087 (Strohl), International Patent Publication No. WO 2006/105338 (Xencor), U.S. Patent Publication No. 2012/0225058 (Xencor), U.S. Patent Publication No. 2012/0251531 (Genentech), and Strop et al. ((2012) J. Mol. Biol. 420: 204-219) describe specific modifications to reduce FcgR or complement binding to the Fc.

Specific, non-limiting examples of known amino acid modifications to reduce FcgR or complement binding to the Fc include those identified in the following TABLE 4:

TABLE 4
Modifications to reduce FcgR or complement binding to the Fc
Mutations

N297A
L234A/L235A
IgG2 V234A/G237A
IgG4 L235A/G237A/E318A
IgG4 S228P/L236E
IgG2/IgG4
IgG2 H268Q/V309L/A330S/A331S
C220S/C226S/C229S/P238S
C226S/C229S/E3233P/L235V/L235A
E. coli production, non glyco
L234F/L235E/P331S
Hinge mutant, e.g., C226S/P230S
D265A
L234A/G237A
L234A/L235A/G237A
L234A/L235A/P329G

Methods of producing antibodies with little or no fucose on the Fe glycosylation site (Asn 297 EU numbering) without altering the amino acid sequence are well known in the art. The GlymaxX® technology (ProBioGen AG) is based on the introduction of a gene for an enzyme which deflects the cellular pathway of fucose biosynthesis into cells used for antibody production. This prevents the addition of the sugar “fucose” to the N-linked antibody carbohydrate part by antibody-producing cells. (von Horsten et al. (2010) Glycobiology 20 (12):1607-18). Examples of cell lines capable of producing defucosylated antibody include CHO-DG44 with stable overexpression of the bacterial oxidoreductase GDP-6-deoxy-D-lyxo-4-hexylose reductase (RMD) (see von Horsten et al. (2010), supra) or Lec13 CHO cells, which are deficient in protein fucosylation (see Ripka et al. (1986) Arch. Biochem. Biophys. 249:533-545; U.S. Pat. Pub. No. 2003/0157108; WO 2004/056312; each of which is incorporated by reference in its entirety), and knockout cell lines, such as alpha-1,6-fucosyltransferase gene or FUT8 knockout CHO cells (see Yamane-Ohnuki et al. (2004) Biotech. Bioeng. 87: 614-622; Kanda et al. (2006) Biotechnol. Bioeng. 94:680-688; and WO 2003/085107; each of which is incorporated by reference in its entirety). Another approach to obtaining antibodies with lowered levels of fucosylation can be found in U.S. Pat. No. 8,409,572, which teaches selecting cell lines for antibody production for their ability to yield lower levels of fucosylation on antibodies.

Examples of cell lines capable of producing defucosylated antibody include CHO-DG44 with stable overexpression of the bacterial oxidoreductase GDP-6-deoxy-D-lyxo-4-hexylose reductase (RMD) (see von Horsten et al. (2010), supra) or Lec13 CHO cells, which are deficient in protein fucosylation (see Ripka et al. (1986), supra; U.S. Pat. Pub. No. 2003/0157108; WO 2004/056312; each of which is incorporated by reference in its entirety), and knockout cell lines, such as alpha-1,6-fucosyltransferase gene or FUT8 knockout CHO cells (see Yamane-Ohnuki et al. (2004), supra; Kanda et al. (2006), supra; and WO 2003/085107; each of which is incorporated by reference in its entirety).

Antibodies can be fully afucosylated (meaning they contain no detectable fucose) or they can be partially afucosylated, meaning that the isolated antibody contains less than 95%, less than 85%, less than 75%, less than 65%, less than 55%, less than 45%, less than 35%, less than 25%, less than 15% or less than 5% of the amount of fucose normally detected for a similar antibody produced by a mammalian expression system.

In some aspects, an antibody provided herein comprises an IgG1 domain with reduced fucose content at position Asn 297 compared to a naturally occurring IgG1 domain. Such Fc domains are known to have improved ADCC. See Shields et al. (2002) J. Biol. Chem. 277:26733-26740, incorporated by reference in its entirety. In some aspects, such antibodies do not comprise any fucose at position Asn 297. The amount of fucose may be determined using any suitable method, for example as described in WO 2008/077546, incorporated by reference in its entirety.

In certain embodiments, an antibody provided herein comprises an Fc region with one or more amino acid substitutions which improve ADCC, such as a substitution at one or more of positions 298, 333, and 334 of the Fc region. In some embodiments, an antibody provided herein comprises an Fc region with one or more amino acid substitutions at positions 239, 332, and 330, as described in Lazar et al. (2006), supra, incorporated by reference in its entirety.

Other illustrative glycosylation variants which may be incorporated into the antibodies provided herein are described, for example, in U.S. Pat. Pub. Nos. 2003/0157108, 2004/0093621, 2003/0157108, 2003/0115614, 2002/0164328, 2004/0093621, 2004/0132140, 2004/0110704, 2004/0110282, 2004/0109865; International Pat. Pub. Nos. 2000/61739, 2001/29246, 2003/085119, 2003/084570, 2005/035586, 2005/035778; 2005/053742, 2002/031140; Okazaki et al. (2004) J. Mol. Biol., 2004, 336:1239-1249; and Yamane-Ohnuki et al. (2004), supra, each of which is incorporated by reference in its entirety.

In some embodiments, an antibody provided herein comprises an Fc region with at least one galactose residue in the oligosaccharide attached to the Fc region. Such antibody variants may have improved CDC function. Examples of such antibody variants are described, for example, in WO 1997/30087; WO 1998/58964; and WO 1999/22764; each of which his incorporated by reference in its entirety.

In some embodiments, an antibody provided herein comprises one or more alterations that improves or diminishes C1q binding and/or CDC. See U.S. Pat. No. 6,194,551; WO 99/51642; and Idusogie et al. (2000) J. Immunol., 164:4178-4184; each of which is incorporated by reference in its entirety.

In certain embodiments, the heavy chain comprises a constant heavy chain sequence selected from the sequences set forth in SEQ ID NOs: 192-235 and 251-407. In certain embodiments, the constant heavy chain sequence, (e.g., a constant heavy chain sequence selected from SEQ ID NOs: 192-235 and 251-306) further comprises a C-terminal lysine (SEQ ID NOs: 307-407). Although a C-terminal lysine may be present in the corresponding coding sequence of the constant heavy chain region, it tends to be cleaved off during manufacture or after administration. Accordingly, sequences of heavy chain constant regions with and without the C-terminal lysine are provided herein.

In certain embodiments, an antibody provided herein comprises a VH sequence and a VL sequence provided in TABLE 2, together with a heavy chain constant region selected from a sequence set forth in SEQ ID NO: 192-235 and 251-407. In certain embodiments, an antibody provided herein comprises a V_{H} sequence and a V_{L} sequence provided in TABLE 2, together with a heavy chain constant region and a light chain constant region set forth in SEQ ID NO: 146.

In certain embodiments, the antibody comprises a VH sequence set forth in SEQ ID NO: 113 and a V_{L} sequence set forth in SEQ ID NO: 146; and wherein the constant heavy chain comprises a human IgG sequence selected from a sequence set forth in SEQ ID NO: 192-235 and 251-407.

In certain embodiments, the Fc region comprises one or more amino acid substitutions, wherein the one or more substitutions result in an increase in one or more of antibody half-life, ADCC activity, ADCP activity, or CDC activity compared with the Fc without the one or more substitutions. In certain embodiments, the one or more amino acid substitutions results in increased antibody half-life at pH 6.0 compared to an antibody comprising a wild-type Fc region. In certain embodiments, the antibody has an increased half-life that is about 10,000-fold, 1,000-fold, 500-fold, 100-fold, 50-fold, 20-fold, 10-fold, 9-fold, 8-fold, 7-fold, 6-fold, 5-fold, 4.5-fold, 4-fold, 3.5-fold, 3-fold, 2.5-fold, 2-fold, 1.95-fold, 1.9-fold, 1.85-fold, 1.8-fold, 1.75-fold, 1.7-fold, 1.65-fold, 1.6-fold, 1.55-fold, 1.50-fold, 1.45-fold, 1.4-fold, 1.35-fold, 1.3-fold, 1.25-fold, 1.2-fold, 1.15-fold, 1.1-fold, or 1.05-fold longer compared to an antibody comprising a wild-type Fc region. In certain embodiments, the antibody has an increased half-life that is about 10,000-fold, 1,000-fold, 500-fold, 100-fold, 50-fold, 20-fold, 10-fold, 9-fold, 8-fold, 7-fold, 6-fold, 5-fold, 4.5-fold, 4-fold, 3.5-fold, 3-fold, 2.5-fold, 2-fold, 1.95-fold, 1.9-fold, 1.85-fold, 1.8-fold, 1.75-fold, 1.7-fold, 1.65-fold, 1.6-fold, 1.55-fold, 1.50-fold, 1.45-fold, 1.4-fold, 1.35-fold, 1.3-fold, 1.25-fold, 1.2-fold, 1.15-fold, 1.1-fold, or 1.05-fold longer compared to dupilumab. In certain embodiments, the antibody has an increased half-life that is about 2.5-fold, 2.4-fold, 2.3-fold, 2.2-fold, 2.1-fold, 2.0-fold, 1.9-fold, or 1.8-fold longer compared to dupilumab.

In certain embodiments, the Fc region comprises one or more amino acid substitutions, wherein the one or more substitutions result in a decrease in one or more of ADCC activity, ADCP activity, or CDC activity compared with the Fc without the one or more substitutions.

In certain embodiments, the one or more amino acid substitutions is selected from the group consisting of S228P (SP), M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W. In certain embodiments, the one or more amino acid substitutions comprises a specific combination of amino acid substitutions selected from the group consisting of M428L/N434S (LS); M252Y/S254T/T256E (YTE); T250Q/M428L; T307A/E380A/N434A; T256D/T307Q (DQ); T256D/T307W (DW); M252Y/T256D (YD); T307Q/Q311V/A378V (QVV); T256D/H285D/T307R/Q311V/A378V (DDRVV); L309D/Q311H/N434S (DHS); S228P/L235E (SPLE); L234A/L235A (LALA), M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS; D265A/LS; LALA/LS; LALAGA/LS; LALAPG/LS; N297A/DHS; D265A/DHS; LALA/DHS; LAGA/DHS; LALAGA/DHS; LALAPG/DHS; SP/YTE; SPLE/YTE; SP/LS; SPLE/LS, SP/DHS; SPLE/DHS; N297A/LA; D265A/LA, LALA/LA, LAGA/LA, LALAGA/LA, LALAPG/LA, N297A/N434A; D265A/N434A; LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.

In certain embodiments, the Fe region binds an Fey Receptor selected from the group consisting of: FcγRI, FcγRIIa, FcγRIIb, FcγRIIc, FcγRIIIa, and FcγRIIIb. In certain embodiments, the Fc region binds an Fcγ Receptor with higher affinity at pH 6.0 compared to an antibody comprising a wild-type Fc region.

### Binding

The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule (i.e., IL-4Rα) and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule. In some embodiments, the affinity of an anti-IL-4Rα antibody for a non-target molecule is less than about 50% of the affinity for IL-4Rα. In some embodiments, the affinity of an anti-IL-4Rα antibody for a non-target molecule is less than about 40% of the affinity for IL-4Rα. In some embodiments, the affinity of an anti-IL-4Rα antibody for a non-target molecule is less than about 30% of the affinity for IL-4Rα. In some embodiments, the affinity of an anti-IL-4Rα antibody for a non-target molecule is less than about 20% of the affinity for IL-4Rα. In some embodiments, the affinity of an anti-IL-4Rα antibody for a non-target molecule is less than about 10% of the affinity for IL-4Rα. In some embodiments, the affinity of an anti-IL-4Rα antibody for a non-target molecule is less than about 1% of the affinity for IL-4Rα. In some embodiments, the affinity of an anti-IL-4Rα antibody for a non-target molecule is less than about 0.10% of the affinity for IL-4Rα.

When used herein in the context of two or more antibodies, the term “competes with” or “cross-competes with” indicates that the two or more antibodies compete for binding to an antigen (e.g., IL-4Rα). In one exemplary assay, IL-4Rα is coated on a surface and contacted with a first anti-IL-4Rα antibody, after which a second anti-IL-4Rα antibody is added. In another exemplary assay, a first anti-IL-4Rα antibody is coated on a surface and contacted with IL-4Rα, and then a second anti-IL-4Rα antibody is added. If the presence of the first anti-IL-4Rα antibody reduces binding of the second anti-IL-4Rα antibody, in either assay, then the antibodies compete with each other. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 25%, at least 50%, at least 60%, at least 70%, at least 80%, at least 85%, at least 90%, at least 95%, or at least 99% as measured in a competitive binding assay. A skilled artisan can select the concentrations of the antibodies used in the competition assays based on the affinities of the antibodies for IL-4 and the valency of the antibodies. The assays described in this definition are illustrative, and a skilled artisan can utilize any suitable assay to determine if antibodies compete with each other. Suitable assays are described, for example, in Cox et al. “Immunoassay Methods,” in Assay Guidance Manual [Internet], Updated Dec. 24, 2014 (ncbi.nlm.nih.gov/books/NBK92434/; accessed Sep. 29, 2015); Silman et al. (2001) Cytometry 44:30-37; and Finco et al. (2011) J. Pharm. Biomed. Anal. 54:351-358; each of which is incorporated by reference in its entirety.

A test antibody competes with a reference antibody if an excess of a test antibody (e.g., at least 2×, 5×, 10×, 20×, or 100×) inhibits or blocks binding of the reference antibody by, e.g., at least 50%, 60%, 70%, 75%, 80%, 85%, 90%, 95%, or 99% as measured in a competitive binding assay. Antibodies identified by competition assay (competing antibody) include antibodies binding to the same epitope as the reference antibody and antibodies binding to an adjacent epitope sufficiently proximal to the epitope bound by the reference antibody for steric hindrance to occur. For example, a second, competing antibody can be identified that competes for binding to IL-4Rα with a first antibody described herein. In certain instances, the second antibody can block or inhibit binding of the first antibody by, e.g., at least 50%, 60%, 70%, 75%, 80%, 85%, 90%, 95%, or 99% as measured in a competitive binding assay. In certain instances, the second antibody can displace the first antibody by greater than 50%, 60%, 70%, 75%, 80%, 85%, 90%, 95%, or 99%.

In certain embodiments, the antibody binds an IL-4Rα sequence set forth in SEQ ID NO: 237-240.

In certain embodiments, the antibody binds to an IL-4Rα sequence set forth in SEQ ID NO: 237-240 with a K_{D} of less than or equal to about 1, 2, 3, 4, 5, 6, 7, 8, 9×10⁻⁹ M, as measured by surface plasmon resonance (SPR). In certain embodiments, the antibody binds to an IL-4Rα sequence set forth in SEQ ID NO: 237-240 with a KD of less than or equal to about 1×10⁻¹⁰ M, as measured by surface plasmon resonance (SPR). In certain embodiments, the antibody binds to human IL-4Rα with a K_{D} of less than or equal to about 1×10⁻⁹ M, as measured by surface plasmon resonance (SPR).

In some embodiments, an antibody provided herein binds IL-4Rα with a K_{D} of less than or equal to about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 1.95, 2, 2.5, 3, 3.5, 4, 4.5, 5, 6, 7, 8, 9, or 10×10⁻⁸ M, as measured by ELISA or any other suitable method known in the art. In some embodiments, an antibody provided herein binds IL-4Rα with a K_{D} of less than or equal to about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 1.95, 2, 2.5, 3, 3.5, 4, 4.5, 5, 6, 7, 8, 9, or 10×10⁻⁹ M, as measured by ELISA or any other suitable method known in the art.

In some embodiments, the K_{D} of the antibody provided herein for the binding of IL-4Rα is between about 0.001-0.01, 0.01-0.1, 0.01-0.05, 0.05-0.1, 0.1-0.5, 0.5-1, 0.25-0.75, 0.25-0.5, 0.5-0.75, 0.75-1, 0.75-2, 1.1-1.2, 1.2-1.3, 1.3-1.4, 1.4-1.5, 1.5-1.6, 1.6-1.7, 1.7-1.8, 1.8-1.9, 1.9-2, 1-2, 1-5, 2-7, 3-8, 3-5, 4-6, 5-7, 6-8, 7-9, 7-10, or 5-10×10⁻⁸ M, as measured by ELISA or any other suitable method known in the art. In some embodiments, an antibody provided herein binds IL-4Rα with a KD of less than or equal to about 1×10⁻⁸ M, or less than or equal to above 1×10⁻⁹ M as measured by ELISA or any other suitable method known in the art.

In some embodiments, the antibody provided herein binds IL-4Rα with a KD of less than or equal to about 10, 9, 8, 7, 6, 5, 4.5, 4, 3.5, 3, 2.5, 2, 1.98, 1.95, 1.9, 1.85, 1.8, 1.75, 1.7, 1.65, 1.6, 1.55, 1.50, 1.45, 1.4, 1.3, 1.2, 1.1, 1, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.01, 0.005, 0.001, 0.0005, or 0.0001×10⁻⁸ M, or less, as measured by ELISA or any other suitable method known in the art. In some embodiments, the antibody provided herein binds IL-4Rα with a K_{D} between 5-3, 4-2, 3-1, 1.9-1.8, 1.8-1.7, 1.7-1.6, 1.6-1.5, 1.9-1.5, 1.5-1, 1-0.8, 1-0.5, 0.9-0.6, 0.7-0.4, 0.6-0.2, 0.5-0.3, 0.3-0.2, 0.2-0.1, 0.1-0.01, 0.01-0.001, or 0.001-0.0001×10⁻⁸ M as measured by ELISA or any other suitable method known in the art.

In some embodiments, the antibody provided herein binds FcRn with an affinity at pH 7.4 compared to pH 6.0 at a ratio (pH 7.4/pH 6.0) of about 10,000, 1,000, 500, 100, 50, 20, 10, 9, 8, 7, 6, 5, 4.5, 4, 3.5, 3, 2.5, 2, 1.95, 1.9, 1.85, 1.8, 1.75, 1.7, 1.65, 1.6, 1.55, 1.50, 1.45, 1.4, 1.3, 1.2, 1.1, or 1.05, as measured by ELISA or any other suitable method known in the art. In some embodiments, the antibody provided herein binds FcRn with an affinity at pH 6.0 compared to pH 7.4 at a ratio (pH 6.0/pH 7.4) of about 1-0.8, 1-0.5, 0.9-0.6, 0.7-0.4, 0.6-0.2, 0.5-0.3, 0.3-0.2, 0.2-0.1, 0.1-0.01, 0.01-0.001, or 0.001-0.0001×10⁻⁸ M as measured by ELISA or any other suitable method known in the art.

### Function

“Effector functions” refer to those biological activities mediated by the Fc region of an antibody, which activities may vary depending on the antibody isotype. Examples of antibody effector functions include receptor ligand blocking, agonism, or antagonism, C1q binding to activate complement dependent cytotoxicity (CDC), Fc receptor binding to activate antibody-dependent cellular cytotoxicity (ADCC), and antibody dependent cellular phagocytosis (ADCP). In some embodiments, the effector function of the anti-IL-4Rα antibody described herein is antagonism and blocks the IL-4Rα binding to IL-4 and/or IL-13.

### Pharmaceutical Compositions

The present application provides compositions comprising the antibodies including pharmaceutical compositions comprising any one or more of the antibodies described herein with one or more pharmaceutically acceptable excipients. In some embodiments the composition is sterile. The pharmaceutical compositions generally comprise an effective amount of an antibody.

These compositions can comprise, in addition to one or more of the antibodies disclosed herein, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

The anti-IL-4Rα antibody that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### Methods

### Methods of Preparation

Antibodies described herein can be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody) or an amino acid sequence comprising the VHH of a single domain antibody. In a further embodiment, one or more vectors (e.g., expression vectors) comprising such nucleic acid are provided. In one embodiment, the nucleic acid is provided in a multicistronic vector. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antigen-binding polypeptide construct, or (2) a first vector comprising a nucleic acid that encodes an amino acid sequence comprising the VL of the antigen-binding polypeptide construct and a second vector comprising a nucleic acid that encodes an amino acid sequence comprising the VH of the antigen-binding polypeptide construct. In one embodiment, the host cell is eukaryotic, e.g. a Chinese Hamster Ovary (CHO) cell, or human embryonic kidney (HEK) cell, or lymphoid cell (e.g., Y0, NS0, Sp20 cell). In one embodiment, a method of making an antibody is provided, wherein the method comprises culturing a host cell comprising nucleic acid encoding the antibody, as provided above, under conditions suitable for expression of the antibody, and optionally recovering the antibody from the host cell (or host cell culture medium).

For recombinant production of the antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

When an antibody or variant thereof is recombinantly produced by the host cells, the protein in certain embodiments is present at about 30%, about 25%, about 20%, about 15%, about 10%, about 5%, about 4%, about 3%, about 2%, or about 1% or less of the dry weight of the cells. When the antibody or variant thereof is recombinantly produced by the host cells, the protein, in certain embodiments, is present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. In certain embodiments, “substantially purified” antibody produced by the methods described herein, has a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

Suitable host cells for cloning or expression of antibody-encoding vectors include prokaryotic or eukaryotic cells described herein.

Recombinant host cells or host cells are cells that include an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome. Host cells can include CHO, derivatives of CHO, NS0, Sp2O, CV-1, VERO-76, HeLa, HepG2, Per. C6, or BHK.

For example, antibody may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross (2004) Nat. Biotech. 22:1409-1414, and Li et al. (2006) Nat. Biotech. 24:210-215.

Suitable host cells for the expression of glycosylated antibodies are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al. (1977) J. Gen Virol. 36:59); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al. Annals N. Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al. (1980) Proc. Natl. Acad. Sci. USA 77:4216); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

In one embodiment, the antibodies described herein are produced in stable mammalian cells, by a method comprising: transfecting at least one stable mammalian cell with: nucleic acid encoding the antibody, in a predetermined ratio; and expressing the nucleic acid in the at least one mammalian cell. In some embodiments, the predetermined ratio of nucleic acid is determined in transient transfection experiments to determine the relative ratio of input nucleic acids that results in the highest percentage of the antibody in the expressed product.

In some embodiments, is the method of producing an antibody in stable mammalian cells as described herein wherein the expression product of the at least one stable mammalian cell comprises a larger percentage of the desired glycosylated antibody as compared to the monomeric heavy or light chain polypeptides, or other antibodies.

In some embodiments, is the method of producing a glycosylated antibody in stable mammalian cells described herein, said method comprising identifying and purifying the desired glycosylated antibody. In some embodiments, the said identification is by one or both of liquid chromatography and mass spectrometry.

If required, the antibodies can be purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can find use in the present invention for purification of antibodies. For example, the bacterial proteins A and G bind to the Fc region. Likewise, the bacterial protein L binds to the Fab region of some antibodies. Purification can often be enabled by a particular fusion partner. For example, antibodies may be purified using glutathione resin if a GST fusion is employed, Ni⁺² affinity chromatography if a His-tag is employed or immobilized anti-flag antibody if a flag-tag is used. For general guidance in suitable purification techniques, see, e.g. incorporated entirely by reference Protein Purification: Principles and Practice, 3rd Ed., Scopes, Springer-Verlag, NY, 1994, incorporated entirely by reference. The degree of purification necessary will vary depending on the use of the antibodies. In some instances, no purification is necessary.

In certain embodiments, the antibodies are purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q and DEAE columns.

In specific embodiments, the proteins described herein are purified using Cation Exchange Chromatography including, but not limited to, SP-sepharose, CM sepharose, poros HS, poros CM, Toyopearl SP, Toyopearl CM, Resource/Source S and CM, Fractogel S and CM columns and their equivalents and comparables.

In addition, antibodies described herein can be chemically synthesized using techniques known in the art (e.g., see Creighton, 1983, Proteins: Structures and Molecular Principles, W. H. Freeman & Co., N.Y and Hunkapiller et al. (1984) Nature, 310:105-111). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4diaminobutyric acid, alpha-amino isobutyric acid, 4aminobutyric acid, Abu, 2-amino butyric acid, g-Abu, e-Ahx, 6amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, alanine, fluoro-amino acids, designer amino acids such as methyl amino acids, C-methyl amino acids, N-methyl amino acids, and amino acid analogs in general. Furthermore, the amino acid can be D (dextrorotary) or L (levorotary).

### Methods of Use

In an aspect, the present application provides methods of contacting IL-4Rα with an anti-IL-4Rα antibody, such as a human or humanized antibody, which results in inhibition of IL-4 binding to an IL-4 receptor alpha expressed on a cell.

In an aspect, the present application provides methods of using the isolated anti-IL-4Rα antibodies described herein for treatment of a disorder or disease in a subject. In certain aspects, described herein is a method for treating a subject in need thereof with an anti-IL-4Rα antibody, the method comprising administering to a mammalian subject a therapeutically effective amount of an anti-IL-4Rα antibody or pharmaceutical composition comprising an anti-IL-4Rα antibody described herein. In certain embodiments, the present application provides methods of treating a disorder or disease associated with elevated levels of IL-4 and/or IgE in a subject.

In certain aspects, described herein are methods for treating a pathology associated with IL-4, IL-13 and/or IL-4Rα activity, the method comprising administering to a mammalian subject a therapeutically effective amount an isolated anti-IL-4Rα antibody or a pharmaceutical composition comprising an isolated anti-IL-4Rα antibody described herein.

In certain aspects, the antibodies and antibody fragments discloses herein are useful for treating diseases and disorders which are improved, inhibited or ameliorated by reducing IL-4, IL-13 and/or IL-4Rα activity. These disorders include those characterized by abnormal or excess expression of IL-4 and/or IL-13, or by an abnormal host response to IL-4 and/or IL-13 production. IL-4 and IL-13 related disorders which are treated by the antibodies or antibody fragments of the disclosure include an inflammatory disorder or disease. Non-limiting examples of disorders which are treated by the antibodies or antibody fragments of the disclosure include, atopic dermatitis (AD), asthma (mild, moderate or severe), chronic sinusitis with nasal polyps, Chronic Rhinosinusitis without Nasal Polyps (CRSsNP), eosinophilic esophagitis (EoE), an Eosinophilic gastrointestinal disorder or disease (ENID) selected from the group consisting of Eosinophilic Gastritis (EoG), Eosinophilic Enteritis (EoN), Eosinophilic Colitis (EoC), and Eosinophilic Gastroenteritis (EGE), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), chronic obstructive pulmonary disease (COPD), an inflammatory bowel disease, such as Crohn disease or ulcerative colitis, lupus, and rheumatoid arthritis.

In certain aspects, described herein are methods for treating an inflammatory disorder or disease in a mammalian subject in need thereof, the method comprising administering to the mammalian subject a therapeutically effective amount the antibody of or a pharmaceutical composition described herein. In certain embodiments, the inflammatory disorder or disease is atopic dermatitis. In certain embodiments, the inflammatory disorder or disease is asthma. In certain embodiments, the inflammatory disorder or disease is nasal polyps.

In certain aspects, described herein are methods for treating a pathology associated with elevated levels of IL-4 and/or IL-13 in a mammalian subject in need thereof, the method comprising administering to the mammalian subject a therapeutically effective amount an antibody or a pharmaceutical composition described herein.

In certain aspects, described herein are methods of reducing biological activity of IL-4, IL13 and/or IL-4Rα in a mammalian subject in need thereof, the method comprising administering to the mammalian subject a therapeutically effective amount an antibody or a pharmaceutical composition described herein.

In certain aspects, described herein are methods of preventing an inflammatory disorder or disease in a mammalian subject in need thereof, the method comprising administering to the mammalian subject a therapeutically effective amount an antibody or a pharmaceutical composition described herein.

### Methods of Administration

In some embodiments, the methods provided herein are useful for the treatment of a disease or disorder in an individual. In an embodiment, the individual is a human and the antibody is an anti-IL-4Rα antibody described herein.

In some embodiments, an antibody is administered intravenously, intramuscularly, subcutaneously, topically, orally, transdermally, intraperitoneally, intraorbitally, by implantation, by inhalation, intrathecally, intraventricularly, or intranasally. An effective amount of an anti-IL-4Rα antibody may be administered for the treatment of a disease or disorder. The appropriate dosage of the anti-IL-4Rα antibody may be determined based on the type of disease or disorder to be treated, the type of the anti-IL-4Rα antibody, the severity and course of the disease or disorder, the clinical condition of the individual, the individual's clinical history and response to the treatment, and the discretion of the attending physician.

In certain embodiments, the anti-IL-4Rα antibody is administered every six weeks or every two months.

In some embodiments, an antibody provided herein is administered with at least one additional therapeutic agent. Any suitable additional therapeutic or immunotherapeutic agent may be administered with an antibody provided herein. Additional therapeutic agents include agents that are used to treat or prevent a disease or disorder such as, but not limited to, an inflammatory disease or disorder associated with elevated levels of IL-4, IL-13 and/or IgE.

The additional therapeutic agent can be administered by any suitable means. In some embodiments, an antibody provided herein and the additional therapeutic agent are included in the same pharmaceutical composition. In some embodiments, an antibody provided herein and the additional therapeutic agent are included in different pharmaceutical compositions.

In embodiments where an antibody provided herein and the additional therapeutic agent are included in different pharmaceutical compositions, administration of the antibody can occur prior to, simultaneously, and/or following, administration of the additional therapeutic agent. In some embodiments, administration of an antibody provided herein and the additional therapeutic agent occur within about one month of each other. In some embodiments, administration of an antibody provided herein and the additional therapeutic agent occur within about one week of each other. In some embodiments, administration of an antibody provided herein and the additional therapeutic agent occur within about one day of each other. In some embodiments, administration of an antibody provided herein and the additional therapeutic agent occur within about twelve hours of each other. In some embodiments, administration of an antibody provided herein and the additional therapeutic agent occur within about one hour of each other.

### Kits and Articles of Manufacture

The present application provides kits comprising any one or more of the antibody compositions described herein and instructions for use. In some embodiments, the kits further contain a component selected from any of secondary antibodies, reagents for immunohistochemistry analysis, pharmaceutically acceptable excipient, package insert, and instruction manual and any combination thereof. In one specific embodiment, the kit comprises a pharmaceutical composition comprising any one or more of the antibody compositions described herein, with one or more pharmaceutically acceptable excipients.

The present application also provides articles of manufacture comprising any one of the antibody compositions or kits described herein. Examples of an article of manufacture include vials (including sealed vials).

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B(1992).

### Methods

### Gene Synthesis and Plasmid Construction

The coding sequences for HC and LC of the antibody were generated by DNA synthesis and PCR, subsequently subcloned into pTT5-based plasmid for protein expression in mammalian cell system. The gene sequences in the expression vectors were confirmed by DNA sequencing.

### Purification of Antibody Construct

Protein purification by affinity chromatography, and ion exchange chromatography was performed using an AKTA pure instrument (GE Lifesciences). Conditional medium expressing target antibody was harvested by centrifugation at 4000 rpm, 50 min, and filtered with a 0.22 μm filter. The harvested supernatants were loaded to a column of Mabselect™ SuRe™ (GE Healthcare). After washing column with Buffer A (PBS, PH 7.4), the protein was eluted with Buffer B (1 M Glycine, pH 2.7), and immediately neutralized with 1/10 volume of Buffer D (1 M sodium citrate, pH 6.0). The affinity purified antibody was then buffer exchanged into 20 mM sodium acetate pH 5.5.

### Measuring Antibody-IL-4 Binding Kinetics Using Surface Plasmon Resonance

A BIACORE® 8K SPR system (GE HealthCare) equipped with Series S Sensor Chip Protein G (Cytiva, Cat. 29179315) was used to determine the binding kinetic rate and affinity constants at 25° C. and in a running buffer of HBS-EP+ (10 mM HEPES pH 7.4, 150 mM NaCl, 3 mM EDTA, 0.05% Surfactant P20). Following a stabilization period in running buffer, the anti-IL13 mAb constructs (diluted to 1 μg/mL were captured onto flow cell 2 (active) for 60 see at a flow rate of 10 uL/min. Recombinant Human IL-4 Protein, His Tag (Acro Cat. IL3-H52H4) was prepared at concentrations of 0, 0.39, 0.78, 1.56, 3.13, 6.25, 12.5 and 0 nM and injected over flow cell 1 (reference) and flow cell 2 (active) for 180 sec at a flow rate of 30 μL/min. Recombinant Cynomolgus IL-4 Protein, His Tag (SINO BIOLOGICAL, Cat. 11057-C07H) was prepared at concentrations of 0, 0.39, 0.78, 1.56, 3.13, 6.25, 12.5, 25 and 0 nM and injected over flow cell 1 (reference) and flow cell 2 (active) for 180 sec at a flow rate of 30 μL/min. Samples were injected in a multi-cycle manner over freshly captured mAb, by regenerating the capture surfaces with injection of glycine pH 1.5 for 30 sec at a flow rate of 30 μL/min. The data was processed and analyzed with BIACORE® Insight Evaluation Software Version 2.0.15.12933 (GE Healthcare) as follows. Responses from flow cell 1 (reference) were subtracted from the responses from flow cell 2 (active). The responses from the two buffer blank injections were then subtracted from the reference subtracted data (2-1) to yield double-referenced data, which were fit to an 1:1 binding model to determine the apparent association (ka) and dissociation rate constants (kd). Their ratio provided the apparent equilibrium dissociation constant or affinity constant (KD=kd/ka).

### Example 1: Affinity Maturation of Anti-IL-4Rα Antibody

Dupilumab was used as a parental antibody for further CDR diversification to identify clones with improvements in potency, manufacturability, and pharmacokinetics.

In order to diversify CDRs while at least maintaining the potency of dupilumab, various mutations in all CDRs of both heavy and light chain were made. Site-directed PCR mutagenesis was employed to generate distinct libraries for each CDR in both heavy and light chain, resulting in 6 unique libraries with an average of 2-3 amino acid substitutions per chain. Individual mutants were displayed as Fabs in a phage display system and within each library, mutants were panned through two rounds of selection. The first round of selection consisted of 1 nM biotinylated hIL-4Rα and the output of that round was subsequently split into four distinct secondary rounds of parallel selection consisting of A) 1 nM biotinylated hIL-4Rα while also being washed for 4 hours at room temperature in buffer containing 100 nM of unlabeled hIL-4Rα, B) 0.1 nM biotinylated hIL-4Rα while also being washed for 4 hours at room temperature in buffer containing 10 nM of unlabeled hIL-4Rα, C) 50 nM biotinylated cyIL-4Rα with no wash, and D) 5 nM biotinylated cyIL-4Rα with no wash.

Mutant clones from the output of each arm of secondary selection were analyzed for k_{off} as a proxy for binding affinity by using periplasmic extracts of each mutant clone using surface plasmon resonance (SPR) and comparing to dupilumab, as well as bioinformatic analyses of sequences to identify patterns of enrichment. Mutants that exhibited no loss or improved binding relative to dupilumab as well as population enrichment throughout the selection were combined into a single library comprising all mutants in both the heavy and light chain. Fabs containing a random combinatorial mix of these individual CDR mutants were again screened in a phage display system, with multiple combinations of selection strategies outlined in TABLE 5.

TABLE 5

| Strategy | Round 1 Selection | Round 2 Selection |
|---|---|---|
| Strategy 1 | Antigen: 50 nM biotinylated cyIL-4Rα | Antigen: 50 nM biotinylated cyIL-4Rα |
| | Wash: No wash | Wash: 2 hr R.T. w/500 nM unlabeled |
| | | cyIL-4Rα |
| Strategy 2* | Antigen: 50 nM biotinylated cyIL-4Rα | Antigen: 5 nM biotinylated cyIL-4Rα |
| | Wash: No wash | Wash: 2 hr R.T. w/100 nM unlabeled |
| | | cyIL-4Rα |
| Strategy 3 | Antigen: 10⁻² nM biotinylated hIL-4Rα | Antigen: 10⁻² nM biotinylated hIL-4Rα |
| | Wash: 2 hr R.T. w/1 nM unlabeled | Wash: 24 hr R.T. w/1 nM unlabeled hIL- |
| | hIL-4Rα | 4Rα |
| Strategy 4 | Antigen: 10⁻² nM biotinylated hIL-4Rα | Antigen: 10⁻³ nM biotinylated hIL-4Rα |
| | Wash: 2 hr R.T. w/1 nM unlabeled | Wash: 24 hr R.T. w/0.1 nM unlabeled |
| | hIL-4Rα | hIL-4Rα |
| Strategy 5 | Antigen: 10⁻² nM biotinylated hIL-4Rα | Antigen: 10⁻⁴ nM biotinylated hIL-4Rα |
| | Wash: 2 hr R.T. w/1 nM unlabeled | Wash: 24 hr R.T. w/0.01 nM unlabeled |
| | hIL-4Rα | hIL-4Rα |

*Outputs from Round 2 selection in this strategy were selected in a third round consisting of 5 nM biotinylated cyIL-4Rα with a 16 hour room temperature wash with 500 nM unlabeled cyIL-4Rα.

### Example 2: Determination of Antibody Affinity to IL-4Rα

Binding affinity (KD) of antibodies to human and cynomolgus monkey IL-4Rα was determined through surface plasmon resonance (SPR) using a Carterra LSA. Briefly, an HC30M sensor chip that was previously functionalized with a polyclonal mixture of goat anti-human Fc antibody and used to capture purified antibodies at a level between 100-1000 RLU. Subsequently, concentrations of antigen ranging from 200 nM to 0.13 nM were injected over the surface at a rate of 2 mL/min. Regeneration of the chip between different concentrations of antigen was performed with 200 mM phosphoric acid and antibody was captured again as previously described. Association and dissociation rate constants were subsequently determined through fitting to a 1:1 Langmuir binding model using the Kinetics Software from Carterra from which a K_{D} value was derived. Results are summarized in TABLE 6.

The majority of antibodies bind to human IL-4Rα with low picomolar affinity comparable to dupilumab. Additionally, a number of antibodies demonstrated improved cross-reactivity to cynomolgus monkey IL-4Rα, some with low-nanomolar affinities.

TABLE 6

| | SPR Hu | Relative | SPR Cyno | Relative |
|---|---|---|---|---|
| | IL-4Rα | to | IL-4Rα | to |
| Antibody | K_{D} (pM) | Dupilumab | K_{D} (nM) | Dupilumab |
| Dupilumab | 11.29 | 1.00 | 628.82 | 1.00 |
| Construct 1 (mAb410) | 14.07 | 1.25 | 106.51 | 0.17 |
| Construct 2 (mAb411) | 18.14 | 1.61 | 64.18 | 0.10 |
| Construct 3 (mAb412) | 8.56 | 0.76 | 85.37 | 0.14 |
| Construct 4 (mAb413) | 14.01 | 1.24 | 128.19 | 0.20 |
| Construct 5 (mAb414) | 23.76 | 2.10 | 115.66 | 0.18 |
| Construct 6 (mAb415) | 11.55 | 1.02 | 59.47 | 0.09 |
| Construct 7 (mAb416) | W.S. | N/A | 1910.04 | 3.04 |
| Construct 8 (mAb417) | 9.40 | 0.83 | 156.60 | 0.25 |
| Construct 9 (mAb418) | 8.62 | 0.76 | 228.36 | 0.36 |
| Construct 10 (mAb419) | 12.47 | 1.10 | 113.75 | 0.18 |
| Construct 11 (mAb420) | 13.16 | 1.17 | 92.20 | 0.15 |
| Construct 12 (mAb421) | 21.55 | 1.91 | 15.12 | 0.02 |
| Construct 13 (mAb422) | 7.37 | 0.65 | 12.63 | 0.02 |
| Construct 15 (mAb423) | 13.68 | 1.21 | 24.59 | 0.04 |
| Construct 16 (mAb424) | 15.81 | 1.40 | 89.28 | 0.14 |
| Construct 17 (mAb425) | 16.86 | 1.49 | 58.27 | 0.09 |
| Construct 18 (mAb426) | 29.96 | 2.65 | 57.99 | 0.09 |
| Construct 19 (mAb427) | 35.98 | 3.19 | 50.17 | 0.08 |
| Construct 20 (mAb428) | 15.90 | 1.41 | 80.80 | 0.13 |
| Construct 21 (mAb429) | 8.57 | 0.76 | 14.41 | 0.02 |
| Construct 22 (mAb430) | 8.66 | 0.77 | 28.66 | 0.05 |
| Construct 23 (mAb431) | 7.62 | 0.67 | 63.29 | 0.10 |
| Construct 24 (mAb432) | 8.91 | 0.79 | 66.90 | 0.11 |
| Construct 25 (mAb433) | 10.66 | 0.94 | 703.39 | 1.12 |
| Construct 26 (mAb434) | 8.83 | 0.78 | 165.92 | 0.26 |
| Construct 27 (mAb435) | 12.64 | 1.12 | 175.72 | 0.28 |
| Construct 28 (mAb436) | 7.77 | 0.69 | 54.67 | 0.09 |
| Construct 29 (mAb437) | 8.10 | 0.72 | 15.65 | 0.02 |
| Construct 30 (mAb439) | 11.02 | 0.98 | 128.21 | 0.20 |
| Construct 31 (mAb443) | 8.29 | 0.73 | 421.26 | 0.67 |
| Construct 32 (mAb446) | 11.76 | 1.04 | 993.01 | 1.58 |
| Construct 33 (mAb450) | 8.75 | 0.78 | 33.92 | 0.05 |
| Construct 34 (mAb459) | 11.39 | 1.01 | 334.21 | 0.53 |
| Construct 35 (mAb467) | 11.15 | 0.99 | 179.65 | 0.29 |
| Construct 36 (mAb469) | 11.23 | 0.99 | 384.70 | 0.61 |
| Construct 37 (mAb470) | 11.58 | 1.03 | 143.47 | 0.23 |
| Construct 38 (mAb471) | 5.89 | 0.52 | 249.24 | 0.40 |
| Construct 39 (mAb473) | 10.42 | 0.92 | 168.06 | 0.27 |
| Construct 40 (mAb476) | 12.55 | 1.11 | 135.61 | 0.22 |
| Construct 41 (mAb478) | 9.73 | 0.86 | 248.24 | 0.39 |
| Construct 42 (mAb481) | 11.12 | 0.98 | 157.72 | 0.25 |
| Construct 43 (mAb482) | 9.43 | 0.84 | 150.22 | 0.24 |
| Construct 44 (mAb485) | 12.52 | 1.11 | 1278.98 | 2.03 |
| Construct 45 (mAb494) | 10.88 | 0.96 | 191.80 | 0.31 |
| Construct 46 (mAb501) | 11.00 | 0.97 | 228.06 | 0.36 |

W.S. Weak Signal, below Carterra sensitivity.
Relative values to Dupilumab, <1.00 is tighter affinity.

### Example 3: Improvement of Antibody Affinity to FcRn at pH 6.0

Binding affinity (K_{D}) of antibodies to human and cynomolgus monkey FcRn at pH 6.0 is a reliable correlate of half-life in vivo. K_{D} of purified antibodies to FcRn is determined through surface plasmon resonance (SPR) using a BIACORE® 8K. Briefly, an SPR chip functionalized with Protein G is used to capture purified antibodies normalized to 0.5 mg/mL, at a flow rate of 10 uL/min for 60 seconds. A paired channel with only buffer is used as reference. Subsequently, concentrations of human or cyno FcRn ranging from 25 nM to 0.39 nM are injected over the surface with captured purified antibody as well as the reference channel. Regeneration of the chip between different concentrations of IL-13 is performed with 10 mM Glycine HCl, pH 1.5 and antibody is again captured as previously described. Affinity measurement as described is done at both pH 7.4 and pH 6.0. Association and dissociation rate constants are subsequently determined through fitting to a 1:1 Langmuir binding model using the BIACORE® Insight Evaluation Software from which a KD value is derived. It is expected that all improved antibodies show enhanced binding to FcRn at pH 6.0 relative to dupilumab and is a strong indicator that such antibodies would have increased half-life in vivo. This is further confirmed in a pharmacokinetics study in cynomolgus monkey.

### Example 4: Inhibition of IL-13 and IL-4 Binding to hIL-13Rα/hIL-4Rα

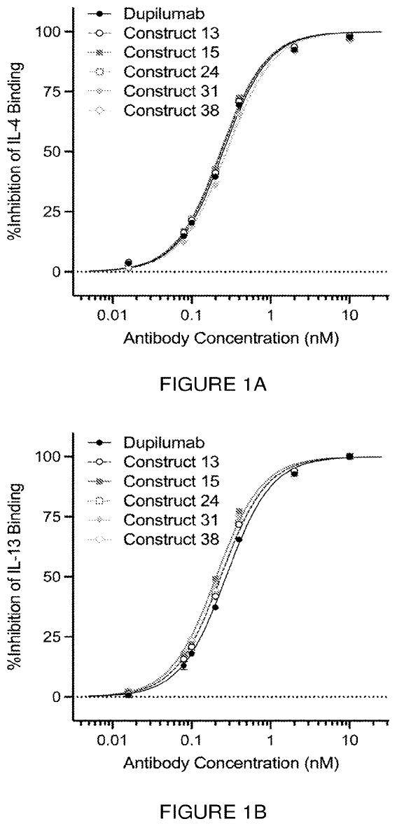

IL-13 and IL-4 binding to cells overexpressing hIL-13Rα/hIL-4Rα were used to evaluate the functional blockade of antibodies against this binding interaction. Briefly, HEK293 previously transduced to stably express both hIL-13Rα and hIL-4Rα were cultured and harvested. Cells were seeded at 200,000 cells in 100 uL per well. Cells were washed and the supernatant discarded. A 100 uL mixture of biotinylated hIL-13 OR biotinylated hIL-4 and purified antibody (1:1 by volume) that had been previously made and incubated for 1 hour was added to resuspend the cells, resulting in a final concentration of 0.05 ug/mL of hIL-13 OR 0.04 ug/mL of hIL-4 and 0-100 nM of purified antibody. The cells were stained in this mixture at 4° C. for 1 hour. Cells were then washed and stained with 100 μL of Alexa Fluor 488-conjugated streptavidin at a 1:1000 dilution to detect binding of biotinylated hIL-13 OR biotinylated hIL-4 on the cell surface. Cells were incubated 4° C. for 1 hour, protected from light. Cells were then washed and the MFI of cells in each well were recorded by FACS using a BD FACSCanto II. Subsequent data were analyzed using GraphPad Prism. IC50 values were determined as the concentration of antibody required to inhibit 50% of the maximum MFI of biotinylated hIL-13 OR biotinylated hIL-4 surface detected with incubation of 0.05 ug/mL of hIL-13 OR 0.04 ug/mL hIL-4 alone. Results are summarized in TABLE 7, TABLE 8, FIG. 1A and FIG. 1B

TABLE 7

| | Relative | Relative |
|---|---|---|
| | Blockade of IL-13 | Blockade of IL-4 |
| Antibody | Binding IC₅₀ (nM) | Binding IC₅₀ (nM) |
| Dupilumab | 1.000 | 1.000 |
| Construct 1 (mAb410) | 0.968 | 1.099 |
| Construct 2 (mAb411) | 0.942 | 0.799 |
| Construct 3 (mAb412) | 0.949 | 0.877 |
| Construct 4 (mAb413) | 0.989 | 1.007 |
| Construct 5 (mAb414) | 1.001 | 1.011 |
| Construct 6 (mAb415) | 0.990 | 1.161 |
| Construct 7 (mAb416) | 1.023 | 1.018 |
| Construct 8 (mAb417) | 0.953 | 1.013 |
| Construct 9 (mAb418) | 0.911 | 0.757 |
| Construct 10 (mAb419) | 0.922 | 0.992 |
| Construct 11 (mAb420) | 0.928 | 0.977 |
| Construct 12 (mAb421) | 0.921 | 1.069 |
| Construct 13 (mAb422) | 0.882 | 1.023 |
| Construct 15 (mAb423) | 0.876 | 0.660 |
| Construct 16 (mAb424) | 0.904 | 0.895 |
| Construct 17 (mAb425) | 1.172 | 1.107 |
| Construct 18 (mAb426) | 0.813 | 0.850 |
| Construct 19 (mAb427) | 1.021 | 0.867 |
| Construct 20 (mAb428) | 1.140 | 1.435 |
| Construct 21 (mAb429) | 0.706 | 0.749 |
| Construct 22 (mAb430) | 0.785 | 0.829 |
| Construct 23 (mAb431) | 0.793 | 1.067 |
| Construct 24 (mAb432) | 0.825 | 0.973 |
| Construct 25 (mAb433) | 1.087 | 1.357 |
| Construct 26 (mAb434) | 0.486 | 0.763 |
| Construct 27 (mAb435) | 0.592 | 0.990 |
| Construct 28 (mAb436) | 0.611 | 0.849 |
| Construct 29 (mAb437) | 0.695 | 0.887 |
| Construct 30 (mAb439) | 0.958 | 0.863 |
| Construct 31 (mAb443) | 0.964 | 1.012 |
| Construct 32 (mAb446) | 1.144 | 1.363 |
| Construct 33 (mAb450) | 1.210 | 1.013 |
| Construct 34 (mAb459) | 1.214 | 1.231 |
| Construct 35 (mAb467) | 1.250 | 1.291 |
| Construct 36 (mAb469) | 1.219 | 1.175 |
| Construct 37 (mAb470) | 1.047 | 1.084 |
| Construct 38 (mAb471) | 0.996 | 0.981 |
| Construct 39 (mAb473) | 1.055 | 1.093 |
| Construct 40 (mAb476) | 1.187 | 1.164 |
| Construct 41 (mAb478) | 1.150 | 1.095 |
| Construct 42 (mAb481) | 1.343 | 1.325 |
| Construct 43 (mAb482) | 1.131 | 1.018 |
| Construct 44 (mAb485) | 1.225 | 1.205 |
| Construct 45 (mAb494) | 1.014 | 1.081 |
| Construct 46 (mAb501) | 1.025 | 1.223 |

All values relative to dupilumab. <1.000 is more potent.

Certain constructs, shown in TABLE 8, were modified to replace the IgG4 YTE heavy chain constant region with an IgG1 YTE LALA heavy chain constant region (SEQ ID NO: 321). These antibodies were tested for their ability to inhibit IL-13 and IL-4 binding to hIL-13Rα/hIL-4Rα as compared to dupilumab. The results are summarized in TABLE 8. The values reflect absolute IC₅₀ measurements.

TABLE 8

| | Blockade of IL-13 | Blockade of IL-4 |
|---|---|---|
| Antibody | Binding IC₅₀ (nM) | Binding IC₅₀ (nM) |
| Dupilumab | 0.274 | 0.251 |
| Construct 13 (mAb422) | 0.235 | 0.238 |
| Construct 15 (mAb423) | 0.204 | 0.230 |
| Construct 24 (mAb432) | 0.234 | 0.234 |
| Construct 31 (mAb443) | 0.209 | 0.279 |
| Construct 38 (mAb471) | 0.226 | 0.271 |

### Example 5: Inhibition of IL-13-Induced and IL-4-Induced Phosphorylation of STAT6 in HT-29 Cells

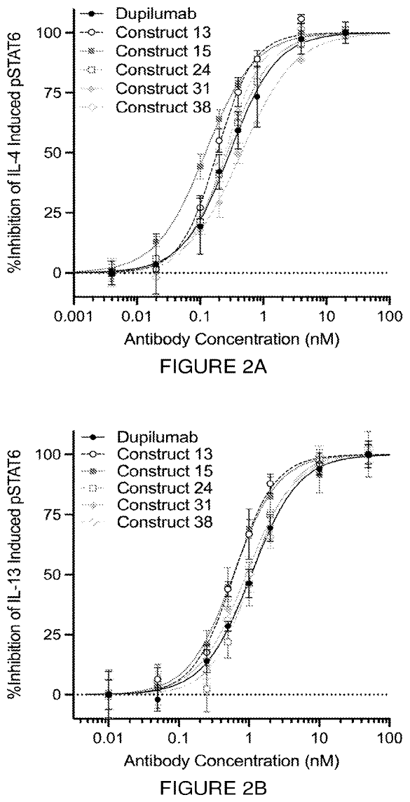

Inhibition of STAT6 phosphorylation in HT-29 cells was used to evaluate the functional activity of antibodies to block IL-13-induced and IL-4-induced biological activity. Briefly, HT-29 cells were starved in RMPI 1640+0.1% FBS overnight. Cells were collected and seeded at 50,000 cells per well in 100 μL. Concurrently, a 100 μL mixture of hIL-13 OR hIL-4 and purified antibody (1:1 by volume) was added to the same well, resulting in a final concentration of 10 ng/mL of hIL-13 OR 5 ng/mL of hIL-4 and 0-50 nM of purified antibody. Cells were incubated at 37° C. for 1 hour and subsequently fixed, permeabilized, and stained with a PE-conjugated anti-pSTAT6 antibody. The MFI of cells in each well were recorded by FACS using a BD FACSCanto II and subsequent data were analyzed using GraphPad Prism. IC50 values were determined as the concentration of antibody required to inhibit 50% of the maximum MFI of pSTAT6 detected with incubation of 10 ng/mL of hIL-13 OR 5 ng/mL of hIL-4 alone. Results are summarized in TABLE 9, TABLE 10, FIG. 2A, and FIG. 2B.

As shown in TABLE 9, clones were identified that demonstrated more effective inhibition of IL-13 and/or IL-4 induced phosphorylation of STAT6 as compared to dupilumab by up to about 2-fold (such as, for example: construct 15, construct 18, construct 21, construct 23, construct 24, construct 27 and construct 28).

TABLE 9

| | Relative | Relative |
|---|---|---|
| | Inhibition of | Inhibition of |
| | IL-13 induced | IL-4 induced |
| Antibody | pSTAT6 IC₅₀ (nM) | pSTAT6 IC₅₀ (nM) |
| Dupilumab | 1.000 | 1.000 |
| Construct 1 (mAb410) | 0.671 | 0.592 |
| Construct 2 (mAb411) | 0.685 | 0.507 |
| Construct 3 (mAb412) | 0.660 | 0.767 |
| Construct 4 (mAb413) | 0.862 | 0.493 |
| Construct 5 (mAb414) | 1.075 | 0.780 |
| Construct 6 (mAb415) | 1.348 | 0.729 |
| Construct 7 (mAb416) | 1.978 | 1.008 |
| Construct 8 (mAb417) | 2.098 | 1.009 |
| Construct 9 (mAb418) | 1.309 | 0.761 |
| Construct 10 (mAb419) | 1.384 | 0.599 |
| Construct 11 (mAb420) | 0.924 | 0.638 |
| Construct 12 (mAb421) | 0.672 | 0.457 |
| Construct 13 (mAb422) | 0.824 | 0.415 |
| Construct 15 (mAb423) | 0.468 | 0.285 |
| Construct 16 (mAb424) | 1.407 | 0.825 |
| Construct 17 (mAb425) | 0.863 | 0.876 |
| Construct 18 (mAb426) | 0.446 | 0.517 |
| Construct 19 (mAb427) | 0.885 | 0.844 |
| Construct 20 (mAb428) | 1.862 | 1.278 |
| Construct 21 (mAb429) | 0.447 | 0.305 |
| Construct 22 (mAb430) | 0.612 | 0.749 |
| Construct 23 (mAb431) | 0.475 | 0.483 |
| Construct 24 (mAb432) | 0.586 | 0.629 |
| Construct 25 (mAb433) | 1.818 | 1.335 |
| Construct 26 (mAb434) | 0.627 | 0.661 |
| Construct 27 (mAb435) | 0.380 | 0.676 |
| Construct 28 (mAb436) | 0.497 | 0.999 |
| Construct 29 (mAb437) | 0.624 | 0.630 |
| Construct 30 (mAb439) | 0.781 | 1.086 |
| Construct 31 (mAb443) | 1.607 | 1.233 |
| Construct 32 (mAb446) | 4.756 | 1.675 |
| Construct 33 (mAb450) | 1.587 | 0.888 |
| Construct 34 (mAb459) | 2.420 | 0.464 |
| Construct 35 (mAb467) | 2.714 | 0.766 |
| Construct 36 (mAb469) | 2.158 | 0.957 |
| Construct 37 (mAb470) | 1.222 | 0.713 |
| Construct 38 (mAb471) | 1.561 | 0.723 |
| Construct 39 (mAb473) | 2.172 | 0.810 |
| Construct 40 (mAb476) | 1.894 | 0.633 |
| Construct 41 (mAb478) | 1.967 | 1.184 |
| Construct 42 (mAb481) | 2.784 | 0.810 |
| Construct 43 (mAb482) | 2.289 | 0.633 |
| Construct 44 (mAb485) | 3.762 | 1.184 |
| Construct 45 (mAb494) | 4.609 | 1.254 |
| Construct 46 (mAb501) | 3.605 | 1.039 |

All values relative to dupilumab. <1.000 is more potent.

The constructs shown in TABLE 10 were modified to replace the IgG4 YTE heavy chain constant region with an IgG1 YTE LALA constant region (SEQ ID NO: 321). These constructs were tested for their ability to inhibit IL-13 and/or IL-4 induced phosphorylation of STAT6 as compared to dupilumab. These results are summarized in TABLE 10. The values reflect absolute IC₅₀ measurements.

TABLE 10

| | Inhibition of | Inhibition of |
|---|---|---|
| | IL-13 induced | IL-4 induced |
| Antibody | pSTAT6 IC₅₀ (nM) | pSTAT6 IC₅₀ (nM) |
| Dupilumab | 1.075 | 0.296 |
| Construct 13 (mAb422) | 0.612 | 0.185 |
| Construct 15 (mAb423) | 0.599 | 0.122 |
| Construct 24 (mAb432) | 1.089 | 0.269 |
| Construct 31 (mAb443) | 0.916 | 0.452 |
| Construct 38 (mAb471) | 1.046 | 0.248 |

### Example 6: Inhibition of IL-13-Induced and IL-4-Induced Release of TARC from A549 Cells

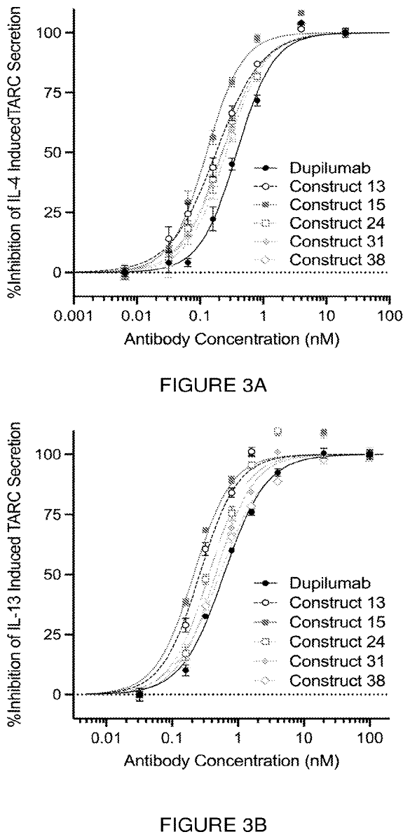

Inhibition of TARC secretion by A549 cells was used to evaluate the functional activity of antibodies to block IL-13-induced and IL-4-induced biological activity. Briefly, A549 cells were seeded at 20,000 cells in 100 μL of DMEM+10% FBS and cultured overnight at 37° C. The next day, the cell culture media was discarded and cells were gently washed with fresh media. A 150 μL mixture of hIL-13, purified antibody, and hTNFa (1:1:1 by volume) were added to the wells, resulting in a final concentration of 20 ng/mL hIL-13, 0-100 nM purified antibody, and 200 ng/mL hTNFa OR 1.5 ng/mL hIL-4, 0-100 nM purified antibody, and 50 ng/mL TNFa. Cells were incubated in this mixture at 37° C. for 20-24 hour. Following incubation, culture supernatant was collected and the amount of TARC present was analyzed using a commercial TARC ELISA kit (R&D Systems), analyzed according to manufacturer's instructions. The determined concentrations of TARC in each well were analyzed using GraphPad Prism. IC50 values were determined as the concentration of antibody required to inhibit 50% of the maximum TARC concentration detected with incubation of only 20 ng/mL of hIL-13 and 200 ng/mL hTNFa OR 1.5 ng/mL hIL-4 and 50 ng/mL hTNFa. Results are summarized in TABLE 11, TABLE 12, FIG. 3A, and FIG. 3B.

As shown in TABLE 11, clones were identified that demonstrated more effective inhibition of IL-13 and/or IL-4 induced TARC as compared to dupilumab by up to about 3-fold (such as, for example: construct 2, and construct 15).

TABLE 11

| | Relative | Relative |
|---|---|---|
| | Inhibition of | Inhibition of |
| | IL-13 induced | IL-4 induced |
| Antibody | TARC IC₅₀ (nM) | TARC IC₅₀ (nM) |
| Dupilumab | 1.000 | 1.000 |
| Construct 1 (mAb410) | 0.485 | 0.427 |
| Construct 2 (mAb411) | 0.314 | 0.267 |
| Construct 3 (mAb412) | 0.506 | 0.521 |
| Construct 4 (mAb413) | 0.614 | 0.266 |
| Construct 5 (mAb414) | 0.897 | 0.552 |
| Construct 6 (mAb415) | 1.435 | 0.879 |
| Construct 7 (mAb416) | 1.136 | 0.802 |
| Construct 8 (mAb417) | 1.053 | 1.113 |
| Construct 9 (mAb418) | 0.942 | 0.593 |
| Construct 10 (mAb419) | 0.856 | 0.556 |
| Construct 11 (mAb420) | 0.877 | 0.609 |
| Construct 12 (mAb421) | 0.521 | 0.380 |
| Construct 13 (mAb422) | 1.012 | 0.507 |
| Construct 15 (mAb423) | 0.350 | 0.453 |
| Construct 16 (mAb424) | 1.026 | 1.629 |
| Construct 17 (mAb425) | 0.789 | 0.491 |
| Construct 18 (mAb426) | 0.447 | 0.407 |
| Construct 19 (mAb427) | 0.834 | 0.756 |
| Construct 20 (mAb428) | 1.828 | 3.628 |
| Construct 21 (mAb429) | 0.313 | 0.342 |
| Construct 22 (mAb430) | 0.535 | 0.488 |
| Construct 23 (mAb431) | 0.579 | 0.557 |
| Construct 24 (mAb432) | 0.594 | 0.445 |
| Construct 25 (mAb433) | 1.781 | 1.174 |
| Construct 26 (mAb434) | 1.734 | 0.763 |
| Construct 27 (mAb435) | 1.833 | 0.547 |
| Construct 28 (mAb436) | 1.808 | 0.580 |
| Construct 29 (mAb437) | 1.392 | 0.693 |
| Construct 30 (mAb439) | 0.592 | 0.68 |
| Construct 31 (mAb443) | 0.703 | 1.37 |
| Construct 32 (mAb446) | 2.403 | 4.36 |
| Construct 33 (mAb450) | 1.075 | 3.20 |
| Construct 34 (mAb459) | 1.351 | 4.32 |
| Construct 35 (mAb467) | 1.654 | 1.92 |
| Construct 36 (mAb469) | 0.999 | 1.78 |
| Construct 37 (mAb470) | 0.868 | 1.16 |
| Construct 38 (mAb471) | 1.634 | 1.17 |
| Construct 39 (mAb473) | 5.227 | 1.65 |
| Construct 40 (mAb476) | 4.703 | 1.60 |
| Construct 41 (mAb478) | 1.353 | 1.53 |
| Construct 42 (mAb481) | 1.616 | 2.02 |
| Construct 43 (mAb482) | 2.304 | 1.56 |
| Construct 44 (mAb485) | 5.717 | 2.53 |
| Construct 45 (mAb494) | 14.466 | 3.09 |
| Construct 46 (mAb501) | 4.662 | 2.49 |

All values relative to dupilumab. <1.000 is more potent.

The constructs shown in TABLE 12 were modified to replace the IgG4 YTE heavy chain constant region with an IgG1 YTE LALA heavy chain constant region (SEQ ID NO: 321). These constructs were tested for their ability to inhibit IL-13 and/or IL-4 induced TARC as compared to dupilumab. These results are summarized in TABLE 12. The values reflect absolute IC₅₀ measurements.

TABLE 12

| | Inhibition | Inhibition |
|---|---|---|
| | of IL-13 induced | of IL-4 induced |
| Antibody | TARC IC₅₀ (nM) | TARC IC₅₀ (nM) |
| Dupilumab | 0.612 | 0.382 |
| Construct 13 (mAb422) | 0.261 | 0.179 |
| Construct 15 (mAb423) | 0.212 | 0.127 |
| Construct 24 (mAb432) | 0.361 | 0.222 |
| Construct 31 (mAb443) | 0.456 | 0.228 |
| Construct 38 (mAb471) | 0.527 | 0.259 |

### Example 7: Inhibition of IL-13-Induced and IL-4-Induced Proliferation of TF-1 Cells

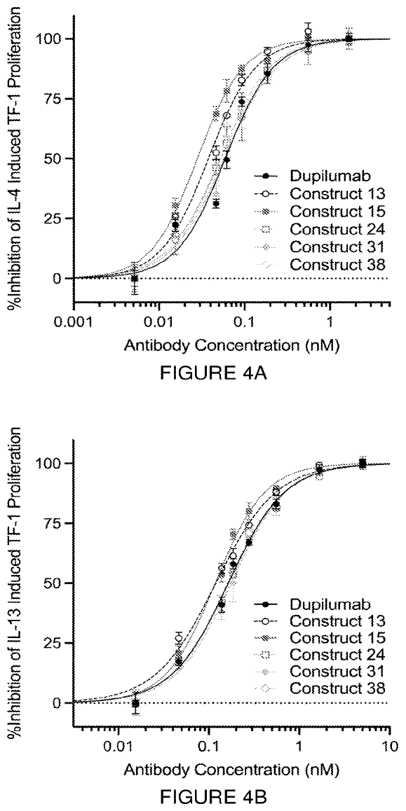

The proliferation or inhibition thereof of TF-1 cells was used to evaluate the functional activity of antibodies to block IL-13-induced and IL-4-induced biological activity. Briefly, TF-1 cells were harvested and starved in RPMI1640+10% FBS without additional cytokine for 4 hours. During this time, a mixture of hIL-13 OR hIL-4 and purified antibody (1:1 by volume) was prepared 50 μL was added per well. Following starvation, TF-1 cells were again harvested and seeded at 15,000 cells in 50 μL per well, resulting in a final concentration of 4 ng/mL of hIL-13 OR 0.5 ng/mL of hIL-4 and 0-5 nM purified antibody. Cells were subsequently incubated at 37° C. for 72 hours and proliferation of cells was quantified using CellTiter-Glo (Promega) according to manufacturer's instructions. Luminescence was recorded by SpectraMax M5 Multimode Plate Reader and data was analyzed using GraphPad Prism. IC50 values were determined as the concentration of antibody required to result in 50% of the maximum luminescence detected when TF-1 cells are incubated and cultured with 4 ng/mL of hIL-13 OR 0.5 ng/mL of hIL-4 alone. Results are summarized in TABLE 13, TABLE 14, FIG. 4A, and FIG. 4B.

TABLE 13

| | Relative Inhibition | Relative Inhibition |
|---|---|---|
| | of IL-13 induced | of IL-4 induced |
| | TF-1 Proliferation | TF-1 Proliferation |
| Antibody | IC₅₀ (nM) | (IC₅₀ nM) |
| Dupilumab | 1.000 | 1.000 |
| Construct 1 (mAb410) | 1.368 | 0.414 |
| Construct 2 (mAb411) | 0.870 | 0.376 |
| Construct 3 (mAb412) | 0.683 | 0.514 |
| Construct 4 (mAb413) | 1.109 | 0.442 |
| Construct 5 (mAb414) | 0.940 | 0.521 |
| Construct 6 (mAb415) | 2.056 | 0.920 |
| Construct 7 (mAb416) | 1.379 | 1.514 |
| Construct 8 (mAb417) | 1.465 | 0.734 |
| Construct 9 (mAb418) | 1.342 | 0.503 |
| Construct 10 (mAb419) | 1.390 | 0.431 |
| Construct 11 (mAb420) | 1.723 | 0.455 |
| Construct 12 (mAb421) | 0.870 | 0.526 |
| Construct 13 (mAb422) | 0.794 | 0.675 |
| Construct 15 (mAb423) | 0.710 | 0.281 |
| Construct 16 (mAb424) | 1.443 | 1.037 |
| Construct 17 (mAb425) | 0.844 | 0.729 |
| Construct 18 (mAb426) | 0.606 | 0.626 |
| Construct 19 (mAb427) | 0.550 | 0.835 |
| Construct 20 (mAb428) | 1.379 | 1.124 |
| Construct 21 (mAb429) | 0.498 | 0.509 |
| Construct 22 (mAb430) | 0.495 | 0.746 |
| Construct 23 (mAb431) | 0.732 | 0.823 |
| Construct 24 (mAb432) | 0.693 | 1.033 |
| Construct 25 (mAb433) | 1.309 | 2.035 |
| Construct 26 (mAb434) | 0.830 | 0.700 |
| Construct 27 (mAb435) | 0.773 | 0.962 |
| Construct 28 (mAb436) | 0.634 | 1.186 |
| Construct 29 (mAb437) | 0.851 | 0.486 |
| Construct 30 (mAb439) | 0.917 | 0.387 |
| Construct 31 (mAb443) | 1.237 | 1.251 |
| Construct 32 (mAb446) | 2.006 | 1.735 |
| Construct 33 (mAb450) | 0.926 | 1.050 |
| Construct 34 (mAb459) | 1.555 | 1.464 |
| Construct 35 (mAb467) | 1.115 | 0.948 |
| Construct 36 (mAb469) | 1.246 | 1.046 |
| Construct 37 (mAb470) | 0.919 | 0.943 |
| Construct 38 (mAb471) | 1.202 | 1.044 |
| Construct 39 (mAb473) | 1.208 | 1.182 |
| Construct 40 (mAb476) | 1.011 | 0.968 |
| Construct 41 (mAb478) | 1.148 | 0.820 |
| Construct 42 (mAb481) | 1.185 | 1.190 |
| Construct 43 (mAb482) | 0.941 | 1.133 |
| Construct 44 (mAb485) | 1.288 | 1.148 |
| Construct 45 (mAb494) | 1.788 | 1.676 |
| Construct 46 (mAb501) | 1.285 | 1.294 |

All values relative to dupilumab. <1.000 is more potent.

The constructs shown in TABLE 14 were modified to replace the IgG4 YTE heavy chain constant region with an IgG1 YTE LALA heavy chain constant region (SEQ ID NO: 321). These constructs were tested for their ability to inhibit IL-13 induced TF-1 proliferation as compared to dupilumab. These results are summarized in TABLE 14. The values reflect absolute IC₅₀ measurements.

TABLE 14

| | Inhibition of | Inhibition of |
|---|---|---|
| | IL-13 induced TF-1 | IL-4 induced TF-1 |
| | Proliferation | Proliferation |
| Antibody | IC₅₀ (nM) | IC₅₀ (nM) |
| Dupilumab | 0.164 | 0.059 |
| Construct 13 (mAb422) | 0.119 | 0.038 |
| Construct 15 (mAb423) | 0.117 | 0.028 |
| Construct 24 (mAb432) | 0.166 | 0.052 |
| Construct 31 (mAb443) | 0.175 | 0.058 |
| Construct 38 (mAb471) | 0.161 | 0.052 |

### Example 8: Improving Developability of Anti-IL-4Rα Antibodies

The manufacturing and clinical performance of antibodies are often improved when the antibodies themselves have improved biophysical characteristics. In order to assess some of these characteristics, purified antibodies were analyzed for their hydrophobic quality and non-specificity. Briefly, for hydrophobic quality, 10 μg of purified antibodies were injected into a hydrophobic interaction chromatography (HIC) Butyl 1.7 μm column in a mobile phase of sodium phosphate to capture antibodies to the column followed by elution of antibodies using ammonium sulphate retention time of antibody was used as a directly proportional measurement of hydrophobicity, i.e., a lower retention time indicates an antibody of lower hydrophobic character.

ELISA was used to assess non-specificity. Briefly, ELISA plates were coated with a 0.15% baculovirus particle (BVP) suspension and incubated at 4° C., overnight. Plates were blocked with 1% BSA for 2 hours at 37° C. 200 nM of purified antibodies were added and incubated for 1 hour at 37° C. An HRP-conjugated goat anti-human IgG Fc antibody was used to detect binding of purified antibodies, incubated for 0.5 hour at 37° C. Between all steps, plates were washed multiple times in PBS-T. Plates were developed using the substrate TMB over the course of 15 minutes, with the reaction being halted through the addition of 1N HCl. Absorbance at 450 nm was recorded by SpectraMax Plus 384 Microplate Reader and data was analyzed using GraphPad Prism. Absorbance of 450 nm of each antibody was used as a directly proportional measurement of non-specificity, i.e., a lower absorbance indicates an antibody with expected low non-specific binding. Results are summarized in TABLE 15.

As shown in TABLE 15 below, nearly all of the antibodies show lower HIC and BVP scores than dupilumab.

TABLE 15

| | Hydrophobic | Baculovirus |
|---|---|---|
| | Interaction | Particle 200 nM |
| Antibody | Retention Time (min) | ELISA Score |
| Dupilumab | 9.24 | 9.8 |
| Construct 1 (mAb410) | 7.99 | 2.0 |
| Construct 2 (mAb411) | N.T. | 4.2 |
| Construct 3 (mAb412) | 8.62 | 3.3 |
| Construct 4 (mAb413) | 8.75 | 2.1 |
| Construct 5 (mAb414) | 7.98 | 1.5 |
| Construct 6 (mAb415) | 8.43 | 1.9 |
| Construct 7 (mAb416) | 7.85 | 1.1 |
| Construct 8 (mAb417) | 8.11 | 1.5 |
| Construct 9 (mAb418) | 7.70 | 1.4 |
| Construct 10 (mAb419) | 8.13 | 3.9 |
| Construct 11 (mAb420) | 8.26 | 2.4 |
| Construct 12 (mAb421) | 8.17 | 4.8 |
| Construct 13 (mAb422) | 8.39 | 3.2 |
| Construct 15 (mAb423) | 8.13 | 2.3 |
| Construct 16 (mAb424) | 8.68 | 3.8 |
| Construct 17 (mAb425) | 8.25 | 1.9 |
| Construct 18 (mAb426) | 7.74 | 3.0 |
| Construct 19 (mAb427) | 8.75 | 2.6 |
| Construct 20 (mAb428) | 8.45 | 2.5 |
| Construct 21 (mAb429) | 9.26 | 7.1 |
| Construct 22 (mAb430) | 8.46 | 6.7 |
| Construct 23 (mAb431) | 7.93 | 3.8 |
| Construct 24 (mAb432) | 7.81 | 3.7 |
| Construct 25 (mAb433) | 7.74 | 1.8 |
| Construct 26 (mAb434) | 8.63 | 3.5 |
| Construct 27 (mAb435) | 8.19 | 2.4 |
| Construct 28 (mAb436) | 8.27 | 2.4 |
| Construct 29 (mAb437) | 8.27 | 2.2 |
| Construct 30 (mAb439) | 6.54 | 2.0 |
| Construct 31 (mAb443) | 7.34 | 1.6 |
| Construct 32 (mAb446) | 7.41 | 1.9 |
| Construct 33 (mAb450) | 7.89 | 3.1 |
| Construct 34 (mAb459) | 7.28 | 1.7 |
| Construct 35 (mAb467) | 7.00 | 4.1 |
| Construct 36 (mAb469) | 6.43 | 1.8 |
| Construct 37 (mAb470) | 7.68 | 7.9 |
| Construct 38 (mAb471) | 6.54 | 1.5 |
| Construct 39 (mAb473) | 6.67 | 2.4 |
| Construct 40 (mAb476) | 7.39 | 1.8 |
| Construct 41 (mAb478) | 7.75 | 4.5 |
| Construct 42 (mAb481) | 8.60 | 1.9 |
| Construct 43 (mAb482) | 6.04 | 2.2 |
| Construct 44 (mAb485) | 6.52 | 2.3 |
| Construct 45 (mAb494) | 8.80 | 1.8 |
| Construct 46 (mAb501) | 8.77 | 2.0 |

N.T.: Not Tested

### Example 9. Improving Solubility and Viscosity of Anti-IL-4Rα Antibodies

Purified antibodies are analyzed for their maximum solubility and corresponding viscosity. Briefly, for maximum solubility, purified antibodies are formulated into a buffer of 20 mM Histidine, 4% sucrose, 0.04% PS80 and pH X, Y, and Z and are concentrated. Concentration at antibody at this point is evaluated and is determined to be the maximum solubility of the antibody at that pH. Subsequently, viscosity of the antibody at that concentration and pH are determined. Several antibodies show improved solubility of greater than 150 mg/mL and greater viscosity relative to Dupilumab, which allows these antibodies to be delivered at a higher dose per administration and reduce the frequency of injections to the patient.

### Example 10. Determination of Antibody Affinity to IL-4Rα

Binding affinity (KD) of exemplary antibodies to human IL-4Rα was determined using the Kinetic Exclusion Assay (KinExA). Briefly, antibody and recombinant human IL-4Rα were equilibrated in solution. For each antibody, equilibration samples were done with a fixed concentration of the antibody ranging from 2.27 μm to 100 pM and a titration of recombinant human IL-4Rα generated from a starting concentration ranging from 9.78 pM to 1.00 nM with 2-fold dilutions for a total set of dilutions ranging from 11 to 13 concentrations. Samples were incubated for a time period ranging from 3.5 hours to 340 hours, to fully reach equilibrium. Once samples reached equilibrium, azlactone beads previously coated with recombinant human IL-4Rα were used to capture any free antibody from the equilibrated solution. Captured antibody was then detected with an Alexa Fluor 647-labeled Goat Anti-Human IgG. The KinExA 4000 instrument was used to capture fluorescent signal and convert to a voltage signal that is directly proportional to the amount of free antibody in all the equilibrated samples, allowing for the determination of the apparent affinity of the antibody. Results are summarized in TABLE 16.

TABLE 16

| | Hu IL-4Rα | 95% Confidence | Relative to |
|---|---|---|---|
| Antibody | K_{D} (fM) | Interval (fM) | Dupilumab |
| Dupilumab | 1120 | 644-1760 | 1.00 |
| Construct 13 (mAb422) | 299 | 160-482 | 0.267 |
| Construct 15 (mAb423) | 10.9 | 2.4-26 | 0.010 |
| Construct 24 (mAb432) | 118 | 52.6-199 | 0.105 |
| Construct 31 (mAb443) | 282 | 138-492 | 0.252 |
| Construct 38 (mAb471) | 421 | 256-636 | 0.376 |

The majority of exemplary antibodies bind to human IL-4Rα with femtomolar affinity compared to dupilumab that binds with low picomolar affinity.

### Example 11. Pharmacokinetic Analysis of Anti-IL-4Rα Antibodies

In vivo pharmacokinetic (PK) studies were performed to evaluate the half-life extension of antibodies over dupilumab. Studies were performed using cynomolgus monkey (Macaca fascicularis), where any matching SQ/IV cohorts were all males, ranging from 2.52 to 3.86 kg in weight. Animals were administered dupilumab, the exemplary antibody Construct 13 (mAb422), or the exemplary antibody Construct 38 (mAb471), where the IgG4 YTE constant heavy chain regions of Construct 13 and Construct 38 were replaced by IgG1 YTE LALA constant regions (SEQ ID NO: 321), by intravenous (IV) bolus or subcutaneous (SQ) injection on Day 0 at a dose of 1, 5 or 25 mg/kg, as shown in TABLE 17, and serum samples were taken regularly throughout the study.

TABLE 17

| | | Dose | Route of | | Half-Life |
|---|---|---|---|---|---|
| Antibody | Group | (mg/kg) | Administration | N | (Days) |
| Dupilumab | 1 | 25 | IV | 55 | 10.88 |
| | 2 | 25 | SC | 55 | 10.95 |
| | 3 | 1 | IV | 14 | 10.10 |
| Construct 13 | 4 | 25 | IV | 14 | 17.62 |
| (mAb422) | 5 | 25 | SC | 14 | 17.49 |
| | 6 | 5 | IV | 14 | 13.71 |
| | 7 | 1 | IV | 14 | 5.43 |
| Construct 38 | 8 | 25 | IV | 15 | 25.60 |
| (mAb471) | 9 | 25 | SC | 15 | 27.18 |
| | 10 | 5 | IV | 15 | 24.02 |
| | 11 | 1 | IV | 15 | 16.74 |

All SC cohort values are adjusted for bioavailability

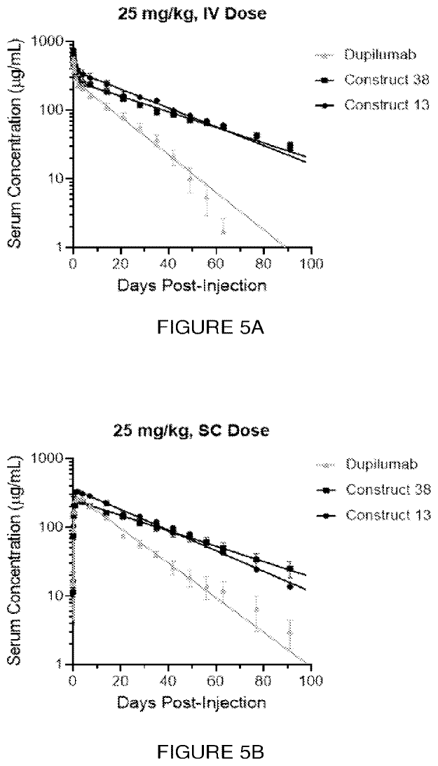

PK parameters were determined from cynomolgus serum samples up to day 91. The PK analysis demonstrated that Construct 13 and Construct 38 had a half-life of 17.62 and 25.60 days, respectively, compared with 10.88 days for dupilumab (25 mg/kg, IV). The results are summarized in TABLE 17. The PK analysis demonstrated that the exemplary antibodies, Construct 13 (mAb422) and Construct 38 (mAb471), had improved half-life compared to those of dupilumab (TABLE 17, FIG. 5A, and FIG. 5B) across different routes of administration. The half-life of Construct 38 when dosed at 5 and 1 mg/kg, IV, remained superior to dupilumab when dosed at 25 mg/kg, IV. The increased half-life of the exemplary antibodies may enable less frequent dosing compared with currently available treatment protocols, reducing the burden of injection and increasing compliance for patients living with COPD and other diseases.

In cases where bioavailability (F) is determined, antibodies disclosed herein are shown to possess equivalent bioavailability to that of dupilumab. The results for the exemplary antibodies and dupilumab are summarized in TABLE 18. Construct 38 (mAb471) showed comparable bioavailability to dupilumab.

TABLE 18

| Antibody | Bioavailability (%) |
|---|---|
| Dupilumab | 100% |
| Construct 13 (mAb422) | 81.26% |
| Construct 38 (mAb471) | 97.51% |

### Example 12. Binning Experiments with (Dupilumab) Anti-IL-4Rα Antibodies

Epitope binning is a technique used to cluster different mAbs based on the specific region of the antigen (in this case IL-4Rα) that is recognized by the antibody. In binning studies with immobilized dupilumab, no response was observed for constructs mAb410, mAb411, mAb412, mAb413, mAb414, mAb415, mAb416, mAb417, mAb418, mAb419, mAb420, mAb421, mAb422, mAb422B, mAb423, mAb424, mAb425, mAb426, mAb427, mAb428, mAb429, mAb431, mAb432, mAb433, mAb434, mAb435, mAb436, mAb437, mAb439, mAb443, mAb450, mAb470, mAb471, and mAb476. This indicated that the recited mAbs and dupilumab binned together and provides evidence that the mAbs likely bind to a similar or the same epitope on IL-4Rα and therefore they are more likely to have the same biological effect.

### Example 13. Half-Life Estimation

Half-life for antibodies is a product of degradation or elimination through three pathways: pinocytosis, target-mediated drug disposition (TMDD) and receptor-mediated endocytosis.

Pinocytosis: Pinocytosis is a non-specific process in which extracellular fluid and substances are brought into the cell, resulting in an internalized vesicle. This internal vesical then fuses with lysosomes. All antibodies are subject to this elimination pathway.

TMDD: This is a receptor-mediated endocytosis process, meaning that the interactions of the antibody with its receptor results in the internalization of the antibody-receptor complex and subsequent degradation via lysosomes, specialized organelles, or areas within the cell that degrade molecules and other biomaterial. TMDD is commonly observed for mAbs with receptor targets, such as dupilumab, which targets IL-4Rα.

Receptor-mediated endocytosis: Binding of antibodies to Fc-gamma-receptors, present on many immune cells, can also trigger an elimination process similar to TMDD. However, third-party preclinical studies have demonstrated that this degradation pathway plays only a minor role in the elimination of antibodies, if it plays any role at all.

To estimate half-life for the disclosed antibodies that bin with dupilumab, the VL and VH domains of dupilumab were combined with an IgG1-YTE constant region (“IL-4Rα tool compound”) and half-life in non-human primates (NHPs) was determined. The IL-4Rα tool compound incorporating YTE amino acid substitutions demonstrated a half-life of 18.5 days versus 10.5 days for dupilumab, an increase of 76%. Accordingly, given the improved half-life of the IL-4Rα tool compound, it is believed that the antibodies disclosed herein will have significantly improved dosing over prior art antibodies (e.g., dupilumab).

Antibody recycling through increased affinity for FcRn, as described above, impacts degradation via pinocytosis, but not elimination via target-mediated drug disposition (TMDD). Accordingly, half-life extensions provided by YTE mutations in antibodies with membrane-bound targets were used in the following analysis.

As one example, CDX-0159 is an antibody targeting KIT (c-KIT/CD117) receptor tyrosine kinase with YTE amino acid substitutions for half-life extension currently in clinical development. In NHPs, half-life was shown to be 22 days for CDX-0159 compared to 4.8 days for CDX-0158, a non-half-life extended antibody directed at the same target. Clinically, CDX-0159 showed a 32-day half-life, suggesting an approximately one-and-a-half times increase over NHP data. Further, CDX-0159 has shown a human half-life that is approximately five times greater than CDX-0158, the non-half-life extended antibody directed at the same target (half-life of CDX-0159 was 32 days versus 6 days for CDX-0158).

As another example, VRDN-002 is an antibody targeting anti-IGF-1 receptor with recycling-based FC modifications for half-life extension (i.e., YTE or LS or similar amino acid substitutions) currently in clinical development. In NHPs, half-life was shown to be 14 days for VRDN-002 compared to 6.4 days for teprotumumab, a non-half-life extended antibody directed at the same target. Clinically, VRDN-002 showed an approximately 30- to 40-day half-life in an interim analysis, suggesting an approximately two to three times increase over NHP data. Further, VRDN-002 has shown a human half-life that is approximately three to four times greater than teprotumumab, the non-half-life extended antibody directed at the same target (half-life of VRDN-002 was approximately 30 to 40 days compared to approximately 10 to 11 days for teprotumumab).

Based on these studies (and depending upon the approach taken), it is expected that the antibodies disclosed herein may have a human half-life of approximately 30 to 60 days, based on a one-and-a-half to three times factor going from NHPs to humans, or of approximately 45 to 70 days, based on a three to five times factor going from non-half-life extended antibodies to half-life extended antibodies directed at the same receptor target.

To further estimate half-life of the antibodies disclosed herein, a two-compartment model was built with first-order absorption and parallel linear and Michaelis-Menten elimination, the latter corresponding to TMDD effects associated with targeting membrane-bound IL4Ra, to predict the concentration (drug levels) of both dupilumab and the antibodies disclosed herein, over time. Parameters included 0.0447 day-1 for elimination rate (ke), 2.74 L for central volume (Vc), 0.306 day-1 for absorption rate (ka) and 64.2% for bioavailability.

It is believed that efficacy in inflammatory conditions, such as COPD, is driven by C_{trough}, or the minimal concentration of the mAb. Therefore, based on the model described above, a C_{trough} for the disclosed antibodies was targeted to be equal to dupilumab's C_{trough} in maintenance with 300 mg SC every two weeks dosing, which was approximately 75 mg/L. Given the overlapping epitopes of dupilumab and the antibodies disclosed herein that bin with dupilumab, and the similarity each in potency across multiple in vitro assays, necessary exposures for potential clinical activity of the antibodies disclosed herein can be predicted. By modeling Kₑₗᵢₘᵢₙₐₜᵢₒₙ and half-life to maintain antibody concentrations above approximately 75 mg/L, at least a 42-day half-life would be required to dose an antibody disclosed herein every six weeks in maintenance, and at least a 59-day half-life would be required to dose an antibody disclosed herein every two months in maintenance.

Accordingly, it is believed that the antibodies disclosed herein can be administered every six weeks if they demonstrate a half-life of at least 42 days and every two months if they demonstrate a half-life of at least 59 days.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

SEQUENCE LISTING

| | | SEQ ID |
|---|---|---|
| Description | Sequence | NO |
| Dupilumab | DYAMT | 1 |
| Kabat HCDR1 | | |
| Kabat HCDR1 | DFAMT | 2 |
| Kabat HCDR1 | TYAMT | 3 |
| Kabat HCDR1 | DSAMT | 4 |
| Dupilumab | SISGSGGNTYYADSVKG | 5 |
| Kabat HCDR2 | | |
| Kabat HCDR2 | SISGSGGNIYYADSVKG | 6 |
| Kabat HCDR2 | SISGSGSSTYYADSVKG | 7 |
| Kabat HCDR2 | SISGSGDNIYYADSVKG | 8 |
| Kabat and | SISGDGSNTYYADSVKG | 9 |
| Chothia HCDR2 | | |
| Kabat HCDR2 | SVSGSGGNTYYADSVKG | 10 |
| Kabat HCDR2 | SISGSGDNTYYADSVKG | 11 |
| Kabat HCDR2 | SISGGGGSTYYADSVKG | 12 |
| Kabat HCDR2 | SISGSGGNKYYADSVKG | 13 |
| Kabat HCDR2 | SVSGDGGNTYYADSVKG | 14 |
| Kabat HCDR2 | SISGGGGNTYYADSVKG | 15 |
| Kabat HCDR2 | SISGSGDGTYYADSVKG | 16 |
| Dupilumab | DRLSITIRPRYYGLDV | 17 |
| Kabat and | | |
| Chothia HCDR3 | | |
| Kabat and | DRLSKTIRPRYYGLDV | 18 |
| Chothia HCDR3 | | |
| Kabat and | DRVSITIRPRYYGLDV | 19 |
| Chothia HCDR3 | | |
| Kabat and | DRLSVTVRPRYYGLDV | 20 |
| Chothia HCDR3 | | |
| Kabat and | DRLSVTIRPRYYGLDV | 21 |
| Chothia HCDR3 | | |
| Kabat and | DRLSITVRPRYYGLDV | 22 |
| Chothia HCDR3 | | |
| Kabat and | DRVSIYVRPRYYGLDV | 23 |
| Chothia HCDR3 | | |
| Kabat and | DRLSIAVRPRYYGLDV | 24 |
| Chothia HCDR3 | | |
| Kabat and | DRLSIYIRPRYYGLDV | 25 |
| Chothia HCDR3 | | |
| Dupilumab | RSSQSLLYSIGYNYLD | 26 |
| Kabat and | | |
| Chothia LCDR1 | | |
| Kabat and | RSSQSVLYSIGYNYVD | 27 |
| Chothia LCDR1 | | |
| Kabat and | TASQSLLYSIGYNYVD | 28 |
| Chothia LCDR1 | | |
| Kabat and | RSSQSLLYSIGYNYVD | 29 |
| Chothia LCDR1 | | |
| Kabat and | RSSQSLLYSIGKNYVD | 30 |
| Chothia LCDR1 | | |
| Kabat and | RSSQGLLYSIGYNYVD | 31 |
| Chothia LCDR1 | | |
| Kabat and | RSTQSLLYSIGKNYVD | 32 |
| Chothia LCDR1 | | |
| Kabat and | TSSQSLLYSIGKNYVD | 33 |
| Chothia LCDR1 | | |
| Kabat and | TSSQSLLYSIGYNYVD | 34 |
| Chothia LCDR1 | | |
| Kabat and | RSSQSVLYSIGKNYVD | 35 |
| Chothia LCDR1 | | |
| Kabat and | TASQSLLYSIGKNYVD | 36 |
| Chothia LCDR1 | | |
| Kabat and | TASQSLLYSIGYNYLD | 37 |
| Chothia LCDR1 | | |
| Kabat and | RSSQSLLYSNRNNYLD | 38 |
| Chothia LCDR1 | | |
| Kabat and | RSSQSLLYSNGYNYLD | 39 |
| Chothia LCDR1 | | |
| Kabat and | RASQSLLYSVGYNYVD | 40 |
| Chothia LCDR1 | | |
| Dupilumab | LGSNRAS | 41 |
| Kabat LCDR2 | | |
| Kabat and | LGSKRAS | 42 |
| Chothia LCDR2 | | |
| Kabat and | LGNKRAS | 43 |
| Chothia LCDR2 | | |
| Kabat and | LGSQRAT | 44 |
| Chothia LCDR2 | | |
| Kabat and | LGNNRAT | 45 |
| Chothia LCDR2 | | |
| Kabat and | KGNNRAT | 46 |
| Chothia LCDR2 | | |
| Kabat and | EGNKRAS | 47 |
| Chothia LCDR2 | | |
| Kabat and | SASIRAS | 48 |
| Chothia LCDR2 | | |
| Kabat and | LSSNRAS | 49 |
| Chothia LCDR2 | | |
| Kabat and | EGNNRAT | 50 |
| Chothia LCDR2 | | |
| Kabat and | EGNNRAS | 51 |
| Chothia LCDR2 | | |
| Kabat and | LGNNRAS | 52 |
| Chothia LCDR2 | | |
| Dupilumab | MQALQTPYT | 53 |
| Kabat, Chothia, | | |
| and IMGT | | |
| LCDR3 | | |
| Kabat, Chothia, | MQSLDTPYT | 54 |
| and IMGT | | |
| LCDR3 | | |
| Kabat, Chothia, | MQALTTPYT | 55 |
| and IMGT | | |
| LCDR3 | | |
| Kabat, Chothia, | MQSLQTPYV | 56 |
| and IMGT | | |
| LCDR3 | | |
| Kabat, Chothia, | MQSLQTPYV | 57 |
| and IMGT | | |
| LCDR3 | | |
| Kabat, Chothia, | MQALDTPYT | 58 |
| and IMGT | | |
| LCDR3 | | |
| Kabat, Chothia, | MQALTTPYA | 59 |
| and IMGT | | |
| LCDR3 | | |
| Kabat, Chothia, | MQALGSPYT | 60 |
| and IMGT | | |
| LCDR3 | | |
| Kabat, Chothia, | MQSLTSPYT | 61 |
| and IMGT | | |
| LCDR3 | | |
| Kabat, Chothia, | MQAYNSPYT | 62 |
| and IMGT | | |
| LCDR3 | | |
| Kabat, Chothia, | MQAFQAPYT | 63 |
| and IMGT | | |
| LCDR3 | | |
| Kabat, Chothia, | MQALQAPYT | 64 |
| and IMGT | | |
| LCDR3 | | |
| Kabat, Chothia, | MQAYQSPYV | 65 |
| and IMGT | | |
| LCDR3 | | |
| Dupilumab | GFTFRDY | 66 |
| Chothia HCDR1 | | |
| Chothia HCDR1 | GFTFRDF | 67 |
| Chothia HCDR1 | GFTFTDF | 68 |
| Chothia HCDR1 | GFTFRTY | 69 |
| Chothia HCDR1 | GFTFRDS | 70 |
| Dupilumab | SGSGGN | 71 |
| Chothia HCDR2 | | |
| Chothia HCDR2 | SGSGSS | 72 |
| Chothia HCDR2 | SGSGDN | 73 |
| Chothia HCDR2 | SGGGGS | 74 |
| Chothia HCDR2 | SGDGGN | 75 |
| Chothia HCDR2 | SGGGGN | 76 |
| Chothia HCDR2 | SGDGSN | 77 |
| Chothia HCDR2 | SGSGDG | 78 |
| Dupilumab | ISGSGGNT | 79 |
| IMGT HCDR2 | | |
| IMGT HCDR2 | ISGSGGNI | 80 |
| IMGT HCDR2 | ISGSGSST | 81 |
| IMGT HCDR2 | ISGSGDNI | 82 |
| IMGT HCDR2 | ISGDGSNT | 83 |
| IMGT HCDR2 | VSGSGGNT | 84 |
| IMGT HCDR2 | ISGSGDNT | 85 |
| IMGT HCDR2 | ISGGGGST | 86 |
| IMGT HCDR2 | ISGSGGNK | 87 |
| IMGT HCDR2 | VSGDGGNT | 88 |
| IMGT HCDR2 | ISGGGGNT | 89 |
| IMGT HCDR2 | ISGSGDGT | 90 |
| Dupilumab | AKDRLSITIRPRYYGLDV | 91 |
| IMGT HCDR3 | | |
| IMGT HCDR3 | AKDRLSKTIRPRYYGLDV | 92 |
| IMGT HCDR3 | AKDRLSITVRPRYYGLDV | 93 |
| IMGT HCDR3 | AKDRVSITIRPRYYGLDV | 94 |
| IMGT HCDR3 | AKDRLSVTVRPRYYGLDV | 95 |
| IMGT HCDR3 | AKDRLSVTIRPRYYGLDV | 96 |
| IMGT HCDR3 | AKDRVSIYVRPRYYGLDV | 97 |
| IMGT HCDR3 | AKDRLSIAVRPRYYGLDV | 98 |
| IMGT HCDR3 | AKDRLSIYIRPRYYGLDV | 99 |
| Dupilimab | QSLLYSIGYNY | 100 |
| IMGT LCDR1 | | |
| IMGT LCDR1 | QSVLYSIGYNY | 101 |
| IMGT LCDR1 | QSLLYSIGKNY | 102 |
| IMGT LCDR1 | QGLLYSIGYNY | 103 |
| IMGT LCDR1 | QSVLYSIGKNY | 104 |
| IMGT LCDR1 | QSLLYSNRNNY | 105 |
| IMGT LCDR1 | QSLLYSNGYNY | 106 |
| IMGT LCDR1 | QSLLYSVGYNY | 107 |
| Dupilimab | LG | 108 |
| IMGT LCDR2 | | |
| IMGT LCDR2 | KG | 109 |
| IMGT LCDR2 | EG | 110 |
| IMGT LCDR2 | SA | 111 |
| IMGT LCDR2 | LS | 112 |
| Heavy Chain | EVQLVESGGGLEQPGGSLRLSCAGSGFTFRDYAMTWV | 113 |
| Variable Domain | RQAPGKGLEWVSSISGSGGNTYYADSVKGRFTISRDNS | |
| Dupilumab | KNTLYLQMNSLRAEDTAVYYCAKDRLSITIRPRYYGL | |
| | DVWGQGTTVTVSS | |
| Heavy Chain | EVQLVESGGGLEQPGGSLRLSCAGSGFTFRDFAMTWV | 114 |
| Variable Domain | RQAPGKGLEWVSSISGSGGNIYYADSVKGRFTISRDNS | |
| 1 | KNTLYLQMNSLRAEDTAVYYCAKDRLSKTIRPRYYGL | |
| (mAb410; | DVWGQGTTVTVSS | |
| mAb411; mAb413; | | |
| mAb421; mAb423; | | |
| mAb469; mAb485;) | | |
| Heavy Chain | EVQLVESGGGLEQPGGSLRLSCAGSGFTFRDFAMTWV | 115 |
| Variable Domain | RQAPGKGLEWVSSISGSGSSTYYADSVKGRFTISRDNS | |
| 2 | KNTLYLQMNSLRAEDTAVYYCAKDRLSITVRPRYYGL | |
| (mAb412) | DVWGQGTTVTVSS | |
| Heavy Chain | EVQLVESGGGLEQPGGSLRLSCAGSGFTFRDFAMTWV | 116 |
| Variable Domain | RQAPGKGLEWVSSISGSGDNIYYADSVKGRFTISRDNS | |
| 3 | KNTLYLQMNSLRAEDTAVYYCAKDRVSITIRPRYYGL | |
| (mAb414) | DVWGQGTTVTVSS | |
| Heavy Chain | EVQLVESGGGLEQPGGSLRLSCAGSGFTFRDFAMTWV | 117 |
| Variable Domain | RQAPGKGLEWVSSISGSGDNIYYADSVKGRFTISRDNS | |
| 4 | KNTLYLQMNSLRAEDTAVYYCAKDRLSVTVRPRYYG | |
| (mAb415; mAb417) | LDVWGQGTTVTVSS | |
| Heavy Chain | EVQLVESGGGLEQPGGSLRLSCAGSGFTFRDFAMTWV | 118 |
| Variable Domain | RQAPGKGLEWVSSISGSGGNIYYADSVKGRFTISRDNS | |
| 5 | KNTLYLQMNSLRAEDTAVYYCAKDRLSITIRPRYYGL | |
| (mAb416) | DVWGQGTTVTVSS | |
| Heavy Chain | EVQLVESGGGLEQPGGSLRLSCAGSGFTFTDFAMTWV | 119 |
| Variable Domain | RQAPGKGLEWVSSISGSGGNIYYADSVKGRFTISRDNS | |
| 6 | KNTLYLQMNSLRAEDTAVYYCAKDRLSVTVRPRYYG | |
| (mAb418) | LDVWGQGTTVTVSS | |
| Heavy Chain | EVQLVESGGGLEQPGGSLRLSCAGSGFTFRDFAMTWV | 120 |
| Variable Domain | RQAPGKGLEWVSSISGDGSNTYYADSVKGRFTISRDNS | |
| 7 | KNTLYLQMNSLRAEDTAVYYCAKDRLSKTIRPRYYGL | |
| (mAb419; mAb420) | DVWGQGTTVTVSS | |
| Heavy Chain | EVQLVESGGGLEQPGGSLRLSCAGSGFTFRDFAMTWV | 121 |
| Variable Domain | RQAPGKGLEWVSSVSGSGGNTYYADSVKGRFTISRDN | |
| 8 | SKNTLYLQMNSLRAEDTAVYYCAKDRLSVTVRPRYY | |
| (mAb422; mAb436) | GLDVWGQGTTVTVSS | |
| Heavy Chain | EVQLVESGGGLEQPGGSLRLSCAGSGFTFRTYAMTWV | 122 |
| Variable Domain | RQAPGKGLEWVSSVSGSGGNTYYADSVKGRFTISRDN | |
| 9 | SKNTLYLQMNSLRAEDTAVYYCAKDRLSVTVRPRYY | |
| (mAb422B) | GLDVWGQGTTVTVSS | |
| Heavy Chain | EVQLVESGGGLEQPGGSLRLSCAGSGFTFRTYAMTWV | 123 |
| Variable Domain | RQAPGKGLEWVSSISGSGDNIYYADSVKGRFTISRDNS | |
| 10 | KNTLYLQMNSLRAEDTAVYYCAKDRLSVTIRPRYYGL | |
| (mAb424) | DVWGQGTTVTVSS | |
| Heavy Chain | EVQLVESGGGLEQPGGSLRLSCAGSGFTFRDFAMTWV | 124 |
| Variable Domain | RQAPGKGLEWVSSISGSGSSTYYADSVKGRFTISRDNS | |
| 11 | KNTLYLQMNSLRAEDTAVYYCAKDRLSVTVRPRYYG | |
| (mAb425) | LDVWGQGTTVTVSS | |
| Heavy Chain | EVQLVESGGGLEQPGGSLRLSCAGSGFTFRDFAMTWV | 125 |
| Variable Domain | RQAPGKGLEWVSSISGSGDNTYYADSVKGRFTISRDNS | |
| 12 | KNTLYLQMNSLRAEDTAVYYCAKDRLSKTIRPRYYGL | |
| (mAb426) | DVWGQGTTVTVSS | |
| Heavy Chain | EVQLVESGGGLEQPGGSLRLSCAGSGFTFRTYAMTWV | 126 |
| Variable Domain | RQAPGKGLEWVSSISGSGDNTYYADSVKGRFTISRDNS | |
| 13 | KNTLYLQMNSLRAEDTAVYYCAKDRVSITIRPRYYGL | |
| (mAb427) | DVWGQGTTVTVSS | |
| Heavy Chain | EVQLVESGGGLEQPGGSLRLSCAGSGFTFRDFAMTWV | 127 |
| Variable Domain | RQAPGKGLEWVSSISGSGDNTYYADSVKGRFTISRDNS | |
| 14 | KNTLYLQMNSLRAEDTAVYYCAKDRLSITVRPRYYGL | |
| (mAb428) | DVWGQGTTVTVSS | |
| Heavy Chain | EVQLVESGGGLEQPGGSLRLSCAGSGFTFRTYAMTWV | 128 |
| Variable Domain | RQAPGKGLEWVSSISGSGGNIYYADSVKGRFTISRDNS | |
| 15 | KNTLYLQMNSLRAEDTAVYYCAKDRVSIYVRPRYYGL | |
| (mAb429) | DVWGQGTTVTVSS | |
| Heavy Chain | EVQLVESGGGLEQPGGSLRLSCAGSGFTFRDFAMTWV | 129 |
| Variable Domain | RQAPGKGLEWVSSISGGGGSTYYADSVKGRFTISRDNS | |
| 16 | KNTLYLQMNSLRAEDTAVYYCAKDRLSVTVRPRYYG | |
| (mAb431) | LDVWGQGTTVTVSS | |
| Heavy Chain | EVQLVESGGGLEQPGGSLRLSCAGSGFTFRDFAMTWV | 130 |
| Variable Domain | RQAPGKGLEWVSSISGSGGNIYYADSVKGRFTISRDNS | |
| 17 | KNTLYLQMNSLRAEDTAVYYCAKDRLSIAVRPRYYGL | |
| (mAb432) | DVWGQGTTVTVSS | |
| Heavy Chain | EVQLVESGGGLEQPGGSLRLSCAGSGFTFRDFAMTWV | 131 |
| Variable Domain | RQAPGKGLEWVSSISGSGDNIYYADSVKGRFTISRDNS | |
| 18 | KNTLYLQMNSLRAEDTAVYYCAKDRLSKTIRPRYYGL | |
| (mAb433) | DVWGQGTTVTVSS | |
| Heavy Chain | EVQLVESGGGLEQPGGSLRLSCAGSGFTFRDFAMTWV | 132 |
| Variable Domain | RQAPGKGLEWVSSISGGGGSTYYADSVKGRFTISRDNS | |
| 19 | KNTLYLQMNSLRAEDTAVYYCAKDRLSIAVRPRYYGL | |
| (mAb434) | DVWGQGTTVTVSS | |
| Heavy Chain | EVQLVESGGGLEQPGGSLRLSCAGSGFTFTDFAMTWV | 133 |
| Variable Domain | RQAPGKGLEWVSSISGSGGNKYYADSVKGRFTISRDNS | |
| 20 | KNTLYLQMNSLRAEDTAVYYCAKDRLSVTVRPRYYG | |
| (mAb435) | LDVWGQGTTVTVSS | |
| Heavy Chain | EVQLVESGGGLEQPGGSLRLSCAGSGFTFRDFAMTWV | 134 |
| Variable Domain | RQAPGKGLEWVSSISGSGGNIYYADSVKGRFTISRDNS | |
| 21 | KNTLYLQMNSLRAEDTAVYYCAKDRLSVTVRPRYYG | |
| (mAb437; | LDVWGQGTTVTVSS | |
| mAb443; mAb471) | | |
| Heavy Chain | EVQLVESGGGLEQPGGSLRLSCAGSGFTFRDFAMTWV | 135 |
| Variable Domain | RQAPGKGLEWVSSVSGDGGNTYYADSVKGRFTISRDN | |
| 22 | SKNTLYLQMNSLRAEDTAVYYCAKDRLSVTVRPRYY | |
| (mAb446) | GLDVWGQGTTVTVSS | |
| Heavy Chain | EVQLVESGGGLEQPGGSLRLSCAGSGFTFRDFAMTWV | 136 |
| Variable Domain | RQAPGKGLEWVSSISGSGGNIYYADSVKGRFTISRDNS | |
| 23 | KNTLYLQMNSLRAEDTAVYYCAKDRVSITIRPRYYGL | |
| (mAb450) | DVWGQGTTVTVSS | |
| Heavy Chain | EVQLVESGGGLEQPGGSLRLSCAGSGFTFRTYAMTWV | 137 |
| Variable Domain | RQAPGKGLEWVSSISGSGGNTYYADSVKGRFTISRDNS | |
| 24 | KNTLYLQMNSLRAEDTAVYYCAKDRVSITIRPRYYGL | |
| (mAb467) | DVWGQGTTVTVSS | |
| Heavy Chain | EVQLVESGGGLEQPGGSLRLSCAGSGFTFRTYAMTWV | 138 |
| Variable Domain | RQAPGKGLEWVSSISGGGGNTYYADSVKGRFTISRDNS | |
| 25 | KNTLYLQMNSLRAEDTAVYYCAKDRLSVTVRPRYYG | |
| (mAb470) | LDVWGQGTTVTVSS | |
| Heavy Chain | EVQLVESGGGLEQPGGSLRLSCAGSGFTFRDSAMTWV | 139 |
| Variable Domain | RQAPGKGLEWVSSISGSGDNTYYADSVKGRFTISRDNS | |
| 26 | KNTLYLQMNSLRAEDTAVYYCAKDRLSITVRPRYYGL | |
| (mAb473) | DVWGQGTTVTVSS | |
| Heavy Chain | EVQLVESGGGLEQPGGSLRLSCAGSGFTFRDFAMTWV | 140 |
| Variable Domain | RQAPGKGLEWVSSVSGDGGNTYYADSVKGRFTISRDN | |
| 27 | SKNTLYLQMNSLRAEDTAVYYCAKDRLSKTIRPRYYG | |
| (mAb476) | LDVWGQGTTVTVSS | |
| Heavy Chain | EVQLVESGGGLEQPGGSLRLSCAGSGFTFRDFAMTWV | 141 |
| Variable Domain | RQAPGKGLEWVSSISGGGGNTYYADSVKGRFTISRDNS | |
| 28 | KNTLYLQMNSLRAEDTAVYYCAKDRVSITIRPRYYGL | |
| (mAb478) | DVWGQGTTVTVSS | |
| Heavy Chain | EVQLVESGGGLEQPGGSLRLSCAGSGFTFRTYAMTWV | 142 |
| Variable Domain | RQAPGKGLEWVSSISGDGSNTYYADSVKGRFTISRDNS | |
| 29 | KNTLYLQMNSLRAEDTAVYYCAKDRVSITIRPRYYGL | |
| (mAb481) | DVWGQGTTVTVSS | |
| Heavy Chain | EVQLVESGGGLEQPGGSLRLSCAGSGFTFRDFAMTWV | 143 |
| Variable Domain | RQAPGKGLEWVSSISGGGGNTYYADSVKGRFTISRDNS | |
| 30 | KNTLYLQMNSLRAEDTAVYYCAKDRLSIAVRPRYYGL | |
| (mAb482) | DVWGQGTTVTVSS | |
| Heavy Chain | EVQLVESGGGLEQPGGSLRLSCAGSGFTFRDFAMTWV | 144 |
| Variable Domain | RQAPGKGLEWVSSISGSGDGTYYADSVKGRFTISRDNS | |
| 31 | KNTLYLQMNSLRAEDTAVYYCAKDRVSITIRPRYYGL | |
| (mAb494) | DVWGQGTTVTVSS | |
| Heavy Chain | EVQLVESGGGLEQPGGSLRLSCAGSGFTFRDSAMTWV | 145 |
| Variable Domain | RQAPGKGLEWVSSISGSGDGTYYADSVKGRFTISRDNS | |
| 32 | KNTLYLQMNSLRAEDTAVYYCAKDRLSIYIRPRYYGL | |
| (mAb501) | DVWGQGTTVTVSS | |
| Light Chain | DIVMTQSPLSLPVTPGEPASISCRSSQSLLYSIGYNYLD | 146 |
| Variable Domain | WYLQKSGQSPQLLIYLGSNRASGVPDRFSGSGSGTDFT | |
| Dupilumab | LKISRVEAEDVGFYYCMQALQTPYTFGQGTKLEIK | |
| Light Chain | DIVMTQSPLSLPVTPGEPASISCRSSQSVLYSIGYNYVD | 147 |
| Variable Domain | WYLQKSGQSPQLLIYLGSKRASGVPDRFSGSGSGTDFT | |
| 1 | LKISRVEAEDVGFYYCMQSLDTPYTFGQGTKLEIK | |
| (mAb410) | | |
| Light Chain | DIVMTQSPLSLPVTPGEPASISCTASQSLLYSIGYNYVD | 148 |
| Variable Domain | WYLQKSGQSPQLLIYLGSKRASGVPDRFSGSGSGTDFT | |
| 2 | LKISRVEAEDVGFYYCMQALTTPYTFGQGTKLEIK | |
| (mAb411) | | |
| Light Chain | DIVMTQSPLSLPVTPGEPASISCRSSQSLLYSIGYNYVD | 149 |
| Variable Domain | WYLQKSGQSPQLLIYLGNKRASGVPDRFSGSGSGTDFT | |
| 3 | LKISRVEAEDVGFYYCMQALTTPYTFGQGTKLEIK | |
| (mAb412) | | |
| Light Chain | DIVMTQSPLSLPVTPGEPASISCTASQSLLYSIGYNYVD | 150 |
| Variable Domain | WYLQKSGQSPQLLIYLGNNRATGVPDRFSGSGSGTDFT | |
| 4 | LKISRVEAEDVGFYYCMQALTTPYTFGQGTKLEIK | |
| (mAb413) | | |
| Light Chain | DIVMTQSPLSLPVTPGEPASISCRSSQSLLYSIGKNYVD | 151 |
| Variable Domain | WYLQKSGQSPQLLIYLGNKRASGVPDRFSGSGSGTDFT | |
| 5 | LKISRVEAEDVGFYYCMQSLQTPYVFGQGTKLEIK | |
| (mAb414) | | |
| Light Chain | DIVMTQSPLSLPVTPGEPASISCRSSQGLLYSIGYNYVD | 152 |
| Variable Domain | WYLQKSGQSPQLLIYLGNKRASGVPDRFSGSGSGTDFT | |
| 6 | LKISRVEAEDVGFYYCMQSLQTPYTFGQGTKLEIK | |
| (mAb415) | | |
| Light Chain | DIVMTQSPLSLPVTPGEPASISCRSTQSLLYSIGKNYVD | 153 |
| Variable Domain | WYLQKSGQSPQLLIYKGNNRATGVPDRFSGSGSGTDF | |
| 7 | TLKISRVEAEDVGFYYCMQALDTPYTFGQGTKLEIK | |
| (mAb416) | | |
| Light Chain | DIVMTQSPLSLPVTPGEPASISCRSTQSLLYSIGKNYVD | 154 |
| Variable Domain | WYLQKSGQSPQLLIYLGNNRATGVPDRFSGSGSGTDFT | |
| 8 | LKISRVEAEDVGFYYCMQALTTPYAFGQGTKLEIK | |
| (mAb417) | | |
| Light Chain | DIVMTQSPLSLPVTPGEPASISCRSSQSLLYSIGKNYVD | 155 |
| Variable Domain | WYLQKSGQSPQLLIYLGNNRATGVPDRFSGSGSGTDFT | |
| 9 | LKISRVEAEDVGFYYCMQALGSPYTFGQGTKLEIK | |
| (mAb4218) | | |
| Light Chain | DIVMTQSPLSLPVTPGEPASISCRSSQSLLYSIGYNYVD | 156 |
| Variable Domain | WYLQKSGQSPQLLIYLGNKRASGVPDRFSGSGSGTDFT | |
| 10 | LKISRVEAEDVGFYYCMQALGSPYTFGQGTKLEIK | |
| (mAb419) | | |
| Light Chain | DIVMTQSPLSLPVTPGEPASISCRSSQSVLYSIGYNYVD | 157 |
| Variable Domain | WYLQKSGQSPQLLIYLGNKRASGVPDRFSGSGSGTDFT | |
| 11 | LKISRVEAEDVGFYYCMQSLQTPYTFGQGTKLEIK | |
| (mAb420) | | |
| Light Chain | DIVMTQSPLSLPVTPGEPASISCRSSQSVLYSIGYNYVD | 158 |
| Variable Domain | WYLQKSGQSPQLLIYLGNKRASGVPDRFSGSGSGTDFT | |
| 12 | LKISRVEAEDVGFYYCMQSLTSPYTFGQGTKLEIK | |
| (mAb421) | | |
| Light Chain | DIVMTQSPLSLPVTPGEPASISCTASQSLLYSIGYNYVD | 159 |
| Variable Domain | WYLQKSGQSPQLLIYLGNKRASGVPDRFSGSGSGTDFT | |
| 13 | LKISRVEAEDVGFYYCMQSLTSPYTFGQGTKLEIK | |
| (mAb423) | | |
| Light Chain | DIVMTQSPLSLPVTPGEPASISCTASQSLLYSIGYNYVD | 160 |
| Variable Domain | WYLQKSGQSPQLLIYLGSKRASGVPDRFSGSGSGTDFT | |
| 14 | LKISRVEAEDVGFYYCMQAYNSPYTFGQGTKLEIK | |
| (mAb425) | | |
| Light Chain | DIVMTQSPLSLPVTPGEPASISCTSSQSLLYSIGKNYVD | 161 |
| Variable Domain | WYLQKSGQSPQLLIYLGNKRASGVPDRFSGSGSGTDFT | |
| 15 | LKISRVEAEDVGFYYCMQSLTSPYTFGQGTKLEIK | |
| (mAb426) | | |
| Light Chain | DIVMTQSPLSLPVTPGEPASISCTSSQSLLYSIGYNYVD | 162 |
| Variable Domain | WYLQKSGQSPQLLIYLGNKRASGVPDRFSGSGSGTDFT | |
| 16 | LKISRVEAEDVGFYYCMQALGSPYTFGQGTKLEIK | |
| (mAb427) | | |
| Light Chain | DIVMTQSPLSLPVTPGEPASISCTSSQSLLYSIGYNYVD | 163 |
| Variable Domain | WYLQKSGQSPQLLIYLGNKRASGVPDRFSGSGSGTDFT | |
| 17 | LKISRVEAEDVGFYYCMQSLQTPYVFGQGTKLEIK | |
| (mAb428) | | |
| Light Chain | DIVMTQSPLSLPVTPGEPASISCTASQSLLYSIGYNYVD | 164 |
| Variable Domain | WYLQKSGQSPQLLIYLGSKRASGVPDRFSGSGSGTDFT | |
| 18 | LKISRVEAEDVGFYYCMQSLTSPYTFGQGTKLEIK | |
| (mAb429) | | |
| Light Chain | DIVMTQSPLSLPVTPGEPASISCRSSQSVLYSIGKNYVD | 165 |
| Variable Domain | WYLQKSGQSPQLLIYLGSQRATGVPDRFSGSGSGTDFT | |
| 19 | LKISRVEAEDVGFYYCMQSLTSPYTFGQGTKLEIK | |
| (mAb431) | | |
| Light Chain | DIVMTQSPLSLPVTPGEPASISCTASQSLLYSIGKNYVD | 166 |
| Variable Domain | WYLQKSGQSPQLLIYLGNNRATGVPDRFSGSGSGTDFT | |
| 20 | LKISRVEAEDVGFYYCMQSLTSPYTFGQGTKLEIK | |
| (mAb432) | | |
| Light Chain | DIVMTQSPLSLPVTPGEPASISCTASQSLLYSIGYNYLD | 167 |
| Variable Domain | WYLQKSGQSPQLLIYEGNKRASGVPDRFSGSGSGTDFT | |
| 21 | LKISRVEAEDVGFYYCMQSLTSPYTFGQGTKLEIK | |
| (mAb433) | | |
| Light Chain | DIVMTQSPLSLPVTPGEPASISCTSSQSLLYSIGYNYVD | 168 |
| Variable Domain | WYLQKSGQSPQLLIYLGNKRASGVPDRFSGSGSGTDFT | |
| 22 | LKISRVEAEDVGFYYCMQSLQTPYTFGQGTKLEIK | |
| (mAb436) | | |
| Light Chain | DIVMTQSPLSLPVTPGEPASISCTSSQSLLYSIGYNYVD | 169 |
| Variable Domain | WYLQKSGQSPQLLIYLGNKRASGVPDRFSGSGSGTDFT | |
| 23 | LKISRVEAEDVGFYYCMQSLTSPYTFGQGTKLEIK | |
| (mAb437) | | |
| Light Chain | DIVMTQSPLSLPVTPGEPASISCRSSQSLLYSNRNNYLD | 170 |
| Variable Domain | WYLQKSGQSPQLLIYSASIRASGVPDRFSGSGSGTDFTL | |
| 24 | KISRVEAEDVGFYYCMQAFQAPYTFGQGTKLEIK | |
| (mAb439) | | |
| Light Chain | DIVMTQSPLSLPVTPGEPASISCRSSQSLLYSNGYNYLD | 171 |
| Variable Domain | WYLQKSGQSPQLLIYLGSNRASGVPDRFSGSGSGTDFT | |
| 25 | LKISRVEAEDVGFYYCMQALQAPYTFGQGTKLEIK | |
| (mAb443) | | |
| Light Chain | DIVMTQSPLSLPVTPGEPASISCRSSQSLLYSNGYNYLD | 172 |
| Variable Domain | WYLQKSGQSPQLLIYLSSNRASGVPDRFSGSGSGTDFT | |
| 26 | LKISRVEAEDVGFYYCMQAFQAPYTFGQGTKLEIK | |
| (mAb446) | | |
| Light Chain | DIVMTQSPLSLPVTPGEPASISCTASQSLLYSIGYNYVD | 173 |
| Variable Domain | WYLQKSGQSPQLLIYLGSKRASGVPDRFSGSGSGTDFT | |
| 27 | LKISRVEAEDVGFYYCMQALGSPYTFGQGTKLEIK | |
| (mAb450) | | |
| Light Chain | DIVMTQSPLSLPVTPGEPASISCRSSQGLLYSIGYNYVD | 174 |
| Variable Domain | WYLQKSGQSPQLLIYLGNKRASGVPDRFSGSGSGTDFT | |
| 28 | LKISRVEAEDVGFYYCMQALDTPYTFGQGTKLEIK | |
| (mAb459) | | |
| Light Chain | DIVMTQSPLSLPVTPGEPASISCTSSQSLLYSIGKNYVD | 175 |
| Variable Domain | WYLQKSGQSPQLLIYEGNNRATGVPDRFSGSGSGTDFT | |
| 29 | LKISRVEAEDVGFYYCMQSLTSPYTFGQGTKLEIK | |
| (mAb467) | | |
| Light Chain | DIVMTQSPLSLPVTPGEPASISCRSSQSLLYSIGKNYVD | 176 |
| Variable Domain | WYLQKSGQSPQLLIYEGNNRASGVPDRFSGSGSGTDFT | |
| 30 | LKISRVEAEDVGFYYCMQSLTSPYTFGQGTKLEIK | |
| (mAb469) | | |
| Light Chain | DIVMTQSPLSLPVTPGEPASISCRASQSLLYSVGYNYVD | 177 |
| Variable Domain | WYLQKSGQSPQLLIYLGNNRASGVPDRFSGSGSGTDFT | |
| 31 | LKISRVEAEDVGFYYCMQSLTSPYTFGQGTKLEIK | |
| (mAb470) | | |
| Light Chain | DIVMTQSPLSLPVTPGEPASISCRSSQSLLYSIGKNYVD | 178 |
| Variable Domain | WYLQKSGQSPQLLIYEGNKRASGVPDRFSGSGSGTDFT | |
| 32 | LKISRVEAEDVGFYYCMQSLTSPYTFGQGTKLEIK | |
| (mAb471) | | |
| Light Chain | DIVMTQSPLSLPVTPGEPASISCRSSQSLLYSIGKNYVD | 179 |
| Variable Domain | WYLQKSGQSPQLLIYLGNKRASGVPDRFSGSGSGTDFT | |
| 33 | LKISRVEAEDVGFYYCMQALGSPYTFGQGTKLEIK | |
| (mAb434) | | |
| Light Chain | DIVMTQSPLSLPVTPGEPASISCRSSQSVLYSIGYNYVD | 180 |
| Variable Domain | WYLQKSGQSPQLLIYLGNKRASGVPDRFSGSGSGTDFT | |
| 34 | LKISRVEAEDVGFYYCMQAYQSPYVFGQGTKLEIK | |
| (mAb473) | | |
| Light Chain | DIVMTQSPLSLPVTPGEPASISCTASQSLLYSIGYNYVD | 181 |
| Variable Domain | WYLQKSGQSPQLLIYLGNKRASGVPDRFSGSGSGTDFT | |
| 35 | LKISRVEAEDVGFYYCMQAYNSPYTFGQGTKLEIK | |
| (mAb478) | | |
| Light Chain | DIVMTQSPLSLPVTPGEPASISCTASQSLLYSIGYNYVD | 182 |
| Variable Domain | WYLQKSGQSPQLLIYLGNKRASGVPDRFSGSGSGTDFT | |
| 36 | LKISRVEAEDVGFYYCMQALTTPYTFGQGTKLEIK | |
| (mAb481) | | |
| Light Chain | DIVMTQSPLSLPVTPGEPASISCTSSQSLLYSIGYNYVD | 183 |
| Variable Domain | WYLQKSGQSPQLLIYLGSKRASGVPDRFSGSGSGTDFT | |
| 37 | LKISRVEAEDVGFYYCMQSLTSPYTFGQGTKLEIK | |
| (mAb482) | | |
| Light Chain | DIVMTQSPLSLPVTPGEPASISCRSTQSLLYSIGKNYVD | 184 |
| Variable Domain | WYLQKSGQSPQLLIYEGNNRASGVPDRFSGSGSGTDFT | |
| 38 | LKISRVEAEDVGFYYCMQSLTSPYTFGQGTKLEIK | |
| (mAb485) | | |
| Light Chain | DIVMTQSPLSLPVTPGEPASISCTASQSLLYSIGYNYVD | 185 |
| Variable Domain | WYLQKSGQSPQLLIYLGNNRATGVPDRFSGSGSGTDFT | |
| 39 | LKISRVEAEDVGFYYCMQSLQTPYTFGQGTKLEIK | |
| (mAb494) | | |
| Light Chain | DIVMTQSPLSLPVTPGEPASISCTSSQSLLYSIGYNYVD | 186 |
| Variable Domain | WYLQKSGQSPQLLIYLGNKRASGVPDRFSGSGSGTDFT | |
| 40 | LKISRVEAEDVGFYYCMQALQTPYTFGQGTKLEIK | |
| (mAb501) | | |
| Dupilumab | GFTFRDYA | 187 |
| IMGT HCDR1 | | |
| IMGT HCDR1 | GFTFRDFA | 188 |
| IMGT HCDR1 | GFTFTDFA | 189 |
| IMGT HCDR1 | GFTFRTYA | 190 |
| IMGT HCDR1 | GFTFRDSA | 191 |
| hIgG1* | ASTKGPSVFPLAPSSKSTSGGTAALGCLVKDYFPEPVT | 192 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPG | |
| hIgG4 | ASTKGPSVFPLAPCSRSTSESTAALGCLVKDYFPEPVTV | 193 |
| | SWNSGALTSGVHTFPAVLQSSGLYSLSSVVTVPSSSLG | |
| | TKTYTCNVDHKPSNTKVDKRVESKYGPPCPSCPAPEFL | |
| | GGPSVFLFPPKPKDTLMISRTPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHNHYTQKSLSLSLG | |
| IgG4-SP | ASTKGPSVFPLAPCSRSTSESTAALGCLVKDYFPEPVTV | 193 |
| | SWNSGALTSGVHTFPAVLQSSGLYSLSSVVTVPSSSLG | |
| | TKTYTCNVDHKPSNTKVDKRVESKYGPPCPPCPAPEFL | |
| | GGPSVFLFPPKPKDTLMISRTPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHNHYTQKSLSLSLG | |
| IgG4-SPLE | ASTKGPSVFPLAPCSRSTSESTAALGCLVKDYFPEPVTV | 194 |
| | SWNSGALTSGVHTFPAVLQSSGLYSLSSVVTVPSSSLG | |
| | TKTYTCNVDHKPSNTKVDKRVESKYGPPCPPCPAPEEL | |
| | GGPSVFLFPPKPKDTLMISRTPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHNHYTQKSLSLSLG | |
| IgG2 | ASTKGPSVFPLAPCSRSTSESTAALGCLVKDYFPEPVTV | 195 |
| | SWNSGALTSGVHTFPAVLQSSGLYSLSSVVTVPSSNFG | |
| | TQTYTCNVDHKPSNTKVDKTVERKCCVECPPCPAPPV | |
| | AGPSVFLFPPKPKDTLMISRTPEVTCVVVDVSHEDPEV | |
| | QFNWYVDGVEVHNAKTKPREEQFNSTFRVVSVLTVV | |
| | HQDWLNGKEYKCKVSNKGLPAPIEKTISKTKGQPREP | |
| | QVYTLPPSREEMTKNQVSLTCLVKGFYPSDIAVEWESN | |
| | GQPENNYKTTPPMLDSDGSFFLYSKLTVDKSRWQQGN | |
| | VFSCSVMHEALHNHYTQKSLSLSP | |
| hIgG1-N297A | ASTKGPSVFPLAPSSKSTSGGTAALGCLVKDYFPEPVT | 196 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYASTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPG | |
| hIgG1-D265A | ASTKGPSVFPLAPSSKSTSGGTAALGCLVKDYFPEPVT | 197 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVAVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPG | |
| hIgG1-LALA | ASTKGPSVFPLAPSSKSTSGGTAALGCLVKDYFPEPVT | 198 |
| | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1-LAGA | ASTKGPSVFPLAPSSKSTSGGTAALGCLVKDYFPEPVT | 199 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 200 |
| LALAGA | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1-LALAPG | ASTKGPSVFPLAPSSKSTSGGTAALGCLVKDYFPEPVT | 201 |
| | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALGAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1-YTE | ASTKGPSVFPLAPSSKSTSGGTAALGCLVKDYFPEPVT | 202 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLYITREPEVTCVVVDVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPG | |
| hIgG1- | ASTKGPSVFPLAPSSKSTSGGTAALGCLVKDYFPEPVT | 203 |
| N297A/YTE | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLYITREPEVTCVVVDVSHE | |
| | DPEVKFNWYVDGVEVHNAKTKPREEQYASTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPG | |
| hIgG1- | ASTKGPSVFPLAPSSKSTSGGTAALGCLVKDYFPEPVT | 204 |
| D265A/YTE | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLYITREPEVTCVVVAVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPG | |
| hIgG1- | ASTKGPSVFPLAPSSKSTSGGTAALGCLVKDYFPEPVT | 205 |
| LALA/YTE | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLYITREPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 206 |
| LAGA/YTE | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLYITREPEVTCVVVDVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPG | |
| hIgG1- | ASTKGPSVFPLAPSSKSTSGGTAALGCLVKDYFPEPVT | 207 |
| LALAGA/YTE | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLYITREPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 208 |
| LALAPG/YTE | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLYITREPEVTCVVVDVSH | |
| | EDPEVKFNWYVDGVEVHNAKTKPREEQYNSTYRVVS | |
| | VLTVLHQDWLNGKEYKCKVSNKALGAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1-LS | ASTKGPSVFPLAPSSKSTSGGTAALGCLVKDYFPEPVT | 209 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVLHEALHSHYTQKSLSLSPG | |
| hIgG1- | ASTKGPSVFPLAPSSKSTSGGTAALGCLVKDYFPEPVT | 210 |
| N297A/LS | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYASTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVLHEALHSHYTQKSLSLSPG | |
| hIgG1- | ASTKGPSVFPLAPSSKSTSGGTAALGCLVKDYFPEPVT | 211 |
| D265A/LS | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVAVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVLHEALHSHYTQKSLSLSPG | |
| hIgG1- | ASTKGPSVFPLAPSSKSTSGGTAALGCLVKDYFPEPVT | 212 |
| LALA/LS | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVLHEALHSHYTQKSLSLSPG | |
| hIgG1- | ASTKGPSVFPLAPSSKSTSGGTAALGCLVKDYFPEPVT | 213 |
| LAGA/LS | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVLHEALHSHYTQKSLSLSPG | |
| hIgG1- | ASTKGPSVFPLAPSSKSTSGGTAALGCLVKDYFPEPVT | 214 |
| LALAGA/LS | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVLHEALHSHYTQKSLSLSPG | |
| hIgG1- | ASTKGPSVFPLAPSSKSTSGGTAALGCLVKDYFPEPVT | 215 |
| LALAPG/LS | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALGAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVLHEALHSHYTQKSLSLSPG | |
| hIgG1-DHS | ASTKGPSVFPLAPSSKSTSGGTAALGCLVKDYFPEPVT | 216 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYNSTYRVVSV | |
| | LTVDHHDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHSHYTQKSLSLSPG | |
| hIgG1- | ASTKGPSVFPLAPSSKSTSGGTAALGCLVKDYFPEPVT | 217 |
| N297A/DHS | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYASTYRVVSV | |
| | LTVDHHDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHSHYTQKSLSLSPG | |
| hIgG1- | ASTKGPSVFPLAPSSKSTSGGTAALGCLVKDYFPEPVT | 218 |
| D265A/DHS | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVAVSHE | |
| | DPEVKFNWYVDGVEVHNAKTKPREEQYNSTYRVVSV | |
| | LTVDHHDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHSHYTQKSLSLSPG | |
| hIgG1- | ASTKGPSVFPLAPSSKSTSGGTAALGCLVKDYFPEPVT | 219 |
| LALA/DHS | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVDHHDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHSHYTQKSLSLSPG | |
| hIgG1- | ASTKGPSVFPLAPSSKSTSGGTAALGCLVKDYFPEPVT | 220 |
| LAGA/DHS | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRTPEVTCVVVDVSH | |
| | EDPEVKFNWYVDGVEVHNAKTKPREEQYNSTYRVVS | |
| | VLTVDHHDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHSHYTQKSLSLSPG | |
| hIgG1- | ASTKGPSVFPLAPSSKSTSGGTAALGCLVKDYFPEPVT | 221 |
| LALAGA/DHS | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLMISRTPEVTCVVVDVSH | |
| | EDPEVKFNWYVDGVEVHNAKTKPREEQYNSTYRVVS | |
| | VLTVDHHDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHSHYTQKSLSLSPG | |
| hIgG1- | ASTKGPSVFPLAPSSKSTSGGTAALGCLVKDYFPEPVT | 222 |
| LALAPG/DHS | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVDHHDWLNGKEYKCKVSNKALGAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHSHYTQKSLSLSPG | |
| hIgG4-YTE | ASTKGPSVFPLAPCSRSTSESTAALGCLVKDYFPEPVTV | 223 |
| | SWNSGALTSGVHTFPAVLQSSGLYSLSSVVTVPSSSLG | |
| | TKTYTCNVDHKPSNTKVDKRVESKYGPPCPSCPAPEFL | |
| | GGPSVFLFPPKPKDTLYITREPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHNHYTQKSLSLSLGK | |
| hIgG4-SP/YTE | ASTKGPSVFPLAPCSRSTSESTAALGCLVKDYFPEPVTV | 224 |
| | SWNSGALTSGVHTFPAVLQSSGLYSLSSVVTVPSSSLG | |
| | TKTYTCNVDHKPSNTKVDKRVESKYGPPCPPCPAPEFL | |
| | GGPSVFLFPPKPKDTLYITREPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHNHYTQKSLSLSLGK | |
| hIgG4- | ASTKGPSVFPLAPCSRSTSESTAALGCLVKDYFPEPVTV | 225 |
| SPLE/YTE | SWNSGALTSGVHTFPAVLQSSGLYSLSSVVTVPSSSLG | |
| | TKTYTCNVDHKPSNTKVDKRVESKYGPPCPPCPAPEEL | |
| | GGPSVFLFPPKPKDTLYITREPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHNHYTQKSLSLSLGK | |
| hIgG4-LS | ASTKGPSVFPLAPCSRSTSESTAALGCLVKDYFPEPVTV | 226 |
| | SWNSGALTSGVHTFPAVLQSSGLYSLSSVVTVPSSSLG | |
| | TKTYTCNVDHKPSNTKVDKRVESKYGPPCPSCPAPEFL | |
| | GGPSVFLFPPKPKDTLMISRTPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVLHEALHSYTQKSLSLSLGK | |
| hIgG4-SP/LS | ASTKGPSVFPLAPCSRSTSESTAALGCLVKDYFPEPVTV | 227 |
| | SWNSGALTSGVHTFPAVLQSSGLYSLSSVVTVPSSSLG | |
| | TKTYTCNVDHKPSNTKVDKRVESKYGPPCPPCPAPEFL | |
| | GGPSVFLFPPKPKDTLMISRTPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVLHEALHSHYTQKSLSLSLGK | |
| hIgG4-SPLE/LS | ASTKGPSVFPLAPCSRSTSESTAALGCLVKDYFPEPVTV | 228 |
| | SWNSGALTSGVHTFPAVLQSSGLYSLSSVVTVPSSSLG | |
| | TKTYTCNVDHKPSNTKVDKRVESKYGPPCPPCPAPEEL | |
| | GGPSVFLFPPKPKDTLMISRTPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVLHEALHSHYTQKSLSLSLGK | |
| hIgG4-DHS | ASTKGPSVFPLAPCSRSTSESTAALGCLVKDYFPEPVTV | 229 |
| | SWNSGALTSGVHTFPAVLQSSGLYSLSSVVTVPSSSLG | |
| | TKTYTCNVDHKPSNTKVDKRVESKYGPPCPSCPAPEFL | |
| | GGPSVFLFPPKPKDTLMISRTPEVTCVVVDVSQEDPEV | |
| | QFNWYVDGVEVHNAKTKPREEQFNSTYRVVSVLTVD | |
| | HHDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHSHYTQKSLSLSLGK | |
| hIgG4-SP/DHS | ASTKGPSVFPLAPCSRSTSESTAALGCLVKDYFPEPVTV | 230 |
| | SWNSGALTSGVHTFPAVLQSSGLYSLSSVVTVPSSSLG | |
| | TKTYTCNVDHKPSNTKVDKRVESKYGPPCPPCPAPEFL | |
| | GGPSVFLFPPKPKDTLMISRTPEVTCVVVDVSQEDPEV | |
| | QFNWYVDGVEVHNAKTKPREEQFNSTYRVVSVLTVD | |
| | HHDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHSHYTQKSLSLSLGK | |
| hIgG4- | ASTKGPSVFPLAPCSRSTSESTAALGCLVKDYFPEPVTV | 23 |
| SPLE/DHS | SWNSGALTSGVHTFPAVLQSSGLYSLSSVVTVPSSSLG | |
| | TKTYTCNVDHKPSNTKVDKRVESKYGPPCPPCPAPEEL | |
| | GGPSVFLFPPKPKDTLMISRTPEVTCVVVDVSQEDPEV | |
| | QFNWYVDGVEVHNAKTKPREEQFNSTYRVVSVLTVD | |
| | HHDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHSHYTQKSLSLSLGK | |
| hIgG2-YTE | ASTKGPSVFPLAPCSRSTSESTAALGCLVKDYFPEPVTV | 232 |
| | SWNSGALTSGVHTFPAVLQSSGLYSLSSVVTVPSSNFG | |
| | TQTYTCNVDHKPSNTKVDKTVERKCCVECPPCPAPPV | |
| | AGPSVFLFPPKPKDTLYITREPEVTCVVVDVSHEDPEV | |
| | QFNWYVDGVEVHNAKTKPREEQFNSTFRVVSVLTVV | |
| | HQDWLNGKEYKCKVSNKGLPAPIEKTISKTKGQPREP | |
| | QVYTLPPSREEMTKNQVSLTCLVKGFYPSDIAVEWESN | |
| | GQPENNYKTTPPMLDSDGSFFLYSKLTVDKSRWQQGN | |
| | VFSCSVLHEALHSHYTQKSLSLSP | |
| hIgG2-LS | ASTKGPSVFPLAPCSRSTSESTAALGCLVKDYFPEPVTV | 233 |
| | SWNSGALTSGVHTFPAVLQSSGLYSLSSVVTVPSSNFG | |
| | TQTYTCNVDHKPSNTKVDKTVERKCCVECPPCPAPPV | |
| | AGPSVFLFPPKPKDTLMISRTPEVTCVVVDVSHEDPEV | |
| | QFNWYVDGVEVHNAKTKPREEQFNSTFRVVSVLTVV | |
| | HQDWLNGKEYKCKVSNKGLPAPIEKTISKTKGQPREP | |
| | QVYTLPPSREEMTKNQVSLTCLVKGFYPSDIAVEWESN | |
| | GQPENNYKTTPPMLDSDGSFFLYSKLTVDKSRWQQGN | |
| | VFSCSVMHEALHNHYTQKSLSLSP | |
| hIgG2-DHS | ASTKGPSVFPLAPCSRSTSESTAALGCLVKDYFPEPVTV | 234 |
| | SWNSGALTSGVHTFPAVLQSSGLYSLSSVVTVPSSNFG | |
| | TQTYTCNVDHKPSNTKVDKTVERKCCVECPPCPAPPV | |
| | AGPSVFLFPPKPKDTLMISRTPEVTCVVVDVSHEDPEV | |
| | QFNWYVDGVEVHNAKTKPREEQFNSTFRVVSVLTVD | |
| | HHDWLNGKEYKCKVSNKGLPAPIEKTISKTKGQPREP | |
| | QVYTLPPSREEMTKNQVSLTCLVKGFYPSDIAVEWESN | |
| | GQPENNYKTTPPMLDSDGSFFLYSKLTVDKSRWQQGN | |
| | VFSCSVMHEALHSHYTQKSLSLSP | |
| IgG4-SP | ASTKGPSVFPLAPCSRSTSESTAALGCLVKDYFPEPVTV | 235 |
| | SWNSGALTSGVHTFPAVLQSSGLYSLSSVVTVPSSSLG | |
| | TKTYTCNVDHKPSNTKVDKRVESKYGPPCPPCPAPEFL | |
| | GGPSVFLFPPKPKDTLMISRTPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHNHYTQKSLSLSLGK | |
| Human kappa | RTVAAPSVFIFPPSDEQLKSGTASVVCLLNNFYPREAK | 236 |
| LC | VQWKVDNALQSGNSQESVTEQDSKDSTYSLSSTLTLS | |
| | KADYEKHKVYACEVTHQGLSSPVTKSFNRGEC | |
| Human IL-4Rα | UniProtKB: P24394 | 237 |
| | MGWLCSGLLFPVSCLVLLQVASSGNMKVLQEPTCVSD | |
| | YMSISTCEWKMNGPTNCSTELRLLYQLVFLLSEAHTCI | |
| | PENNGGAGCVCHLLMDDVVSADNYTLDLWAGQQLL | |
| | WKGSFKPSEHVKPRAPGNLTVHTNVSDTLLLTWSNPY | |
| | PPDNYLYNHLTYAVNIWSENDPADFRIYNVTYLEPSLR | |
| | IAASTLKSGISYRARVRAWAQCYNTTWSEWSPSTKWH | |
| | NSYREPFEQHLLLGVSVSCIVILAVCLLCYVSITKIKKE | |
| | WWDQIPNPARSRLVAIIIQDAQGSQWEKRSRGQEPAKC | |
| | PHWKNCLTKLLPCFLEHNMKRDEDPHKAAKEMPFQG | |
| | SGKSAWCPVEISKTVLWPESISVVRCVELFEAPVECEEE | |
| | EEVEEEKGSFCASPESSRDDFQEGREGIVARLTESLFLD | |
| | LLGEENGGFCQQDMGESCLLPPSGSTSAHMPWDEFPS | |
| | AGPKEAPPWGKEQPLHLEPSPPASPTQSPDNLTCTETPL | |
| | VIAGNPAYRSFSNSLSQSPCPRELGPDPLLARHLEEVEP | |
| | EMPCVPQLSEPTTVPQPEPETWEQILRRNVLQHGAAAA | |
| | PVSAPTSGYQEFVHAVEQGGTQASAVVGLGPPGEAGY | |
| | KAFSSLLASSAVSPEKCGFGASSGEEGYKPFQDLIPGCP | |
| | GDPAPVPVPLFTFGLDREPPRSPQSSHLPSSSPEHLGLEP | |
| | GEKVEDMPKPPLPQEQATDPLVDSLGSGIVYSALTCHL | |
| | CGHLKQCHGQEDGGQTPVMASPCCGCCCGDRSSPPTT | |
| | PLRAPDPSPGGVPLEASLCPASLAPSGISEKSKSSSSFHP | |
| | APGNAQSSSQTPKIVNFVSVGPTYMRVS | |
| Cynomolgus | UniProtKB: G7Q0S7_MACFA | 238 |
| Monkey IL-4Rα | MGWLCSGLLFPVSCLVLLQVASSGSMKVLQEPACVSD | |
| | YMSISTCEWKMGGPTNCSAELRLLYQLVFQSSETHTC | |
| | VPENNGGVGCVCHLLMDDVVSMDNYTLDLWAGQQL | |
| | LWKGSFKPSEHVKPRAPGNLTVHTNVSDTVLLTWSNP | |
| | YPPDNYLYNDLTYAVNIWSENDPAYSRIHNVTYLKPTL | |
| | RIPASTLKSGISYRARVRAWAQHYNTTWSEWSPSTKW | |
| | YNSYREPFEQRLLWGVSAACVFILFFCLSCYFSVTKIKK | |
| | EWWDQIPNPARSHLVAIIIQDAQESQWEKRSRGQEAA | |
| | KCPYWKNCLTKLLPCFLEHNMKRDEDPHKAVKDLPFR | |
| | GSGKSAWCPVEISKTVLWPESISVVRCVELFEAPVECK | |
| | EEEEVEEEKGSFCTSSESNRDDFQEGREGIVARLTESLF | |
| | LDLLGGENGGFFQQDMGESCLLPPLGSTSAHVPWDEF | |
| | PSAGPKEVPPWGKEQPLHQEPSPPASPTQSPDNPTCTE | |
| | MPLVISSNPAYRSFSNSLSQSPCPRELGPDPLLARHLEE | |
| | VDPEMPCAPQLSEPTTVAPAEPETWEQILRRNVLQHGA | |
| | AAAPASAPTSGYREFVHAVQQGGIQASAVAGLGPPGE | |
| | AGYKAFSSLLASSAVSPGECGFGASSGEEGYKPFQDLT | |
| | PGCPGDPAPVPVPLFTFGLDREPPHSPQSSHLPSNSPEH | |
| | LALEPGEKVEDMQKPPLPPEQATDPLGDNLGSGIVYSA | |
| | LTCHLCGHLKQCHGQEDGGQAPVVASPCCGCCCGDR | |
| | SSPPTTPLRAPDPSLGGVPLEASLCLASLAPSGISEKSKS | |
| | SLSFHPAPGSAQSSSQTPQIVNFVSVGPTCMRVS | |
| Mouse IL-4Rα | UniProtKB: P16382 | 239 |
| | MGRLCTKFLTSVGCLILLLVTGSGSIKVLGEPTCFSDYI | |
| | RTSTCEWFLDSAVDCSSQLCLHYRLMFFEFSENLTCIPR | |
| | NSASTVCVCHMEMNRPVQSDRYQMELWAEHRQLWQ | |
| | GSFSPSGNVKPLAPDNLTLHTNVSDEWLLTWNNLYPS | |
| | NNLLYKDLISMVNISREDNPAEFIVYNVTYKEPRLSFPI | |
| | NILMSGVYYTARVRVRSQILTGTWSEWSPSITWYNHF | |
| | QLPLIQRLPLGVTISCLCIPLFCLFCYFSITKIKKIWWDQI | |
| | PTPARSPLVAIIIQDAQVPLWDKQTRSQESTKYPHWKT | |
| | CLDKLLPCLLKHRVKKKTDFPKAAPTKSLQSPGKAGW | |
| | CPMEVSRTVLWPENVSVSVVRCMELFEAPVQNVEEEE | |
| | DEIVKEDLSMSPENSGGCGFQESQADIMARLTENLFSD | |
| | LLEAENGGLGQSALAESCSPLPSGSGQASVSWACLPM | |
| | GPSEEATCQVTEQPSHPGPLSGSPAQSAPTLACTQVPLV | |
| | LADNPAYRSFSDCCSPAPNPGELAPEQQQADHLEEEEP | |
| | PSPADPHSSGPPMQPVESWEQILHMSVLQHGAAAGSTP | |
| | APAGGYQEFVQAVKQGAAQDPGVPGVRPSGDPGYKA | |
| | FSSLLSSNGIRGDTAAAGTDDGHGGYKPFQNPVPNQSP | |
| | SSVPLFTFGLDTELSPSPLNSDPPKSPPECLGLELGLKGG | |
| | DWVKAPPPADQVPKPFGDDLGFGIVYSSLTCHLCGHL | |
| | KQHHSQEEGGQSPIVASPGCGCCYDDRSPSLGSLSGAL | |
| | ESCPEGIPPEANLMSAPKTPSNLSGEGKGPGHSPVPSQT | |
| | TEVPVGALGIAVS | |
| Rat IL-4Rα | UniProtKB: Q63257 | 240 |
| | MGWLCTKFLSSVSCLILLWVTGSGGIKVLGDPTCFSDY | |
| | IRTSTCEWQLDSTVDCSSQLLLDYRLLFEFSENLTCTPK | |
| | NSADTVCVCQMAIEEPIQADTYWLELWSERGQLWQGS | |
| | FKPSDNVKPPAPDNLTLHTNVSNALLLMWSNPYPSNN | |
| | FLHKGLICMVNISREDNPAEFKVYNVTYTEPKLSFPVN | |
| | TLTSGVRYRARVRVLSQSFPGIWSEWSPSITWYNHFQL | |
| | PLLQRLPLGVSISCICILLFCLTCYFSIIKIKKIWWDQIPTP | |
| | ARSPLAAIIIQDTKVSLWEKQTRSQESTKSRHWKTCLT | |
| | KLLPCLLEHRVKKERESPKAAKTKPLQSPEKAGWYPA | |
| | EVSRTVLWPENVHVSVVRCMELFEAPVQNVEEEEDEM | |
| | VKGDLSMSPENSGGGFQESQADIMARLTENLFSDLLG | |
| | AENGGVGQSSMAESSSLLPSESGQASTSWACFPTGPSE | |
| | TTCQVTGQQPPHPDPERATGTACTQVPLVISDNPAYRS | |
| | FSDFSSPAPNPGELASEQKQAGHLEEGDLLSPVDPHSSG | |
| | PPMQQAESWEQILHMSVLQHGTAGSTPAPTSGYQEFV | |
| | QAVKQGASQDAGVPGVGPSGDTGYKAFSSLLSSSGVC | |
| | TDTAAAGTDSGCGGYKPFQNPVPNQSPNSMPLFTFGL | |
| | DMELPPSPLNSAPPNSTPECLGLELGLKGGTWLKAPPP | |
| | SEQVPKPFGDDLGLGIVYSSLTCHLCGHLKQHHSQEED | |
| | GQIHVVASPGCGCCYDEKSPSLGNLSGTLESCPGEMSQ | |
| | EANLTLAPRTPSNLSGVGKGPGHSPVPSQTTEVPVGTL | |
| | GVTVS | |
| Human IL-4Rα | MGWLCSGLLFPVSCLVLLQVASSGN | 241 |
| Signal Chain | | |
| Cynomolgus | MGWLCSGLLFPVSCLVLLQVASSGS | 242 |
| Monkey IL-4Rα | | |
| Signal Chain | | |
| Mouse IL-4Rα | MGRLCTKFLTSVGCLILLLVTGSGS | 243 |
| Signal Chain | | |
| Rat IL-4Rα | MGWLCTKFLSSVSCLILLWVTGSGG | 244 |
| Signal Chain | | |
| Human IL-4Rα | MKVLQEPTCVSDYMSISTCEWKMNGPTNCSTELRLLY | 245 |
| Main Chain | QLVFLLSEAHTCIPENNGGAGCVCHLLMDDVVSADNY | |
| | TLDLWAGQQLLWKGSFKPSEHVKPRAPGNLTVHTNVS | |
| | DTLLLTWSNPYPPDNYLYNHLTYAVNIWSENDPADFRI | |
| | YNVTYLEPSLRIAASTLKSGISYRARVRAWAQCYNTT | |
| | WSEWSPSTKWHNSYREPFEQHLLLGVSVSCIVILAVCL | |
| | LCYVSITKIKKEWWDQIPNPARSRLVAIIIQDAQGSQWE | |
| | KRSRGQEPAKCPHWKNCLTKLLPCFLEHNMKRDEDPH | |
| | KAAKEMPFQGSGKSAWCPVEISKTVLWPESISVVRCVE | |
| | LFEAPVECEEEEEVEEEKGSFCASPESSRDDFQEGREGI | |
| | VARLTESLFLDLLGEENGGFCQQDMGESCLLPPSGSTS | |
| | AHMPWDEFPSAGPKEAPPWGKEQPLHLEPSPPASPTQS | |
| | PDNLTCTETPLVIAGNPAYRSFSNSLSQSPCPRELGPDP | |
| | LLARHLEEVEPEMPCVPQLSEPTTVPQPEPETWEQILRR | |
| | NVLQHGAAAAPVSAPTSGYQEFVHAVEQGGTQASAV | |
| | VGLGPPGEAGYKAFSSLLASSAVSPEKCGFGASSGEEG | |
| | YKPFQDLIPGCPGDPAPVPVPLFTFGLDREPPRSPQSSH | |
| | LPSSSPEHLGLEPGEKVEDMPKPPLPQEQATDPLVDSL | |
| | GSGIVYSALTCHLCGHLKQCHGQEDGGQTPVMASPCC | |
| | GCCCGDRSSPPTTPLRAPDPSPGGVPLEASLCPASLAPS | |
| | GISEKSKSSSSFHPAPGNAQSSSQTPKIVNFVSVGPTYM | |
| | RVS | |
| Cynomolgus | MKVLQEPACVSDYMSISTCEWKMGGPTNCSAELRLLY | 246 |
| Monkey IL-4Rα | QLVFQSSETHTCVPENNGGVGCVCHLLMDDVVSMDN | |
| Main Chain | YTLDLWAGQQLLWKGSFKPSEHVKPRAPGNLTVHTN | |
| | VSDTVLLTWSNPYPPDNYLYNDLTYAVNIWSENDPAY | |
| | SRIHNVTYLKPTLRIPASTLKSGISYRARVRAWAQHYN | |
| | TTWSEWSPSTKWYNSYREPFEQRLLWGVSAACVFILFF | |
| | CLSCYFSVTKIKKEWWDQIPNPARSHLVAIIIQDAQESQ | |
| | WEKRSRGQEAAKCPYWKNCLTKLLPCFLEHNMKRDE | |
| | DPHKAVKDLPFRGSGKSAWCPVEISKTVLWPESISVVR | |
| | CVELFEAPVECKEEEEVEEEKGSFCTSSESNRDDFQEG | |
| | REGIVARLTESLFLDLLGGENGGFFQQDMGESCLLPPL | |
| | GSTSAHVPWDEFPSAGPKEVPPWGKEQPLHQEPSPPAS | |
| | PTQSPDNPTCTEMPLVISSNPAYRSFSNSLSQSPCPRELG | |
| | PDPLLARHLEEVDPEMPCAPQLSEPTTVAPAEPETWEQ | |
| | ILRRNVLQHGAAAAPASAPTSGYREFVHAVQQGGIQA | |
| | SAVAGLGPPGEAGYKAFSSLLASSAVSPGECGFGASSG | |
| | EEGYKPFQDLTPGCPGDPAPVPVPLFTFGLDREPPHSPQ | |
| | SSHLPSNSPEHLALEPGEKVEDMQKPPLPPEQATDPLG | |
| | DNLGSGIVYSALTCHLCGHLKQCHGQEDGGQAPVVAS | |
| | PCCGCCCGDRSSPPTTPLRAPDPSLGGVPLEASLCLASL | |
| | APSGISEKSKSSLSFHPAPGSAQSSSQTPQIVNFVSVGPT | |
| | CMRVS | |
| Mouse IL-4Rα | IKVLGEPTCFSDYIRTSTCEWFLDSAVDCSSQLCLHYRL | 247 |
| Main Chain | MFFEFSENLTCIPRNSASTVCVCHMEMNRPVQSDRYQ | |
| | MELWAEHRQLWQGSFSPSGNVKPLAPDNLTLHTNVSD | |
| | EWLLTWNNLYPSNNLLYKDLISMVNISREDNPAEFIVY | |
| | NVTYKEPRLSFPINILMSGVYYTARVRVRSQILTGTWS | |
| | EWSPSITWYNHFQLPLIQRLPLGVTISCLCIPLFCLFCYF | |
| | SITKIKKIWWDQIPTPARSPLVAIIIQDAQVPLWDKQTR | |
| | SQESTKYPHWKTCLDKLLPCLLKHRVKKKTDFPKAAP | |
| | TKSLQSPGKAGWCPMEVSRTVLWPENVSVSVVRCME | |
| | LFEAPVQNVEEEEDEIVKEDLSMSPENSGGCGFQESQA | |
| | DIMARLTENLFSDLLEAENGGLGQSALAESCSPLPSGS | |
| | GQASVSWACLPMGPSEEATCQVTEQPSHPGPLSGSPAQ | |
| | SAPTLACTQVPLVLADNPAYRSFSDCCSPAPNPGELAP | |
| | EQQQADHLEEEEPPSPADPHSSGPPMQPVESWEQILHM | |
| | SVLQHGAAAGSTPAPAGGYQEFVQAVKQGAAQDPGV | |
| | PGVRPSGDPGYKAFSSLLSSNGIRGDTAAAGTDDGHG | |
| | GYKPFQNPVPNQSPSSVPLFTFGLDTELSPSPLNSDPPKS | |
| | PPECLGLELGLKGGDWVKAPPPADQVPKPFGDDLGFGI | |
| | VYSSLTCHLCGHLKQHHSQEEGGQSPIVASPGCGCCYD | |
| | DRSPSLGSLSGALESCPEGIPPEANLMSAPKTPSNLSGE | |
| | GKGPGHSPVPSQTTEVPVGALGIAVS | |
| Rat IL-4Rα | IKVLGDPTCFSDYIRTSTCEWQLDSTVDCSSQLLLDYRL | 248 |
| Main Chain | LFEFSENLTCTPKNSADTVCVCQMAIEEPIQADTYWLE | |
| | LWSERGQLWQGSFKPSDNVKPPAPDNLTLHTNVSNAL | |
| | LLMWSNPYPSNNFLHKGLICMVNISREDNPAEFKVYN | |
| | VTYTEPKLSFPVNTLTSGVRYRARVRVLSQSFPGIWSE | |
| | WSPSITWYNHFQLPLLQRLPLGVSISCICILLFCLTCYFSI | |
| | IKIKKIWWDQIPTPARSPLAAIIIQDTKVSLWEKQTRSQE | |
| | STKSRHWKTCLTKLLPCLLEHRVKKERESPKAAKTKPL | |
| | QSPEKAGWYPAEVSRTVLWPENVHVSVVRCMELFEAP | |
| | VQNVEEEEDEMVKGDLSMSPENSGGGFQESQADIMAR | |
| | LTENLFSDLLGAENGGVGQSSMAESSSLLPSESGQAST | |
| | SWACFPTGPSETTCQVTGQQPPHPDPERATGTACTQVP | |
| | LVISDNPAYRSFSDFSSPAPNPGELASEQKQAGHLEEGD | |
| | LLSPVDPHSSGPPMQQAESWEQILHMSVLQHGTAGSTP | |
| | APTSGYQEFVQAVKQGASQDAGVPGVGPSGDTGYKA | |
| | FSSLLSSSGVCTDTAAAGTDSGCGGYKPFQNPVPNQSP | |
| | NSMPLFTFGLDMELPPSPLNSAPPNSTPECLGLELGLKG | |
| | GTWLKAPPPSEQVPKPFGDDLGLGIVYSSLTCHLCGHL | |
| | KQHHSQEEDGQIHVVASPGCGCCYDEKSPSLGNLSGTL | |
| | ESCPGEMSQEANLTLAPRTPSNLSGVGKGPGHSPVPSQ | |
| | TTEVPVGTLGVTVS | |
| Dupilumab | EVQLVESGGGLEQPGGSLRLSCAGSGFTFRDYAMTWV | 249 |
| Heavy Chain full | RQAPGKGLEWVSSISGSGGNTYYADSVKGRFTISRDNS | |
| length sequence | KNTLYLQMNSLRAEDTAVYYCAKDRLSITIRPRYYGL | |
| | DVWGQGTTVTVSSASTKGPSVFPLAPCSRSTSESTAAL | |
| | GCLVKDYFPEPVTVSWNSGALTSGVHTFPAVLQSSGL | |
| | YSLSSVVTVPSSSLGTKTYTCNVDHKPSNTKVDKRVES | |
| | KYGPPCPPCPAPEFLGGPSVFLFPPKPKDTLMISRTPEVT | |
| | CVVVDVSQEDPEVQFNWYVDGVEVHNAKTKPREEQF | |
| | NSTYRVVSVLTVLHQDWLNGKEYKCKVSNKGLPSSIE | |
| | KTISKAKGQPREPQVYTLPPSQEEMTKNQVSLTCLVKG | |
| | FYPSDIAVEWESNGQPENNYKTTPPVLDSDGSFFLYSR | |
| | LTVDKSRWQEGNVFSCSVMHEALHNHYTQKSLSLSLG | |
| Dupilumab | DIVMTQSPLSLPVTPGEPASISCRSSQSLLYSIGYNYLD | 250 |
| Light Chain full | WYLQKSGQSPQLLIYLGSNRASGVPDRFSGSGSGTDFT | |
| length sequence | LKISRVEAEDVGFYYCMQALQTPYTFGQGTKLEIKRTV | |
| | AAPSVFIFPPSDEQLKSGTASVVCLLNNFYPREAKVQW | |
| | KVDNALQSGNSQESVTEQDSKDSTYSLSSTLTLSKADY | |
| | EKHKVYACEVTHQGLSSPVTKSFNRGEC | |
| hIgG1-LA | ASTKGPSVFPLAPSSKSTSGGTAALGCLVKDYFPEPVT | 251 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVLHEALHAHYTQKSLSLSPG | |
| hIgG1- | ASTKGPSVFPLAPSSKSTSGGTAALGCLVKDYFPEPVT | 252 |
| N297A/LA | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYASTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVLHEALHAHYTQKSLSLSPG | |
| hIgG1- | ASTKGPSVFPLAPSSKSTSGGTAALGCLVKDYFPEPVT | 253 |
| D265A/LA | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVAVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVLHEALHAHYTQKSLSLSPG | |
| hIgG1- | ASTKGPSVFPLAPSSKSTSGGTAALGCLVKDYFPEPVT | 254 |
| LALA/LA | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVLHEALHAHYTQKSLSLSPG | |
| hIgG1- | ASTKGPSVFPLAPSSKSTSGGTAALGCLVKDYFPEPVT | 255 |
| LAGA/LA | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVLHEALHAHYTQKSLSLSPG | |
| hIgG1- | ASTKGPSVFPLAPSSKSTSGGTAALGCLVKDYFPEPVT | 256 |
| LALAGA/LA | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVLHEALHAHYTQKSLSLSPG | |
| hIgG1- | ASTKGPSVFPLAPSSKSTSGGTAALGCLVKDYFPEPVT | 257 |
| LALAPG/LA | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALGAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVLHEALHAHYTQKSLSLSPG | |
| hIgG1-N434A | ASTKGPSVFPLAPSSKSTSGGTAALGCLVKDYFPEPVT | 258 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHAHYTQKSLSLSPG | |
| hIgG1-N297A/ | ASTKGPSVFPLAPSSKSTSGGTAALGCLVKDYFPEPVT | 259 |
| N434A | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYASTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHAHYTQKSLSLSPG | |
| hIgG1-D265A/ | ASTKGPSVFPLAPSSKSTSGGTAALGCLVKDYFPEPVT | 260 |
| N434A | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVAVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHAHYTQKSLSLSPG | |
| hIgG1-LALA/ | ASTKGPSVFPLAPSSKSTSGGTAALGCLVKDYFPEPVT | 261 |
| N434A | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHAHYTQKSLSLSPG | |
| hIgG1-LAGA/ | ASTKGPSVFPLAPSSKSTSGGTAALGCLVKDYFPEPVT | 262 |
| N434A | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHAHYTQKSLSLSPG | |
| hIgG1- | ASTKGPSVFPLAPSSKSTSGGTAALGCLVKDYFPEPVT | 263 |
| LALAGA/ | VSWNSGALTSGVHTFPAVLQSSGLYSLSSVVTVPSSSL | |
| N434A | GTQTYICNVNHKPSNTKVDKKVEPKSCDKTHTCPPCP | |
| | APEAAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHAHYTQKSLSLSPG | |
| hIgG1- | ASTKGPSVFPLAPSSKSTSGGTAALGCLVKDYFPEPVT | 264 |
| LALAPG/ | VSWNSGALTSGVHTFPAVLQSSGLYSLSSVVTVPSSSL | |
| N434A | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALGAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHAHYTQKSLSLSPG | |
| hIgG1-N434W | ASTKGPSVFPLAPSSKSTSGGTAALGCLVKDYFPEPVT | 265 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHWHYTQKSLSLSPG | |
| hIgG1-N297A/ | ASTKGPSVFPLAPSSKSTSGGTAALGCLVKDYFPEPVT | 266 |
| N434W | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYASTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHWHYTQKSLSLSPG | |
| hIgG1-D265A/ | ASTKGPSVFPLAPSSKSTSGGTAALGCLVKDYFPEPVT | 267 |
| N434W | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVAVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHWHYTQKSLSLSPG | |
| hIgG1-LALA/ | ASTKGPSVFPLAPSSKSTSGGTAALGCLVKDYFPEPVT | 268 |
| N434W | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHWHYTQKSLSLSPG | |
| hIgG1-LAGA/ | ASTKGPSVFPLAPSSKSTSGGTAALGCLVKDYFPEPVT | 269 |
| N434W | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHWHYTQKSLSLSPG | |
| hIgG1- | ASTKGPSVFPLAPSSKSTSGGTAALGCLVKDYFPEPVT | 270 |
| LALAGA/ | VSWNSGALTSGVHTFPAVLQSSGLYSLSSVVTVPSSSL | |
| N434W | GTQTYICNVNHKPSNTKVDKKVEPKSCDKTHTCPPCP | |
| | APEAAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHWHYTQKSLSLSPG | |
| hIgG1- | ASTKGPSVFPLAPSSKSTSGGTAALGCLVKDYFPEPVT | 271 |
| LALAPG/ | VSWNSGALTSGVHTFPAVLQSSGLYSLSSVVTVPSSSL | |
| N434W | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALGAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHWHYTQKSLSLSPG | |
| hIgG1/DQ | ASTKGPSVFPLAPSSKSTSGGTAALGCLVKDYFPEPVT | 272 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRDPEVTCVVVDVSH | |
| | EDPEVKFNWYVDGVEVHNAKTKPREEQYNSTYRVVS | |
| | VLQVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 273 |
| N297A/DQ | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRDPEVTCVVVDVSH | |
| | EDPEVKFNWYVDGVEVHNAKTKPREEQYASTYRVVS | |
| | VLQVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 274 |
| D265A/DQ | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRDPEVTCVVVAVSH | |
| | EDPEVKFNWYVDGVEVHNAKTKPREEQYNSTYRVVS | |
| | VLQVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 275 |
| LALA/DQ | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRDPEVTCVVVDVSH | |
| | EDPEVKFNWYVDGVEVHNAKTKPREEQYNSTYRVVS | |
| | VLQVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 276 |
| LAGA/DQ | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRDPEVTCVVVDVSH | |
| | EDPEVKFNWYVDGVEVHNAKTKPREEQYNSTYRVVS | |
| | VLQVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 277 |
| LALAGA/DQ | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLMISRDPEVTCVVVDVSH | |
| | EDPEVKFNWYVDGVEVHNAKTKPREEQYNSTYRVVS | |
| | VLQVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 278 |
| LALAPG/DQ | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRDPEVTCVVVDVSH | |
| | EDPEVKFNWYVDGVEVHNAKTKPREEQYNSTYRVVS | |
| | VLQVLHQDWLNGKEYKCKVSNKALGAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1/DW | ASTKGPSVFPLAPSSKSTSGGTAALGCLVKDYFPEPVT | 279 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRDPEVTCVVVDVSH | |
| | EDPEVKFNWYVDGVEVHNAKTKPREEQYNSTYRVVS | |
| | VLWVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 280 |
| N297A/DW | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRDPEVTCVVVDVSH | |
| | EDPEVKFNWYVDGVEVHNAKTKPREEQYASTYRVVS | |
| | VLWVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 281 |
| D265A/DW | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRDPEVTCVVVAVSH | |
| | EDPEVKFNWYVDGVEVHNAKTKPREEQYNSTYRVVS | |
| | VLWVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 282 |
| LALA/DW | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRDPEVTCVVVDVSH | |
| | EDPEVKFNWYVDGVEVHNAKTKPREEQYNSTYRVVS | |
| | VLWVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 283 |
| LAGA/DW | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRDPEVTCVVVDVSH | |
| | EDPEVKFNWYVDGVEVHNAKTKPREEQYNSTYRVVS | |
| | VLWVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 284 |
| LALAGA/DW | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLMISRDPEVTCVVVDVSH | |
| | EDPEVKFNWYVDGVEVHNAKTKPREEQYNSTYRVVS | |
| | VLWVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 285 |
| LALAPG/DW | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRDPEVTCVVVDVSH | |
| | EDPEVKFNWYVDGVEVHNAKTKPREEQYNSTYRVVS | |
| | VLWVLHQDWLNGKEYKCKVSNKALGAPIEKTISKAK | |
| | GQPREPQVYTLPPSRDELTKNQVSLTCLVKGFYPSDIA | |
| | VEWESNGQPENNYKTTPPVLDSDGSFFLYSKLTVDKSR | |
| | WQQGNVFSCSVMHEALHNHYTQKSLSLSPG | |
| hIgG1/YD | ASTKGPSVFPLAPSSKSTSGGTAALGCLVKDYFPEPVT | 286 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLYISRDPEVTCVVVDVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPG | |
| hIgG1- | ASTKGPSVFPLAPSSKSTSGGTAALGCLVKDYFPEPVT | 287 |
| N297A/YD | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLYISRDPEVTCVVVDVSHE | |
| | DPEVKFNWYVDGVEVHNAKTKPREEQYASTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPG | |
| hIgG1- | ASTKGPSVFPLAPSSKSTSGGTAALGCLVKDYFPEPVT | 288 |
| D265A/YD | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLYISRDPEVTCVVVAVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPG | |
| hIgG1- | ASTKGPSVFPLAPSSKSTSGGTAALGCLVKDYFPEPVT | 289 |
| LALA/YD | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLYISRDPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 290 |
| LAGA/YD | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLYISRDPEVTCVVVDVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPG | |
| hIgG1- | ASTKGPSVFPLAPSSKSTSGGTAALGCLVKDYFPEPVT | 291 |
| LALAGA/YD | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLYISRDPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 292 |
| LALAPG/YD | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLYISRDPEVTCVVVDVSH | |
| | EDPEVKFNWYVDGVEVHNAKTKPREEQYNSTYRVVS | |
| | VLTVLHQDWLNGKEYKCKVSNKALGAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPG | |
| hIgG1/QVV | ASTKGPSVFPLAPSSKSTSGGTAALGCLVKDYFPEPVT | 293 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYNSTYRVVSV | |
| | LQVLHVDWLNGKEYKCKVSNKALPAPIEKTISKAKGQ | |
| | PREPQVYTLPPSRDELTKNQVSLTCLVKGFYPSDIVVE | |
| | WESNGQPENNYKTTPPVLDSDGSFFLYSKLTVDKSRW | |
| | QQGNVFSCSVMHEALHNHYTQKSLSLSPG | |
| hIgG1- | ASTKGPSVFPLAPSSKSTSGGTAALGCLVKDYFPEPVT | 294 |
| N297A/QVV | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYASTYRVVSV | |
| | LQVLHVDWLNGKEYKCKVSNKALPAPIEKTISKAKGQ | |
| | PREPQVYTLPPSRDELTKNQVSLTCLVKGFYPSDIVVE | |
| | WESNGQPENNYKTTPPVLDSDGSFFLYSKLTVDKSRW | |
| | QQGNVFSCSVMHEALHNHYTQKSLSLSPG | |
| hIgG1- | ASTKGPSVFPLAPSSKSTSGGTAALGCLVKDYFPEPVT | 295 |
| D265A/QVV | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVAVSHE | |
| | DPEVKFNWYVDGVEVHNAKTKPREEQYNSTYRVVSV | |
| | LQVLHVDWLNGKEYKCKVSNKALPAPIEKTISKAKGQ | |
| | PREPQVYTLPPSRDELTKNQVSLTCLVKGFYPSDIVVE | |
| | WESNGQPENNYKTTPPVLDSDGSFFLYSKLTVDKSRW | |
| | QQGNVFSCSVMHEALHNHYTQKSLSLSPG | |
| hIgG1- | ASTKGPSVFPLAPSSKSTSGGTAALGCLVKDYFPEPVT | 296 |
| LALA/QVV | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLQVLHVDWLNGKEYKCKVSNKALPAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 297 |
| LAGA/QVV | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRTPEVTCVVVDVSH | |
| | EDPEVKFNWYVDGVEVHNAKTKPREEQYNSTYRVVS | |
| | VLQVLHVDWLNGKEYKCKVSNKALPAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 298 |
| LALAGA/QVV | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLMISRTPEVTCVVVDVSH | |
| | EDPEVKFNWYVDGVEVHNAKTKPREEQYNSTYRVVS | |
| | VLQVLHVDWLNGKEYKCKVSNKALPAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 299 |
| LALAPG/QVV | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLQVLHVDWLNGKEYKCKVSNKALGAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPG | |
| hIgG1/DDRVV | ASTKGPSVFPLAPSSKSTSGGTAALGCLVKDYFPEPVT | 300 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRDPEVTCVVVDVSH | |
| | EDPEVKFNWYVDGVEVDNAKTKPREEQYNSTYRVVS | |
| | VLRVLHVDWLNGKEYKCKVSNKALPAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 301 |
| N297A/DDRVV | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRDPEVTCVVVDVSH | |
| | EDPEVKFNWYVDGVEVDNAKTKPREEQYASTYRVVS | |
| | VLRVLHVDWLNGKEYKCKVSNKALPAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 302 |
| D265A/DDRVV | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRDPEVTCVVVAVSH | |
| | EDPEVKFNWYVDGVEVDNAKTKPREEQYNSTYRVVS | |
| | VLRVLHVDWLNGKEYKCKVSNKALPAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 303 |
| LALA/DDRVV | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRDPEVTCVVVDVSH | |
| | EDPEVKFNWYVDGVEVDNAKTKPREEQYNSTYRVVS | |
| | VLRVLHVDWLNGKEYKCKVSNKALPAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 304 |
| LAGA/DDRVV | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRDPEVTCVVVDVSH | |
| | EDPEVKFNWYVDGVEVDNAKTKPREEQYNSTYRVVS | |
| | VLRVLHVDWLNGKEYKCKVSNKALPAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 305 |
| LALAGA/DDRVV | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLMISRDPEVTCVVVDVSH | |
| | EDPEVKFNWYVDGVEVDNAKTKPREEQYNSTYRVVS | |
| | VLRVLHVDWLNGKEYKCKVSNKALPAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPG | |
| hIgG1- | ASTKGPSVFPLAPSSKSTSGGTAALGCLVKDYFPEPVT | 306 |
| LALAPG/DDRVV | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRDPEVTCVVVDVSH | |
| | EDPEVKFNWYVDGVEVDNAKTKPREEQYNSTYRVVS | |
| | VLRVLHVDWLNGKEYKCKVSNKALGAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPG | |
| hIgG1* | ASTKGPSVFPLAPSSKSTSGGTAALGCLVKDYFPEPVT | 307 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPGK | |
| hIgG4 | ASTKGPSVFPLAPCSRSTSESTAALGCLVKDYFPEPVTV | 308 |
| | SWNSGALTSGVHTFPAVLQSSGLYSLSSVVTVPSSSLG | |
| | TKTYTCNVDHKPSNTKVDKRVESKYGPPCPSCPAPEFL | |
| | GGPSVFLFPPKPKDTLMISRTPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHNHYTQKSLSLSLGK | |
| IgG4-SP | ASTKGPSVFPLAPCSRSTSESTAALGCLVKDYFPEPVTV | 309 |
| | SWNSGALTSGVHTFPAVLQSSGLYSLSSVVTVPSSSLG | |
| | TKTYTCNVDHKPSNTKVDKRVESKYGPPCPPCPAPEFL | |
| | GGPSVFLFPPKPKDTLMISRTPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHNHYTQKSLSLSLGK | |
| IgG4-SPLE | ASTKGPSVFPLAPCSRSTSESTAALGCLVKDYFPEPVTV | 310 |
| | SWNSGALTSGVHTFPAVLQSSGLYSLSSVVTVPSSSLG | |
| | TKTYTCNVDHKPSNTKVDKRVESKYGPPCPPCPAPEEL | |
| | GGPSVFLFPPKPKDTLMISRTPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHNHYTQKSLSLSLGK | |
| IgG2 | ASTKGPSVFPLAPCSRSTSESTAALGCLVKDYFPEPVTV | 311 |
| | SWNSGALTSGVHTFPAVLQSSGLYSLSSVVTVPSSNFG | |
| | TQTYTCNVDHKPSNTKVDKTVERKCCVECPPCPAPPV | |
| | AGPSVFLFPPKPKDTLMISRTPEVTCVVVDVSHEDPEV | |
| | QFNWYVDGVEVHNAKTKPREEQFNSTFRVVSVLTVV | |
| | HQDWLNGKEYKCKVSNKGLPAPIEKTISKTKGQPREP | |
| | QVYTLPPSREEMTKNQVSLTCLVKGFYPSDIAVEWESN | |
| | GQPENNYKTTPPMLDSDGSFFLYSKLTVDKSRWQQGN | |
| | VFSCSVMHEALHNHYTQKSLSLSPK | |
| hIgG1-N297A | ASTKGPSVFPLAPSSKSTSGGTAALGCLVKDYFPEPVT | 312 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYASTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPGK | |
| hIgG1-D265A | ASTKGPSVFPLAPSSKSTSGGTAALGCLVKDYFPEPVT | 313 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVAVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPGK | |
| hIgG1-LALA | ASTKGPSVFPLAPSSKSTSGGTAALGCLVKDYFPEPVT | 314 |
| | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1-LAGA | ASTKGPSVFPLAPSSKSTSGGTAALGCLVKDYFPEPVT | 315 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 316 |
| LALAGA | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1-LALAPG | ASTKGPSVFPLAPSSKSTSGGTAALGCLVKDYFPEPVT | 317 |
| | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALGAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1-YTE | ASTKGPSVFPLAPSSKSTSGGTAALGCLVKDYFPEPVT | 318 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLYITREPEVTCVVVDVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPGK | |
| hIgG1- | ASTKGPSVFPLAPSSKSTSGGTAALGCLVKDYFPEPVT | 319 |
| N297A/YTE | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLYITREPEVTCVVVDVSHE | |
| | DPEVKFNWYVDGVEVHNAKTKPREEQYASTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPGK | |
| hIgG1- | ASTKGPSVFPLAPSSKSTSGGTAALGCLVKDYFPEPVT | 320 |
| D265A/YTE | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLYITREPEVTCVVVAVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPGK | |
| hIgG1- | ASTKGPSVFPLAPSSKSTSGGTAALGCLVKDYFPEPVT | 321 |
| LALA/YTE | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLYITREPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 322 |
| LAGA/YTE | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLYITREPEVTCVVVDVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPGK | |
| hIgG1- | ASTKGPSVFPLAPSSKSTSGGTAALGCLVKDYFPEPVT | 323 |
| LALAGA/YTE | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLYITREPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 324 |
| LALAPG/YTE | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLYITREPEVTCVVVDVSH | |
| | EDPEVKFNWYVDGVEVHNAKTKPREEQYNSTYRVVS | |
| | VLTVLHQDWLNGKEYKCKVSNKALGAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1-LS | ASTKGPSVFPLAPSSKSTSGGTAALGCLVKDYFPEPVT | 325 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVLHEALHSHYTQKSLSLSPGK | |
| hIgG1- | ASTKGPSVFPLAPSSKSTSGGTAALGCLVKDYFPEPVT | 326 |
| N297A/LS | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYASTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVLHEALHSHYTQKSLSLSPGK | |
| hIgG1- | ASTKGPSVFPLAPSSKSTSGGTAALGCLVKDYFPEPVT | 327 |
| D265A/LS | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVAVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVLHEALHSHYTQKSLSLSPGK | |
| hIgG1- | ASTKGPSVFPLAPSSKSTSGGTAALGCLVKDYFPEPVT | 328 |
| LALA/LS | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVLHEALHSHYTQKSLSLSPGK | |
| hIgG1- | ASTKGPSVFPLAPSSKSTSGGTAALGCLVKDYFPEPVT | 329 |
| LAGA/LS | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVLHEALHSHYTQKSLSLSPGK | |
| hIgG1- | ASTKGPSVFPLAPSSKSTSGGTAALGCLVKDYFPEPVT | 330 |
| LALAGA/LS | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVLHEALHSHYTQKSLSLSPGK | |
| hIgG1- | ASTKGPSVFPLAPSSKSTSGGTAALGCLVKDYFPEPVT | 331 |
| LALAPG/LS | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALGAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVLHEALHSHYTQKSLSLSPGK | |
| hIgG1-DHS | ASTKGPSVFPLAPSSKSTSGGTAALGCLVKDYFPEPVT | 332 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYNSTYRVVSV | |
| | LTVDHHDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHSHYTQKSLSLSPGK | |
| hIgG1- | ASTKGPSVFPLAPSSKSTSGGTAALGCLVKDYFPEPVT | 333 |
| N297A/DHS | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYASTYRVVSV | |
| | LTVDHHDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHSHYTQKSLSLSPGK | |
| hIgG1- | ASTKGPSVFPLAPSSKSTSGGTAALGCLVKDYFPEPVT | 334 |
| D265A/DHS | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVAVSHE | |
| | DPEVKFNWYVDGVEVHNAKTKPREEQYNSTYRVVSV | |
| | LTVDHHDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHSHYTQKSLSLSPGK | |
| hIgG1- | ASTKGPSVFPLAPSSKSTSGGTAALGCLVKDYFPEPVT | 335 |
| LALA/DHS | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVDHHDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHSHYTQKSLSLSPGK | |
| hIgG1- | ASTKGPSVFPLAPSSKSTSGGTAALGCLVKDYFPEPVT | 336 |
| LAGA/DHS | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRTPEVTCVVVDVSH | |
| | EDPEVKFNWYVDGVEVHNAKTKPREEQYNSTYRVVS | |
| | VLTVDHHDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHSHYTQKSLSLSPGK | |
| hIgG1- | ASTKGPSVFPLAPSSKSTSGGTAALGCLVKDYFPEPVT | 337 |
| LALAGA/DHS | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLMISRTPEVTCVVVDVSH | |
| | EDPEVKFNWYVDGVEVHNAKTKPREEQYNSTYRVVS | |
| | VLTVDHHDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHSHYTQKSLSLSPGK | |
| hIgG1- | ASTKGPSVFPLAPSSKSTSGGTAALGCLVKDYFPEPVT | 338 |
| LALAPG/DHS | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVDHHDWLNGKEYKCKVSNKALGAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHSHYTQKSLSLSPGK | |
| hIgG4-YTE | ASTKGPSVFPLAPCSRSTSESTAALGCLVKDYFPEPVTV | 339 |
| | SWNSGALTSGVHTFPAVLQSSGLYSLSSVVTVPSSSLG | |
| | TKTYTCNVDHKPSNTKVDKRVESKYGPPCPSCPAPEFL | |
| | GGPSVFLFPPKPKDTLYITREPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHNHYTQKSLSLSLGK | |
| hIgG4-SP/YTE | ASTKGPSVFPLAPCSRSTSESTAALGCLVKDYFPEPVTV | 340 |
| | SWNSGALTSGVHTFPAVLQSSGLYSLSSVVTVPSSSLG | |
| | TKTYTCNVDHKPSNTKVDKRVESKYGPPCPPCPAPEFL | |
| | GGPSVFLFPPKPKDTLYITREPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHNHYTQKSLSLSLGK | |
| hIgG4- | ASTKGPSVFPLAPCSRSTSESTAALGCLVKDYFPEPVTV | 341 |
| SPLE/YTE | SWNSGALTSGVHTFPAVLQSSGLYSLSSVVTVPSSSLG | |
| | TKTYTCNVDHKPSNTKVDKRVESKYGPPCPPCPAPEEL | |
| | GGPSVFLFPPKPKDTLYITREPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHNHYTQKSLSLSLGK | |
| hIgG4-LS | ASTKGPSVFPLAPCSRSTSESTAALGCLVKDYFPEPVTV | 342 |
| | SWNSGALTSGVHTFPAVLQSSGLYSLSSVVTVPSSSLG | |
| | TKTYTCNVDHKPSNTKVDKRVESKYGPPCPSCPAPEFL | |
| | GGPSVFLFPPKPKDTLMISRTPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVLHEALHSYTQKSLSLSLGK | |
| hIgG4-SP/LS | ASTKGPSVFPLAPCSRSTSESTAALGCLVKDYFPEPVTV | 343 |
| | SWNSGALTSGVHTFPAVLQSSGLYSLSSVVTVPSSSLG | |
| | TKTYTCNVDHKPSNTKVDKRVESKYGPPCPPCPAPEFL | |
| | GGPSVFLFPPKPKDTLMISRTPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVLHEALHSHYTQKSLSLSLGK | |
| hIgG4-SPLE/LS | ASTKGPSVFPLAPCSRSTSESTAALGCLVKDYFPEPVTV | 344 |
| | SWNSGALTSGVHTFPAVLQSSGLYSLSSVVTVPSSSLG | |
| | TKTYTCNVDHKPSNTKVDKRVESKYGPPCPPCPAPEEL | |
| | GGPSVFLFPPKPKDTLMISRTPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVLHEALHSHYTQKSLSLSLGK | |
| hIgG4-DHS | ASTKGPSVFPLAPCSRSTSESTAALGCLVKDYFPEPVTV | 345 |
| | SWNSGALTSGVHTFPAVLQSSGLYSLSSVVTVPSSSLG | |
| | TKTYTCNVDHKPSNTKVDKRVESKYGPPCPSCPAPEFL | |
| | GGPSVFLFPPKPKDTLMISRTPEVTCVVVDVSQEDPEV | |
| | QFNWYVDGVEVHNAKTKPREEQFNSTYRVVSVLTVD | |
| | HHDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHSHYTQKSLSLSLGK | |
| hIgG4-SP/DHS | ASTKGPSVFPLAPCSRSTSESTAALGCLVKDYFPEPVTV | 346 |
| | SWNSGALTSGVHTFPAVLQSSGLYSLSSVVTVPSSSLG | |
| | TKTYTCNVDHKPSNTKVDKRVESKYGPPCPPCPAPEFL | |
| | GGPSVFLFPPKPKDTLMISRTPEVTCVVVDVSQEDPEV | |
| | QFNWYVDGVEVHNAKTKPREEQFNSTYRVVSVLTVD | |
| | HHDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHSHYTQKSLSLSLGK | |
| hIgG4- | ASTKGPSVFPLAPCSRSTSESTAALGCLVKDYFPEPVTV | 347 |
| SPLE/DHS | SWNSGALTSGVHTFPAVLQSSGLYSLSSVVTVPSSSLG | |
| | TKTYTCNVDHKPSNTKVDKRVESKYGPPCPPCPAPEEL | |
| | GGPSVFLFPPKPKDTLMISRTPEVTCVVVDVSQEDPEV | |
| | QFNWYVDGVEVHNAKTKPREEQFNSTYRVVSVLTVD | |
| | HHDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHSHYTQKSLSLSLGK | |
| hIgG2-YTE | ASTKGPSVFPLAPCSRSTSESTAALGCLVKDYFPEPVTV | 348 |
| | SWNSGALTSGVHTFPAVLQSSGLYSLSSVVTVPSSNFG | |
| | TQTYTCNVDHKPSNTKVDKTVERKCCVECPPCPAPPV | |
| | AGPSVFLFPPKPKDTLYITREPEVTCVVVDVSHEDPEV | |
| | QFNWYVDGVEVHNAKTKPREEQFNSTFRVVSVLTVV | |
| | HQDWLNGKEYKCKVSNKGLPAPIEKTISKTKGQPREP | |
| | QVYTLPPSREEMTKNQVSLTCLVKGFYPSDIAVEWESN | |
| | GQPENNYKTTPPMLDSDGSFFLYSKLTVDKSRWQQGN | |
| | VFSCSVLHEALHSHYTQKSLSLSPK | |
| hIgG2-LS | ASTKGPSVFPLAPCSRSTSESTAALGCLVKDYFPEPVTV | 349 |
| | SWNSGALTSGVHTFPAVLQSSGLYSLSSVVTVPSSNFG | |
| | TQTYTCNVDHKPSNTKVDKTVERKCCVECPPCPAPPV | |
| | AGPSVFLFPPKPKDTLMISRTPEVTCVVVDVSHEDPEV | |
| | QFNWYVDGVEVHNAKTKPREEQFNSTFRVVSVLTVV | |
| | HQDWLNGKEYKCKVSNKGLPAPIEKTISKTKGQPREP | |
| | QVYTLPPSREEMTKNQVSLTCLVKGFYPSDIAVEWESN | |
| | GQPENNYKTTPPMLDSDGSFFLYSKLTVDKSRWQQGN | |
| | VFSCSVMHEALHNHYTQKSLSLSPK | |
| hIgG2-DHS | ASTKGPSVFPLAPCSRSTSESTAALGCLVKDYFPEPVTV | 350 |
| | SWNSGALTSGVHTFPAVLQSSGLYSLSSVVTVPSSNFG | |
| | TQTYTCNVDHKPSNTKVDKTVERKCCVECPPCPAPPV | |
| | AGPSVFLFPPKPKDTLMISRTPEVTCVVVDVSHEDPEV | |
| | QFNWYVDGVEVHNAKTKPREEQFNSTFRVVSVLTVD | |
| | HHDWLNGKEYKCKVSNKGLPAPIEKTISKTKGQPREP | |
| | QVYTLPPSREEMTKNQVSLTCLVKGFYPSDIAVEWESN | |
| | GQPENNYKTTPPMLDSDGSFFLYSKLTVDKSRWQQGN | |
| | VFSCSVMHEALHSHYTQKSLSLSPK | |
| IgG4-SP | ASTKGPSVFPLAPCSRSTSESTAALGCLVKDYFPEPVTV | 351 |
| | SWNSGALTSGVHTFPAVLQSSGLYSLSSVVTVPSSSLG | |
| | TKTYTCNVDHKPSNTKVDKRVESKYGPPCPPCPAPEFL | |
| | GGPSVFLFPPKPKDTLMISRTPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNV | |
| | FSCSVMHEALHNHYTQKSLSLSLGK | |
| hIgG1-LA | ASTKGPSVFPLAPSSKSTSGGTAALGCLVKDYFPEPVT | 352 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVLHEALHAHYTQKSLSLSPGK | |
| hIgG1- | ASTKGPSVFPLAPSSKSTSGGTAALGCLVKDYFPEPVT | 353 |
| N297A/LA | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYASTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVLHEALHAHYTQKSLSLSPGK | |
| hIgG1- | ASTKGPSVFPLAPSSKSTSGGTAALGCLVKDYFPEPVT | 354 |
| D265A/LA | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVAVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVLHEALHAHYTQKSLSLSPGK | |
| hIgG1- | ASTKGPSVFPLAPSSKSTSGGTAALGCLVKDYFPEPVT | 355 |
| LALA/LA | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVLHEALHAHYTQKSLSLSPGK | |
| hIgG1- | ASTKGPSVFPLAPSSKSTSGGTAALGCLVKDYFPEPVT | 356 |
| LAGA/LA | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVLHEALHAHYTQKSLSLSPGK | |
| hIgG1- | ASTKGPSVFPLAPSSKSTSGGTAALGCLVKDYFPEPVT | 357 |
| LALAGA/LA | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVLHEALHAHYTQKSLSLSPGK | |
| hIgG1- | ASTKGPSVFPLAPSSKSTSGGTAALGCLVKDYFPEPVT | 358 |
| LALAPG/LA | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALGAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVLHEALHAHYTQKSLSLSPGK | |
| hIgG1-N434A | ASTKGPSVFPLAPSSKSTSGGTAALGCLVKDYFPEPVT | 359 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHAHYTQKSLSLSPGK | |
| hIgG1-N297A/ | ASTKGPSVFPLAPSSKSTSGGTAALGCLVKDYFPEPVT | 360 |
| N434A | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYASTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHAHYTQKSLSLSPGK | |
| hIgG1-D265A/ | ASTKGPSVFPLAPSSKSTSGGTAALGCLVKDYFPEPVT | 361 |
| N434A | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVAVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHAHYTQKSLSLSPGK | |
| hIgG1-LALA/ | ASTKGPSVFPLAPSSKSTSGGTAALGCLVKDYFPEPVT | 362 |
| N434A | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHAHYTQKSLSLSPGK | |
| hIgG1-LAGA/ | ASTKGPSVFPLAPSSKSTSGGTAALGCLVKDYFPEPVT | 363 |
| N434A | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHAHYTQKSLSLSPGK | |
| hIgG1- | ASTKGPSVFPLAPSSKSTSGGTAALGCLVKDYFPEPVT | 364 |
| LALAGA/ | VSWNSGALTSGVHTFPAVLQSSGLYSLSSVVTVPSSSL | |
| N434A | GTQTYICNVNHKPSNTKVDKKVEPKSCDKTHTCPPCP | |
| | APEAAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHAHYTQKSLSLSPGK | |
| hIgG1- | ASTKGPSVFPLAPSSKSTSGGTAALGCLVKDYFPEPVT | 365 |
| LALAPG/ | VSWNSGALTSGVHTFPAVLQSSGLYSLSSVVTVPSSSL | |
| N434A | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALGAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHAHYTQKSLSLSPGK | |
| hIgG1-N434W | ASTKGPSVFPLAPSSKSTSGGTAALGCLVKDYFPEPVT | 366 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHWHYTQKSLSLSPGK | |
| hIgG1-N297A/ | ASTKGPSVFPLAPSSKSTSGGTAALGCLVKDYFPEPVT | 367 |
| N434W | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYASTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHWHYTQKSLSLSPGK | |
| hIgG1-D265A/ | ASTKGPSVFPLAPSSKSTSGGTAALGCLVKDYFPEPVT | 368 |
| N434W | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVAVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHWHYTQKSLSLSPGK | |
| hIgG1-LALA/ | ASTKGPSVFPLAPSSKSTSGGTAALGCLVKDYFPEPVT | 369 |
| N434W | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHWHYTQKSLSLSPGK | |
| hIgG1-LAGA/ | ASTKGPSVFPLAPSSKSTSGGTAALGCLVKDYFPEPVT | 370 |
| N434W | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHWHYTQKSLSLSPGK | |
| hIgG1- | ASTKGPSVFPLAPSSKSTSGGTAALGCLVKDYFPEPVT | 371 |
| LALAGA/ | VSWNSGALTSGVHTFPAVLQSSGLYSLSSVVTVPSSSL | |
| N434W | GTQTYICNVNHKPSNTKVDKKVEPKSCDKTHTCPPCP | |
| | APEAAGAPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHWHYTQKSLSLSPGK | |
| hIgG1- | ASTKGPSVFPLAPSSKSTSGGTAALGCLVKDYFPEPVT | 372 |
| LALAPG/ | VSWNSGALTSGVHTFPAVLQSSGLYSLSSVVTVPSSSL | |
| N434W | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLTVLHQDWLNGKEYKCKVSNKALGAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHWHYTQKSLSLSPGK | |
| hIgG1/DQ | ASTKGPSVFPLAPSSKSTSGGTAALGCLVKDYFPEPVT | 373 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRDPEVTCVVVDVSH | |
| | EDPEVKFNWYVDGVEVHNAKTKPREEQYNSTYRVVS | |
| | VLQVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 374 |
| N297A/DQ | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRDPEVTCVVVDVSH | |
| | EDPEVKFNWYVDGVEVHNAKTKPREEQYASTYRVVS | |
| | VLQVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 375 |
| D265A/DQ | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRDPEVTCVVVAVSH | |
| | EDPEVKFNWYVDGVEVHNAKTKPREEQYNSTYRVVS | |
| | VLQVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 376 |
| LALA/DQ | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRDPEVTCVVVDVSH | |
| | EDPEVKFNWYVDGVEVHNAKTKPREEQYNSTYRVVS | |
| | VLQVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 377 |
| LAGA/DQ | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRDPEVTCVVVDVSH | |
| | EDPEVKFNWYVDGVEVHNAKTKPREEQYNSTYRVVS | |
| | VLQVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 378 |
| LALAGA/DQ | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLMISRDPEVTCVVVDVSH | |
| | EDPEVKFNWYVDGVEVHNAKTKPREEQYNSTYRVVS | |
| | VLQVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 379 |
| LALAPG/DQ | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRDPEVTCVVVDVSH | |
| | EDPEVKFNWYVDGVEVHNAKTKPREEQYNSTYRVVS | |
| | VLQVLHQDWLNGKEYKCKVSNKALGAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1/DW | ASTKGPSVFPLAPSSKSTSGGTAALGCLVKDYFPEPVT | 380 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRDPEVTCVVVDVSH | |
| | EDPEVKFNWYVDGVEVHNAKTKPREEQYNSTYRVVS | |
| | VLWVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 381 |
| N297A/DW | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRDPEVTCVVVDVSH | |
| | EDPEVKFNWYVDGVEVHNAKTKPREEQYASTYRVVS | |
| | VLWVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 382 |
| D265A/DW | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRDPEVTCVVVAVSH | |
| | EDPEVKFNWYVDGVEVHNAKTKPREEQYNSTYRVVS | |
| | VLWVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 383 |
| LALA/DW | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRDPEVTCVVVDVSH | |
| | EDPEVKFNWYVDGVEVHNAKTKPREEQYNSTYRVVS | |
| | VLWVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 384 |
| LAGA/DW | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRDPEVTCVVVDVSH | |
| | EDPEVKFNWYVDGVEVHNAKTKPREEQYNSTYRVVS | |
| | VLWVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 385 |
| LALAGA/DW | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLMISRDPEVTCVVVDVSH | |
| | EDPEVKFNWYVDGVEVHNAKTKPREEQYNSTYRVVS | |
| | VLWVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 386 |
| LALAPG/DW | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRDPEVTCVVVDVSH | |
| | EDPEVKFNWYVDGVEVHNAKTKPREEQYNSTYRVVS | |
| | VLWVLHQDWLNGKEYKCKVSNKALGAPIEKTISKAK | |
| | GQPREPQVYTLPPSRDELTKNQVSLTCLVKGFYPSDIA | |
| | VEWESNGQPENNYKTTPPVLDSDGSFFLYSKLTVDKSR | |
| | WQQGNVFSCSVMHEALHNHYTQKSLSLSPGK | |
| hIgG1/YD | ASTKGPSVFPLAPSSKSTSGGTAALGCLVKDYFPEPVT | 387 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLYISRDPEVTCVVVDVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPGK | |
| hIgG1- | ASTKGPSVFPLAPSSKSTSGGTAALGCLVKDYFPEPVT | 388 |
| N297A/YD | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLYISRDPEVTCVVVDVSHE | |
| | DPEVKFNWYVDGVEVHNAKTKPREEQYASTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPGK | |
| hIgG1- | ASTKGPSVFPLAPSSKSTSGGTAALGCLVKDYFPEPVT | 389 |
| D265A/YD | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLYISRDPEVTCVVVAVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPGK | |
| hIgG1- | ASTKGPSVFPLAPSSKSTSGGTAALGCLVKDYFPEPVT | 390 |
| LALA/YD | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLYISRDPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 391 |
| LAGA/YD | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLYISRDPEVTCVVVDVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPGK | |
| hIgG1- | ASTKGPSVFPLAPSSKSTSGGTAALGCLVKDYFPEPVT | 392 |
| LALAGA/YD | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLYISRDPEVTCVVVDVSH | |
| | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 393 |
| LALAPG/YD | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLYISRDPEVTCVVVDVSH | |
| | EDPEVKFNWYVDGVEVHNAKTKPREEQYNSTYRVVS | |
| | VLTVLHQDWLNGKEYKCKVSNKALGAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| hIgG1/QVV | ASTKGPSVFPLAPSSKSTSGGTAALGCLVKDYFPEPVT | 394 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYNSTYRVVSV | |
| | LQVLHVDWLNGKEYKCKVSNKALPAPIEKTISKAKGQ | |
| | PREPQVYTLPPSRDELTKNQVSLTCLVKGFYPSDIVVE | |
| | WESNGQPENNYKTTPPVLDSDGSFFLYSKLTVDKSRW | |
| | QQGNVFSCSVMHEALHNHYTQKSLSLSPGK | |
| hIgG1- | ASTKGPSVFPLAPSSKSTSGGTAALGCLVKDYFPEPVT | 395 |
| N297A/QVV | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYASTYRVVSV | |
| | LQVLHVDWLNGKEYKCKVSNKALPAPIEKTISKAKGQ | |
| | PREPQVYTLPPSRDELTKNQVSLTCLVKGFYPSDIVVE | |
| | WESNGQPENNYKTTPPVLDSDGSFFLYSKLTVDKSRW | |
| | QQGNVFSCSVMHEALHNHYTQKSLSLSPGK | |
| hIgG1- | ASTKGPSVFPLAPSSKSTSGGTAALGCLVKDYFPEPVT | 396 |
| D265A/QVV | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVAVSHE | |
| | DPEVKFNWYVDGVEVHNAKTKPREEQYNSTYRVVSV | |
| | LQVLHVDWLNGKEYKCKVSNKALPAPIEKTISKAKGQ | |
| | PREPQVYTLPPSRDELTKNQVSLTCLVKGFYPSDIVVE | |
| | WESNGQPENNYKTTPPVLDSDGSFFLYSKLTVDKSRW | |
| | QQGNVFSCSVMHEALHNHYTQKSLSLSPGK | |
| hIgG1- | ASTKGPSVFPLAPSSKSTSGGTAALGCLVKDYFPEPVT | 397 |
| LALA/QVV | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLQVLHVDWLNGKEYKCKVSNKALPAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 398 |
| LAGA/QVV | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRTPEVTCVVVDVSH | |
| | EDPEVKFNWYVDGVEVHNAKTKPREEQYNSTYRVVS | |
| | VLQVLHVDWLNGKEYKCKVSNKALPAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 399 |
| LALAGA/QVV | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLMISRTPEVTCVVVDVSH | |
| | EDPEVKFNWYVDGVEVHNAKTKPREEQYNSTYRVVS | |
| | VLQVLHVDWLNGKEYKCKVSNKALPAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 400 |
| LALAPG/QVV | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRTPEVTCVVVDVSH | |
| | EDPEVKFNWYVDGVEVHNAKTKPREEQYNSTYRVVS | |
| | VLQVLHVDWLNGKEYKCKVSNKALGAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPGK | |
| hIgG1/DDRVV | ASTKGPSVFPLAPSSKSTSGGTAALGCLVKDYFPEPVT | 401 |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRDPEVTCVVVDVSH | |
| | EDPEVKFNWYVDGVEVDNAKTKPREEQYNSTYRVVS | |
| | VLRVLHVDWLNGKEYKCKVSNKALPAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 402 |
| N297A/DDRVV | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRDPEVTCVVVDVSH | |
| | EDPEVKFNWYVDGVEVDNAKTKPREEQYASTYRVVS | |
| | VLRVLHVDWLNGKEYKCKVSNKALPAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 403 |
| D265A/DDRVV | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRDPEVTCVVVAVSH | |
| | EDPEVKFNWYVDGVEVDNAKTKPREEQYNSTYRVVS | |
| | VLRVLHVDWLNGKEYKCKVSNKALPAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 404 |
| LALA/DDRVV | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRDPEVTCVVVDVSH | |
| | EDPEVKFNWYVDGVEVDNAKTKPREEQYNSTYRVVS | |
| | VLRVLHVDWLNGKEYKCKVSNKALPAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 405 |
| LAGA/DDRVV | VSWNSGALTSGVHTFPAVLQSSGLYSLSSVVTVPSSSL | |
| | GTQTYICNVNHKPSNTKVDKKVEPKSCDKTHTCPPCP | |
| | APELAGAPSVFLFPPKPKDTLMISRDPEVTCVVVDVSH | |
| | EDPEVKFNWYVDGVEVDNAKTKPREEQYNSTYRVVS | |
| | VLRVLHVDWLNGKEYKCKVSNKALPAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 406 |
| LALAGA/DDRVV | VSWNSGALTSGVHTFPAVLQSSGLYSLSSVVTVPSSSL | |
| | GTQTYICNVNHKPSNTKVDKKVEPKSCDKTHTCPPCP | |
| | APEAAGAPSVFLFPPKPKDTLMISRDPEVTCVVVDVSH | |
| | EDPEVKFNWYVDGVEVDNAKTKPREEQYNSTYRVVS | |
| | VLRVLHVDWLNGKEYKCKVSNKALPAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPGK | |
| hIgG1- | ASTKGPSVFPLAPSSKSTSGGTAALGCLVKDYFPEPVT | 407 |
| LALAPG/DDRVV | VSWNSGALTSGVHTFPAVLQSSGLYSLSSVVTVPSSSL | |
| | GTQTYICNVNHKPSNTKVDKKVEPKSCDKTHTCPPCP | |
| | APEAAGGPSVFLFPPKPKDTLMISRDPEVTCVVVDVSH | |
| | EDPEVKFNWYVDGVEVDNAKTKPREEQYNSTYRVVS | |
| | VLRVLHVDWLNGKEYKCKVSNKALGAPIEKTISKAKG | |
| | QPREPQVYTLPPSRDELTKNQVSLTCLVKGFYPSDIVV | |
| | EWESNGQPENNYKTTPPVLDSDGSFFLYSKLTVDKSR | |
| | WQQGNVFSCSVMHEALHNHYTQKSLSLSPGK | |

1-173. (canceled)

174. An isolated antibody that binds Interleukin 4a Receptor (IL-4Rα), wherein the antibody comprises:
a CDR-H1 comprising the sequence set forth in SEQ ID NO: 2;
a CDR-H2 comprising the sequence set forth in SEQ ID NO: 6;
a CDR-H3 comprising the sequence set forth in SEQ ID NO: 20;
a CDR-L1 comprising the sequence set forth in SEQ ID NO: 30;
a CDR-L2 comprising the sequence set forth in SEQ ID NO: 47; and
a CDR-L3 comprising the sequence set forth in SEQ ID NO: 61.

175. An isolated antibody that binds IL-4Rα, wherein the antibody comprises:
a CDR-H1 comprising the sequence set forth in SEQ ID NO: 2;
a CDR-H2 comprising the sequence set forth in SEQ ID NO: 6;
a CDR-H3 comprising the sequence set forth in SEQ ID NO: 20;
a CDR-L1 comprising the sequence set forth in SEQ ID NO: 30;
a CDR-L2 comprising the sequence set forth in SEQ ID NO: 47; and
a CDR-L3 comprising the sequence set forth in SEQ ID NO: 61;
and wherein the antibody comprises a human IgG1 Fc region with LALA and/or YTE mutations.

176. The isolated antibody of claim 174, wherein the antibody comprises an Fc region comprising one or more amino acid substitutions selected from the group consisting of S228P (SP), M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, N434W, M428L/N434S (LS), M252Y/S254T/T256E (YTE), T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA), M428L/N434A (LA) L235A/G237A (LAGA), L234A/L235A/G237A (LALAGA), L234A/L235A/P329G (LALAPG), N297A, D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.

177. The isolated antibody of claim 175, wherein the LALA mutations are at positions 242 and 243 (L242A/L243A) and the YTE mutations are at positions 260, 262, and 264 (M260Y/S262T/T264E).

178. An isolated antibody that binds IL-4Rα, wherein the antibody comprises a heavy chain variable region sequence set forth in SEQ ID NO: 134, a light chain variable region sequence set forth in SEQ ID NO: 178, a constant heavy chain sequence set forth in SEQ ID NO: 205 or SEQ ID NO: 321, and a constant light chain sequence set forth in SEQ ID NO: 236.

179. The isolated antibody of claim 174, wherein the antibody comprises a heavy chain variable region sequence set forth in SEQ ID NO: 134 and a light chain variable region sequence set forth in SEQ ID NO: 178.

180. The isolated antibody of claim 174, wherein the antibody comprises a constant heavy chain sequence set forth in SEQ ID NO: 205 or SEQ ID NO: 321 and a constant light chain sequence set forth in SEQ ID NO: 236.

181. An isolated antibody that binds IL-4Rα, wherein the antibody comprises a heavy chain variable region sequence set forth in SEQ ID NO: 134 and a light chain variable region sequence set forth in SEQ ID NO: 178, wherein the antibody comprises a human IgG1 Fc region with LALA and/or YTE mutations.

182. The isolated antibody of claim 181, wherein the antibody comprises a human IgG1 Fc region with LALA and YTE mutations.

183. The isolated antibody of claim 174, wherein the antibody is a humanized, human, or chimeric antibody.

184. The isolated antibody of claim 183, wherein the antibody is a humanized antibody.

185. The isolated antibody of claim 183, wherein the antibody comprises a human IgG1 Fc region.

186. The isolated antibody of claim 174, wherein the antibody is a monoclonal antibody.

187. The isolated antibody of claim 174, wherein the antibody binds an IL-4Rα sequence selected from a sequence set forth in any one of SEQ ID NOs: 237-240.

188. The isolated antibody of claim 174, wherein the antibody binds to an IL-4Rα sequence selected from a sequence set forth in any one of SEQ ID NOs: 237-240 with a K_{D} of less than or equal to about 1×10⁻⁹ M, as measured by surface plasmon resonance (SPR).

189. The isolated antibody of claim 174, wherein the antibody is formulated for subcutaneous injection.

190. The isolated antibody of claim 174, wherein the antibody is formulated for intravenous injection.

191. The isolated antibody of claim 175, wherein the antibody comprises a human IgG1 Fc region with LALA and YTE mutations.

192. A composition comprising the isolated antibody of claim 174 and a pharmaceutically acceptable excipient.

193. A method for treating an inflammatory disorder or disease in a mammalian subject in need thereof, the method comprising administering to the mammalian subject the isolated antibody of claim 174.

194. The method of claim 193, wherein the inflammatory disorder or disease is atopic dermatitis.

195. The method of claim 193, wherein the inflammatory disorder or disease is selected from the group consisting of asthma, idiopathic pulmonary fibrosis, alopecia areata, chronic sinusitis with nasal polyps, Chronic Rhinosinusitis without Nasal Polyps (CRSsNP), eosinophilic esophagitis (EoE), an Eosinophilic gastrointestinal disorder or disease (ENID), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), inflammatory bowel disease, lupus, and rheumatoid arthritis.

196. The method of claim 195, wherein the ENID is selected from the group consisting of Eosinophilic Gastritis (EoG), Eosinophilic Enteritis (EoN), Eosinophilic Colitis (EoC), and Eosinophilic Gastroenteritis (EGE).

197. A kit comprising:
(a) the isolated antibody that binds IL-4Rα of claim 174; and
(b) instructions for use.

198. The kit of claim 197, for use in treating an inflammatory disorder or disease selected from the group consisting of atopic dermatitis, asthma, idiopathic pulmonary fibrosis, alopecia areata, chronic sinusitis with nasal polyps, Chronic Rhinosinusitis without Nasal Polyps (CRSsNP), eosinophilic esophagitis (EoE), an Eosinophilic gastrointestinal disorder or disease (ENID), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), inflammatory bowel disease, lupus, and rheumatoid arthritis.

199. An isolated polynucleotide or set of polynucleotides encoding the isolated antibody of claim 174.

200. A vector or set of vectors comprising the polynucleotide or set of polynucleotides of claim 199.

201. A host cell comprising the vector or set of vectors of claim 200.

202. The host cell of claim 201, wherein the host cell is a Chinese hamster ovary (CHO) cell.

203. A method of producing an antibody, the method comprising expressing the antibody with the host cell of claim 202 and isolating the expressed antibody.
